(19) United States
(12) Patent Application Publication
(10) Pub. No.: US 2026/0284229 A1
(43) Pub. Date: Sep. 24, 2026

(54) METHODS AND SYSTEM FOR OPTOGENETIC STIMULATION OF ADIPOCYTES

(71) Applicant: MaineHealth, Portland, ME (US)

(72) Inventor: Aaron C. Brown, Portland, ME (US)

(21) Appl. No.: 19/163,947

(22) PCT Filed: Mar. 8, 2024
(86) PCT No.: PCT/US2024/019198

§ 371 Date: Sep. 10, 2025

Related U.S. Application Data
(60) Provisional application No. 63/451,449, filed on Mar. 10, 2023.

(51) Int. Cl. A61K 48/00; A61N 5/06; A61P 3/04; C07K 14/47; C12N 5/077; C12N 9/22; C12N 9/88; C12N 15/11; C12N 15/86; C12N 15/90

(52) U.S. Cl. CPC A61K 48/0058; A61K 48/0066; A61K 48/0083; A61N 5/0601; A61P 3/04; C07K 14/4702; C12N 5/0653; C12N 9/226; C12N 9/88; C12N 15/111; C12N 15/86; C12N 15/907; A61N 2005/0612; C12N 2310/20; C12N 2510/00; C12N 2740/15043; C12N 2750/14143; C12N 2800/22; C12N 2830/002; C12N 2830/008; C12Y 406/01001

(57) ABSTRACT

Provided herein are, inter alia, methods, compositions, and kits for inducing thermogenesis in adipocyte populations. Also included are methods and compositions for increasing thermogenesis in adipocyte populations (e.g., brown/beige/white adipocyte populations) in a subject, as well as methods and compositions for treating subjects who are overweight, obese, or who have diabetes.

### RELATED APPLICATIONS

The instant application claims priority to U.S. Provisional Application No. 63/451,449, filed Mar. 10, 2023, the entire contents of which are expressly incorporated by reference herein.

### GOVERNMENT SUPPORT

This invention was made with government support under Grant No. P20GM121301, awarded by the National Institutes of Health (NIH) Centers of Biomedical Research Excellence (COBRE), Grant No. 1R01DK124261 awarded by the National Institute of Diabetes and Digestive and Kidney Diseases (NIDDK), Grant No. P20GM106391 awarded by the NIH COBRE, and Grant No. U54GM115516 awarded by the NIH COBRE. The government has certain rights in the invention.

### SEQUENCE LISTING

The application contains a Sequence Listing which has been submitted electronically in .XML format and is hereby incorporated by reference in its entirety. Said .XML copy, created on Mar. 5, 2024, is named “136191-00220.xml” and is 265,511 bytes in size. The sequence listing contained in this .XML file is part of the specification and is hereby incorporated by reference herein in its entirety.

### BACKGROUND

Periods of prolonged overeating result in excess lipid storage in white adipocytes, which can lead to inflammation, cellular stress, insulin resistance and metabolic syndrome¹. Conversely, brown adipose tissue (BAT) activity correlates positively with energy expenditure and reduced risk for metabolic syndrome and cardiometabolic diseases, making it an appealing therapeutic target tissue²⁻⁵. Brown adipocytes are rich in specialized mitochondria that expend energy to generate heat and maintain core body temperature⁶. They are primarily activated in response to norepinephrine secreted from sympathetic nerve terminals that engage β-adrenergic surface receptors⁷. Receptor activation triggers a signaling cascade leading to nutrient combustion that starts with cellular uptake of glucose and free fatty acids from the circulation and lipolysis of lipids derived from inner multilocular lipid droplets^{8,9}. This process provides free fatty acids for B-oxidation that drive the electron transport chain to produce a proton-gradient within the inner mitochondrial membrane⁹. Brown adipocytes are unique in that β-adrenergic activation also leads to the expression of uncoupling protein-1 (UCP1), an inner mitochondrial membrane protein, that dissipates the mitochondrial electrochemical gradient from ATP synthesis by releasing protons back into the mitochondrial matrix, resulting in the release of energy through heat production². Unfortunately, modifying obesity and diabetes in humans through drug-based stimulation of β-adrenergic receptors has been met with limited success as it is a risk factor for cardiovascular disease, prompting the development of alternative brown adipocyte activation strategies¹⁰⁻¹³.

Although thermogenic adipose tissue has intrinsic anti-obesity properties, we lack a complete understanding of how UCP1-dependent thermogenesis is regulated. Levels of UCP1 regulation include its transcriptional activation, mRNA and protein turnover and its functional activation within the inner mitochondrial membrane, where it is inherently inactive in the absence of any accompanying stimulus^{14,15}. To activate transcription, β-adrenergic receptors, coupled to Gₛ alpha subunits, activate membrane-associated adenylyl cyclases that lead to a rise in cAMP, a central second messenger regulating brown adipocyte function¹⁶. Increased cAMP activates cAMP-dependent protein kinase A (PKA) leading to the induction of lipolysis and liberation of free fatty acids, which are necessary for functional activation of UCP1-dependent proton leak¹⁸. PKA also stimulates phosphorylation of CREB and activation of a set of thermogenic transcription factors that increase mitochondrial biogenesis, electron transport chain activity and induce Ucp1 transcription¹⁷.

A number of small molecule compounds have been employed to activate brown adipocytes directly through stimulation of β-adrenergic receptors (e.g., norepinephrine, isoproterenol, CL316,243, mirabegron) or adenylyl cyclase (forskolin). However, many of these compounds have off target effects and are not easily reversible¹⁸. To circumvent off target effects, Gₛ-coupled designer receptors exclusively activated by designer drugs (DREADDs) have been designed to be activated by highly specific synthetic ligands such as clozapine-N-oxide (CNO). When Gₛ-coupled DREADDs are conditionally expressed in adipose tissue of obese mice treated with CNO, metabolic improvements are observed including increased energy expenditure, normalized glucose homeostasis and decreased body weight and fat mass^{19,20}. However, whether high affinity DREADD ligands such as CNO or Compound 21 (C21) are truly inert has been called into question as their chronic use has been linked to off target behavioral effects²¹.

### SUMMARY OF THE INVENTION

Accordingly, in one aspect, the present invention provides a cell comprising at least one non-native gene or gene cassette encoding one or more adenylyl cyclase enzymes, wherein the adenylyl cyclase enzyme comprises an optogenetic moiety.

In one embodiment, the gene or gene cassette is operably linked to a promoter that is not associated with the adenylyl cyclase enzyme in nature.

In one embodiment, the promoter is inducible. In one embodiment, the promoter is induced in adipocytes. In one embodiment, the adipocyte-specific promoter is AP2 or AdipoQ. In one embodiment, the promoter is selected from the group consisting of EF1α promoter, CAG promoter, and CMV promoter.

In one embodiment, the adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa). In one embodiment, the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007. In one embodiment, the adenylyl cyclase enzyme is from Beggiatoa sp. PS.

In one embodiment, the amino acid sequence of the adenylyl cyclase enzyme comprises one or more amino acid substitutions relative to a wild type adenylyl cyclase sequence.

In one embodiment, the one or more amino acid substitutions are at positions corresponding to position 27, 123, 198, 266, 267, 278, 197, and/or 201 in the wild type adenylyl cyclase enzyme sequence.

In one embodiment, the wild type adenylyl cyclase enzyme amino acid sequence comprises SEQ ID NO: 1 or SEQ ID NO: 31.

In one embodiment, the amino acid sequence of the adenylyl cyclase enzyme comprises an alanine (A) at a position corresponding to position 27 in the sequence of SEQ ID NO: 1 or SEQ ID NO: 31.

In one embodiment, the amino acid sequence of the adenylyl cyclase enzyme comprises SEQ ID NO: 2.

In one embodiment, the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of any one of SEQ ID NOs: 32, 33, or 62. In one embodiment, the gene or gene cassette encoding the adenylyl cyclase enzyme encodes a polypeptide sequence that has at least 90%, 91%, 92%, 93%, 94%, 95%, 96%, 97%, 98%, or 99% identity to, comprises, or consists of SEQ ID NOs: 1, 2, or 31.

In one embodiment, the optogenetic moiety and adenylyl cyclase enzyme are found in the same polypeptide in nature. In one embodiment, the optogenetic moiety is fused to the adenylyl cyclase enzyme, and is not associated with the adenylyl cyclase enzyme in nature. In one embodiment, the optogenetic moiety and the adenylyl cyclase enzyme are derived from different organisms.

In one embodiment, the gene or gene cassette encoding the adenylyl cyclase enzyme and/or the optogenetic moiety is codon optimized for expression in a mammalian cell.

In one embodiment, the cell is a mammalian cell. In one embodiment, the mammalian cell is a human cell.

In one embodiment, the cell is a pluripotent stem cell. In one embodiment, the pluripotent stem cell is a preadipocyte. In one embodiment, the pluripotent stem cell or preadipocyte is capable of differentiating into an adipocyte. In one embodiment, the adipocyte is a brown adipocyte, white adipocyte, or beige adipocyte.

In another aspect, the present invention provides a method of generating the cell as provided herein, wherein a cell which is capable of differentiation into an adipocyte is transfected with a vector comprising the one or more gene cassettes encoding one or more adenylyl cyclase enzymes comprising the optogenetic moiety.

In one embodiment, the transfected cell is differentiated into an adipocyte, wherein the adipocyte is a brown adipocyte, white adipocyte, or beige adipocyte.

In one embodiment, the transfected cell, adipocyte, brown adipocyte, white adipocyte, or beige adipocyte is expanded in culture.

In one embodiment, the vector is a plasmid, a lentivirus vector or an AAV vector.

In one embodiment, the vector further comprises a gene encoding a polypeptide to induce integration of the gene or the gene cassette encoding the one or more adenylyl cyclase enzymes comprising the optogenetic moiety into the genome of the cell. In one embodiment, the gene encoding a polypeptide to induce integration of the gene or gene cassette into the genome is a DNA endonuclease. In one embodiment, the DNA endonuclease is a Cas endonuclease. In one embodiment, the cas endonuclease is a Cas9 endonuclease

In another aspect, the present invention provides a system for activating uncoupling protein-1 (UCP-1) in a cell, the system comprising: a) a delivery vector comprising a non-native nucleotide sequence encoding one or more adenylyl cyclase enzymes, wherein the adenylyl cyclase enzyme comprises an optogenetic moiety, wherein the delivery vector provides for delivery of the one or more adenylyl cyclase enzymes comprising the optogenetic moiety to a cell; b) a light source; and c) a control device.

In another aspect, the present invention provides a system for increasing the levels of uncoupling protein-1 (UCP-1) in a cell, the system comprising: a) a delivery vector comprising a non-native nucleotide sequence encoding one or more adenylyl cyclase enzymes, wherein the adenylyl cyclase enzyme comprises an optogenetic moiety, wherein the delivery vector provides for delivery of the one or more adenylyl cyclase enzymes comprising the optogenetic moiety to a cell; b) a light source; and c) a control device.

In one embodiment, the delivery vector is a plasmid, a lentivirus vector or an AAV vector.

In one embodiment, the non-native nucleotide sequence is operably linked to a promoter that is not associated with the adenylyl cyclase enzyme in nature. In one embodiment, the promoter is inducible. In one embodiment, the promoter is induced in adipocytes. In one embodiment, the adipocyte-specific promoter is AP2 or AdipoQ. In one embodiment, the promoter is selected from the group consisting of EF1α promoter, CAG promoter, and CMV promoter.

In one embodiment, the adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa). In one embodiment, the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007. In one embodiment, the adenylyl cyclase enzyme is from Beggiatoa sp. PS.

In one embodiment, the amino acid sequence of the adenylyl cyclase enzyme comprises one or more amino acid substitutions relative to a wild type adenylyl cyclase sequence. In one embodiment, the one or more amino acid substitutions are at positions corresponding to position 27, 123, 198, 266, 267, 278, 197, and/or 201 in the wild type adenylyl cyclase enzyme sequence.

In one embodiment, the wild type adenylyl cyclase enzyme amino acid sequence comprises SEQ ID NO: 1 or SEQ ID NO: 30. In one embodiment, the amino acid sequence of the adenylyl cyclase enzyme comprises an alanine (A) at a position corresponding to position 27 in the sequence of SEQ ID NO: 1 or SEQ ID NO: 31. In one embodiment, the amino acid sequence of the adenylyl cyclase enzyme comprises SEQ ID NO: 2.

In one embodiment, the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of any one of SEQ ID NOs: 32, 33, or 62. In one embodiment, the gene or gene cassette encoding the adenylyl cyclase enzyme encodes an amino acid sequence that has at least 90%, 91%, 92%, 93%, 94%, 95%, 96%, 97%, 98%, or 99% identity to, comprises, or consists of SEQ ID NOs: 1, 2, or 31.

In one embodiment, the optogenetic moiety and adenylyl cyclase enzyme are found in the same polypeptide in nature. In one embodiment, the optogenetic moiety is fused to the adenylyl cyclase enzyme, and is not associated with the adenylyl cyclase enzyme in nature. In one embodiment, the optogenetic moiety and the adenylyl cyclase enzyme are derived from different organisms.

In one embodiment, the gene or gene cassette encoding the adenylyl cyclase enzyme comprising the optogenetic moiety is codon optimized for expression in a mammalian cell.

In one embodiment, the cell is a pluripotent cell or preadipocyte. In one embodiment, the pluripotent stem cell or preadipocyte is capable of differentiating into an adipocyte.

In one embodiment, the light source is a light-emitting diode or a laser. In one embodiment, the light source produces transient blue light, continuous blue light, or oscillating pulses of blue light. In one embodiment, the radiation intensity of the blue light produced by the light source is at least 0.05 mW/cm², at least 0.1 mW/cm², at least 1 mW/cm², at least 2 mW/cm², at least 5 mW/cm², at least 10 mW/cm².

In one embodiment, the control device is implantable.

In one embodiment, the system activates UCP-1 in a subject.

In another aspect, the present invention provides a method for activating uncoupling protein-1 (UCP-1) in a brown adipocyte of a subject, the method comprising: a) administering to the subject the cell of as provided herein, or the cell generated by the methods as provided herein; b) activating the optogenetic moiety with a light source, wherein the adenylyl cyclase enzyme comprising the optogenetic moiety is activated, and wherein the light source is controlled by a control device; thereby activating UCP-1 in the cell of the subject.

In another aspect, the present invention provides a method for increasing the levels of uncoupling protein-1 (UCP-1) in a brown adipocyte of a subject, the method comprising: a) administering to the subject the cell of as provided herein, or the cell generated by the methods as provided herein; b) activating the optogenetic moiety with a light source, wherein the adenylyl cyclase enzyme comprising the optogenetic moiety is activated, and wherein the light source is controlled by a control device; thereby increasing the levels of UCP-1 in the cell of the subject.

In another aspect, the present invention provides a method for promoting the transcription of uncoupling protein-1 (UCP-1) in a cell, the method comprising: a) administering to the subject the cell of as provided herein, or the cell generated by the methods as provided herein; b) activating the optogenetic moiety with a light source, wherein the adenylyl cyclase enzyme comprising the optogenetic moiety is activated, and wherein the light source is controlled by a control device; thereby promoting the transcription of UCP-1 in the cell of the subject.

In another aspect, the present invention provides a method for promoting the transcription of uncoupling protein-1 (UCP-1) in a cell, the method comprising: a) administering to the subject the cell of as provided herein, or the cell generated by the methods as provided herein; b) activating the optogenetic moiety with a light source, wherein the adenylyl cyclase enzyme comprising the optogenetic moiety is activated, and wherein the light source is controlled by a control device; thereby promoting the transcription of UCP-1 in the cell of the subject.

In one embodiment, activating the optogenetic moiety with a light source modulates the expression of at least one gene with a mitochondrial function. In one embodiment, the at least one gene has a mitochondrial function. In one embodiment, the at least one gene has a function in mitochondrial biogenesis; ATP synthesis, electron transport, or mitophagy. In one embodiment, the at least one gene is selected from, Pgc1a, Tfam, Atp5g1, Atp5g3, Ndufs4, Ndufb8/complex I, Sdhb/complex II, Uqcr2/complex III, Cox412/complex IV, Bnip3, Bnip3l, Pink1, or Mff.

In one embodiment, activating the optogenetic moiety with a light source modulates the uptake of fatty acids and insulin-independent glucose uptake in the cell. In one embodiment, the fatty acid and insulin-independent glucose uptake is increased in the cell.

In one embodiment, activating the optogenetic moiety with a light source, results in increased thermogenesis in the cells compared to cells that are not treated with a light source.

In another aspect, the present invention provides a method of treating a metabolic disease or disorder in a subject, the method comprising: a) administering to the subject the cell as provided herein, or the cell generated by the method as provided herein; b) activating the optogenetic moiety with a light source, wherein the adenylyl cyclase enzyme is activated, and wherein the light source is controlled by a control device; thereby treating the subject.

In one embodiment, the metabolic disease or disorder is selected from obesity, diabetes or hyperlipidemia. In one embodiment, the subject is obese and in need of treatment.

In one embodiment, the cells as provided herein or the cells generated by the methods as provided herein are derived from the subject or a donor.

In one embodiment, the optogenetic moiety is activated by transient blue light, continuous blue light, or oscillating pulses of blue light. In one embodiment, the administered cells are treated with the light source for at least 10 minutes, at least 30 minutes, at least 1 hour, at least 2 hours, at least 3 hours, at least 4 hours, at least 5 hours, or at least 6 hours. In one embodiment, the administered cells are treated with the light source for about 10 milliseconds to about 500 milliseconds every second for at least 1 hour, at least 2 hours, at least 3 hours, at least 4 hours, or at least 5 hours. In one embodiment, the administered cells are treated with the light source for between about 10 minutes and 6 hours, about 30 minutes and about 6 hours, about 1 hour to about 6 hours, about 2 hours to about 6 hours, about 3 hours to about 6 hours, about 3 hours to about 6 hours, about 4 hours to about 6 hours, about 5 hours to about 6 hours. In one embodiment, the radiation intensity of the light source is at least 0.05 mW/cm², at least 0.1 mW/cm², at least 1 mW/cm², at least 2 mW/cm², at least 5 mW/cm², at least 10 mW/cm². In one embodiment, the treatment with the light source is administered once a day, twice a day, three times a day, once a week, twice a week, three times a week, 4 times a week, 5 times a week, 6 times a week, or every day for at least 1 month, at least 2 months, at least 3 months, at least 4 months, at least 5 months, at least 6 months, at least 7 months, at least 8 months, at least 9 months, at least 10 months, at least 11 months, or at least 1 year.

In one embodiment, further wherein a cell as provided herein, or a cell generated by the method as provided herein are grown on a scaffold prior to being administered to the subject.

In another aspect, the present invention provides a method of treating a subject in need thereof, the method comprising: a) administering to a cell of the subject a nucleic acid encoding one or more adenylyl cyclase enzymes, wherein the adenylyl cyclase enzyme comprises an optogenetic moiety; b) activating the optogenetic moiety with a light source, wherein the adenylyl cyclase enzyme is activated, and wherein the light source is controlled by a control device; thereby treating the subject.

In one embodiment, the delivery vector is a plasmid, a lentivirus vector or an AAV vector.

In one embodiment, the nucleotide sequence encoding the one or more adenylyl cyclase enzymes is operably linked to a promoter that is not associated with the adenylyl cyclase enzyme in nature. In one embodiment, the promoter is inducible. In one embodiment, the promoter is induced in adipocytes. In one embodiment, the adipocyte-specific promoter is AP2 or AdipoQ. In one embodiment, the promoter is selected from the group consisting of EF1α promoter, CAG promoter, and CMV promoter.

In one embodiment, the adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa). In one embodiment, the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007. In one embodiment, the adenylyl cyclase enzyme is from Beggiatoa sp. PS.

In one embodiment, the amino acid sequence of the adenylyl cyclase enzyme comprises one or more amino acid substitutions relative to a wild type adenylyl cyclase sequence.

In one embodiment, the one or more amino acid substitutions are at positions corresponding to position 27, 123, 198, 266, 267, 278, 197, and/or 201 in the wild type adenylyl cyclase enzyme amino acid sequence.

In one embodiment, the wild type adenylyl cyclase enzyme amino acid sequence comprises SEQ ID NO: 1 or SEQ ID NO: 30. In one embodiment, the amio acid sequence of the adenylyl cyclase enzyme comprises an alanine (A) at a position corresponding to position 27 in the sequence of SEQ ID NO: 1 or SEQ ID NO: 31. In one embodiment, the amino acid sequence of the adenylyl cyclase enzyme comprises SEQ ID NO: 2.

In one embodiment, the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of any one of SEQ ID NOs: 32, 33, or 62. In one embodiment, the gene or gene cassette encoding the adenylyl cyclase enzyme encodes an amino acid sequence that has at least 90%, 91%, 92%, 93%, 94%, 95%, 96%, 97%, 98%, or 99% identity to, comprises, or consists of SEQ ID NOs: 1, 2, or 31.

In one embodiment, the optogenetic moiety and adenylyl cyclase enzyme are found in the same polypeptide in nature. In one embodiment, the optogenetic moiety is fused to the adenylyl cyclase enzyme, and is not associated with the adenylyl cyclase enzyme in nature. In one embodiment, the optogenetic moiety and the adenylyl cyclase enzyme are derived from different organisms.

In one embodiment, the gene or gene cassette encoding the adenylyl cyclase enzyme and/or the optogenetic moiety is codon optimized for expression in a mammalian cell.

In one embodiment, the cell is a pluripotent cell or preadipocyte.

In one embodiment, the light source is a light-emitting diode or a laser. In one embodiment, the control device is implantable.

In one embodiment, the optogenetic moiety is activated by transient blue light, continuous blue light, or oscillating pulses of blue light. In one embodiment, the cells are treated with the light source for at least 10 minutes, at least 30 minutes, at least 1 hour, at least 2 hours, at least 3 hours, at least 4 hours, at least 5 hours, or at least 6 hours. In one embodiment, the cells are treated with the light source for about 10 milliseconds to about 500 milliseconds every second for at least 1 hour, at least 2 hours, at least 3 hours, at least 4 hours, or at least 5 hours. In one embodiment, the cells are treated with the light source for between about 10 minutes and 6 hours, about 30 minutes and about 6 hours, about 1 hour to about 6 hours, about 2 hours to about 6 hours, about 3 hours to about 6 hours, about 3 hours to about 6 hours, about 4 hours to about 6 hours, about 5 hours to about 6 hours. In one embodiment, the treatment with the light source is administered once a day, twice a day, three times a day, once a week, twice a week, three times a week, 4 times a week, 5 times a week, 6 times a week, or every day for at least 1 month, at least 2 months, at least 3 months, at least 4 months, at least 5 months, at least 6 months, at least 7 months, at least 8 months, at least 9 months, at least 10 months, at least 11 months, or at least 1 year. In one embodiment, the light source produces transient blue light, continuous blue light, or oscillating blue light. In one embodiment, the radiation intensity of the light source is at least 0.05 mW/cm², at least 0.1 mW/cm², at least 1 mW/cm², at least 2 mW/cm², at least 5 mW/cm², at least 10 mW/cm².

### BRIEF DESCRIPTION OF THE DRAWINGS

FIGS. 1A-1J Blue light stimulation of bPAC-27A increases CAMP signaling in brown preadipocytes. (FIG. 1A) bPAC lentiviral plasmid construct developed for these studies. (FIG. 1B) Flow sorting increases mRFP-bPAC positive preadipocytes to greater than 90 percent. (FIG. 1C) CAMP production (Relative Luminescence Units-RLU) in brown preadipocytes treated with forskolin (black bars) or blue light (blue bars). (FIG. 1D) cGMP detection by ELISA after 30 minutes of blue light stimulation. Atrial natriuretic peptide (ANP)+IBMX served as positive control. (FIG. 1E) Western blot showing CREB phosphorylation 45 min after initiation of blue light pulsing for the indicated times. Unphosphorylated CREB serves as loading control. (FIG. 1F) qPCR of the CREB target gene Nr4a3, 2 hrs after initiation of blue light pulsing for the indicated times. (FIG. 1G) qPCR indicates clonal brown preadipocyte populations (Td1, Td3, Td6) with high bPAC expression. (FIG. 1H) Clones stained with BODIPY after differentiation into mature adipocytes. (FIGS. 1I-1J) qPCR of differentiated clones shows maintenance of bPAC expression (FIG. 1I) compared to non-clonal (mixed) preadipocytes with varying levels of Ucp1 transcription (FIG. 1J) after their differentiation.

FIGS. 2A-2H Blue light stimulation bPAC increases transcription of Ucp1 in brown adipocytes. (FIG. 2A) Pulsing strategies to determine blue light duration for stimulation of Ucp1 expression. Ucp1 qPCR time course of Td1-bPAC+ brown adipocytes pulsed with blue light (FIG. 2B) or treated with forskolin (FIG. 2C). Ucp1 qPCR of Td1-bPAC+ (FIG. 2D) or bPAC-(FIG. 2E) brown adipocytes pulsed with oscillating blue light pulses for 4 hrs. (FIG. 2F) Blue light stimulation of Td1-bPAC+ brown adipocytes increases expression of genes associated with mitochondrial function. (FIG. 2G) qPCR of Td1-bPAC+ brown adipocytes pulsed with blue light beginning at day 10 can maintain (30 minutes/day) or increase (60 minutes/day) Ucp1 mRNA by day 18 compared to dark exposure. (FIG. 2H) Western blot analysis of the indicated bPAC+ brown adipocytes pulsed with blue light beginning at day 10 display increased UCP1 protein expression by day 18 compared to dark exposure. Black and blue bars represent forskolin and blue light treatments, respectively.

FIGS. 3A-3G. Blue light stimulation of bPAC in brown adipocytes increases fuel uptake and thermogenesis in a UCP1-dependent manner. (FIG. 3A) Quantitative summary of Td1-bPAC+ brown preadipocytes and adipocytes treated with blue light and/or forskolin for 30 min before fatty acid uptake measurement. Means±SD (n=8) shown. (FIG. 3B) Glucose uptake analysis of Td1-bPAC+ brown adipocytes treated with and without blue light for 30 minutes. Means±SD (n=6) shown. (FIG. 3C) Accumulated heat (left) and total heat (right) produced over 16 hrs in brown adipocyte cultures of the indicated genotypes treated with and without 30 min blue light. Means±SEM (n=4) shown. (FIG. 3D) Immunofluorescence microscopy of preadipocytes (clone Td1) transduced with and without lentivirus containing scramble control or Ucp1-shRNA lentivirus (upper panels, EGFP) after blasticidin selection for 7 days. mRFP-bPAC expression (middle panels) and differentiation into lipid containing adipocytes (BODIPY stain, bottom panels) shown. (FIG. 3E) Ucp1 qPCR of scramble control and Ucp1-shRNA transduced mature adipocytes. Accumulated heat (FIG. 3F) and total heat (FIG. 3G) produced over 16 hrs in brown adipocyte cultures of the indicated genotypes treated with and without 30 min blue light. Means±SEM (n=4) shown.

FIG. 4. The establishment of an optogenetic model to activate UCP1-dependent thermogenesis is shown as a schematic. A light-inducible adenylyl cyclase enzyme comprising an optogenetic moiety is used to increase intracellular levels of CAMP, UCP1 expression, fuel uptake and UCP1-dependent heat production in brown adipocytes.

### DETAILED DESCRIPTION OF THE INVENTION

Provided herein, are, inter alia, methods, compositions, systems and kits for producing adipocyte populations such as beige adipocyte populations, as well as methods and compositions for treating metabolic disorders such as obesity and type II diabetes.

An alternative approach to chemical modulation of cell signaling is optogenetics, a state-of-the-art technique that uses light-sensitive proteins to control specific cellular processes in genetically modified cells and can be used for precise temporal, spatial, reversible control of signaling. Optogenetic stimulation of the blue light-activated channelrhodopsin-2 (ChR2) in sympathetic nerves has been shown to increase brown adipose tissue thermogenesis and lower blood glucose levels²². As a more direct approach, to determine if brown adipocytes could be genetically modified for light inducible activation of UCP1-dependent thermogenesis, we took advantage of a bacterial photoactivatable adenylyl cyclase (bPAC), previously cloned from the soil bacterium Beggiatoa for chemical free stimulation of adenylyl cyclase²³. Upon stimulation with blue light, bPAC has been shown to increase CAMP levels and alter cellular physiology in cells from numerous species including Escherichia coli, Drosophila melanogaster (nervous system), Xenopus laevis (oocytes), Mus musculus (sperm), Rattus norvegicus (pyramidal neurons) and Homo sapiens (HEK-293T cells) 23-25. Here, we demonstrate that blue light stimulation of bPAC in brown adipocytes promotes fuel uptake (glucose and fatty acids) and upregulation and activation of UCP1, concomitant with heat production. Overall, this novel system allows for precise, temporal control of UCP1-dependent thermogenesis in brown adipocytes and could be exploited therapeutically to reverse obesity-related disorders.

### Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

In embodiments, a “metabolic disease” or “metabolic syndrome” or “metabolic condition” is a disorder in which an error of metabolism, an imbalance in metabolism, or a sub-optimal metabolism occur. In embodiments, a metabolic disease described herein is a disease that can be treated through the modulation of metabolism, although the disease itself may or may not be caused by a specific metabolic defect. Such metabolic diseases may involve, for example, glucose and fatty acid oxidation pathways.

In embodiments, subjects with metabolic diseases to which the present disclosure can be applied are subjects who are diagnosed with, suspected of having, or at risk of having, a metabolic disease or condition. In embodiments, such subjects are in need of treatment with, e.g., a beige adipocyte population, IL-4, and/or a TGF-β inhibitor. In embodiments, methods of the disclosure may be applied to a subject who, at the time of treatment, has been diagnosed as having a metabolic disease or condition, or a subject who is considered to be at risk for having or developing a metabolic disease or condition.

Non-limiting examples of subjects in need of treatment for a metabolic disease include subjects who are overweight, or who have obesity, non-alcoholic fatty liver disease, diabetes, insulin-resistance, alcoholic fatty liver disease, dyslipidemia, steatosis (e.g., liver steatosis, heart steatosis, kidney steatosis, muscle steatosis), abetalipoproteinemia, glycogen storage disease, Weber-Christian disease, lipodystrophy; a liver disease such as liver inflammation, hepatitis, steatohepatitis, Hepatitis C, Genotype 3 Hepatitis C, Alpha 1-antitrypsin deficiency, acute fatty liver of pregnancy, and Wilson disease; a kidney disease; or a heart disease such as hypertension, ischemia, heart failure, or cardiomyopathy.

The term “obesity” as used herein is defined in the World Health Organization (WHO) classifications of weight. In embodiments, “underweight” for a human is having a BMI of less than 18.5 (subjects with such a BMI may optionally be referred to as “thin”). In embodiments, a healthy BMI is 18.5-24.9 (subjects with such a BMI may optionally be referred to as having “normal” weight). In embodiments, grade 1 overweight is 25.0-29.9 BMI (subjects with such a BMI may optionally be referred to as “overweight”). In embodiments grade 2 overweight is 30.0-39.0 BMI (subjects with such a BMI may optionally be referred to as “obese”). In embodiments, grade 3 overweight is greater than or equal to 40.0 BMI (subjects with such a BMI may optionally be referred to as “morbidly obese”). BMI is body mass index (morbid obesity) and is kg/m². Other measures of obesity include, but are not limited to, waist circumference, skinfold thickness and bioimpedance, which is based on the principle that lean mass conducts current better than fat mass because it is primarily an electrolyte solution.

The term “obesity-related condition” refers to any disease or condition that is caused by or associated with (e.g., by biochemical or molecular association) obesity or that is caused by or associated with weight gain and/or related biological processes that precede clinical obesity. Examples of obesity-related conditions include, but are not limited to, diabetes (e.g., type 1 diabetes, type 2 diabetes, and gestational diabetes), Syndrome X, hyperglycemia, hyperinsulinemia, impaired glucose tolerance, impaired fasting glucose, dyslipidemia, hypertriglyceridemia, insulin resistance, hypercholesterolemia, atherosclerosis, coronary artery disease, peripheral vascular disease, and hypertension.

By, “small molecule” may be referred to broadly as an organic, inorganic or organometallic compound with a low molecular weight compound (e.g., a molecular weight of less than about 2,000 Da or less than about 1,000 Da). The small molecule may have a molecular weight of less than about 2,000 Da, a molecular weight of less than about 1,500 Da, a molecular weight of less than about 1,000 Da, a molecular weight of less than about 900 Da, a molecular weight of less than about 800 Da, a molecular weight of less than about 700 Da, a molecular weight of less than about 600 Da, a molecular weight of less than about 500 Da, a molecular weight of less than about 400 Da, a molecular weight of less than about 300 Da, a molecular weight of less than about 200 Da, a molecular weight of less than about 100 Da, or a molecular weight of less than about 50 Da.

By “UCP1” is meant uncoupling protein 1 (also referred to as thermogenin). In embodiments, UCP1 is a protein found in the mitochondria. In embodiments, UCP1 is used to generate heat. UCPs are transmembrane proteins that decrease the proton gradient generated in oxidative phosphorylation, by increasing the permeability of the inner mitochondrial membrane, allowing protons that have been pumped into the intermembrane space to return to the mitochondrial matrix.

The term “disease” refers to any deviation from the normal health of a mammal and includes a state when disease symptoms are present, as well as conditions in which a deviation (e.g., metabolic dysfunction or metabolic disorder) has occurred, but symptoms are not yet manifested.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of′ may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

### Methods of Stimulating UCP-1 in Brown Adipocytes

Accordingly, in one aspect, the present invention provides a cell comprising at least one non-native gene or gene cassette encoding one or more adenylyl cyclase enzymes, wherein the adenylyl cyclase enzyme comprises an optogenetic moiety.

In one embodiment, the nucleic acid sequence encoding a adenylyl cyclase enzyme has at least about 80% identity with the sequence of SEQ ID NO: 32. Accordingly, in one embodiment, the nucleic acid sequence encoding a adenylyl cyclase enzyme has at least about 90% identity with the sequence of SEQ ID NO: 32. Accordingly, in one embodiment, the nucleic acid sequence encoding a adenylyl cyclase enzyme has at least about 95% identity with the sequence of SEQ ID NO: 32. Accordingly, in one embodiment, the nucleic acid sequence encoding a adenylyl cyclase enzyme has at least about 85%, 86%, 87%, 88%, 89%, 90%, 91%, 92%, 93%, 94%, 95%, 96%, 97%, 98%, or 99% identity with the sequence of SEQ ID NO: 32. In another embodiment, the nucleic acid sequence encoding a adenylyl cyclase enzyme comprises the sequence of SEQ ID NO: 32. In yet another embodiment, the nucleic acid sequence encoding a adenylyl cyclase enzyme consists of the sequence of SEQ ID NO: 32.

In one embodiment, the nucleic acid sequence encoding a adenylyl cyclase enzyme encodes a polypeptide has at least about 80% identity with SEQ ID NO: 1. In one embodiment, the nucleic acid sequence encoding a adenylyl cyclase enzyme encodes a polypeptide has at least about 90% identity with SEQ ID NO: 1. In one embodiment, the nucleic acid sequence encoding a adenylyl cyclase enzyme encodes a polypeptide has at least about 95% identity with SEQ ID NO: 1. Accordingly, in one embodiment, the nucleic acid sequence encoding a adenylyl cyclase enzyme encodes a polypeptide has at least about 85%, 86%, 87%, 88%, 89%, 90%, 91%, 92%, 93%, 94%, 95%, 96%, 97%, 98%, or 99% identity with SEQ ID NO: 1. In another embodiment, the nucleic acid sequence encoding a adenylyl cyclase enzyme encodes a polypeptide comprises SEQ ID NO: 1. In yet another embodiment, the nucleic acid sequence encoding a adenylyl cyclase enzyme encodes a polypeptide consists of SEQ ID NO: 1.

In one embodiment, the nucleic acid sequence encoding a adenylyl cyclase enzyme has at least about 80% identity with the sequence of SEQ ID NO: 62. Accordingly, in one embodiment, the nucleic acid sequence encoding a adenylyl cyclase enzyme has at least about 90% identity with the sequence of SEQ ID NO: 62. Accordingly, in one embodiment, the nucleic acid sequence encoding a adenylyl cyclase enzyme has at least about 95% identity with the sequence of SEQ ID NO: 22. Accordingly, in one embodiment, the nucleic acid sequence encoding a adenylyl cyclase enzyme has at least about 85%, 86%, 87%, 88%, 89%, 90%, 91%, 92%, 93%, 94%, 95%, 96%, 97%, 98%, or 99% identity with the sequence of SEQ ID NO: 22. In another embodiment, the nucleic acid sequence encoding a adenylyl cyclase enzyme comprises the sequence of SEQ ID NO: 22. In yet another embodiment, the nucleic acid sequence encoding a adenylyl cyclase enzyme consists of the sequence of SEQ ID NO: 22.

In one embodiment, the nucleic acid sequence encoding a adenylyl cyclase enzyme encodes a polypeptide has at least about 80% identity with SEQ ID NO: 31. In one embodiment, the nucleic acid sequence encoding a adenylyl cyclase enzyme encodes a polypeptide has at least about 90% identity with SEQ ID NO: 31. In one embodiment, the nucleic acid sequence encoding a adenylyl cyclase enzyme encodes a polypeptide has at least about 95% identity with SEQ ID NO: 31. Accordingly, in one embodiment, the nucleic acid sequence encoding a adenylyl cyclase enzyme encodes a polypeptide has at least about 85%, 86%, 87%, 88%, 89%, 90%, 91%, 92%, 93%, 94%, 95%, 96%, 97%, 98%, or 99% identity with SEQ ID NO: 31. In another embodiment, the nucleic acid sequence encoding a adenylyl cyclase enzyme encodes a polypeptide comprises SEQ ID NO: 31. In yet another embodiment, the nucleic acid sequence encoding a adenylyl cyclase enzyme encodes a polypeptide consists of SEQ ID NO: 31.

In one embodiment, the nucleic acid sequence encoding a adenylyl cyclase enzyme has at least about 80% identity with the sequence of SEQ ID NO: 33. Accordingly, in one embodiment, the nucleic acid sequence encoding a adenylyl cyclase enzyme has at least about 90% identity with the sequence of SEQ ID NO: 33. Accordingly, in one embodiment, the nucleic acid sequence encoding a adenylyl cyclase enzyme has at least about 95% identity with the sequence of SEQ ID NO: 33. Accordingly, in one embodiment, the nucleic acid sequence encoding a adenylyl cyclase enzyme has at least about 85%, 86%, 87%, 88%, 89%, 90%, 91%, 92%, 93%, 94%, 95%, 96%, 97%, 98%, or 99% identity with the sequence of SEQ ID NO: 33. In another embodiment, the nucleic acid sequence encoding a adenylyl cyclase enzyme comprises the sequence of SEQ ID NO: 33. In yet another embodiment, the nucleic acid sequence encoding a adenylyl cyclase enzyme consists of the sequence of SEQ ID NO: 33.

In one embodiment, the nucleic acid sequence encoding a adenylyl cyclase enzyme encodes a polypeptide has at least about 80% identity with SEQ ID NO: 2. In one embodiment, the nucleic acid sequence encoding a adenylyl cyclase enzyme encodes a polypeptide has at least about 90% identity with SEQ ID NO: 2. In one embodiment, the nucleic acid sequence encoding a adenylyl cyclase enzyme encodes a polypeptide has at least about 95% identity with SEQ ID NO: 2. Accordingly, in one embodiment, the nucleic acid sequence encoding a adenylyl cyclase enzyme encodes a polypeptide has at least about 85%, 86%, 87%, 88%, 89%, 90%, 91%, 92%, 93%, 94%, 95%, 96%, 97%, 98%, or 99% identity with SEQ ID NO: 2. In another embodiment, the nucleic acid sequence encoding a adenylyl cyclase enzyme encodes a polypeptide comprises SEQ ID NO: 2. In yet another embodiment, the nucleic acid sequence encoding a adenylyl cyclase enzyme encodes a polypeptide consists of SEQ ID NO: 2.

As used herein, the term “optogenetics” is a biological technique to control the activity of cell (e.g., adipocytes) with light. This is achieved by expression of light-sensitive polypeptides, such as ion channels, pumps or enzymes specifically in the target cells. In some embodiments, the light-sensitive polypeptide is an adenylyl cyclase. In some embodiments, the adenylyl cyclase is a photoactivated adenylyl cyclase (PAC). For example, a photoactivated adenylyl cyclase (PAC) is a polypeptide consisting of an adenylyl cyclase enzyme domain directly linked to a BLUF (blue light receptor using FAD) type light sensor domain. When illuminated with blue light, the enzyme domain becomes active and converts ATP to CAMP, an important second messenger in many cells. In the unicellular flagellate Euglena gracilis, PACα and PACβ (euPACs) serve as a photoreceptor complex that senses light for photophobic responses and phototaxis. Small but potent PACs were identified in the genome of the bacteria Beggiatoa (bPAC) and Oscillatoria acuminate (OaPAC). While natural bPAC has some enzymatic activity in the absence of light, variants with no dark activity have been engineered (PACmn).

In some embodiment, the optogenetic moiety comprises a BLUF domain. In one embodiment, the nucleic acid sequence encoding the BLUF domain has at least about 80% identity with the sequence of SEQ ID NO: 65. Accordingly, in one embodiment, the nucleic acid sequence encoding the BLUF domain has at least about 90% identity with the sequence of SEQ ID NO: 65. Accordingly, in one embodiment, the nucleic acid sequence encoding the BLUF domain has at least about 95% identity with the sequence of SEQ ID NO: 65. Accordingly, in one embodiment, the nucleic acid sequence encoding the BLUF domain has at least about 85%, 86%, 87%, 88%, 89%, 90%, 91%, 92%, 93%, 94%, 95%, 96%, 97%, 98%, or 99% identity with the sequence of SEQ ID NO: 65. In another embodiment, the nucleic acid sequence encoding the BLUF domain comprises the sequence of SEQ ID NO: 65. In yet another embodiment, the nucleic acid sequence encoding the BLUF domain consists of the sequence of SEQ ID NO: 65.

In some embodiment, the optogenetic moiety comprises a BLUF domain. In one embodiment, the nucleic acid sequence encoding the BLUF domain encodes a polypeptide has at least about 80% identity with SEQ ID NO: 64. In one embodiment, the nucleic acid sequence encoding the BLUF domain encodes a polypeptide has at least about 90% identity with SEQ ID NO: 64. In one embodiment, the nucleic acid sequence encoding the BLUF domain encodes a polypeptide has at least about 95% identity with SEQ ID NO: 64. Accordingly, in one embodiment, the nucleic acid sequence encoding the BLUF domain encodes a polypeptide has at least about 85%, 86%, 87%, 88%, 89%, 90%, 91%, 92%, 93%, 94%, 95%, 96%, 97%, 98%, or 99% identity with SEQ ID NO: 64. In another embodiment, the nucleic acid sequence encoding the BLUF domain encodes a polypeptide comprises SEQ ID NO: 64. In yet another embodiment, the nucleic acid sequence encoding the BLUF domain encodes a polypeptide consists of SEQ ID NO: 64.

In some embodiments, the BLUF domain can be derived from sequences such as a BLUF domain from YcgF found in E. coli. Exemplary amino acid sequences for the BLUF domain are listed below.

| BLUF Domain and origin | Accession No. |
|---|---|
| BLUF domain regulated adenylyl cyclase [synthetic construct] | ADC33126.2 |
| Chain A, Beta subunit of photoactivated adenylyl cyclase [Beggiatoa sp. PS] | 5M27_A |
| Chain A, Beta subunit of photoactivated adenylyl cyclase [Beggiatoa sp. PS] | 5MBK_A |
| Chain A, Beta subunit of photoactivated adenylyl cyclase [Beggiatoa sp. PS] | 5MBG_A |
| Chain A, Beta subunit of photoactivated adenylyl cyclase [Beggiatoa sp. PS] | 5MBJ_A |
| Chain A, Beta subunit of photoactivated adenylyl cyclase [Beggiatoa sp. PS] | 5MBH_A |
| BLUF domain-containing protein [Pseudomonadota bacterium] | MDY6994672.1 |
| family 3 adenylate cyclase [Beggiatoa sp. IS2] | OQW94152.1 |
| BLUF domain-containing protein [Candidatus Parabeggiatoa sp.] | MEC4583593.1 |
| BLUF domain-containing protein [Argonema antarcticum] | WP_249064968.1 |
| BLUF domain-containing protein [Argonema galeatum] | WP_249103204.1 |
| BLUF domain-containing protein [Laspinema sp. D2d] | WP_261208488.1 |
| BLUF domain-containing protein [Oscillatoriales] | WP_190471735.1 |
| BLUF domain-containing protein [Laspinema sp. D2c] | MCT7958757.1 |
| BLUF domain-containing protein [Phormidium pseudopriestleyi] | WP_207088110.1 |
| BLUF domain-containing protein [Oscillatoria sp. HE19RPO] | WP_254564190.1 |
| BLUF domain-containing protein [Laspinema sp. D2b] | MCT7961659.1 |
| BLUF domain-containing protein [Laspinema sp. D2a] | MCT7969545.1 |
| BLUF domain-containing protein [unclassified Laspinema] | WP_261199945.1 |
| BLUF domain-containing protein [Phormidium sp. CCY1219] | WP_324633340.1 |
| BLUF domain-containing protein [Laspinema sp. D3c] | WP_261264186.1 |
| TPA: BLUF domain-containing protein [Oscillatoriaceae cyanobacterium | HIK09240.1 |
| M33_DOE_052] | |
| Chain A, Photoactivated adenylyl cyclase [Cyanobacteriota] | 5X4T_A |
| BLUF domain-containing protein [Oscillatoria acuminata] | WP_015149803.1 |
| Chain A, Family 3 adenylate cyclase [Oscillatoria acuminata PCC 6304] | 4YUS_A |
| BLUF domain-containing protein [[Phormidium] sp. ETS-05] | WP_199247919.1 |
| BLUF domain-containing protein [Oxynema aestuarii] | WP_168567585.1 |
| BLUF domain-containing protein [Oxynema sp. CENA135] | WP_200666374.1 |
| BLUF domain-containing protein [Planktothricoides raciborskii] | WP_190879674.1 |
| BLUF domain-containing protein [Cyanobacteriota bacterium] | MDY6804544.1 |
| BLUF domain-containing protein [Thiotrichaceae bacterium] | MEC4315055.1 |
| family 3 adenylate cyclase [Oscillatoria sp. SIO1A7] | NER35755.1 |
| family 3 adenylate cyclase [Desulfobacteraceae bacterium IS3] | OQX02467.1 |
| BLUF domain-containing protein [Desulfonema limicola] | WP_207687430.1 |
| BLUF domain-containing protein [Pseudanabaena sp. M135S2SP2A07QC] | MCA6553699.1 |
| family 3 adenylate cyclase [Pseudanabaena sp.] | PZU97124.1 |
| BLUF domain-containing protein [Pseudanabaena sp. M090S1SP1A06QC] | MCA6613851.1 |
| BLUF domain-containing protein [Pseudanabaena yagii] | WP_169362325.1 |
| BLUF domain-containing protein [Planktothricoides sp. SR001] | WP_054465978.1 |
| BLUF domain-containing protein [Pseudanabaena sp. lw0831] | WP_201324811.1 |
| TPA: family 3 adenylate cyclase [Desulfobacteraceae bacterium] | HAO21720.1 |
| BLUF domain-containing protein [Pseudanabaena sp. Salubria-1] | MCL1495131.1 |
| BLUF domain-containing protein [Thiotrichaceae bacterium] | MCV6637782.1 |
| family 3 adenylate cyclase [Desulfobacteraceae bacterium IS3] | OQX12545.1 |
| BLUF domain-containing protein [Pseudanabaena sp. SR411] | WP_094532923.1 |
| family 3 adenylate cyclase [Oscillatoriales cyanobacterium CG2_30_44_21] | OIP77085.1 |
| BLUF domain-containing protein [Pseudanabaena sp. UWO311] | WP_142655035.1 |
| BLUF domain-containing protein [Herpetosiphon sp.] | MBP8251513.1 |
| BLUF domain-containing protein [Phormidium tenue] | WP_190576548.1 |
| family 3 adenylate cyclase [Pseudanabaena frigida] | PZO43318.1 |
| BLUF domain-containing protein [Pseudanabaena sp. ABRG5-3] | WP_126385562.1 |
| BLUF domain-containing protein [Thiotrichaceae bacterium] | MDD2816840.1 |
| BLUF domain-containing protein [Pseudanabaena mucicola] | WP_190403445.1 |
| BLUF domain-containing protein [Pseudanabaena sp. Chao 1811] | WP_271251948.1 |
| family 3 adenylate cyclase [Pseudanabaena biceps] | NUN64992.1 |
| BLUF domain-containing protein [Pseudanabaena cinerea] | WP_190352189.1 |
| BLUF domain-containing protein [Pseudanabaena galeata] | WP_323263625.1 |
| BLUF domain-containing protein [Pseudanabaena sp. BC1403] | WP_103670160.1 |
| BLUF domain-containing protein [Pseudanabaena] | WP_281008382.1 |
| BLUF domain-containing protein [Pseudanabaena sp. FACHB-1998] | WP_190399663.1 |
| BLUF domain-containing protein [Pseudanabaena sp. CAN_BIN31] | MCY7331516.1 |
| BLUF domain-containing protein [Pseudanabaenaceae cyanobacterium | MDX2257260.1 |
| bins.39] | |
| BLUF domain-containing protein [Pseudanabaena sp. ABRG5-3] | WP_126385561.1 |
| BLUF domain-containing protein [Pseudanabaena sp. Chao 1811] | WP_271251949.1 |

In one embodiment, the gene or gene cassette is operably linked to a promoter that is not associated with the adenylyl cyclase enzyme in nature. In one embodiment, the promoter is inducible. In one embodiment, the promoter is induced in adipocytes. In one embodiment, the adipocyte-specific promoter is AP2 or AdipoQ. In one embodiment, the promoter is selected from the group consisting of EF1α promoter, CAG promoter, and CMV promoter.

In one embodiment, the photoactivated adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa). In one embodiment, the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007. In one embodiment, the adenylyl cyclase enzyme is from Beggiatoa sp. PS.

In one embodiment, the sequence of the adenylyl cyclase enzyme comprises one or more amino acid substitutions relative to a wild type adenylyl cyclase sequence. In one embodiment, the one or more amino acid substitutions are at positions corresponding to position 27, 123, 198, 266, 267, 278, 197, and/or 201 in the wild type adenylyl cyclase enzyme sequence. In one embodiment, the wild type adenylyl cyclase enzyme sequence comprises SEQ ID NO: 1 or SEQ ID NO: 31. In one embodiment, the sequence of the adenylyl cyclase enzyme sequence comprises an alanine (A) at a position corresponding to position 27 in the sequence of SEQ ID NO: 1 or SEQ ID NO: 31. In one embodiment, the sequence of the adenylyl cyclase enzyme comprises SEQ ID NO: 2.

In one embodiment, the gene or gene cassette encoding the adenylyl cyclase enzyme comprises a sequence that has at least 90%, 91%, 92%, 93%, 94%, 95%, 96%, 97%, 98%, or 99% identity to, comprises, or consists of any one of SEQ ID NOs: 32, 33, or 62. In one embodiment, the gene or gene cassette encoding the adenylyl cyclase enzyme encodes a polypeptide sequence that has at least 90%, 91%, 92%, 93%, 94%, 95%, 96%, 97%, 98%, or 99% identity to, comprises, or consists of SEQ ID NOs: 1, 2, or 31.

In some embodiments, the nucleic acid sequence of the wild type adenylyl cyclase and/or the nucleic acid sequence of the adenylyl cyclase comprising one or more amino acid substitutions is codon optimized for expression in human cells. In one embodiment, the gene or gene cassette encoding the adenylyl cyclase enzyme is codon optimized for expression in a mammalian cell.

In one embodiment, the optogenetic moiety and adenylyl cyclase enzyme are found in the same polypeptide in nature. In one embodiment, the optogenetic moiety is fused to the adenylyl cyclase enzyme, and is not associated with the adenylyl cyclase enzyme in nature. In some embodiments, the adenylyl cyclase and optogenetic moiety are fused with a peptide linker. In some embodiments, the optogenetic moiety is engineered to activated the adenylyl cyclase enzyme after light induction.

In one embodiment, the cell is a mammalian cell. In one embodiment, the mammalian cell is a human cell. In one embodiment, the cell is a pluripotent stem cell. In one embodiment, the pluripotent stem cell is a preadipocyte. In one embodiment, the pluripotent stem cell or preadipocyte is capable of differentiating into an adipocyte. In one embodiment, the adipocyte is a brown adipocyte, white adipocyte, or beige adipocyte.

In another aspect, the present invention provides a method of generating the cell as provided herein, wherein a cell which is capable of differentiation into an adipocyte is transfected with a vector comprising the one or more gene cassettes encoding one or more adenylyl cyclase enzymes. In one embodiment, the transfected cell is differentiated into an adipocyte, wherein the adipocyte is a brown adipocyte.

In one embodiment, the transfected cell, adipocyte, brown adipocyte, white adipocyte or beige adipocyte is expanded in culture.

In one embodiment, the vector is plasmid, lentivirus vector or an AAV vector.

In one embodiment, the vector further comprises a gene encoding a polypeptide to induce integration of the gene or gene cassette encoding the one or more adenylyl cyclase enzymes into the genome of the cell. In one embodiment, the gene encoding a polypeptide to induce integration of the gene or gene cassette into the genome is a DNA endonuclease. In one embodiment, the DNA endonuclease is a Cas9 endonuclease.

In another aspect, the present invention provides a system for activating uncoupling protein-1 (UCP-1) in a cell, the system comprising: a) a delivery vector comprising a non-native nucleotide sequence encoding one or more adenylyl cyclase enzymes, wherein the adenylyl cyclase enzyme comprises an optogenetic moiety, wherein the delivery vector provides for delivery of the one or more adenylyl cyclase enzymes to a cell; b) a light source; and c) a control device.

In one embodiment, the delivery vector is a plasmid, lentivirus vector or an AAV vector.

In one embodiment, the vector further comprises a gene encoding a polypeptide to induce integration of the gene or gene cassette encoding the one or more adenylyl cyclase enzymes into the genome of the cell. In one embodiment, the gene encoding a polypeptide to induce integration of the gene or gene cassette into the genome is a DNA endonuclease. In one embodiment, the DNA endonuclease is a Cas9 endonuclease.

In one embodiment, the non-native nucleotide sequence is operably linked to a promoter that is not associated with the adenylyl cyclase enzyme in nature.

In one embodiment, the promoter is inducible. In one embodiment, the promoter is selected from the group consisting of EF1α promoter, CAG promoter, and CMV promoter.

In one embodiment, the adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa). In one embodiment, the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007. In one embodiment, the adenylyl cyclase enzyme is from Beggiatoa sp. PS.

In one embodiment, the sequence of the adenylyl cyclase enzyme comprises one or more amino acid substitutions relative to a wild type adenylyl cyclase sequence.

In one embodiment, the one or more amino acid substitutions are at positions corresponding to position 27, 123, 198, 266, 267, 278, 197, and/or 201 in the wild type adenylyl cyclase enzyme sequence. In one embodiment, the wild type adenylyl cyclase enzyme sequence comprises SEQ ID NO: 1. In one embodiment, the sequence of the adenylyl cyclase enzyme sequence comprises an alanine (A) at a position corresponding to position 27 in the sequence of SEQ ID NO: 1. In one embodiment, the sequence of the adenylyl cyclase enzyme comprises SEQ ID NO: 2.

In one embodiment, the cell is a pluripotent cell or preadipocyte. In one embodiment, the pluripotent stem cell or preadipocyte is capable of differentiating into an adipocyte. In one embodiment, the light source is a light-emitting diode or a laser. In one embodiment, the control device is implantable. In one embodiment, the light source produces transient blue light, continuous blue light, or oscillating blue light. In one embodiment, the radiation intensity of the light source is at least 0.05 mW/cm², at least 0.1 mW/cm², at least 1 mW/cm², at least 2 mW/cm², at least 5 mW/cm², at least 10 mW/cm². In one embodiment, the system activates UCP-1 in a subject.

In another aspect, the present invention provides a method for activating uncoupling protein-1 (UCP-1) in a brown adipocyte of a subject, the method comprising: a) administering to the subject any one cell as provided for herein or the cell generated by any one method as provided for herein; b) activating the optogenetic moiety with a light source, wherein the adenylyl cyclase enzyme is activated, and wherein the light source is controlled by a control device; thereby activating UCP-1 in the cell of the subject.

In another aspect, the present invention provides a method for increasing the levels of uncoupling protein-1 (UCP-1) in a brown adipocyte of a subject, the method comprising: a) administering to the subject any one cell as provided for herein or the cell generated by any one method as provided for herein; b) activating the optogenetic moiety with a light source, wherein the adenylyl cyclase enzyme is activated, and wherein the light source is controlled by a control device; thereby activating UCP-1 in the cell of the subject.

In another aspect, the present invention provides a method for promoting the transcription of uncoupling protein-1 (UCP-1) in a brown adipocyte of a subject, the method comprising: a) administering to the subject any one cell as provided for herein or the cell generated by any one method as provided for herein; b) activating the optogenetic moiety with a light source, wherein the adenylyl cyclase enzyme is activated, and wherein the light source is controlled by a control device; thereby activating UCP-1 in the cell of the subject.

In another aspect, the present invention provides a method of treating a metabolic disease or disorder in a subject, the method comprising: a) administering to the subject any one cell as provided for herein or the cell generated by any one method as provided for herein; b) activating the optogenetic moiety with a light source, wherein the adenylyl cyclase enzyme is activated, and wherein the light source is controlled by a control device; thereby treating the subject.

In one embodiment, the metabolic disease or disorder is selected from obesity, diabetes or hyperlipidemia. In one embodiment, the subject is obese and in need of treatment.

In one embodiment, any one cell as provided for herein or the cell generated by any one method as provided for herein are derived from the subject or a donor.

In one embodiment, the administered cells are treated with the light source for at least 10 minutes, at least 30 minutes, at least 1 hour, at least 2 hours, at least 3 hours, at least 4 hours, at least 5 hours, or at least 6 hours. In some embodiments, the administered cells are treated with the light source for at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 21 minutes, at least 22 minutes, at least 23 minutes, at least 24 minutes, at least 25 minutes, at least 26 minutes, at least 27 minutes, at least 28 minutes, at least 29 minutes, at least 30 minutes, at least 31 minutes, at least 32 minutes, at least 33 minutes, at least 34 minutes, at least 35 minutes, at least 36 minutes, at least 37 minutes, at least 38 minutes, at least 39 minutes, at least 40 minutes, at least 41 minutes, at least 42 minutes, at least 43 minutes, at least 44 minutes, at least 45 minutes, at least 46 minutes, at least 47 minutes, at least 48 minutes, at least 49 minutes, at least 50 minutes, at least 51 minutes, at least 52 minutes, at least 53 minutes, at least 54 minutes, at least 55 minutes, at least 56 minutes, at least 57 minutes, at least 58 minutes, at least 59 minutes, or at least 60 minutes.

In some embodiments, the administered cells are treated with the light source for at least 1 hour, at least 1 hour 5 minutes, at least 1 hour 10 minutes, at least 1 hour 15 minutes, at least 1 hour 20 minutes, at least 1 hour 25 minutes, at least 1 hour 30 minutes, at least 1 hour 35 minutes, at least 1 hour 40 minutes, at least 1 hour 45 minutes, at least 1 hour 50 minutes, at least 1 hour 55 minutes, at least 2 hours, at least 2 hours 5 minutes, at least 2 hours 10 minutes, at least 2 hour 15 minutes, at least 2 hours 20 minutes, at least 2 hours 25 minutes, at least 2 hours 30 minutes, at least 2 hours 35 minutes, at least 2 hours 40 minutes, at least 2 hours 45 minutes, at least 2 hours 50 minutes, at least 2 hours 55 minutes, at least 3 hours, at least 3 hours 5 minutes, at least 3 hours 10 minutes, at least 3 hour 15 minutes, at least 3 hours 20 minutes, at least 3 hours 25 minutes, at least 3 hours 30 minutes, at least 3 hours 35 minutes, at least 3 hours 40 minutes, at least 3 hours 45 minutes, at least 3 hours 50 minutes, at least 3 hours 55 minutes, at least 4 hours, at least 4 hours 4 minutes, at least 4 hours 10 minutes, at least 4 hour 15 minutes, at least 4 hours 20 minutes, at least 4 hours 25 minutes, at least 4 hours 30 minutes, at least 4 hours 35 minutes, at least 4 hours 40 minutes, at least 4 hours 45 minutes, at least 4 hours 50 minutes, at least 4 hours 55 minutes, at least 5 hours, at least 5 hours 5 minutes, at least 5 hours 10 minutes, at least 5 hour 15 minutes, at least 5 hours 20 minutes, at least 5 hours 25 minutes, at least 5 hours 30 minutes, at least 5 hours 35 minutes, at least 5 hours 40 minutes, at least 5 hours 45 minutes, at least 5 hours 50 minutes, at least 5 hours 55 minutes, or at least 6 or more hours.

In some embodiments, the administered cells are treated with the light source for between about 10 minutes and about 6 hours, such as between about 15 minutes and about 6 hours, about 20 minutes and about 6 hours, about 25 minutes and about 6 hours, about 30 minutes and about 6 hours, about 35 minutes and about 6 hours, about 40 minutes and about 6 hours, about 45 minutes and about 6 hours, about 50 minutes and about 6 hours, about 55 minutes and about 6 hours, about 10 minutes and about 5 hours, about 15 minutes and about 5 hours, about 20 minutes and about 5 hours, about 25 minutes and about 5 hours, about 30 minutes and about 5 hours, about 35 minutes and about 5 hours, about 40 minutes and about 5 hours, about 45 minutes and about 5 hours, about 50 minutes and about 5 hours, about 55 minutes and about 5 hours, about 10 minutes and about 4 hours, about 15 minutes and about 4 hours, about 20 minutes and about 4 hours, about 25 minutes and about 4 hours, about 30 minutes and about 4 hours, about 35 minutes and about 4 hours, about 40 minutes and about 4 hours, about 45 minutes and about 4 hours, about 50 minutes and about 4 hours, about 55 minutes and about 4 hours, about 10 minutes and about 3 hours, about 15 minutes and about 3 hours, about 20 minutes and about 3 hours, about 25 minutes and about 3 hours, about 30 minutes and about 3 hours, about 35 minutes and about 3 hours, about 40 minutes and about 3 hours, about 45 minutes and about 3 hours, about 50 minutes and about 3 hours, about 55 minutes and about 3 hours, about 10 minutes and about 2 hours, about 15 minutes and about 2 hours, about 20 minutes and about 2 hours, about 25 minutes and about 2 hours, about 30 minutes and about 2 hours, about 35 minutes and about 2 hours, about 40 minutes and about 2 hours, about 45 minutes and about 2 hours, about 50 minutes and about 2 hours, about 55 minutes and about 2 hours, about 10 minutes and about 1 hour, about 15 minutes and about 1 hour, about 20 minutes and about 1 hour, about 25 minutes and about 1 hour, about 30 minutes and about 1 hour, about 35 minutes and about 1 hour, about 40 minutes and about 1 hour, about 45 minutes and about 1 hour, about 50 minutes and about 1 hour, about 55 minutes and about 1 hour, about 1 hour and about 6 hours, about 2 hours and about 6 hours, about 3 hours and about 6 hours, about 3 hours and about 6 hours, about 4 hours and about 6 hours, or about 5 hours and about 6 hours.

In some embodiments, the administered cells are treated with the light source for about 10 milliseconds to about 500 milliseconds every second for a total time of at least 10 minutes, at least 15 minutes, at least 20 minutes, at least 25 minutes at least 30 minutes, at least 35 minutes, at least 40 minutes, at least 45 minutes, at least 50 minutes, at least 55 minutes, at least 1 hour, at least 1 hour 5 minutes, at least 1 hour 10 minutes, at least 1 hour 15 minutes, at least 1 hour 20 minutes, at least 1 hour 25 minutes, at least 1 hour 30 minutes, at least 1 hour 35 minutes, at least 1 hour 40 minutes, at least 1 hour 45 minutes, at least 1 hour 50 minutes, at least 1 hour 55 minutes, at least 2 hours, at least 2 hours 5 minutes, at least 2 hours 10 minutes, at least 2 hour 15 minutes, at least 2 hours 20 minutes, at least 2 hours 25 minutes, at least 2 hours 30 minutes, at least 2 hours 35 minutes, at least 2 hours 40 minutes, at least 2 hours 45 minutes, at least 2 hours 50 minutes, at least 2 hours 55 minutes, at least 3 hours, at least 3 hours 5 minutes, at least 3 hours 10 minutes, at least 3 hour 15 minutes, at least 3 hours 20 minutes, at least 3 hours 25 minutes, at least 3 hours 30 minutes, at least 3 hours 35 minutes, at least 3 hours 40 minutes, at least 3 hours 45 minutes, at least 3 hours 50 minutes, at least 3 hours 55 minutes, at least 4 hours, at least 4 hours 4 minutes, at least 4 hours 10 minutes, at least 4 hour 15 minutes, at least 4 hours 20 minutes, at least 4 hours 25 minutes, at least 4 hours 30 minutes, at least 4 hours 35 minutes, at least 4 hours 40 minutes, at least 4 hours 45 minutes, at least 4 hours 50 minutes, at least 4 hours 55 minutes, at least 5 hours, at least 5 hours 5 minutes, at least 5 hours 10 minutes, at least 5 hour 15 minutes, at least 5 hours 20 minutes, at least 5 hours 25 minutes, at least 5 hours 30 minutes, at least 5 hours 35 minutes, at least 5 hours 40 minutes, at least 5 hours 45 minutes, at least 5 hours 50 minutes, at least 5 hours 55 minutes, or at least 6 or more hours.

In some embodiments, the administered cells are treated with the light source at a frequency of every second, every two seconds, every three seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, every 20 seconds, every 21 seconds, every 22 seconds, every 23 seconds, every 24 seconds, every 25 seconds, every 26 seconds, every 27 seconds, every 28 seconds, every 29 seconds, every 30 seconds, every 31 seconds, every 32 seconds, every 33 seconds, every 34 seconds, every 35 seconds, every 36 seconds, every 37 seconds, every 38 seconds, every 39 seconds, every 40 seconds, every 41 seconds, every 42 seconds, every 43 seconds, every 44 seconds, every 45 seconds, every 46 seconds, every 47 seconds, every 48 seconds, every 49 seconds, every 50 seconds, every 51 seconds, every 52 seconds, every 53 seconds, every 54 seconds, every 55 seconds, every 56 seconds, every 57 seconds, every 58 seconds, every 59 seconds, or every 60 or more seconds.

In some embodiments, the administered cells are treated with the light source for a duration of about 10 milliseconds to about 500 milliseconds, such as about 15 milliseconds to about 500 milliseconds, about 20 milliseconds to about 500 milliseconds, about 25 milliseconds to about 500 milliseconds, about 30 milliseconds to about 500 milliseconds, about 35 milliseconds to about 500 milliseconds, about 40 milliseconds to about 500 milliseconds, about 45 milliseconds to about 500 milliseconds, about 50 milliseconds to about 500 milliseconds, about 55 milliseconds to about 500 milliseconds, about 60 milliseconds to about 500 milliseconds, about 65 milliseconds to about 500 milliseconds, about 70 milliseconds to about 500 milliseconds, about 75 milliseconds to about 500 milliseconds, about 80 milliseconds to about 500 milliseconds, about 85 milliseconds to about 500 milliseconds, about 90 milliseconds to about 500 milliseconds, about 95 milliseconds to about 500 milliseconds, about 100 milliseconds to about 500 milliseconds, about 15 milliseconds to about 400 milliseconds, about 20 milliseconds to about 400 milliseconds, about 25 milliseconds to about 400 milliseconds, about 30 milliseconds to about 400 milliseconds, about 35 milliseconds to about 400 milliseconds, about 40 milliseconds to about 400 milliseconds, about 45 milliseconds to about 400 milliseconds, about 50 milliseconds to about 400 milliseconds, about 55 milliseconds to about 400 milliseconds, about 60 milliseconds to about 400 milliseconds, about 65 milliseconds to about 400 milliseconds, about 70 milliseconds to about 400 milliseconds, about 75 milliseconds to about 400 milliseconds, about 80 milliseconds to about 400 milliseconds, about 85 milliseconds to about 400 milliseconds, about 90 milliseconds to about 400 milliseconds, about 95 milliseconds to about 400 milliseconds, about 100 milliseconds to about 400 milliseconds, about 15 milliseconds to about 300 milliseconds, about 20 milliseconds to about 300 milliseconds, about 25 milliseconds to about 300 milliseconds, about 30 milliseconds to about 300 milliseconds, about 35 milliseconds to about 300 milliseconds, about 40 milliseconds to about 300 milliseconds, about 45 milliseconds to about 300 milliseconds, about 50 milliseconds to about 300 milliseconds, about 55 milliseconds to about 300 milliseconds, about 60 milliseconds to about 300 milliseconds, about 65 milliseconds to about 300 milliseconds, about 70 milliseconds to about 300 milliseconds, about 75 milliseconds to about 300 milliseconds, about 80 milliseconds to about 300 milliseconds, about 85 milliseconds to about 300 milliseconds, about 90 milliseconds to about 300 milliseconds, about 95 milliseconds to about 300 milliseconds, about 100 milliseconds to about 300 milliseconds, about 15 milliseconds to about 200 milliseconds, about 20 milliseconds to about 200 milliseconds, about 25 milliseconds to about 200 milliseconds, about 30 milliseconds to about 200 milliseconds, about 35 milliseconds to about 200 milliseconds, about 40 milliseconds to about 200 milliseconds, about 45 milliseconds to about 200 milliseconds, about 50 milliseconds to about 200 milliseconds, about 55 milliseconds to about 200 milliseconds, about 60 milliseconds to about 200 milliseconds, about 65 milliseconds to about 200 milliseconds, about 70 milliseconds to about 200 milliseconds, about 75 milliseconds to about 200 milliseconds, about 80 milliseconds to about 200 milliseconds, about 85 milliseconds to about 200 milliseconds, about 90 milliseconds to about 200 milliseconds, about 95 milliseconds to about 200 milliseconds, about 100 milliseconds to about 200 milliseconds, about 15 milliseconds to about 100 milliseconds, about 20 milliseconds to about 100 milliseconds, about 25 milliseconds to about 100 milliseconds, about 30 milliseconds to about 100 milliseconds, about 35 milliseconds to about 100 milliseconds, about 40 milliseconds to about 100 milliseconds, about 45 milliseconds to about 100 milliseconds, about 50 milliseconds to about 100 milliseconds, about 55 milliseconds to about 100 milliseconds, about 60 milliseconds to about 100 milliseconds, about 65 milliseconds to about 100 milliseconds, about 70 milliseconds to about 100 milliseconds, about 75 milliseconds to about 100 milliseconds, about 80 milliseconds to about 100 milliseconds, about 85 milliseconds to about 100 milliseconds, about 90 milliseconds to about 100 milliseconds, or about 95 milliseconds to about 100 milliseconds every at least second.

In some embodiments, the treatment with the light source is administered once a day, twice a day, or three times a day. In some embodiments, the treatment with the light source is administered up to once a week, twice a week, three times a week, 4 times a week, 5 times a week, 6 times a week, 7 times a week, 8 times a week, 9 times a week, 10 times a week, 11 times a week, 12 times a week, 13 times a week, 14 times a week, 15 times a week, 16 times a week, 17 times a week, 18 times a week, 19 times a week, 20 times a week, or 21 times a week.

In some embodiments, the treatment with the light source is administered every day, every 2 days, every 3 days, every 4 days, every 5 days, every 6 days, every 7 days, every week, every 2 weeks, every 3 weeks, every 4 weeks, every 5 weeks, every 6 weeks, every 7 weeks, every 8 weeks, every 9 weeks, every 10 weeks, every 11 weeks, or every 12 or more weeks.

In some embodiments, the treatment with the light source is administered for at least 1 month, at least 2 months, at least 3 months, at least 4 months, at least 5 months, at least 6 months, at least 7 months, at least 8 months, at least 9 months, at least 10 months, at least 11 months, or at least 1 or more year.

In one embodiment, further wherein any one cell as provided for herein or the cell generated by any one method as provided for herein are grown on a scaffold prior to being administered to the subject.

In another aspect, the present invention provides a method of treating a subject in need thereof, the method comprising: a) administering to a cell of the subject a nucleic acid encoding one or more adenylyl cyclase enzymes, wherein the adenylyl cyclase enzyme comprises an optogenetic moiety; b) activating the optogenetic moiety with a light source, wherein the adenylyl cyclase enzyme is activated, and wherein the light source is controlled by a control device; thereby treating the subject. In one embodiment, the delivery vector is a plasmid, lentivirus vector or an AAV vector.

In one embodiment, the nucleotide sequence encoding one or more adenylyl cyclase enzymes is operably linked to a promoter that is not associated with the adenylyl cyclase enzyme in nature. In one embodiment, the promoter is inducible. In one embodiment, the promoter is induced in adipocytes. In one embodiment, the adipocyte-specific promoter is AP2 or AdipoQ. In one embodiment, the promoter is selected from the group consisting of EF1α promoter, CAG promoter, and CMV promoter.

In one embodiment, the adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa). In one embodiment, the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007. In one embodiment, the adenylyl cyclase enzyme is from Beggiatoa sp. PS.

In one embodiment, the amino acid sequence of the adenylyl cyclase enzyme comprises one or more amino acid substitutions relative to a wild type adenylyl cyclase sequence.

In one embodiment, the one or more amino acid substitutions are at positions corresponding to position 27, 123, 198, 266, 267, 278, 197, and/or 201 in the wild type adenylyl cyclase enzyme sequence.

In one embodiment, the wild type adenylyl cyclase enzyme sequence comprises SEQ ID NO: 1 or SEQ ID NO: 30. In one embodiment, the sequence of the adenylyl cyclase enzyme sequence comprises an alanine (A) at a position corresponding to position 27 in the sequence of SEQ ID NO: 1 or SEQ ID NO: 31. In one embodiment, the sequence of the adenylyl cyclase enzyme comprises SEQ ID NO: 2.

In one embodiment, the gene or gene cassette encoding the adenylyl cyclase enzyme comprises a sequence that has at least 90%, 91%, 92%, 93%, 94%, 95%, 96%, 97%, 98%, or 99% identity to, comprises, or consists of any one of SEQ ID NOs: 32, 33, or 62. In one embodiment, the gene or gene cassette encoding the adenylyl cyclase enzyme encodes a polypeptide sequence that has at least 90%, 91%, 92%, 93%, 94%, 95%, 96%, 97%, 98%, or 99% identity to, comprises, or consists of SEQ ID NOs: 1, 2, or 31.

In one embodiment, the optogenetic moiety and adenylyl cyclase enzyme are found in the same polypeptide in nature. In one embodiment, the optogenetic moiety is fused to the adenylyl cyclase enzyme, and is not associated with the adenylyl cyclase enzyme in nature.

In one embodiment, the gene or gene cassette encoding the adenylyl cyclase enzyme is codon optimized for expression in a mammalian cell.

In one embodiment, the cell is a pluripotent cell or preadipocyte.

In one embodiment, the light source is a light-emitting diode or a laser. In one embodiment, the control device is implantable. In one embodiment, the light source produces transient blue light, continuous blue light, or oscillating blue light. In one embodiment, the radiation intensity of the light source is at least 0.05 mW/cm², at least 0.1 mW/cm², at least 1 mW/cm², at least 2 mW/cm², at least 5 mW/cm², at least 10 mW/cm².

In one embodiment, the cells are treated with the light source for at least 10 minutes, at least 30 minutes, at least 1 hour, at least 2 hours, at least 3 hours, at least 4 hours, at least 5 hours, or at least 6 hours. In one embodiment, the cells are treated with the light source for about 10 milliseconds to about 500 milliseconds every second for at least 1 hour, at least 2 hours, at least 3 hours, at least 4 hours, or at least 5 hours. In one embodiment, the cells are treated with the light source for between about 10 minutes and 6 hours, about 30 minutes and about 6 hours, about 1 hour to about 6 hours, about 2 hours to about 6 hours, about 3 hours to about 6 hours, about 3 hours to about 6 hours, about 4 hours to about 6 hours, about 5 hours to about 6 hours. In one embodiment, the treatment with the light source is administered once a day, twice a day, three times a day, once a week, twice a week, three times a week, 4 times a week, 5 times a week, 6 times a week, or every day for at least 1 month, at least 2 months, at least 3 months, at least 4 months, at least 5 months, at least 6 months, at least 7 months, at least 8 months, at least 9 months, at least 10 months, at least 11 months, or at least 1 year.

### EXAMPLES

### Example 1

### Methods and Materials

### Experimental Model and Subject Details

### Cell Culture

ThermoMouse preadipocytes were generated from C57BL/6J interscapular brown adipose tissue as previously described²⁶.

Plasmid construction. To develop brown adipocytes capable of light induced activation, we utilized a variant of wild-type bPAC that contains a serine to alanine mutation at position 27 (bPAC). This variant has peak blue light absorption at 456 nm and reduced basal dark activity for tighter light inducible control compared to wild-type bPAC23,42. The parental bPAC plasmid pAAV-hSyn1-bPAC-cMyc-S27A-2A-tDimer was a gift from Thomas Oertner (Addgene plasmid #85398; http://n2t.net/addgene: 85398; RRID: Addgene_85398). pCD-EF1α-MCS-T2A (PGK-Puro) (SBI, CD822A-1) was modified via In-Fusion@ cloning to pCD-EF1α-MCS-T2A (PGK-Hygro) (Takara Bio USA, Inc., 102518). The 2 kb bPAC fragment was amplified with Q5 DNA polymerase (NEB, M0492S), gel purified and In-Fusion cloned into pCD-EF1α-MCS-T2A (PGK-Hygro) at Not1 and packaged into 3^{rd} generation lentivirus.

Cell culture and generation of clonal cell lines. ThermoMouse brown preadipocytes were expanded in basal medium consisting of DMEM supplemented with 10% FBS (ATCC, 30-2020), 1× GlutaMAX (Gibco, 35050061) and 1× Antibiotic-Antimycotic (Gibco, 15240062). Brown preadipocytes were transduced with in house generated lentivirus containing the EF1α-bPAC-T2A-mRFP1-PGK-Hygro plasmid. Virus was removed 24 hours after transduction and cells were allowed to expand for an additional 24 hours. Preadipocytes were dissociated from cell culture plates using TrypLE (Gibco, 12563029), pelleted (300×g) and resuspended in autoMacs Running Buffer (Miltenyi, 130-091-221) prior to flow sorting of the bulk population by mRFP1 (561 nm/594 nm) on a BD Biosystems FACSAria. Transduced cells were subsequently expanded and selected with and without 200 ng/ml hygromycin B (InvivoGen, ant-hg-1) for up to 7 days. Single cells from the hygromycin B selected, transduced bPAC+preadipocytes were sorted by mRFP1+ expression into 96 well plates using a BD Biosystems FACsAria. Single cells were expanded into colonies and passed into increasing larger well/plate sizes while selected on hygromycin B (200 ng/ml). Samples from clones that continued to proliferate and maintain mRFP1 expression as determined by immunofluorescence microscopy on the EVOS M5000 Imaging System (ThermoFisher Scientific, AMF5000) were lysed and tested by qPCR for expression of bPAC. shRNA-scramble and shRNA-Ucp1 virus was custom generated using the plasmids described in the Key Resources Table by VectorBuilder. Brown preadipocytes were transduced with shRNA lentivirus for 24 hours and cells were allowed to expand for an additional 24 hours prior to 7 days of blasticidin selection (2 ug/ml). EGFP expression in stably transduced cells was verified by immunofluorescence microscopy.

For differentiation into brown adipocytes, cells were grown to 100% confluence in Geltrex coated Krystal™ 24 Well Glass Bottom Microplates (Southern Labware, 324041) and treated with brown adipocyte induction medium for 48 hours, consisting of basal medium supplemented with insulin (170 nM), T3 (2 nM), rosiglitazone (1 uM), IMBX (0.5 mM), dexamethasone (5 μM), indomethacin (125 μM) and ascorbic acid (50 ug/ml)⁴³. Following induction, medium was replaced with growth medium supplemented with insulin (170 nM), T3 (2 nM), rosiglitazone (1 uM) and ascorbic acid (50 ug/ml) for 72 hours. Cells were then maintained in basal medium until experiments were performed as described in the text.

BODIPY staining. Culture medium was replaced with BODIPY diluted 1:2000 in fresh culture medium for 30 minutes at 37° C. Following 30 minutes, culture medium with BODIPY was removed and cells were gently washed once with PBS. PBS was removed and replaced with fresh cell culture medium prior to imaging on an EVOS Fluorescence Microscope using the 482 nm channel.

Quantitative PCR (qPCR). RNA was extracted from monolayer cultures with the RNeasy Micro Kit (Qiagen, 74004). cDNA synthesis was performed using qScript cDNA SuperMix (Quanta Biosciences, 95048-100). qPCR was carried out using AzuraView GreenFast qPCR Blue Mix LR (Azura Genomics, AZ-2320) and amplified on a CFX384 real-time PCR machine (BioRad). Fold changes (relative mRNA) were calculated using the ΔΔCT method, normalized to endogenous Actb levels and displayed relative to vehicle treated or day 0 controls. Specificities of primer sets were determined by melt curve analysis on qPCR-generated amplicons. Average values (+SD) of three replicates are shown for each graph.

Western blot. Cells were lysed in RIPA buffer containing Halt Protease & Phosphatase Inhibitor Cocktail (Thermo Fisher Scientific, 78440) and centrifuged at 15,000×g for 5 minutes to pellet cell debris. The supernatant was transferred to a new tube and cell lysates were denatured at 95° C. for 5 minutes. Protein concentrations were determined using the Pierce BCA Protein Assay Kit (Thermo Fisher Scientific, 23225) following manufacturer's instructions to normalize gel loading. Protein was separated on 4-20% Criterion TGX precast gels (BioRad, 5671094) and then transferred onto PVDF membranes. Membranes were incubated overnight at 4° C. in 1× Detector Block Solution (KPL, 72-01-03). Primary antibodies against CREB (Cell Signaling Technology, 9197), phospho-CREB (Ser133) (Cell Signaling Technology, 9198), UCP1 (Cell Signaling Technology, 72298) and β-tubulin (Cell Signaling Technology, 2146) were diluted at a ratio of 1:1000 in TBS with Tween 20 and 0.2× Detector Block Solution and incubated overnight at 4° C. Membranes were washed 3 times with TBS with Tween 20 (5 minutes per wash). Following washes, HRP secondaries diluted at a ratio of 1:1200 were incubated for 2 hours at room temperature and washed 3 times with TBS with Tween 20 (5 minutes per wash). Membranes were developed with Clarity Western ECL Substrate (BioRad, 1705061) and detected using a Bio-Rad ChemiDoc Touch Imaging System.

Multiplate photoirradiation system (MPS) and light pulsing. Blue light pulsing was conducted using an in-house manufactured 24-well microplate photo irradiation system (MPS) configured with blue LEDs (466 nm) with a 5-volt, 40-amp, AC power supply as described in²⁷. Cells were pulsed with blue light (466 nm) in a 5% CO₂ cell culture incubator set at 37° C. with light intensities between 0.4 to 1 mW/cm² for up to 6 hours as described in the text.

CAMP, fatty acid and glucose uptake assays. The luciferase-based GloSensor CAMP Assay (Promega, E1290) was used to monitor intracellular cAMP in brown preadipocytes (clonal line Td1). bPAC+ brown preadipocytes were transfected in basal medium with the pGloSensor™-20F CAMP Plasmid (Promega, E1171) in tissue culture grade 96 well Krystal microplates (Porvair Sciences, 214006) using the TransIT-X2 Transfection Reagent (Mirus, MIR 6004). 48 hours after transfection, cells were incubated with GloSensor cAMP Reagent for 2 hours. 10 minutes after forskolin (5 μm) or blue light treatments, relative luciferase units (RLU) were measured using the GloMax Explorer Microplate Reader & Luminometer at 0.3-1.0 second integrations (Promega).

The Glucose Uptake-Glo™ Assay (Promega, J1341) was used to measure glucose uptake in mature brown adipocytes (clonal line Td1). 24 hours prior to the assay, basal medium was replaced with serum free DMEM containing 0.5% BSA, 10 mM HEPES and 1× GlutaMAX for 24 hours. On the day of the assay, medium was replaced with 100 μl of serum and glucose free DMEM for 1 hour at 37° C. and pulsed with blue light and/or treated with a range of insulin concentrations for 30 minutes. Glucose uptake analysis was performed by following the Glucose Uptake-Glo Assay Kit manufacturer's instructions and analyzed for luminescence at 0.3-1.0 second integrations using the GloMax Explorer Microplate Reader & Luminometer (Promega).

The QBT Fatty Acid Uptake Assay (Molecular Devices, R8132) was used to measure fatty acid uptake in mature brown adipocytes (clonal line Td1). 24 hours prior to the assay, basal medium was replaced with 100 μl of serum free DMEM containing 10 mM HEPES and 1× GlutaMAX. Brown adipocytes were treated with 30 minutes of blue light and/or forskolin (5 μm) at 37° C. Medium was removed and 100 ul of 1× Loading Buffer containing 0.2% fatty acid free BSA was added per well. Plates were transferred immediately to a FlexStation 3 Multi-Mode Microplate Reader (Molecular Devices) pre-set to 37° C. for readings in bottom mode with excitation and emission parameters set at 485/515 nm.

Microcalorimetry. Total metabolic activity was measured on the calScreener™ (Symcel SverigeAB, Spånga, Sweden), an advanced isothermal microcalorimetry based instrument for accurate and sensitive measures of heat production in real-time³⁰. Brown preadipocytes were expanded and differentiated in 200 ul of medium in tissue culture treated calWell insert plates (Symcel, 1900402) coated with Geltrex (ThermoFisher Scientific, A1413201). Immediately after blue light stimulation, calWell inserts containing brown adipocytes were transferred to screw-capped titanium vials. The calPlate containing 48 calWell inserts was inserted into two temperature equilibrium zones for a total of 30 minutes prior to entering the final measurement position. Data was collected after an additional 15-30 minutes stabilization time to allow for frictional heat loss due to final sample positioning. Data was continuously collected with the calView software and normalized to internal reference wells containing medium only.

Statistical Analysis. P values were calculated in Prism 9 (GraphPad Software) using a two-tailed, unpaired Student t test with P<0.05 considered significant. One-way ordinary ANOVA was used to analyze the difference between the means when analyzing two or more groups. In the case of multiple comparisons, Tukey correction was used to determine P values after one-way ANOVA analysis. Significance was defined as p<0.05. Error bars represent the mean±SD (standard deviation) of 3 replicates unless otherwise noted in the figure legends with *P<0.05, ** P<0.01, *** P<0.001 and **** P<0.0001.

TABLE 1

| | SEQ | | SEQ | |
|---|---|---|---|---|
| | ID | | ID | |
| Primers | NO | Forward | NO | Reverse |
| Ucp1 | 66 | CGACTCAGTCCAAGAGT | 67 | GCCGGCTGAGATCTTGTTTC |
| | | ACTTCTCTTC | | |
| Nr4a3 | 68 | ACGCCGAAACCGATGT | 69 | CTCCTGTTGTAGTGGGCTCTTTG |
| | | CAGTAC | | |
| bPAC | 70 | CCAGCGGATCGGCAAG | 71 | GGTTTTCAGGCACAGGATGT |
| | | GTGG | | |
| Pgc1a | 72 | GAATCAAGCCACTACA | 73 | CATCCCTCTTGAGCCTTTCGTG |
| | | GACACCG | | |
| Tfam | 74 | GAGGCAAAGGATGATT | 75 | GAGGCAAAGGATGATTCGGCTC |
| | | CGGCTC | | |
| Atp5g1 | 76 | CATCGACACAGCAGCC | 77 | CCAGAATGGCATAGGAGAAGAG |
| | | AAGTTC | | C |
| Atp5g3 | 78 | GTAGGAGTTGCTGGTTC | 79 | GCTTCAGACAAGGCAAATCCCA |
| | | TGGTG | | G |
| Ndufs4 | 80 | GCTCGCAATAACATGCA | 81 | GGTCAGAACCATGTTGGAGAGG |
| | | GTCTGG | | |
| Ndufb8 | 82 | CGCCAAGAAGTATAAC | 83 | CCTCTCATGCTGTGATCGGTTG |
| | | ATGCGAG | | |
| Sdhb | 84 | TGCGGACCTATGGTGTT | 85 | CCAGAGTATTGCCTCCGTTGATG |
| | | GGATG | | |
| Uqcr2 | 86 | ATGCCACCTTCTACCGT | 87 | GTTTCCACTCGCTGCCATTGAC |
| | | CCTTC | | |
| Cox412 | 88 | ACAGTGATGGGCTGCGT | 89 | CTGTTGGGCTTTCCGTTCTTCC |
| | | CTTCT | | |
| Bnip3 | 90 | GCTCCAAGAGTTCTCAC | 91 | GTTTTTCTCGCCAAAGCTGTGGC |
| | | TGTGAC | | |
| Bnip3l | 92 | GCATGAGGAAGAGTGG | 93 | AAGGTGTGCTCAGTCGTTTTCCA |
| | | AGCCAT | | |
| Pink1 | 94 | CGACAACATCCTTGTGG | 95 | CATTGCCACCACGCTCTACACT |
| | | AGTGG | | |
| Mff | 96 | CTACTCGTAGGGCTTAC | 97 | GCTCCTTCAATGGCTGCATCTAG |
| | | CAGCA | | |

### Results

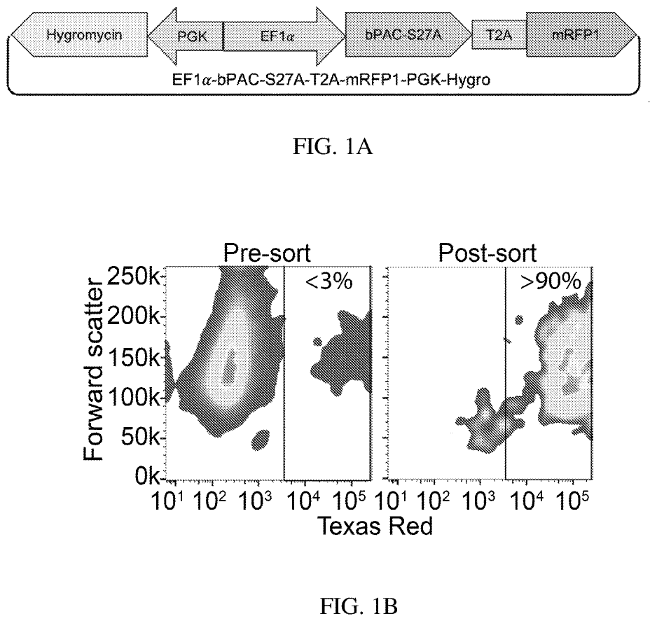

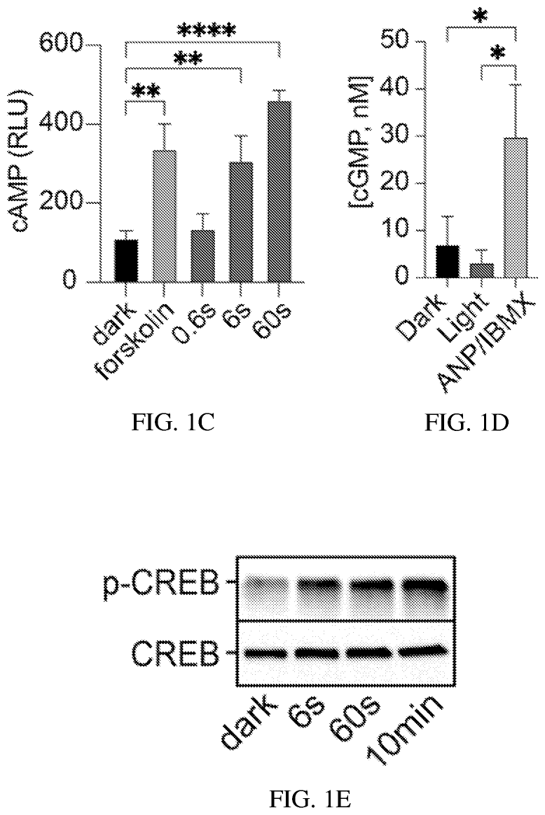

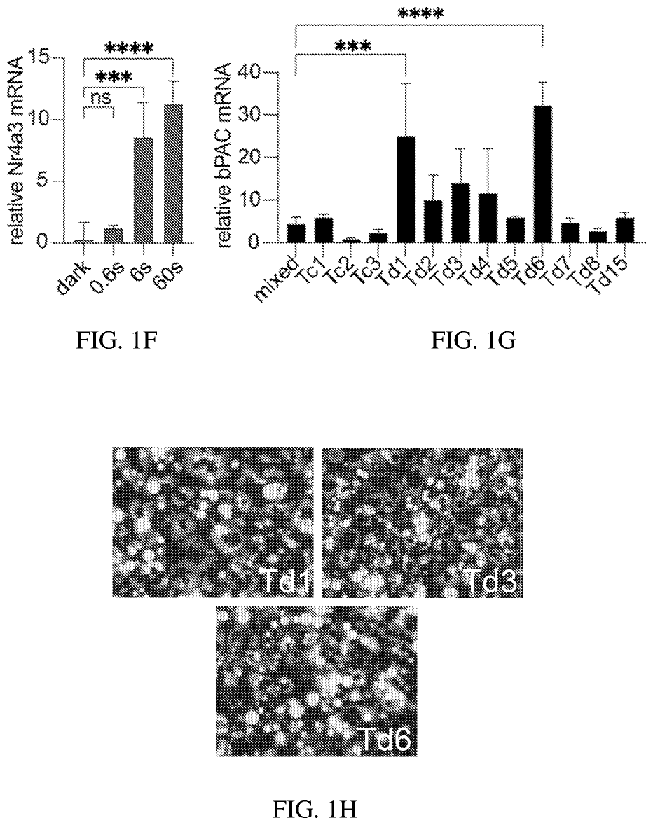

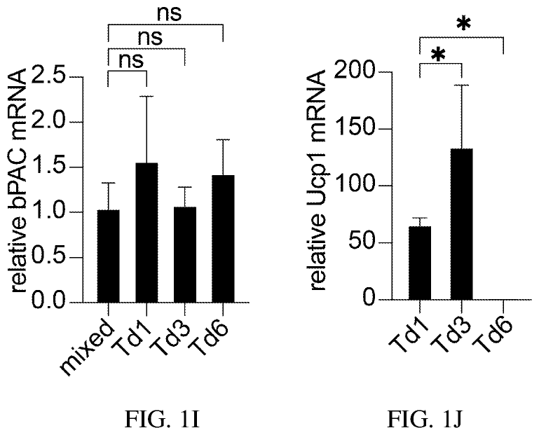

Lentiviral transduction of a bPAC-mRFP1 expression plasmid with hygromycin selection was used to generate stably expressing, immortalized brown preadipocytes derived from the ThermoMouse (FIG. 1A)^{23,26}. Since initial transduction of bPAC-mRFP1 in preadipocytes was very low (<3%), cells were purified by flow sorting for mRFP1 expression to increase purity to greater than 90% (FIG. 1B). Purified brown preadipocytes selected with hygromycin were transiently transfected with the pGloSensor-20F cAMP plasmid to monitor intracellular changes in cAMP concentration in real-time via luminescence. Blue light activation of brown preadipocytes was conducted using an in house manufactured microplate photo irradiation system (MPS) configured with blue LEDs (466 nm) 27. Significant increases in cAMP were observed after short blue light pulses that were equivalent to that observed with forskolin, a stimulator of endogenous adenylyl cyclase (FIG. 1C). The specificity of our approach was confirmed by the absence of cGMP production after blue light stimulation compared to known stimulators of cGMP (ANP, IBMX) (FIG. 1D). Additionally, brown preadipocytes treated with blue light showed a dose dependent increase in phosphorylation of the cAMP response element-binding protein (CREB) and expression of the immediate-early CREB target gene Nr4a3 (FIGS. 1E-1F), demonstrating that activation of bPAC stimulates cAMP signaling²⁸.

Transduced preadipocytes represent a mixed population of transgenic and non-transgenic cells that could phenotypically drift over multiple passages. To generate brown preadipocyte clonal cell lines that maintain bPAC expression and differentiate into brown adipocytes, we performed single cell fluorescence-activated cell sorting for mRFP1 on the mixed, hygromycin selected preadipocyte population. Expanded preadipocyte clones Td1, Td3 and Td6 were selected for further analysis based upon high bPAC expression when compared to the parental mixed culture (FIG. 1G). These clones differentiated robustly into mature adipocytes as determined by lipid staining and expressed bPAC at levels similar to the original mixed preadipocyte population, with varying levels of Ucp1 expression (FIG. 1H-1J). Clonal line Td1 was chosen for the majority of experiments moving forward since it consistently displayed high levels of bPAC and Ucp1 expression.

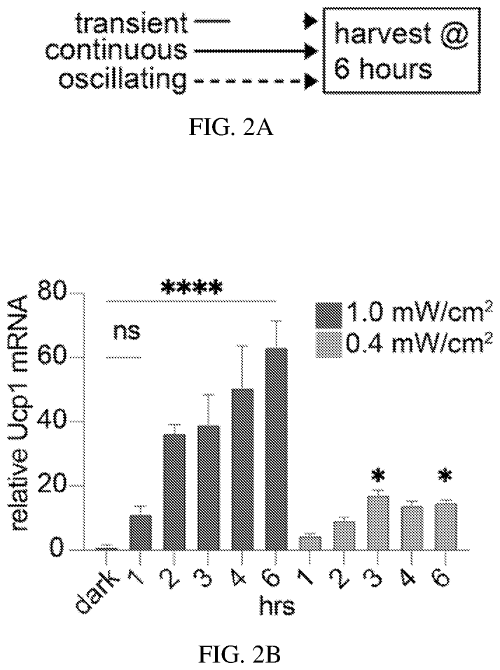

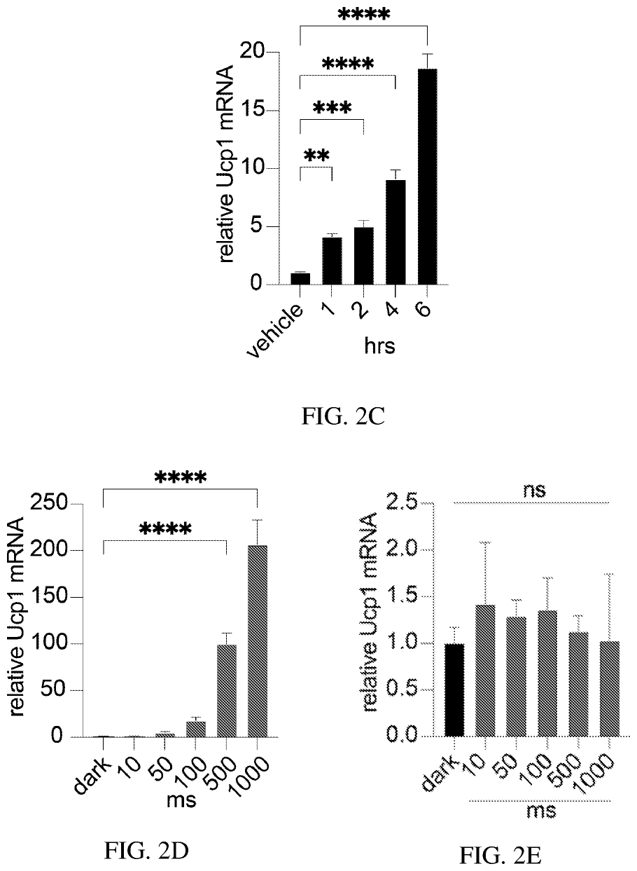

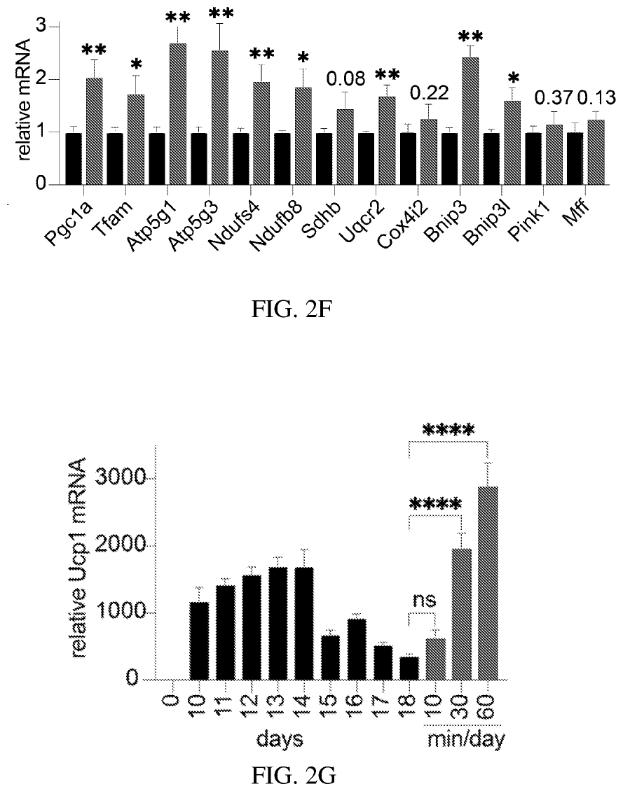

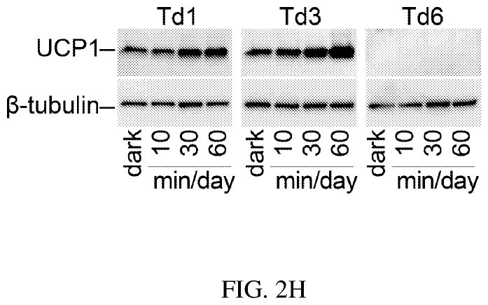

To test if bPAC activation promotes Ucp1 transcription, we tested different temporal patterns of light treatments (FIG. 2A). Differentiated brown adipocytes (clone Td1) were treated with transient or continuous blue light for 1 to 6 hr at time zero and harvested at 6 hr. The length of the initial light pulse was proportional to the increase in Ucp1 expression, similar to results obtained with transient or continuous forskolin treatment (FIG. 2B-2C). Lowering blue light intensity resulted in a plateaued effect on Ucp1 transcription, demonstrating that light intensity can have a profound effect on bPAC activation (FIG. 2B). Oscillating blue light pulses between 10 and 500 ms every second for 4 hr were also tested. Similar to transient light pulsing, Ucp1 expression increased in a dose dependent manner, illustrating that continuous, uninterrupted light exposure maximizes Ucp1 expression (FIG. 2D). Stimulation of bPAC negative brown adipocytes confirmed that blue light by itself does not stimulate Ucp1 expression (FIG. 2E). We next tested bPAC induced expression of hallmark genes involved in mitochondrial biology, including biogenesis (Pgc1a, Tfam), ATP synthesis (Atp5g1, Atp5g3), electron transport (Ndufs4, Ndufb8/complex I, Sdhb/complex II, Uqcr2/complex III and Cox4i2/complex IV) and mitophagy (Bnip3, Bnip31, Pink1, and Mff). These genes are necessary for increasing mitochondrial content, function and turnover in activated brown adipocytes and 4 hr continuous blue light stimulation largely increased their expression (1.5 to 2.5-fold) even though these genes are not known to be direct transcriptional targets of CREB signaling (FIG. 2F). Thus, light-induced stimulation of cAMP signaling increases expression of a program of genes that are necessary for proper mitochondrial function in brown adipocytes.

Brown adipocytes in cell culture lose expression of UCP1 due to absence of stimuli that activates downstream cAMP signaling, similar to those in mice housed at thermoneutrality. bPAC brown adipocytes showed peak Ucp1 expression at 14 days after initiation of differentiation, which rapidly decreased thereafter (FIG. 2G). To determine if activation of bPAC could lead to extended maintenance of Ucp1 expression, brown adipocytes were pulsed once per day with continuous blue light for 8 days, starting at day 10 and samples were harvested 24 hours after the last light pulse (FIG. 2G). 30-60 min of continuous blue light per day was sufficient to either maintain or increase Ucp1 transcript through day 18 compared to the previous peak (day 14). UCP1 protein levels were also higher in brown adipocytes after 8 days of 30-60 min blue light per day compared to cells receiving no light (FIG. 2H). Td6 adipocytes failed to express UCP1 protein, consistent with qPCR analysis of Ucp1 transcript (FIG. 1J). These data reveal that intermittent, optogenetic stimulation of cAMP signaling (30-60 min/day) is sufficient to prolong Ucp1 expression in brown adipocytes.

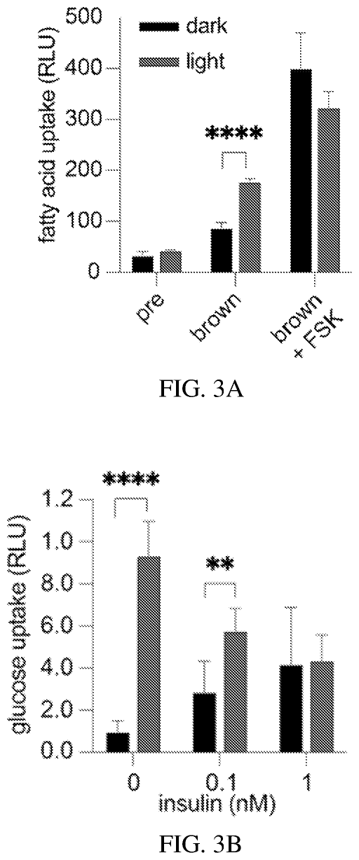

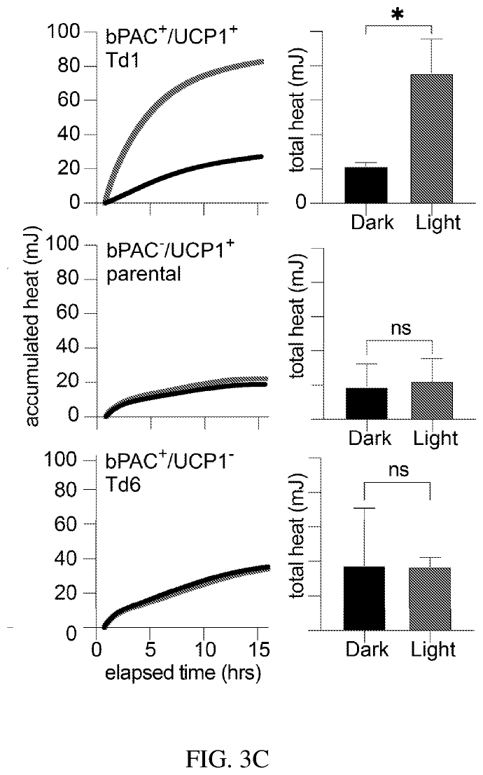

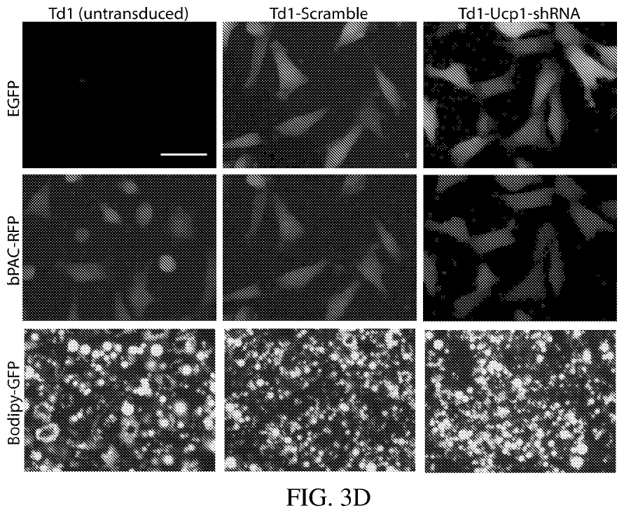

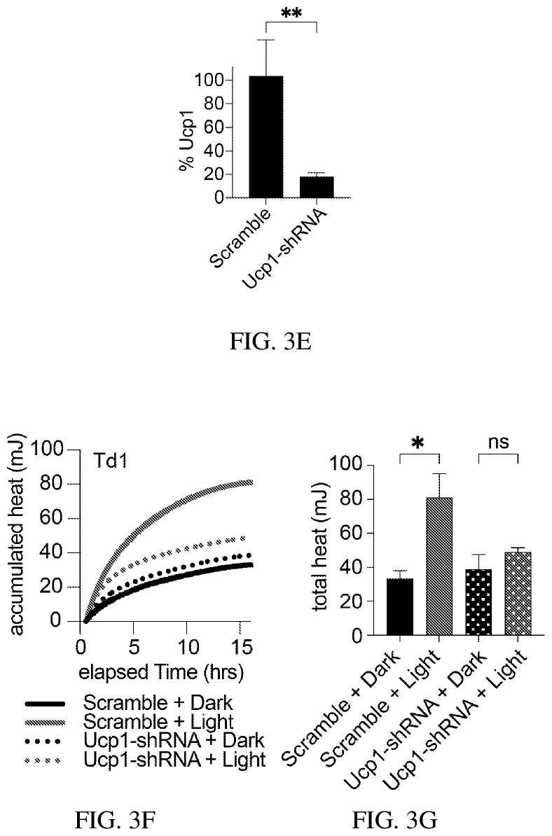

Upon activation, brown adipocytes increase uptake of fatty acids and glucose to be used as fuel for thermogenesis. Stimulation of Td1-bPAC+ brown adipocytes with 30 min of continuous blue light resulted in increased fatty acid and insulin-independent glucose uptake (FIGS. 3A-3B). The addition of insulin in combination with blue light led to lower levels of glucose uptake compared to blue light stimulation alone, possibly due to negative crosstalk between insulin-independent and -dependent glucose uptake pathways²⁹. To determine if blue light stimulation of bPAC results in increased thermogenesis due to UCP1 activation, we used highly sensitive calScreener-based microcalorimetry to measure heat production³⁰. Brown adipocytes were pulsed with 30 min of blue light, placed in the calScreener and heat accumulation was measured over time (FIG. 3C). bPAC+/UCP1+ brown adipocytes (clone Td1) responded robustly to blue light with a 4-fold greater accumulation of total heat compared to dark controls. No significant increases were observed in either the parental bPAC negative or UCP1 negative (clone Td6) adipocytes pulsed with blue light, suggesting that UCP1 may be the primary driver of bPAC stimulated heat production. To confirm the UCP1-dependent nature of the light inducible bPAC in brown adipocytes, the Td1 clonal cell line was transduced with short hairpin RNA (shRNA) that targets Ucp1 for degradation. Preadipocytes transduced with Scramble-EGFP: T2A: Bsd control or Ucp1-shRNA-EGFP: T2A: Bsd lentivirus were selected for blasticidin (Bsd) resistance for 7 days. The resulting preadipocytes stably express both the newly introduced viral constructs (EGFP), maintained expression of mRFP1-bPAC and differentiated into mature lipid containing adipocytes (FIG. 3D). The Ucp1-shRNA lentivirus reduced Ucp1 expression in mature adipocytes by greater than 80 percent compared to scramble control cells and this was associated with an absence of detectable light-induced heat production (FIGS. 3E-3G). Overall, these results demonstrate that blue light stimulated bPAC-S27A is able to increase UCP1-dependent thermogenesis in brown adipocytes.

### Discussion

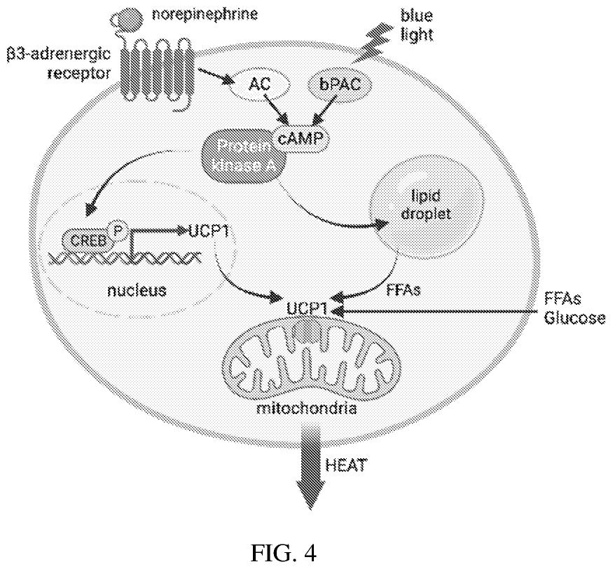

Energy intake that chronically exceeds energy expenditure is a primary driver of increased adiposity, obesity and adipose tissue dysfunction. Although the balance between energy intake and expenditure can be shifted favorably in the obese through diet and exercise, the success rate of lifestyle interventions is generally modest and transient³¹. Brown adipocytes become metabolically activated in response to cold-stimulated release of norepinephrine by the sympathetic nervous system, where they expend energy stored in glucose and lipids to generate heat. In brown adipose tissue, UCP1 is a key mechanistic component for increasing energy expenditure and heat production and its upregulation is associated with reduced obesity and improved insulin sensitivity³². To take advantage of brown adipocytes for therapeutics, new models that circumvent the cardiovascular risks associated with β-adrenergic agonist-based activation of brown adipocytes are needed¹⁰. Here, we established an optogenetic model to activate UCP1-dependent thermogenesis in which a light inducible adenylyl cyclase, originally cloned and genetically modified from the bacterium Beggiatoa, was used to increase intracellular levels of cAMP, UCP1 expression, fuel uptake and UCP1-dependent heat production in brown adipocytes (FIG. 4). The kinetics associated with this optogenetic model suggest that 30 min of light-induced activation per day is sufficient to increase UCP1 expression and thermogenesis in cultured brown adipocytes. Interestingly, a recent study demonstrated that transfer of mice from thermoneutrality to 18° C. for ≤30 minutes per day was sufficient to induce structural changes in brown adipose tissue with significant increases in UCP1 expression over a period of 4 weeks³³.

Prior to this study, it was not clear if photoactivatable adenylyl cyclases isolated from other organisms such as bacteria or algae were powerful enough to stimulate cAMP levels for increasing or maintaining thermogenesis in brown adipocytes. Indeed, the discovery of new PACs may offer enhanced stimulation of thermogenesis in brown adipocytes with less light intensity or shorter treatment times than that of bPAC. For example, a plasma membrane-anchored PAC (PACmn) was recently engineered from bPAC that displays a 4000-fold increase in cAMP after 2s of blue light stimulation, greatly exceeding forskolin activation of endogenous cyclases³⁴.

Our optogenetics approach also shows that UCP1 is the primary driver of heat production in Td1-bPAC+ brown adipocytes, as UCP1 deficient brown adipocytes lacked significant increases in heat production when stimulated with blue light. At this time, we are unable to conclude if blue-light activation of bPAC in other adipocyte models could additionally co-activate UCP1-independent pathways of thermogenesis, such as Ca2+ or creatine futile cycling^{35,36}. For example, it was recently shown in thermogenic adipocytes that ADRA1A-Gα_{q} signaling stimulates creatine futile signaling in a coordinated fashion with ADRB1-Gαₛ production of CAMP, in part through promoting creatine futile cycling gene expression³⁷. It is also noteworthy that we did not observe blue light mediated thermogenesis in bPAC negative brown adipocytes as several studies have shown that beige and brown adipocytes have a natural light sensing mechanism via the blue-light-responsive opsin, OPN3, which is necessary for enhanced thermogenic capacity and cold tolerance in mice^{38,39}. Whether this particular mechanism could be harnessed to substantially increase thermogenesis to levels necessary for weight loss in the absence of cold is unknown.

A fundamental concern for therapeutic development has been the selective modulation of UCP1 expression in brown adipocytes without adversely affecting regulation of other tissues. For example, because β3-adrenergic receptor agonists are associated with cardiovascular risks¹⁰, localized cell-based therapies, such as optogenetics, could limit off target effects and provide an effective and safer alternative to drugs for stimulation of energy expenditure in brown adipocytes. A strategy to overexpress bPAC in transplanted brown adipocytes for stimulation of UCP1-dependent thermogenesis may have merit as CRISPR based overexpression of UCP1 in human white adipocytes improved glucose tolerance and insulin sensitivity and increased energy expenditure when transplanted into mice⁴⁰. One advantage of using precise optogenetic control of thermogenesis is that energy expenditure can be modulated bidirectionally to prevent side effects, such as excess heat production that could be detrimental to adipocyte function or proper maintenance of euthermia. Recently, implantation of a wireless optogenetic device adjacent to subcutaneous adipose tissue in ChR2 transgenic mice was used to activate the non-canonical Ca2+ cycling thermogenesis program and protect mice from obesity⁴¹. In the future, combining multiple optogenetic activators of different thermogenic pathways may further enhance adipocyte energy expenditure in a precise manner for reversal of obesity and its related comorbidities.

### Example 2: Exemplary Sequences

| Description | |
|---|---|
| SEQ ID NO | Sequence |
| PAC wt (aa) | MKRLVYISKISGHLSLEEIQRIGKVSIKNNQRDNITGVLLYLQGLFFQILEGE |
| Codon | NEKVDKLYKKILVDDRHTNILCLKTEYDITDRMFPNWAMKTINLNENSEL |
| optimized | MIQPIKSLLQTITQSHRVLEKYMPARVIYLINQGINPLTVEPQLVEKIIFFSDI |
| Beggiatoa sp. | LAFSTLTEKLPVNEVVILVNRYFSICTRIISAYGGEVTKFIGDCVMASFTKEQ |
| SEQ ID NO: 1 | GDAAIRTSLDIISELKQLRHHVEATNPLHLLYTGIGLSYGHVIEGNMGSSLK |
| | MDHTLLGDAVNVAARLEALTRQLPYALAFTAGVKKCCQAQWTFINLGAH |
| | QVKGKQEAIEVYTVNEAQKYYDTLQITQLIRQTLENDKPRTY |
| PAC (S27A) | MMKRLVYISKISGHLSLEEIQRIGKVAIKNNQRDNITGVLLYLQGLFFQILE |
| (aa) | GENEKVDKLYKKILVDDRHTNILCLKTEYDITDRMFPNWAMKTINLNENS |
| Codon | ELMIQPIKSLLQTITQSHRVLEKYMPARVIYLINQGINPLTVEPQLVEKIIFFS |
| optimized | DILAFSTLTEKLPVNEVVILVNRYFSICTRIISAYGGEVTKFIGDCVMASFTK |
| Beggiatoa sp. | EQGDAAIRTSLDIISELKQLRHHVEATNPLHLLYTGIGLSYGHVIEGNMGSS |
| SEQ ID NO: 2 | LKMDHTLLGDAVNVAARLEALTRQLPYALAFTAGVKKCCQAQWTFINLG |
| | AHQVKGKQEAIEVYTVNEAQKYYDTLQITQLIRQTLENDKPRTY |
| Photoactivated | MYILVWKKGQQIKTFHTLDEAAQFKAASNIDEAQMFSVTVAPAISASGGS |
| adenylate | NEATNLRRLMYLSKSTNPEECNPQFLAEMARVATIRNREIGVSGFLMYSSP |
| cyclase subunit | FFFQVIEGTDEDLDFLFAKISADPRHERCIVLANGPCTGRMYGDWHMKDS |
| beta, 4.6.1.1, | HMDSITTHPAMKTILYQIARSFSSMWSYLPKSAGNMLLLGKDPAAQPPEP |
| Photoactivated | MSVVVTFIYLVEFGSILSNPNLTEQAAEVLSTFVDVCVKNVEGSGGNIAKFI |
| adenylyl cyclase | TGICMAYWPINRTEEALTAIQQISEDLAQLRSQQAPGSAVSLMYSQAGVH |
| subunit beta | YGRAMLCNAGSRKSDFTLLGDCINTTSRIATLAKKLKTPLLFSFEVRCLLG |
| pacB | DEMREEIEGAGMHQVKGRDKPVVVYQFPGPELDVEMVRQKIEQFTPGRF |
| Euglena gracilis | RCQMPVVEYEALPISQRPPIFDDTPKGNPRPRTPGYGGRQRSDSLVDRLIMI |
| 859 AA | AKLAGPSVSATGDTTLTTLTYISQATRPMSRLDLSAIMRTATRRNAQQSIT |
| SEQ ID NO: 3 | GTLLHVNGLFVQTLEGPKDAVVNLYLRIRQDPRHTDVTTVHMAPLQERV |
| | YPSEWTLTSATEEMLATFPPLQDVLSQLAKSFTSLETYVPSTVVRYLTAGN |
| | NPRNLMPVSCGVVMLATDICSFTSLTEKSSLTEVWMICNTFIDACTSAICQE |
| | GGEVIKLIGDCVTAYFPGNNADSAVAAAQELFTFCRQLREAFVDVLDVRG |
| | CVSCGVGLDYGQVVMAQCGSLGLTEYVVAGAVSARVMEVEAITREVGY |
| | AIVVTEPVADRLSPQLRDHGIVPTPQAIEGLPCYGIAGEEFELDVDSIKRGIK |
| | ALHAARSGEKPLTEPEEAKPDFRVSPGRVRHGDSGRRSNSAQGKRSIQVR |
| Photoactivated | MYILVWKEGQQIRTFQDLEECGQFQTASNITDGQIFSINVTPTMSKGGETG |
| adenylate | ETQLRRLMYLSASTEPEKCNAEYLADMAHVATLRNKQIGVSGFLLYSSPFF |
| cyclase subunit | FQVIEGTDEDLDFLFAKISADPRHERCIVLANGPCTGRMYGEWHMKDSHI |
| alpha, 4.6.1.1, | DNITKHPAIKTILFQIARSFSSMWSYLPKNAANMLLLGKNPNKQAPEPMSV |
| Photoactivated | VVTFIYLVEFSSILAHPGLTEQCADILAAFVDACVRNVEGTGGQVAKFITGI |
| adenylyl cyclase | CMAYWPINRAEDALVGLQQLSEDLAELRSQQPPGSALSLIYSRCGVHYGR |
| subunit alpha | ALLCNAGFRKADFTLLGDCINTASRITSLSVKLKVPLLLSFEVRCLLGDEM |
| pacA | REELESSGLHKVKGRDKPVQVYQFNAPELDSAMVRAKIEQFNPGRYRALC |
| Euglena gracilis | PVKPYESLHPAQRPPIFDDTPRENQPKLSQVQRRDSLVDRLSLIAKLAFPSS |
| 1,019 AA | MMAGGEGQLITLTYISQAAHPMSRLDLASIQRIAFARNESSNITGSLLYVSG |
| SEQ ID NO: 4 | LFVQTLEGPKGAVVSLYLKIRQDKRHKDVVAVFMAPIDERVYGSPLDMTS |
| | ATEEMLATFPPLQDVLSQLAKSFISLETYVPSTVVRYLTAGNNPRNLQPVS |
| | VEVVMLATDICSFTPLSEKCSLTEVWTICNTFIDACTSAICNEGGEVIKLIGD |
| | CVTAYFPPTGADNAVHACQEIVSFCAQLRDAFHDVLDCRSVVACGVGLDF |
| | GQVIMAQCGSLGMTEFVVAGEVSARVMEVEALTREAGRAIVITEPVADRL |
| | SPKLRDTGIVPCQEGVDGVPCYGILGPEWELDVATIKKNIYGFHDARALAA |
| | MKKVDDGTNAPGRGAPAGGIPSSPKVRPPGRTNSVSSYTPDPNEALDPRM |
| | AESVFLDMCHQRGDTANNSIAVKLRQAANDDRLDLGRMLQGPHELMPV |
| | MQAIKHLTNLRMLNMSDNFVDDNNVGELVESCIPMRSLQVLDLSNNPGLT |
| | KVIALKRLIKHNTQVREILLNGTRIAPTEQRKLQSSMNVNRLCASTDLKGS |
| | HKYEH |
| Photoactivated | MYILVWKRGQQIKTFHTLDEAANFKAASNIDEAQMFSITVAPAISASGGSN |
| adenylate | EATNLRRLMYLSKSTNPEECNPQFLAEMARVATLRNREIGVSGFLMYSSPF |
| cyclase subunit | FFQVIEGTDEDLDFLFAKISADPRHERCIVLANGPCTGRMYGDWHMKDSHI |
| beta, 4.6.1.1, | DSITTHPAMKTILYQIARSFSSMWSYLPKSAGNMLLLGKDPAAQPPEPMSV |
| Photoactivated | VVTFIYLVEFGSILSNPNLTEQAAEVLSTFVDVCVKNVEGSGGNIAKFITGI |
| adenylyl cyclase | CMAYWPINRTEDALNAIQQISEDLAQLRSQQAPGSAISLMYSQAGVHYGR |
| subunit beta | AMLCNAGSRKSDFTLLGDCINTTSRIATLAKKLKTPLLFSFEVRCLLGDEM |
| pacB | REEVEGAGMHQVKGRDKPVVVYQFPGPELDVEMVRQKIEHFTPGRFRCQ |
| Euglena longa | MPVVEYESLPISQRPPIFDDTPKGKPRPRTPGYGGRQRSDSLVDRLIMIAKL |
| (Euglenophycean | AGPSISATGDSTLTTLTYISQATRPMSRLDLSAIMRTATRRNAQLSITGTLL |
| alga) (Astasia | YVNGLFVQTLEGPKDAMVNLYLKIRQDPRHTDVVTVHMAPIQERAYPAE |
| longa) | WTLTSATEEMLATFPPLQDVLSQLAKSFTSLETYVPSTVVRYLTAGNNPRN |
| 860 AA | LMPVSCGVVMLATDICSFTSLTEKSSLTEVWMICNTFIDACTSAICQEGGE |
| SEQ ID NO: 5 | VIKLIGDCVTAYFPGNGADSAVAAAQELFTFCRQLREAFVDVLDVRGCVS |
| | CGVGLDYGQVVMAQCGSLGLTEYVVAGAVSARVMEVESITREVGYAIVI |
| | TEPVADRLSPQLRDHGIIPTPQAIEGLPCYGIAGEEFELDVDSIKRGIKALHA |
| | ARSGEKPLALEPEEAKQDYRVSPGRMRHGDSGRRSNSAQGKRSTQVR |
| Photoactivated | MYILVWKEGQQIKTFQDLEECGQFQIASNITDGQIFSVSVTPTMSKGGETG |
| adenylate | ETQLRRLMYLSASTEPEKCNAEYLADMAHVATLRNKQIGVSGFLLYSSPFF |
| cyclase subunit | FQVIEGTDEDLDFLFAKISSDPRHERCIVLANGPCTGRMYGEWHMKDSHID |
| alpha, 4.6.1.1, | NITKHPAIKTILFQIARSFSSMWSYLPKNAANMLLLGKNPNKQAPEPMSVV |
| Photoactivated | VTFIYLVEFSSILAHPGLTEQCADILAAFVDACVRNVEGTGGQVAKFITGIC |
| adenylyl cyclase | MAYWPINRAEDALVGLQQLSEDLAELRSQQPPGSALSLIYSRCGVHYGRA |
| subunit alpha | LLCNAGFRKADFTLLGDCINTASRITSLSVKLKVPLLLSFEVRCLLGDEMRE |
| pacA | ELESAGLHKVKGRDKPVQVYQFNAPELDSAVVRTKIEQFNPGRYRALCPV |
| Euglena longa | KPYDSLHPAQRPPIFDDTPRDSQPKVNQMQRRDSLVDRLSMIAKLAFPSSM |
| (Euglenophycean | MVGGESQLITLTYISQAAHPMSRLDLSSIQRVSFSRNESSNITGSLLYVNGL |
| alga) (Astasia | FVQTLEGPKSAVVSLYLKIRQDKRHKDVVAVFMAPIEERVYGSPLDMTAA |
| longa) | TEEMLATFPPLQDVLSQLAKSFISLETYVPSTVVRYLTAGNNPRNLQPVSV |
| 1,019 AA | EVVMLATDICSFTPLSERCSLTEVWTICNTFIDACTSAICNEGGEVIKLIGDC |
| SEQ ID NO: 6 | VTAYFPPTAADNAVHACQEIVSFCAQLRDAFRDVLDCRSVVACGVGLDFG |
| | QVIMAQCGSLGMTEFVVAGEVSARVMEVEALTREAGRAIVITEPVADRLS |
| | PKLRDTGIVPCQEGVDGVPCYGILGVEWELDVAIIKKNIYGFHEARAQAAL |
| | KKVDDGTNAPGRGAPAAGVPSSPKARALGRTSSVSSYTPDLNETLDPRMA |
| | ESVENDLCNQRGDAPNNSIAAKLRQAANDDRLDLGRMLQGPHELMPVLQ |
| | AIKQLTNLRMLNMSDNFVDDNNVGELAESCIPMRSLQVLDLSNNPGLTKV |
| | IALKRLIKHNTQIREILLNGTRIAPMEQRKLQSSMNVNRMCASTDSKSSHK |
| | YDH |
| Photoactivated | MYNRVYKERQEIRTIQGLEASGQFQTASNITDIQIYSINVTPTMSKGGETGE |
| adenylate | TQLRRLMYLSASTEPEKCNAEYLADMAHVATLRNKAIGVIRFLLYSSPFFF |
| cyclase subunit | QVIEGTDEDLDFLFAKISADPRHERCIVLANGPCTGRMYGEWHMKDSHID |
| alpha-like | NITNNPAIKTILFQIARSFTSMWSYLPKNAANMLLLGKNPNKQGPEPRSVV |
| protein ST- | LTFIYLVEFSSILAHPGLTEQCADILAAFVEASVRNCEGTWGLVAKFITGTC |
| pacA | VAYWPINRAEDALVGLQQLSEDLAVLRSYQPPGSALSRIYSRRGVHYGRA |
| Euglena gracilis | LLCNAGFRKADFTLLGDCINTASRITSLSVKLKVPLLLSFEVRCLLGDEMRE |
| 1,019 AA | ELERSALHKVKGRDKPVQVYQFNAPELDSALVRAKIEQFNPGRYRALCPV |
| SEQ ID NO: 7 | KPYESLHPAQRPPIFDDTPRENQPKLSQVQRRDSLVDRLSLIAKLAFPSSMM |
| | AGGEGQLITLTYISQAAHPMSRFDLASIQRIAFARNESSNITGSLLYVSGLFV |
| | QTLDDPKGAVESLYLKIRQDKRHKDVVAVFMAPIDERVYGTPLDMTSATE |
| | EMLATFPPLQDVLSQLAKSFISLETYVPSTVVRYLTAGNNPRNLQPVSVEV |
| | VMLATDICSFTPLSEKCSLTEVWTICNTFIDACTSAICNEGGEVIKLIGDCVT |
| | AYFAPTGADNAVHACQEIVSFSAQLRDAFHDVLDCRSVVACGVGLDFGQ |
| | VIMAQCGSLRLTEFVVAGEVSARVMAVEALTREAGRAIVITEPVSDRLSAK |
| | LRDTGIVPCQEGLHAVLLWYPGPRMGIGCANHQEDHLRVPRRPGPGRHEE |
| | GGRRHQRPGPGRPRRGHPFLPQGSTTRPHQFSVQLHPLPKLGTGSSDGGIG |
| | VSLHVYSAGRYRQQLHRRQIAAGGQRRSVGPGQDVARATRAHACDAGH |
| | QAPDQPANAQYERQFRGGQQRWRTCGIVHSNAFVTGVGLVQQPWLDEG |
| | HRPEAPHQSLHPGPGDPSEQHPHRPNRATEPGLLVARQPAVCLHRPEGFAQ |
| | ICTF |
| Photoactivated | MYILVWKKGQQIKTFHTLDEAAQFKAASNIDEAQMFSVTVAPAISASGGS |
| adenylate | NEATNLRRLMYLSKSTNPEECNPQFLAEMARVATIRNREIGVSGFLMYSSP |
| cyclase subunit | FSFQVIEGTDEDLDFLFAKISADPRHERCIVLANGPCTGRMYGDWHMKDS |
| beta-like protein | HMDSITTHPAMKTILYQIARSFSSMWSYLPKSAGNMLLLGKDPAAQPPEP |
| FB | MSVVVTFIYLVEFGSILSNPNLTDQAAEVLSTFVDVCVKNVEGSGGNIAKFI |
| pacB | TGICMAYWPINRTEEALTAIQQISEDLAQLRSQQAPGSAVSLMYSQAGVH |
| Euglena gracilis | YGRPMLCNAGRRKSDFTLLGDCINTTSRIATLAKKFKTPLLFSQEVRCLLR |
| 859 AA | DEMREEIVGAGMHQVKGRDKPVVVYQFPGPELDVEMVRQKIEQFTPGRF |
| SEQ ID NO: 8 | RCQMPVVEYEGLPNSQRPPIFDDTPKANRRPRTPGYGGRQRSDSQVDRPIM |
| | IAKLAGPSVSATGDTTLTTLTYISQATRPMSRQDLSAIMRTATRRNAQQSIT |
| | GTLLHVNGLFVQTLEGPKDAVANLYLRVRQDPRQTDVTTVHMAPLQERV |
| | YPSEWTLTSATAEMLATFPPLQDVLAQLAKSFTSLETYVPSTVVRHLTAGN |
| | NPRNLMPVSCGVVMLATDICSFTSLTEKSSLTEVWMICNTFIDACTSAICQ |
| | DGREVIKLIGDCVTAYSPGNNADSAVAAAQELFTFCRQLREAFVDVLDVR |
| | GCVSCGVGLEYGQVVMAQCGSMGLTEYVVAGAVSARVMEVEAITREVG |
| | YAIVVTEPVADRLSPQLRDHGIVPTPQAIEGLPCYGIAGEEFELDVDSIKRGI |
| | KALHAARSGEKPLTEPEAAKPDFRVSPGRVRHGDSGRRSNSAQGKRSIQV |
| | R |
| Photoactivated | MYILVWKEGQQIRTFQDLEECGQFQTASNITDGQIFSINVTPTMSKGGETG |
| adenylate | ETQLRRLMYLSASTEPEKCYAEYLADMAHVATLRNKQIGVSGFLLYSSPFF |
| cyclase subunit | FEVIEGTDEELDFLFAKISADPRHVRCIVLANGPCTGRLYGEWHMKDSHID |
| alpha-like | NITKHPAIKTILFQIARSFSCMWSYLPKNAANMLLLGKNPNKQAPEPMSVV |
| protein FB | VTFIYLVEFSSILAHPGQTEQCADILAAFVDACVRNVEGTGAQVAKFITGIC |
| pacA | IAYWPINRAEDALIGLQQLSEDLAELRSQQRPGSALSLIYSRCGVHYGRAL |
| Euglena gracilis | LCNAGFRKADFTLLGDCINTASRITSLSVKLKVPLLLSFEVRCVLGDEMRE |
| 1,019 AA | VLETSGLHKVKGRDQPVQVYQFNARELDSAMVRAKIEQLNPGSYRALCP |
| SEQ ID NO: 9 | VKPYESLLPAQRPPIFDDTPRENQPKLSQVQRRDSLVDRLSLIAKLAFPTSM |
| | MAGGEGQLITLTYISQAAHPMTRLDLASIQRIAFARNESSNITGSLLHVSGL |
| | FVQTLEGPKGAVVSLYLKIRQDKRHKDVVADFMAPSEGREYGSPLDMTS |
| | ATEEMLTSFPPLQELLTHLAKPFISLETYVPTTVVRYLTAGNNPRNLQPVSV |
| | EVVLLATDICSFTPLSEKCSLTEVWTICKTIFVAFIRAIFNEGGEVIKLIGDCV |
| | TAYFPPTGADKAVHACQEIVTFCAQLGDAFHDVLDCRSVVACGVGLDFG |
| | QVIMAQCGSLGMTEFVVAGEVSARVMEVVALTREAGRAIVITEPLPDRLA |
| | PKLRDTGIIPCQEGVDGVPCYGNLGPEWELDVATIKKNIYGFHDARALAA |
| | MKKVDVGTNAPGRGAPAGGIPSSPKVRPPGRTNSVSSYTPDPNEALDPPM |
| | AESVFLDMCHQRGDTANNSIAVKLRQAANDDRLDLGRMLQGPHELMPV |
| | MQAIKHLTNLRMLNMSDNFVVDNNVGELVESCIPMRSLQVLDLSNNPGLT |
| | KVIALKRLIKHNTQVREFLLNGTRIAPTEQRKLQSSMNVNRLCASTDLKGS |
| | HKYEH |
| Photoactivated | MYILVWKEGQQIRTFQDLEESVQFRTASNITDGQIFSINVTPTMGKGGETG |
| adenylate | ETQLRRLMYLSASTEPENCNADYVGDMAHFATLRNKSIGVSGFYMYSAPF |
| cyclase subunit | LFQVIQGTDEDLDFLCANGSADPRHQRCIELADGPTTGRMYGWWHLKDS |
| alpha-like | HIDNITKDPAIKTILIEIARSFSSMWSYLPRNAANMVLLGKNPNKQAPEPMS |
| protein 1224- | VVVTVIYLVEFSVIMAHPGLSEQCPDILPAFVDACVPHVDGTGGEVAKFIIA |
| 5/1F | ICMAYWPINSDEHALVGLQQLSDDLAELRSLQEDGSARSLIYSRCGLHFGR |
| pacA | AMLCNAGFRKADFTLLGDWINSASRITSLSVNSKVPLLLSFLVRCLLGDEM |
| Euglena gracilis | RVELERSGLHKVGGRVKPVQVYQFNAPELDTATVRGEIKQFNPGRERALC |
| 1,019 AA | PVKPYESLHPAHPDPIFDDTPRENQPELSQVQRRDSLVDRLSQIAKLAFPSS |
| SEQ ID NO: 10 | MMAGGEGQLITLTYISQAAHPVSRLDLASIQRIAFARNESSNITKSLLYVSG |
| | LFVQTLEGTKGAVVSLYLKNRQDKRHKDVVAAFMAPIDEGVYGSPLDMT |
| | SATEEMLATFTPLHDVLSQLAKSFISLETYVPSTVVRHLAAGNNPRNLQPV |
| | SVEVVMLATAISSFTPLSEKCSLTEVWTICNTFIVACTSAICNEGGEVIKLIG |
| | DCVPAYFPPTNADNAVHACQEIVSFCAQLRAAFHDVLDCRSVVACGVGL |
| | DYGQVIMVQCGSLGMTEFVVAGEVRARVLEVEALTREAGRAIVITEPVPE |
| | RQSPKLRDTGIVPCQEGVDGARCYGILGPEWELDVATIKKNINGFLKDARA |
| | LAANKKVDDGTNISCRGGNPPAGGIPTSPKVRPPGRINSVSSYTPDPKQAL |
| | DPRMAESVFLDMCHQGDTVNNSIAVKLGLAANEDGFDGGWILTWYVEL |
| | MPVKQAIKVLTQLRIGNMSDNFVDDNNVGELLEKCIPTRSLQVLDLSNNP |
| | GLTKVIALKPKHNTQVREILLNGTRIAPTEQRKQQTSKNVNRLCASTDLKG |
| | SHKYEH |
| Photoactivated | MYILVWKKGQQIKTFHTLDEAAQFKAASNIDEAQMFSVTVASAISASGGS |
| adenylate | TEATNLRRLMYLSKSTNPEECNPQFLAEMARVAFIRNREIRVSGFLMYSSP |
| cyclase subunit | FFIQVIVETDEDLDFLFAKISADPRHEMCIVLANGPGTGRMYGDWHMKDS |
| beta-like protein | HLDSITTHTAMKTILYQIARSFSSMWIYLQKSAANMLMLGKDPAAQPPER |
| ST- | RSVVVTFIYLVEFCSIFSHPNLTEKTADVLSTFVYVCVKNVEGSGGNIAKFI |
| pacB | PAICMTYWPIIRTKEALTAIQQISEDLAQLRSQQAQGSAVSLRYSQAGVHY |
| Euglena gracilis | GRGMQCNAGSRKSDFTLLGDCINTTSRIATLAKKLKTPLLFGFEVRCLLGD |
| 859 AA | EKREEIEGAGIHQVKGRDKPVVVYQFPGPDLDVAMVRQKIEQFTPGRFRC |
| SEQ ID NO: 11 | QMPVVEYEALPISQRPPIFDDTPKSNPRPRTPGYGGRQRSDSLVDRLIRIAN |
| | LSGFSESATGDTTLTTLTYISQATRPMSRLDLSAIMPTATRRNAQQSNIGTV |
| | LHENGLFVQTLEGPKDAVVNLYLRIRQDPRHTDVTTVHMAPLQERVYTSE |
| | WTLTLATEEMLATFPPVQDVLSQLAKSYTSLETYVPSTVVRYLTAGNNPR |
| | NLMPVSCGVVMLATDISSFTSLTEISSFTEVWMICNTFIDACTSAICQEGGE |
| | VIKLIGDCVTPYFPGNNADLAVAAAQELFIFCRQLREAFVDVLDVRGCVSC |
| | AVGMDYGQVVMAQCGLLRLSDYVAAGAVSAGVVEVEAITREVGYQFVV |
| | TVPDADRVQPQFRDYGIEPTPQAIGGLPCYGIVREVFDLPVESIKRGIKAMH |
| | AARSGEQPLTEPEQAKPDFRVSPGRDRHGVSGRRSNSSQGKGSIQVG |
| Putative alpha | MYVLVWKNGQQVKTFETLDGANSYKTGSNIADDDSNIFHIDVIPAQSAGS |
| subunit of | AGGGNVDKPLRRLMYLSKTTEAELCTPEYLADMAHVATLRNMEIGVSGF |
| photoactivated | LLYSAPFFFQVIEGTDEDLDFLFAKIGADPRHERCIVLANGPCTGRLYGDW |
| adenylyl | HMKDSHIENITSHPAIKTILYQIARSFSSMWSYLPKNAANLLLLGKNPNKQT |
| cyclase, 4.6.1.1 | PEPTSVVVTFIYLVEFSSILAHPGLTEQVADVLSHFVDICVRNIEGSGGAIAK |
| Eutreptiella | FITGITMAYWPINRAEDALLALQQINEDLAQLRSQQPPGSALSLMYGRAGV |
| gymnastica | HYGRALLCNAGVRKSDFTLLGDCINTASRVTSLATQLKSPLLFSFEVRCLL |
| 1,015 AA | GDDMRDELESAGMHKVKGRDKPVAVYQFPCEPLDNDMVRAKIEAFNPG |
| SEQ ID NO: 12 | RYRAQFPVVPYDSLHGVHKPPIFDDSPKDPLATRIAQAAGERKDSLIDRLI |
| | MISNLAIPASMAGGGNNQLITLTYISHAVKPMSRLDLAAINRNAVRRNAAS |
| | KITGTLLYVNGLFVGTLEGPKDAVLTLYNKVRRDARHEDVVTVHLAPLDE |
| | RVYDVPFSVTAATDSMLSKFPPLQDVLSQLAKSFISLETYVPSTVVRWLTA |
| | GNNPRGLHAVSTEVVMFATDICSFTPLSENCSLVEVWLICNTFIDACTHSIC |
| | NKGGEVIKLIGDCVTAYFPPDGADAAVLACQEVVTFCAQLREAFEEVLDC |
| | RSVVACGVGLDYGQVVMAQCGSEGMTEYVVAGEVSARVMEVEALTRD |
| | VARAIVVTEPVADRLSPAMRDNGIIPCSQGVDGIPCYGILGEEWELDIPTIK |
| | ANIKRFHATRAEIARQKEEEAAAAEAGKPKAKARADSRFGPRGSVNSYCP |
| | DPNEVLDPRMAEDVFVEACAARGDAPNLALKAKMRQAANDDRLDLARA |
| | LQGPHELSPLTTALKQLCHLRSLNMSDNFIDDNNVVDLVEACLPMENLQV |
| | LDLSNNPGITKIIALKRLVKHNVQIREINLNGTRVAPTEQRKLQSSINVNRL |
| | CASEKR |
| Putative alpha | MYILVWKEGQQIKTFQDLEECGQFQTASNISDGQTFSINVSPVMSKGGETG |
| subunit of | ETQLRRLMYLSASTEPERCDGEYLAEMAHVATLRNSEIGVSGFLLYSAPFF |
| photoactivated | FQVIEGTDEDLDFLFAKIGADPRHERCIVLANGPCTGRLYGEWHMKDSHID |
| adenylyl | NITKHPAIKTILYQIARSFSSMWSYLPKNAANMLLLGKNPNKQAPEPMSVV |
| cyclase, 4.6.1.1 | VTFIYLVEFSSILAHPGLTEQCADVLAAFVDACVRNVEGTGGQIAKFITGIC |
| Colacium | MAYWPINRAEDALVGLQQLSEDLAELRSQQPPGSALSLVYSRCGVHYGR |
| sideropus | ALLCNAGTRKSDFTLLGDCINTASRITSLSVKLNVPLLLSFEVRCLLGDEMR |
| 1,017 AA | EELESAGLHKVKGRDKPVQVYQFNAPELDSAQVRAKIESFNPGRFRPMCP |
| SEQ ID NO: 13 | VKPYDSLHPSQRPPIFDDTPRESMPKLSVVQRRDSLVDRLSLIAKLAFPPNM |
| | MNGGDNQLITLTYISQASYPMSRLDLAAIQRVGCRRNESAQITGSLIYVNG |
| | LFVQTLEGPKSAVITLYLKIRQDSRHKDVVIVFMAPIEERVYSTALDMTSA |
| | TEEMLATFPPLQDVLSQLAKSFISLETYVPSTVVRYLTAGNNPRSLQPVSVE |
| | VVMLATDICSFTPLSENCSLTEVWTICNTFIDACTSAICSEGGEVIKLIGDCV |
| | TAYFPPTSADNAVAACQEIVTFCTQLREAFSDVLDCRSVIACGVGLDYGQ |
| | VVMAQCGSLGMTEFVVAGEVSARVMEVEALTREAGFTIVVTEPVADRLS |
| | PKLRDTGIIPCHEGVDGVPCYGIAGDEWVLDIKTIKANIYGFHEARAQAAL |
| | RKVDDGTRAPGHAAPASPKAQKGAGRANSVASYTPDVNELLDPRMAESV |
| | FQELCQQRGDVPNVGISAKLRQAAQDDRLDLGRTLQGPHELGPVVQAVK |
| | QLTNLKTLNLSDNFVDDGSVGEIVEACLPMRNLQVLDLSNNPGLTKIIALK |
| | RLIKHNPAVREILLNGTRVAPTEQRKLQSSINVNRLCSSTDAKGGSHKYES |
| | H |
| Putative alpha | MYILVWKDGQQVKNFQDLEECGAFQTAANIVDGQIFNVGVTSAMNKGG |
| subunit of | DTGEVQLRRLMYLSASTEPEECTTEYLAELARASSLRNKEIGVSGFLLYSA |
| photoactivated | PFFFQVIEGTDEDLDFLFAKISSDPRHEKCIVLANGPCTGRLYGEWHMKDS |
| adenylyl | HIDNITKHPAIKTILFQIAKSFSSMWSYLSKNTANMLLLGKQPNKQAPEPMS |
| cyclase, 4.6.1.1 | VVISFIYLVEFSSILAHPGLTEQCADVLAAFVDVCVRNIEGTGGHVAKFITGI |
| Euglena | CMAYWPSNRAEDALVGLQQLSDDLAELRSQQPPGSALSLVYSRCGVHYG |
| quartana | RALLCNAGIKKADFTLLGDCINTASRITSLAGKLKVPLLLSFEVRCLLGAE |
| 1,027 AA | MREELESAGMHKVKGREQPVQVFYFNAAEVDSTAVRAKIEQFNPGRYRA |
| SEQ ID NO: 14 | LCPVKPYDSLHPSQKPPIFDDTPRDSMPRSGHLQRQDSLVDRLSMIAKLAF |
| | PPSMAAGGESQLIGLTYISQASHAMSRLDLSDIQRVAHRRNEQVNISACLL |
| | YINGLFVQTLEGPKSAVVSLYLKIRQDPRHKDVVVVHMAPLEERMYRGPL |
| | DTTCATDEMLATFPPLQDVLSQLAKSFISLETYVPSTIVRYLTSGNNPRNLQ |
| | PVSLEVVMLATDICSFTPLSEHCSLTEIWTICNTFIDACTSAICSQGGEVIKLI |
| | GDCVTAYFPPTSADNAVYACQDIVAFCANLREAFRDVLDCRSVVACGVG |
| | LDFGQVIMAQCGSSGLTEFAVAGEVSARVMEVEALTREAGRAIVLTEPVA |
| | DHLSPKLRDNGLVPCQEGVDGVPCYGILGAEWELDVVTIKRNIQNFHAAR |
| | AVAAVQKVDDGMNAPGRALPVASMPAISSPSPKVRTAGRAASVASYAPD |
| | LNDLLDPRMAEVVFQELCQQRGEIPNNAVLLRLRQAAHEERADLGRSLQG |
| | PHELPPLTQALKQLTNVRALNMSDNFVDDTSIGDLIESCLAMRSLQMLDLS |
| | NNPGLTKVIALKRLIKHNPQLREIHLNGTRIAPTEQRKLQSSLNVNRLCASA |
| | ESKLGSHKYEGAAH |
| Putative alpha | MYILVWKEGQQIKTFQDLEECGQFQTACNISDGQIFSVNVAPTMSKGGES |
| subunit of | GEQLLRRLMYLSKSTEPEACNAEYLAQMAFVATCVNSTIGVSGFLLYSAPF |
| photoactivated | FFQVIEGTDDDLDWLYAKISRDPRHERCIVLANGPCTGRMYGEWHMKDS |
| adenylyl | HIDNITKHPAIKTILFQIARSFSSMWSYLPKNAANMLLLGKEPNKQAPEPMS |
| cyclase, 4.6.1.1 | VVITFIYLVEFSSILAHPGLTEQCADILEAFVDACVRNIEGTGGQVAKFITGI |
| Euglena stellata | CMAYWPINRAEDAIVGLQQLSEDLAELRSQQPPGSALSLVYSRCGVHYGR |
| 1,017 AA | ALLCNAGFRKADFTLLGDCINTASRITSLSVKLKVPLLLSFEVRCLLGDEM |
| SEQ ID NO: 15 | REELENSGLHKVKGRDKPVQVYQFNAPELDSAMVRAKIEQFNPGRYRAL |
| | CPVKPYDSLSATQKPPIFDDTMRDGHNKLSAAQRRDSLVDRLSLIAKLAFP |
| | PSMLAGGDGQLITLTYISQASRPMSRLDLSAIHRVAMRRNESSNISGCLLY |
| | VNGLFVQTLEGPKSAVVSLYLKIRQDGRHKDVVTVYMAPLEERVYATAL |
| | DMTAATEDMLSTFPPLQDVLSQLARSFISLETYVPSTIVRFLTAGNNPRNLQ |
| | PVSVDVVMLATDICSFTPLSERCSLTEVWTICNTFIDACTSAICNEGGEVIKL |
| | IGDCVTAYFPPHAADNAVSACQEIVTFCTHLREAFKDVLDCRSVIACGVGL |
| | DFGQVIMAQCGSLGMTEFVVAGEVSARVMEVEALTREAGRAIVVTEPVA |
| | DRLSSRLRDSGIVPCQEGVDGVPCYGILGPEYELDIVTVKANIYGFHDARA |
| | QAALRKADDGTNAPGKSGPAVPSSPKVHHAQGRAGSVASYTPDPNEVLD |
| | PRMAEMVFQDMCQHRGDQPSNAILMKLRNAAQDDRLDLGRTLQGPHEL |
| | GPLTQAIKQLTNLKMVNMSDNFIDDNTIGDLVEACLSMRSLQVLDLSNNP |
| | GLTKTIALKRLIKHNPHVREVLLNGTRIAPTEQRKLQSSMNVNRLTASDKG |
| | GSHKYDH |
| Putative beta | MYILVWKKGQQIKTFHTLDEAAQFKAASNIDDAQMFSINVAPAVSASGGS |
| subunit of | NEATNLRRLMYLSKSTEPEKCDVPYLAEMAGIATLRNREIGVSGFLMYSSP |
| photoactivated | FFFQVIEGTDEDLDFLFAKIGADPRHERCIVLANGPCTGRMYGDWHMKDS |
| adenylyl | HIDSITTHPAMKTILYQIARSFSSMWSYLPKSAGNMLLLGKDPASQPPEPMS |
| cyclase, 4.6.1.1 | VVVTFIYLVEFGSILSNPNLTEQAAEVLSTFVDVCVKNVEGSGGNIAKFITG |
| Colacium | ICMAYWPINRAEDALTSIQQISEDLAQLRSQQAPGSAVSLMYSQAGVHYG |
| sideropus | RAMLCNAGTRKSDFTLLGDCINTTSRVATLAKKLKTPLLFSFEVRCLLGDE |
| 860 AA | MREEIEGAGMHQVKGRDKPVVVYQFPGEELDVDEVRHKIETFNAGRFRC |
| SEQ ID NO: 16 | QMPVVEYDTLHPSQRPPIFDETPKGNPRPRTPGYGGTQRRDSLVDRLIMIA |
| | KLAGTTASTTGDTTLITLTYISQASRPMSRLDLSAIMRASTRRNTQQNITGT |
| | LLHVNGLFVQTLEGPRDAVMNLYLRIRQDPRHTDVVTVHMANLQERVYP |
| | PEWTLTSASEDMLAAFPPLQDVMSQLAKSFTSLETYVPSTVVRYLTAGNN |
| | PRNLMPISCGVVMLATDICSFTSLTEKSSLTEVWMICNTFIDACTSAICQEG |
| | GEVIKLIGDCVTAYFPGSAADAAVAAAQELYLFCTQLREAFVDVLDVRGC |
| | VSCGVGLDYGQVVMAQCGTLGLTEYVVAGAVSARVMEVEALTREVGHA |
| | IVVTEPVADRLSPQLRDSGIIPCPQAIEGIPCYGIAGEQFELDIDSIKRGIKAL |
| | HAARSGEKVAVAEPEEKGGDFRVSPGRVRRSDSGRRSNSAQGKRGTPVR |
| Putative beta | MFLLVWKKGQQMATFHTLEESNEFKAASNIEDSHVFHLKLTPTTSGAGGA |
| subunit of | NNATGFRRLMYLSKSTEAEEVNSTYLAEMAGIATIRNQEIGVSGFLMYSDP |
| photoactivated | FFFQVIEGTDEDLDFLFAKIGADPRHERCIVLANGPCTGRLYGDWHMKDS |
| adenylyl | HIDSITSHPAMKTILYQIARSFSSMWSYLPKSAGHMLLLGKDPNKQPPEPM |
| cyclase, 4.6.1.1 | SVVVTFIYLVEFSSILSHPGLTEQCAEVLSTFVDVCVRNVEGSGGNIAKFIT |
| Eutreptiella | GICMAYWPINRAEDALTALQQINEDLAQLRGAQAPGSALSLMYSRSGVHY |
| gymnastica | GRAMLCNAGTRKSDFTLLGDCINTASRVASLAVTLKSPLLFSFEVRCLLGD |
| 862 AA | EMRDEIESAGMHQVKGRDKPVVVYQFPSPILDSDVVRQKIEQFNPGRYRC |
| SEQ ID NO: 17 | SMPVLDYETMNPAKRPPIFDNAPKGNPKIGKKYNAAAHRDSLVDRLTEIA |
| | RLAFPGSQAGGDSTLISLTYLSNSVRPMSRLDLAAIQRTALRRNASQGITGC |
| | LLYVNGLFVQTIEGPRDNVINLYLKIREDPRHKDVVTVYMVPIQDRVYQE |
| | AWTMTTASEDMLATFPPLQDVLSQLAKSFTSLETYVPSTVVRYLTAGNNP |
| | RDLMPVSVECVMFATDICSFTMLTEKSSLEEVWMICNTFIDACTSAICAEG |
| | GEVIKLIGDCVTAYFPSERADNAVAASQELYMFCVHMREAFQEILDVRACI |
| | ACGVGLDYGQVVMAQAGSLGLTEYAVAGAVSARVMEVEALSRDVGRAI |
| | VCTEPVVDRLSPHLRDSGVIPCPQAIEGIPCYGIAGEDYELDIRSIKAGIKQL |
| | HAARSLEDQAAAAEEEAAQAQQQFRVSPGRVRRSDAGRRSNSAQGRRMA |
| | PVR |
| Putative beta | MYILVWKKGQQIKSFHTLDEAAEFKAASNIDEAQMFSITVAPAISASGGTN |
| subunit of | EATNLRRLMYLSKSTNPEECDPAFLAEMAGIATIRNKEIGVSGFLMYSSPFF |
| photoactivated | FQVIEGTDEDLDFLFAKISADPRHEKCIVLANGPCTGRMYGDWHMKDSHI |
| adenylyl | DSITTHPAMKTILYQIARSFSSMWSYLPKSAGNMLLLGKDPAAQPPEPMSV |
| cyclase, 4.6.1.1 | IVTFIYLVEFGSILSNPDLTEQAAEVLGTFVDVCVKNVEGSGGNIAKFITGIC |
| Euglena stellate | MAYWPINRAEDALTSIQQISEDLAQLRSQQAPGSAITLMYSQAGVHYGRA |
| 861 AA | MLCNAGTRKSDFTLLGDCINTTSRVATLAKKLKTPLLFSFEVRCMLGDEM |
| SEQ ID NO: 18 | REEIEGAGMHQVKGRVKPVVVYQFPGPELDVEMVRQKIDQFNPGRFRCQ |
| | MPVVDYDSLPVSQRPPIFDDTPKGNPRPRTPGYGGTQRSDSLVDRLIMIAK |
| | LAGPSASAGGDTSLTTLTYISLATRPMSRLDLSAIMRTATRRNSSQGITGTL |
| | LYINGLFVQTLEGPKDAVVNLYLRIRQDPRHTAVVTVHQAALQERVYPPE |
| | WTMTSATEEMLATFPPLQDVLSQLAKSFTSLETYVPSTVVRYLTAGNNPR |
| | NLMPVSCSVVMLATDICSFTSLTEKSSLTEVWMICNTFIDACTSAICQEGGE |
| | VIKLIGDCVTAYFPGNAADAAIAAAQELFMFCTTLRQAFVDVLDVRGCVA |
| | CGVGLDYGQVVMGQCGTMGLTEFVVAGAVSARVMEVEALTREVGRAIV |
| | VTEPVADRLSPQMRDTGIIPCPQAIEGLPCYGIAGDDYELDIDEIKRSIKHLH |
| | AARSGEKMPSADHEENKGNDFRVSPGRVRRSDSGRRSNSAQGKRGTPVR |
| Putative beta | MVFLLVWKNGTQLKSFLTLDDATQYKTASSIDDAQIYNVAFTPAVSGDGG |
| subunit of | SNEATNLRRLMYLSKSTEPELVNTQYLAEMAHIATLRNQCIGVSGFLMYS |
| photoactivated | HPFFFQVIEGTDEDLDFLFAKIGIDPRHERCIVLANGPCTGRLYGDWHMKD |
| adenylyl | SHIDSITSHPAMKTILYQIARSFSSMWSYLPKSAGNTLLLGKDPAAQPPEPM |
| cyclase, 4.6.1.1 | SVVVTFIYLVEFSSILSHPGLTEQCAEVLSTFVDVCVKNVEGSGGNIAKFIT |
| Eutreptia viridis | GICMAYWPINRAEDALTSIAQISEDLGQLRGQQPPGSALSLMYSRAGVHY |
| 867 AA | GRAMLCNAGSRKSDFTLLGDCINTASRVASLAVTLKSPLLFSFEVRCLLGD |
| SEQ ID NO: 19 | EMRDEIEGAGMHSVKGKDKPVVVYQFPGPELDSGMVRQKIEEFNPGRYR |
| | CGMPVMEYESLHVSEQPPIFDDTPKGTPKPGTRAWGGTNRKDSLADKLAL |
| | ISQLAGPLQTSGDTTLTTLTYISHSTRPITRMDLASIQRCALRRNAQHNITGC |
| | LLYVTGLFVQTLEGPKDAVINLYHKIRADARHTDVVTVHQGPIQDRVYAS |
| | AWALTNTSEEMLSAFPPLQDVLSQLAKSFTSLETYVPSTIVRYLTAGNNPR |
| | NLRPVSVGVVMLATDICSFTMLTEKSSLTEVWMICNTFIDACTNAVITNGG |
| | EVIKLIGDCVTAYFPGHGADGAVAAAQEIFSFCTQLREAFADALDVRSAIS |
| | AGIGLDFGQVVMAQAGSLGVTEFAVAGSVSARVMEVEALTRDVGRAIVV |
| | TEPVADRLSPQLRDRGLIPCPQAIEGIPCYGLAGDDFELDIASIKGRIRNLHA |
| | MRAGEKPNLPDDHEQEDNYKQAQQDFRVSPGRVRRADSGRRSNSAQGRR |
| | SAPVR |
| Putative alpha | MYVLIWKSGQQIQTFPDLEAASQFKAASNIEDSFIFSVSVTPTMLAGGAAG |
| subunit of | ESGLRRLMYLSKATEPDECTPEFLAELAHVATLRNQEIGVSGFLLYSAPFFF |
| photoactivated | QVIEGTDEDLDFLFGKIGLDTRHEACIVLANGPCSGRMYGEWHMKDSHID |
| adenylyl | NITKHPAIKTILYQIARSFSSMWAYLPKQAGNLLLLGKEPNKQPPEPMSAV |
| cyclase, 4.6.1.1 | VSFIYLVEFSTILANPNLTEQVADILIAFVDSCVKNVEGSGGSVAKFITGICM |
| Eutreptia viridis | AYWPINRAEDALLGLQAISDDLAELRAHQPPGSALSLVYSRAGVHYGRAL |
| 1,027 AA | LCNAGMQKSDFTLLGDCINTASRVTSLSVQLKVPLLFSFEVRCLLGDDMR |
| SEQ ID NO: 20 | EELESLGMHKVKGRDKPVAVYQFAGAPELDSAMVKAKVEQFNPGRYRA |
| | QCPVRELESLHPDVRPPIFDDTPREASMPKAGAGERRDSLADRLTMIAKLA |
| | FPTIGGGTNSQMITLTYVSFSSRPMSRLDLAAIQRVGLRRNSQVDITGSLLY |
| | VAPLFVQTLEGPKDAVISVYMKIRADPRHRDVVAVYMAPQEERVYNSAFE |
| | LTTATEEMLSAFPPLQDVLSQLAKSFISLETYVPSTVVRYLTAGNNPRSLQA |
| | ISTGVVMFATDICSFTPLSEKCSLTEVWLICNTFIDACTAAIVQERGEVIKLI |
| | GDCVTAYFPPTNADGAVMACQEIVAFCTGLRAAFSDVLDVRQVVACGVG |
| | LDYGDVVMAQCGSMGMTEFVVAGEVSARVMEVEALTREVGRAIVITEPV |
| | ADRLSPKMRDTGIAPCPEGVDGIPCYGIVGDDWDLDIVTIKKNIYSFHDRR |
| | DAVAHAKAAAAQEALDAAAPSPKKGGRGSGRSSSVSSYAADPNEVLDPR |
| | MAEAAYTQGCQQRGEQPTMAIVNKLRGAGNDTRMDLGRALQGPHELMA |
| | LCTALKHLTHLRLLNMSDNFVDDNSIAEIVEACLPMKSLAVLDLSNNPGLT |
| | KILALKRLVKHNPGIKEIILQGTRIAPPEQRKLQSSINVNRLCASSPNGAESK |
| | AGHKYDQSMSSSHH |
| Photoactivated | MYILVWKKGQQIKTFHTLDEAAQFKAASNIDEAQVFSVTDAPDISASGGS |
| adenylate | NEARNRRRLMYLTNSTNPEECNPQFVGEMARVATIQNREIGARGFLMYSC |
| cyclase subunit | RFLLQVIEETVEDPDSLFAKFSPEPRHERWIELDNGPCTGRMYGDWNMKD |
| beta-like protein | SHLDSITTHPAMLTILFQMARSISCLWSYLQKSERIMLLFGKDPGAQRPEPM |
| 1224-5/1F | RVVVTFTYLVDFGSMLSIPNLTEQAAEVLTTFVDGCVKNVEGSGGSIAKFT |
| pacB | TGICMAQWPINRTVEALTAIQQISEDLAQLRSQQAPGSAVSLMYSQAGLH |
| Euglena gracilis | YGRGMLCNAGSRKSDFTLLDFINTTSRIATLANKLKTPLLFHSFDVRCLLG |
| 859 AA | DEMREEIEGAGMHQVKGRDKPVGVYQFPGPELDVEMVLRQKVEQFTPGR |
| SEQ ID NO: 21 | FRCQMPVVEYEALPIWQRPPIVDDTPKGYPRPRTMPGYGGRQRSDSLVDR |
| | LIMIAKLAGPSVSATGDTTLTTLTYISQATRKPMSRLHLSALMGTATRRNA |
| | QQSITGTLLDVNGLFGQTLEGPKVAVVMYLSIRQDARQTDVTTVHMAPLQ |
| | ERVCPSEWTLTSATEEMLATFPPLQDVLSQLAKSCTSLETYVRSTVGYLTA |
| | GNNRRNLMPVSCGVVMLATDIGSFTSLTEESSLTEVWMICNTFIDACTSAV |
| | CQEGGEVIKRIGDCVTAYFPGNNAESAVAAAQELFTFCRQLREAFVDVVD |
| | VRGCVSCGVGLDYSQVVMAQCGSLGLTEYVVAGVSARVMEVDAMTREV |
| | GYAIGVTEPYVTDRLSPQSRDHGIVPTPQAIELPCYGIAGEQLELDVDSIKR |
| | GIKALHAARSGEMPVTEPEEALRLFRVSPGRVRHGDTGRRSNSAQGKRSIQ |
| | VR |
| Photoactivated | MYILVWKKGQQIKTFHTLDEAAQFKAASNFDEAPMFSVTVAPAISASGGS |
| adenylate | NEATNLRRLMYLSKGTNPEECNPQFVAELARVATIRNREIGLSGFLMSSSP |
| cyclase subunit | FLFQAIEGTDQDLDFLFAKISADPRHERCIVLANRPCTGRMSGVWHMKDS |
| beta-like protein | HMDSITTHDAMMTILYQIARSFSSMLTYLPKRAGNMLLSGLDPAAQPPEPK |
| 1224-5/9F | SVVVTFIYLVAYGSLLSNPYSTEQPAEVLSTFVDVCVKNLEGSGGIITKFIT |
| pacB | GICMASWPFNRTEEALTAIQQISEDLAQLPSQQAPGSSPSLMYSQAGVHYG |
| Euglena gracilis | RAMLCNAGSGKSDFTLLGDWINTTSRIATLAKKLTSPVLFSFQVRCLLGDE |
| 855 AA | MREEIEGAVMDQVKGRDKPVVEYQFPGPERDVEVVGQKIEQFTPGRFRCQ |
| SEQ ID NO: 22 | MRVGQYEALPISQRPPIFDDTPKCNPRPRTPGCEGRQRSHTLVDPHIMMAK |
| | LPGPSVSATGDTTVPTLTYISHATRPMSRLDLSAIMGTATRINALQRITGTL |
| | LHGNGLFVQTQEGPKEAEINLLLRIRQDTRRSDVATDHMPQPQERVYPSE |
| | WTLTSGKEEILAIFPPLQDVLSRLAKSFTAQETYVPSRVVSYLTAGNNPREL |
| | MPVSCGVVMTDICSLTSLTENSCLSEVWMICNTFIDACPSAICQEGAEVIKL |
| | IGNCVTAYFPGNNADSAVASAQELFTFCREHRQAIVDVLDVRGCVSYGVA |
| | LDNGQVVMAQCGSEGLTEYVVAGAVSARVMEVEAITRGTCPRWATPLW |
| | SADRLSPQLRDHGIVPTPQAIEGLPCYGIAGEEFELDVDSIKLGIKALHAAR |
| | SGEKPLTEPEEAKLDFSPGRVRHGDSGRRSNSAQGKLSIQVR |
| Putative | MRFSVYSLYALACLLAVGGAILTGIILLDTFDGQQRNSENAQFTAGKGQV |
| membrane- | QSMQGVIANRMTRMQTITEEHSRQIMMFLQSLPSNAIQPDNVVQAVNHSIF |
| bound adenylyl | DSWVPSVKANNSLNGYGLTFTYTNETTGNSFDRTFWVYWDLMTTGVYD |
| cyclase, 4.6.1.1 | YIYAYTNMSDGYIHAYETLWPNFSTPQLGLDMYRFRSTVLAASIKDDDFFT |
| Euglena gracilis | TARVWVSADGNSYWYFTHMRTFQFQGVYANVQSWDVAISWLDLMRTG |
| 846 AA | LTPEANVVAFDSQGYVMAATNKDELARLSQCRGSFNSGNLAATCISYFAR |
| SEQ ID NO: 23 | NHPILEIRNLYNALHTPVWDDLGAAPIPLEQATLSLSGKRYMAIIATLYSKA |
| | DLRTTVVWYQPWFVLQTDTVSVTALVCILTMLSTFVLTLLGVFGVLRPLQ |
| | ALGAAMRAVAGTLKEGDGSKETVLEPRKPNVFREVDDIGADFETIVVDFL |
| | GFSNTNARDNKYAPKDPNAPFAVVFTDIQSSTALWGKNPAEMSRCVQAH |
| | HELIRELIKQHRLYEVKTVGDSFMVTTTSAQAALRFALDVQTTLFEHDWD |
| | WDGADELYQHTGVVFTKVCDGPFRSESEYGELWNGLRVRIGIHFGMGDV |
| | LYDEVSKGYDYYGNVVNAAARIEALAHGGQVVVTEALLAALPAKLDSSV |
| | GLTRLLGKFPLRGVAEPPALVEVTPTALRGRTYPLLRTELTTTESPVVGQEI |
| | QSVSSDTPKSTDRRSSGPTTASHASDRCPLQQLAEDFARTHTLVRSSVLPA |
| | EVVAQQLLTLYSIVEDLLMPLAPAQCTAVLKALAKGWGFTGPMPKTDAA |
| | HRMRLVHRMSETTQVFTRLGNVTRSHTPMHAGCTPSHSGGGFIHVELV |
| Photoactivated | MDSMQSNLPHREMARNNFGINTDGILTKKTTTNYLLNGGGEQDRAISVPT |
| adenylyl cyclase | SVVQHNEINPSEAAKTVTELSFSGNKSPSTLTQARMMKRCSKQQLLSKLPK |
| Naegleria | KPFLRIQYASHCCSGKISESVKNEILSVAQRENEIRGITGVLICVNDMFFQIIE |
| australiensis | GHTQHVETLYHNILKDKRHYDVVCVDKMYFDSESERYFPDWCMQSLDM |
| 474 AA | ESLFDHCLDPSLSTAVGAAFRAFKTLLVSLLNTQQVFRNYAQPVISKYLSE |
| SEQ ID NO: 24 | GENPMIKESIETDKVILFIDLVNYTSITEKLSKTRKPSQVVEILNKFFGCVME |
| | IVEDYCGEITKLIGDCVMVYFDGSECQEAVNCSIEILERLEILRQTSEDPFVK |
| | MIYCTIGISRGSVIFGNVGSVGRKLDFTLIGDSVNIASRLQSFASISPYFLIFD |
| | ESVKGRLDKDSCDIIEVGPIELKGKEKTVNTYTVEHELNRKPPKKEMDRIM |
| | SLASLSEYC |
| Photoactivated | MSSQTPPYIRMQYVSLSTEGNISESTKGKILATAARENPRYNITGVLICVRD |
| adenylyl cyclase | MFFQIIEGRIEDIDRLYVNIKKDKRHNNIACIDKTYFHREDERMFPSWDMK |
| Naegleria | SIEVDSLFPTKKEVVICGEYCNPLDPSHQKVIETAFSAFTTMMGSMFRAQQ |
| australiensis | VFKNYAQPSVSRFVRDGLNPLAIESVETEKVVLFIDLVNYTTLTEIISQSRKT |
| 384 AA | SEVVRILNVFFSYVMECVENFGGDITKLIGDCVMCHFDGNDCQKAVDCSL |
| SEQ ID NO: 25 | KILESLDNLRKETNDPFEKLIFCTIGISKGNVILGNVGTVGRKLDYTVIGDSV |
| | NVSARLQSFASSTEYYLIFDGNVKNGLDRVDSVVSCGSIELKGKKNAVNA |
| | YTIENVYNRRVLAEDLKAMYCTLN |
| Photoactivated | MSSSQTPPYIRMQYVSLSTEGIISESTKAKILATAARENPRYNITGVLICVRD |
| adenylyl cyclase | MFFQIIEGRIEDIDRLYVNIKKDNRHNNIACIDKTYFHREDERMFPSWDMK |
| Naegleria | SIEVDSLFPTKKEVVICGEYCNPLDPSHQKVIETAFSAFTTLMGSMFRAQQV |
| australiensis | FKNYAQPSVSRFVRDGLNPIAIESVETEKVVLFIDLVNYTTLTEIISQSRKTS |
| 385 AA | EVVRILNVFFSCVMECVEDFGGDITKLIGDCVMCHFDGNDCQTAVDCSLKI |
| SEQ ID NO: 26 | LESLDNLRKETNDPFEKLIFCTIGISKGNVILGNVGTVGRKLDYTVIGDSVN |
| | VSARLQSFASSTEYYLIFDENVKNGLDRVDSVVSCGSIELKGKKNAVNAY |
| | TIENVYNRRVLAEDLKAMYCTLN |
| Putative beta | MHVLIWKKGQQIKTFHTLDEAAQFKAASNIDDAQIFNVSVTPAISASGGSN |
| subunit of | EATNLRRLMYLSKSTNPEECTPQFLAELGRISSIRNKEIGVSGFLMYSAPFFF |
| photoactivated | QVIEGTDEDLDFLFSKISMDDRHDRCIVLANGPCTGRMYGDWHMKDSHID |
| adenylyl | SITTHPAMKTILYQIARSFSSMWSYLPKSTGNMLLLGKDPAAQPPVPMSVV |
| cyclase, 4.6.1.1 | VTFLYLVEFGTILSKPNLTEQAAEVLSTFVDVCVKNVEGSGGNIAKFITGV |
| Euglena | CMAYWPINRAEDAIQAIQQISDDLAQLRSQQPPGSAISLMYSQAGVHYGR |
| quartana | ALLCNAGTRKSDFTLLGDCINTTSRIATLAKKLKTPLLFSFEVRCLLGDEM |
| 873 AA | REEIESAGMHQVKGRDKPVVVYQFPGATLDVEVVKQKIEQFNPGRFRCQ |
| SEQ ID NO: 27 | MPVVDYDTLLPSQRPPIFDETQRDVIKPRVPGMQRRDSLADRLAMIAKLA |
| | GTTGGAAGAGDSTLVTLTYISQTVRPMSRLDLAAIMRVATRRNAQQGITG |
| | TLLHVSGLFVQTLEGPRSAVLGLYMRIRQDPRHKDVVTVHLAPISERVYPP |
| | DWTLTTATEQMLSTFPPLQDVLSQLAKSFTSLETYVPSTVVRYLTAGNNPR |
| | NLVPVSCGVVMLATDICSFTSLTEKSSLTEVWLICNTFIDACTNAICQAGGE |
| | VIKLIGDCVTAYFPGTAADNAVSAAQELITFCRELREAFVDVLDVRGCIYC |
| | GVGLDFGQVVMAQCGTLGLSEYAVAGAVSARVMEVEALTREVGRAIVLT |
| | EPLADRLCPQMQEMGIVPSPQAIDGLPCYGVAGDEFVLDVDTIKRSIKNFH |
| | AARSGEKLNVSGLANGDLEESMTGSPLRATINDFRVSPGRVRRGDSVRRSP |
| | SIAGRYGVATR |
| Beta subunit of | MKRLTYISSFSRSLSKREIEAIGHISQTNNSREGITGVLLSCNGIFFQILEGDE |
| photoactivated | DRIDRLYEKILRDDRHSNILCLKSELNVLARKFPDWSMEVIVLDENNDILL |
| adenylyl cyclase | QPIKILLQTLGESQTILANYTQSGVLQAIENGINPLTLKPLYSDKIILFADIISF |
| ABRG53 0929 | SGITEKLQVAEVVDLLNQFFTICTRAITARGGEVTKFIGDCVMAYFDGDRA |
| Pseudanabaena | DDSLQASLDILAELELLRNQAPANSPLRVLYIGIGLSKGIVLEGNIGSQTKK |
| sp. ABRG5-3 | EYTVIGDAVNVAARIEALTRQLNRFLVFSASVRAGLSDRWKPVRLGDFHT |
| 354 AA | KGRESIVEVYSVDDVLTFQKSDFHKLKQEIKQHLELISCQA |
| SEQ ID NO: 28 | MKRLTYISNFSRSLTRKEIENIEIISQKNNFREGITGILLSCSGIFFQILEGDDH |
| Beta subunit of | RVDSLYEKILKDDRHHQVLCLKSEANITERFFPDWSMKVITIDENSEILLKPI |
| photoactivated | KVLLQSLGESKMALANYTHSGVLQAIENGINPLTLTPRYTEKIIFFADIMGF |
| adenylyl cyclase | ASMTEKLQVTEVAELLNQFFTICTSEIAAKGGEVTKFIGDCVMAYFDGDR |
| ABRG53 0928 | ANEALQASLDILSELEILRDRSPVNSPLRVLYVGIGLSKGTVLEGNIGSQTK |
| Pseudanabaena | KEYAIIGDAVNVAARIESLTRKLNRFLVFSAAVREGLSDRWKLLKMGDFY |
| sp. ABRG5-3 | AKGREAIVEVYSIDNPLASQKSDFHHLKQEINEQLELIGKQTLVSYKQRKT |
| 370 AA | LFKLYDR |
| SEQ ID NO: 29 | |
| Putative | MRISVYVLYIGACLLAIGAAILSGVILLNTFAGQQQQSEDAQYTTGRGQVQ |
| membrane- | AIQGVIANQMSRMVASVDDRSRQLLMTLKELPDDALEPQNAVASLNHSIF |
| bound adenylyl | TTWVPSIMANDTVLGNGLTIMYVNESNGQLYDRTFSVYRELLKSDQWNY |
| cyclase, 4.6.1.1 | LYGYTFPDELSHVFFINWLDYSTPRLGKDVYQVNFTTVKAPVYQKDNFFN |
| Euglena gracilis | HVMPWGSTDGNTFWYFTYMRSFYTHGVWLNMQCWDVTVNWLSLMQS |
| 841 AA | GLTAGASVASYDSQGYVVTATNKDELRRLAQCIETGDVGSSCISEGGCNC |
| SEQ ID NO: 30 | LTTFAKFHPIAELRDLYLALHTPLWDDLDAPPIPLQQQDIQLSGQRYMAIV |
| | ATLFAKSGFRVTVVWYQPWVLTDTSAVGVTALICVLTMLSTELLTLLGIFG |
| | VLRPLMALGSAMRVVAHTLRKGDAEREAVLQPRRPNIFREVDTIGQDFETI |
| | VVDFLGFSSTNARDNKHAPKDPNKPFVVIFTDIQSSTGLWGRDPAQMSRC |
| | VQVHHEIIRTLIKDHHLYEVKTAGDSFMITTASPPDALRFALDLQQALYDY |
| | DWDWDECDDFYRETTLALWPAALGAKYADMWNGLRVRVGIHFGLGDV |
| | VYDEVSKGYDYYGNVVNTAARIEALAHGGQIVVTESLLAALPTPLDPRVS |
| | RTVSLGTVPLRGVTAPPALVEVQMASLATRTFPPLRVEHANEAAKQECIN |
| | VPDLLSDKAGSDGLLTGRLGFQTPSLQSDSLVQVAEDLAKTHAMVRRGYF |
| | SAEAVTQHLLVLYRIVEDLLTPLAPSQFATVTKALARGWGVAPPKSKAEF |
| | GPVGLRLMQRMSETTTVLKNLAAPHHRAQSAGPDARLLDMEATA |
| Beta subunit of | MMKRLVYISKISGHLSLEEIQRIGKVSIKNNQRDNITGVLLYLQGLFFQILE |
| photoactivated | GENEKVDKLYKKILVDDRHTNILCLKTEYDITDRMFPNWAMKTINLNENS |
| adenylyl cyclase | ELMIQPIKSLLQTITQSHRVLEKYMPARVIYLINQGINPLTVEPQLVEKIIFFS |
| BGP 1043 | DILAFSTLTEKLPVNEVVILVNRYFSICTRIISAYGGEVTKFIGDCVMASFTK |
| Beggiatoa sp. | EQGDAAIRTSLDIISELKQLRHHVEATNPLHLLYTGIGLSYGHVIEGNMGSS |
| PS | LKMDHTLLGDAVNVAARLEALTRQLPYALAFTAGVKKCCQAQWTFINLG |
| 350 AA | AHQVKGKQEAIEVYTVNEAQKYYDTLQITQLIRQTLENDK |
| SEQ ID NO: 31 | |

TABLE 3
Nucleotide Sequences

| Description | |
|---|---|
| SEQ ID NO | Sequence |
| PAC wt (nt) | atgatgaagcggctggtgtacatcagcaagatcagcggccacctgagcctggaagagatccagoggatcggcaagg |
| Codon | tgtccatcaagaacaaccagcgggacaacatcaccggcgtgctgctgtacctgcagggcctgttcttccagatcct |
| optimized | ggaaggcgagaacgagaaggtggacaagctgtacaagaaaatcctggtggacgaccggcacaccaacatcctgtgc |
| Beggiatoa sp. | ctgaaaaccgagtacgacatcaccgaccggatgttccccaactgggccatgaaaaccatcaacctgaacgagaaca |
| SEQ ID NO: 32 | gcgagctgatgatccagcccatcaagagcctgctgcagaccatcacccagagccaccgggtgctggaaaagtacat |
| | gcccgccagagtgatctacctgatcaaccagggcatcaaccccctgaccgtggagccccagctggtcgagaagatc |
| | atcttcttcagcgacatcctggccttcagcaccctgaccgagaagctgcccgtgaacgaggtggtcatcctggtca |
| | accggtacttcagcatctgcaccoggatcatcagegcctacggcggcgaagtgaccaagttcatcggcgactgcgt |
| | gatggccagcttcaccaaagagcagggcgacgccgccatccggaccagcctggacatcatctccgagctgaagcag |
| | ctgcggcaccacgtggaggccaccaaccccctgcacctgctgtacaccggcatcggcctgagctacggccacgtga |
| | tcgagggcaacatgggcagcagcctgaagatggaccacaccctgctgggggacgccgtgaacgtggccgccaggct |
| | ggaagccctgacaagacagctgccctacgccctggcctttacagccggcgtgaagaagtgctgccaggcccagtgg |
| | accttcatcaacctgggagcccaccaggtcaagggcaagcaggaagccatcgaggtctacaccgtgaatgaggccc |
| | agaagtactacgacaccctgcagatcacccagctgatccggcagaccctggaaaacgacaagccgcggacgtac |
| PAC (S27A) | atgatgaagcggctggtgtacatcagcaagatcagcggccacctgagcctggaagagatccagcggatcggcaagg |
| (nt) | tggccatcaagaacaaccagcgggacaacatcaccggcgtgctgctgtacctgcagggcctgttcttccagatcct |
| Codon | ggaaggcgagaacgagaaggtggacaagctgtacaagaaaatcctggtggacgaccggcacaccaacatcctgtgc |
| optimized | ctgaaaaccgagtacgacatcaccgaccggatgttccccaactgggccatgaaaaccatcaacctgaacgagaaca |
| Beggiatoa sp. | gcgagctgatgatccagcccatcaagagcctgctgcagaccatcacccagagccaccgggtgctggaaaagtacat |
| SEQ ID NO: 33 | gcccgccagagtgatctacctgatcaaccagggcatcaaccccctgaccgtggagccccagctggtcgagaagatc |
| | atcttcttcagcgacatcctggccttcagcaccctgaccgagaagctgcccgtgaacgaggtggtcatcctggtca |
| | accggtacttcagcatctgcacccggatcatcagcgcctacggcggcgaagtgaccaagttcatcggcgactgcgt |
| | gatggccagcttcaccaaagagcagggcgacgccgccatccggaccagcctggacatcatctccgagctgaagcag |
| | ctgcggcaccacgtggaggccaccaaccccctgcacctgctgtacaccggcatcggcctgagctacggccacgtga |
| | tcgagggcaacatgggcagcagcctgaagatggaccacaccctgctgggggacgccgtgaacgtggccgccaggct |
| | ggaagccctgacaagacagctgccctacgccctggcctttacagccggcgtgaagaagtgctgccaggcccagtgg |
| | accttcatcaacctgggagcccaccaggtcaagggcaagcaggaagccatcgaggtctacaccgtgaatgaggccc |
| | agaagtactacgacaccctgcagatcacccagctgatccggcagaccctggaaaacgacaagccgcggacgtac |
| Photoactivated | cactttctgagtgtctattttttttcgcttcaaagcaatgtacattcttgtctggaagaagggccagcaaatcaaa |
| adenylate | acttttcatacgcttgatgaggcagctcagttcaaggcagcttcaaacattgatgaagcacaaatgttcagtgtga |
| cyclase subunit | ctgttgcaccagccatcagcgcctcagggggaagcaatgaagcaacaaacctgcggaggctgatgtatttatcgaa |
| beta, 4.6.1.1, | gtcaacaaaccctgaagagtgcaacccacaattccttgcagaaatggcccgagttgcaaccattcgaaacagagaa |
| Photoactivated | attggagtcagtggtttcctcatgtactcctctcccttctttttccaggtcattgaagggactgatgaggaccttg |
| adenylyl cyclase | atttcttatttgcgaagatttctgctgacccacgacatgaaaggtgcattgtccttgctaacgggccatgtactgg |
| subunit beta | gcgcatgtatggggactggcatatgaaagactcccacatggacagtatcacgacgcatcctgcaatgaagacaatt |
| pacB | ttgtatcagattgcccgatctttctcaagtatgtggagctatctcccgaagagtgcaggcaacatgcttctgttgg |
| Euglena gracilis | gcaaagacccagctgcgcaaccaccagaacccatgagtgtggtggtcaccttcatctaccttgtggagtttggctc |
| mRNA | tatcttgtcgaaccccaatttgacagagcaggcagctgaagtgctgagcacttttgttgatgtctgtgtgaagaat |
| SEQ ID NO: 34 | gtggagggcagtggtggcaacatcgccaagttcatcacagggatttgcatggcatactggccgatcaacagaacgg |
| | aagaagccctgaccgccatccagcagatcagtgaagacctggcccagctccgcagccagcaggcaccggggtcggc |
| | cgtctcgctgatgtactcgcaggccggggtgcactatggccgggccatgctgtgcaacgccggaagccggaagtcg |
| | gactttactttgcttggcgattgcatcaacaccacgtcacgtattgcaacgctggccaaaaagttaaagaccccac |
| | tgctgttttcgtttgaagttcgatgcctattgggtgacgagatgcgggaagagattgagggtgctgggatgcatca |
| | ggtcaaaggaagggataagccagtggttgtttaccaattccctgggcctgagctggatgtcgagatggtgcgccag |
| | aagatcgagcagttcacccctggtcgcttccggtgccaaatgcccgtggtcgagtacgaggctttgccgatttcgc |
| | agcgcccccccatcttcgacgacacccccaagggcaacccccggccacggacccccggctatgggggccggcagcg |
| | cagtgacagcctggtggaccgcctcattatgattgccaaactggccggcccctctgtgtcggccacaggggacacc |
| | acgctgaccaccctgacgtacatctcgcaagccactcggccaatgtcgcggctggacctgtccgccatcatgcgca |
| | ccgccacccgccgcaacgcccagcagagcatcaccgggaccctgctgcacgtgaacggcctgttcgtccagacgct |
| | ggagggcccgaaggatgcggtggtcaatttgtacctcaggatccgccaggaccccaggcacaccgacgtcacgacc |
| | gtccacatggccccacttcaggagcgggtgtacccgtcggagtggaccctcacctcagccacagaggagatgctgg |
| | caacattcccacccctgcaagatgtgttgtcccagttggcgaagtcgttcacctcgctggagacttacgtgccttc |
| | cacggtggtgaggtatttgacagctgggaacaacccaaggaatctgatgcctgtttcatgtggcgttgtcatgttg |
| | gccaccgacatttgcagcttcacaagcttgacagagaaatcctccttgacagaggtttggatgatctgcaacacat |
| | ttattgatgcgtgcacctccgccatctgccaggaaggaggagaggtgatcaaactgattggtgactgtgtgacagc |
| | atatttccccggaaacaatgcagattcggctgtggctgcagcccaggagctgttcactttctgccgccagctccga |
| | gaagcgtttgtggatgtcctcgatgtgcggggatgtgtctcgtgcggcgtcggcctggattacggccaggtggtca |
| | tggcccaatgcggttcattgggactgacagagtatgtggtggccggggccgtcagtgcccgggtgatggaggtgga |
| | ggccattacccgcgaggtgggctacgccattgtggtcacggaacctgtcgccgaccggctctccccacagttgcgg |
| | gaccacggcatcgtcccgacgccccaggccatcgaggggctgccctgctatggcatcgccggtgaggagttcgagc |
| | ttgacgtcgattccatcaagcggggcatcaaggcgctgcatgcggcgcgcagggggagaagcccctgaccgagccc |
| | gaggaggcgaagccggacttccgggtctccccggggcgggtgcgccacggcgactcgggccggcgctccaactccg |
| | cccaggggaagcgcagcatccaagtccgctgaccgacgtgcccgtgacatgaccggatctgtgcatttggcccttt |
| | tgaatacatcaggggcacagtgcactgagaatggctgctcagtgtttcccctccacgcttcagcgatcacccgacg |
| | cgcacgtgagtggttcctcccccctggaacaacatacttttgtgttgtcgtgtgctttgcaactccaaaaaccaac |
| | ctcct |
| Photoactivated | cactttctgagtgtctattttttttcggtttgcggcaacatgtacatccttgtttggaaagaaggccagcagatca |
| adenylate | ggactttccaagatttggaggagtgtgggcagttccaaactgcatccaatataacagatggtcagatattcagtat |
| cyclase subunit | caatgtcacacccaccatgagcaagggaggagaaaccggagagacgcaactccggcggctcatgtatctctccgca |
| alpha, 4.6.1.1, | tcaactgagccagaaaaatgcaatgctgagtaccttgctgatatggcgcacgttgcaactctcaggaacaaacaaa |
| Photoactivated | ttggtgtcagcggcttcttgttgtacagttcccctttctttttccaagttattgaaggaactgacgaagacctgga |
| adenylyl cyclase | cttccttttcgcaaaaatctctgctgatccgagacatgagagatgtattgtgcttgcaaatggtccctgtacaggg |
| subunit alpha | cgcatgtatggggagtggcacatgaaagactcccacattgacaacataacgaagcacccagccattaagaccatcc |
| pacA | tcttccaaattgcaaggtcattctccagcatgtggtcctacctgccaaagaatgcggcgaacatgttgcttctcgg |
| Euglena gracilis | gaagaacccaaacaagcaagcccctgaacccatgagtgtggtggtcactttcatctatctggtggagttctcttcc |
| mRNA | attttggcccaccctggactgactgagcagtgtgcagacatccttgctgcgtttgtggacgcctgtgttcgcaacg |
| SEQ ID NO: 35 | tcgaaggtacagggggccaagtggcaaagttcatcactgggatttgcatggcgtattggccaataaacagggccga |
| | agatgctctggtcgggttgcagcaactgtcggaggacttggccgagctgcgcagccagcaacctcctggatctgcg |
| | ctgtccctgatttactcccggtgtggagtgcattacggtcgagctttgctgtgcaatgctggtttccgaaaggctg |
| | atttcaccctccttggcgactgcatcaacactgcctctoggatcacctcgttgtccgtcaaactcaaggtgccact |
| | tctcctttctttcgaagtgaggtgccttctgggtgatgaaatgcgagaggagcttgaaagttctgggctgcacaag |
| | gtgaaaggccgtgacaagcctgtgcaagtgtaccaattcaatgctccagagctggacagcgcgatggttcgtgcga |
| | agattgagcagttcaaccctggaaggtacagggcattgtgcccagtcaagccgtatgagagtttgcacccagctca |
| | gagaccaccaatctttgatgacaccccacgagagaaccagccaaaactcagccaagttcagcgacgcgacagcctt |
| | gtggatcgcctcagcttgattgcaaagcttgctttcccttcgagcatgatggcaggtggtgaaggccagctcatca |
| | cattgacatacatttoccaagcagcccatcccatgtcccgtttggatttggcatcaatccagaggattgcatttgc |
| | tcggaatgaatcctcaaatatcacaggctcattgctttatgtgagcggtttgtttgtacagacattggaaggtccc |
| | aagggcgctgtggtttccctgtatctcaagatcaggcaagacaagcgacacaaagatgtggttgcggtcttcatgg |
| | cccccatcgatgaacgggtgtatggcagtcccttggatatgacctccgccactgaggaaatgttggccaccttocc |
| | acccctgcaggacgtgctctcgcagctggccaagtcgttcatctccctcgagacctatgtcccctccacggtggtg |
| | cgctacctcaccgccggcaacaatccccgcaatctgcaacctgtttcggtggaagttgtcatgcttgccactgaca |
| | tctgcagcttcactccactgtccgagaagtgttccctgacagaggtgtggaccatttgcaacaccttcattgacgc |
| | ctgcaccagtgccatctgcaatgaaggtggggaggtcattaaattgattggtgattgtgtgacagcatatttcccc |
| | ccgacaggtgccgacaacgctgtccatgcatgccaggagatcgtcagcttctgtgctcagctgcgagatgccttcc |
| | acgacgtcttggactgccggtctgtggtggcttgtggggggggctggacttcgggcaggtcatcatggcccagtgc |
| | ggcagtttggggatgacagagtttgtggtggctggggaggtcagcgctcgtgtgatggaggttgaagccctcacgc |
| | gcgaggcagggcgtgcgatcgtcatcaccgaaccagttgcggatcgactgtctcccaaactgcgtgacacgggcat |
| | cgtcccctgccaggaaggagtggacggcgtcccttgctatggaatcctgggccctgaatgggaattggatgtggca |
| | accatcaagaagaacatttacgggttccacgacgcccgggccctggccgccatgaagaaggtggacgacggcacca |
| | acgccccgggccggggcgcccccgccgggggcatcccctcctcccccaaggttcgaccaccaggccgcaccaattc |
| | agtgtccagctacacccctgaccccaatgaggcactggatcctcggatggcggaatcggtgtttcttgacatgtgt |
| | catcagcggggagataccgccaacaactccatcgccgtcaaattgcggcaggcggccaacgacgatcggttggacc |
| | tgggcaggatgttgcaagggccacacgagctcatgcctgtgatgcaggccatcaagcacctgaccaacctgcgaat |
| | gctcaatatgagcgacaatttcgtggatgacaacaacgttggagaacttgtggaatcgtgcattccaatgcgttcg |
| | ttacaggtgttggacttgtccaacaaccctggcttgacgaaggtcatcgccctgaagcgcctcatcaagcataaca |
| | cccaggtccgggagatccttctgaacggcacccgcatcgccccaaccgagcaacgcaagctgcagtcctcgatgaa |
| | cgtcaaccggctgtgtgcctccaccgacctgaagggttcgcacaaatatgaacattaacgatacggcgctgtttgt |
| | acgtctggcctagcacatcacgacgtcactgctttcgcttgccccccccctccgtgtgctggtttacatgttgtga |
| | gttgcgggggagtgggattacaactcccagcatcgcagcggcattcgcacgtatatgccctgtgactggattccat |
| | ttccaccgtttcgtttgtgacaccgtaac |
| Photoactivated | tccaaaaatgtacatccttgtgtggaaaaggggtcaacaaatcaaaactttccacacacttgatgaagcagcgaat |
| adenylate | ttcaaggcagcttcaaacattgatgaggcgcagatgttcagcataactgttgcaccagccatcagtgcttcgggcg |
| cyclase subunit | gaagcaatgaagcaaccaatctgcgaaggctcatgtacctctcgaagtcaacaaatcctgaagagtgcaatccaca |
| beta, 4.6.1.1, | attccttgcagaaatggcccgagtggccaccctcagaaacagagaaattggagtcagcggattcctcatgtattcc |
| Photoactivated | tctcccttctttttccaggtgattgaagggacggatgaggacctcgattttctgtttgcaaagatatctgcagacc |
| adenylyl cyclase | ccagacatgaaaggtgcattgttcttgcaaatgggccctgcactggtcgcatgtatggtgactggcatatgaaaga |
| subunit beta | ctcccatattgacagtatcacaacacatcctgcaatgaagacaatcttatatcagatcgcccgctccttctcaagt |
| pacB | atgtggagctatttgccaaagagcgcaggcaacatgcttctgttgggaaaagacccagctgcacagccaccagaac |
| Euglena longa | cgatgagtgtggttgtcacctttatctaccttgtggaatttggttccatcttgtccaatcccaatttgacagagca |
| (Euglenophycean | agcagctgaggtgctcagcacgtttgttgacgtttgtgtcaagaacgtggagggcagcggtggcaatatcgcaaaa |
| alga) (Astasia | ttcatcactggcatctgtatggcatactggccaatcaacaggacagaggatgccctgaatgcaatacagcagatta |
| longa) | gtgaagaccttgcacaacttcgcagccagcaggctcctggatctgcaatctcactcatgtattcccaggctggtgt |
| mRNA | tcattatgggcgagccatgttgtgcaacgcagggagccgaaagtctgacttcaccttgcttggagattgcatcaac |
| SEQ ID NO: 36 | accacatcacggattgcaacactggcaaagaagctgaagaccccgttgctcttttcatttgaagttcgatgtttgc |
| | tgggtgatgagatgcgtgaagaagtcgagggtgctgggatgcatcaagtgaaaggaagggacaagccggttgttgt |
| | ttaccagttccctgggcctgagctggatgttgagatggtgcgacaaaagattgagcatttcaccccaggtcgcttc |
| | cgctgccagatgcctgtggtggagtacgaatcattgccaatttcacagaggccaccaatctttgatgacaccccaa |
| | agggcaagcctaggccccggaccccaggctatggtggccggcaacggagtgacagccttgtggaccgcctcattat |
| | gattgccaagctggctggcccctctatctcggccacaggagacagcacgttgacaaccttgacatacatctcgcaa |
| | gccacgcgacccatgtcacgacttgacctgtcagccattatgeggactgccacccgccgcaatgcccagctgagca |
| | tcactggaacacttctctatgtaaatggcttgtttgtgcaaacactggaggggccgaaggatgccatggtgaattt |
| | gtatttgaaaatccgccaggaccccaggcacactgacgtggtcaccgttcacatggccccaattcaggagcgggca |
| | tatccagcagaatggacactcacctctgccactgaagagatgctggccaccttoccacccctgcaagatgtgcttt |
| | cccagctggccaagtcttttacctcgttggagacctatgtgccctcaaccgttgtaaggtatttgacagctgggaa |
| | caacccaagaaacctgatgcctgtctcatgcggagttgtcatgttggccactgacatctgcagcttcaccagcttg |
| | acggagaagtcatccctcacagaggtgtggatgatctgcaacacattcattgatgcgtgcacttcagctatctgtc |
| | aggaaggtggagaggtgattaagttgattggtgattgtgtgacagcatatttccctggaaatggagctgattctgc |
| | tgtggctgctgcccaggagctgttcactttctgccgccagctcagagaggcatttgtggatgtcctcgatgtgcgg |
| | gggtgtgtgtcatgtggcgttgggcttgattatggccaggtggtgatggcccagtgtggctcactggggctgacag |
| | agtatgttgtggctggggccgtcagtgcccgggtcatggaggtggagtccatcacccgcgaggtgggctatgccat |
| | cgtcatcacagagccagtggctgaccggctttcaccacagttgcgggaccatggtatcatccccacaccccaagct |
| | atcgaaggtctcccctgctatggcattgcgggtgaggagttcgagcttgatgtcgattccatcaagcgaggaatca |
| | aagccctgcacgcagcaaggagcggggagaagccattggctttggagcctgaggaggcgaagcaggactaccgggt |
| | gtcccccggtcggatgcgccatggtgactctggacgacgttcaaactcagcccaggggaagcggagcacccaagtg |
| | cggtaagtgcggtgagcccatggaacaagtgcatagaaagagggcccaaggaccttcccttgaagtgcatctgggt |
| | tcaggtgctgtacattgaacacctccagtgacgtggttttctttcaggcctccgaatgcccaaatgcggaaacgtt |
| | gtaccagtgtgtatttgtgcctctcccatgctgcaacccccagtccggtgagtcccaccattg |
| Photoactivated | gcagggaaaatgtacattctcgtttggaaggagggccagcagatcaagacctttcaagatttggaagagtgtgggc |
| adenylate | agtttcagattgcctcaaatatcaccgatggtcagatattcagcgtcagtgtcacaccaaccatgagcaaaggagg |
| cyclase subunit | cgaaacaggtgagacacaactacgtcggctgatgtatctgtcagcatcaacagaaccagagaaatgcaacgctgag |
| alpha, 4.6.1.1, | taccttgctgacatggcacatgtggcaacacttcgcaacaaacagattggagtcagcggtttcttgctctacagct |
| Photoactivated | caccattcttctttcaagtcattgaaggaactgatgaagacttggacttcctgtttgccaaaatctcttctgatcc |
| adenylyl cyclase | aaggcatgaaaggtgcattgtgcttgcaaatggtccttgcacaggccgaatgtatggggaatggcacatgaaagac |
| subunit alpha | tctcacattgacaacatcacgaaacaccctgcaataaagaccattctctttcaaattgcaaggtcattttcaagca |
| pacA | tgtggtcatacctgccgaagaatgcagcaaatatgctgctattggggaagaatcccaacaagcaagccccagagcc |
| Euglena longa | aatgagtgtggtggtcactttcatatatctggttgaattctcctccatcttggcacaccctggactgacagagcag |
| (Euglenophycean | tgtgctgacatccttgctgcttttgtggatgcatgcgttcgcaacgttgaagggactggtggccaagttgcaaagt |
| alga) (Astasia | tcataacgggaatttgcatggcatattggccgatcaacagagcagaggatgcattggtgggcttgcagcagttgtc |
| longa) | ggaggatttggcagagctgcgtagccagcagcctcctggctctgccctgtccctcatttactcacggtgcggagtt |
| mRNA | cattatggccgggctttgctgtgcaatgcaggattccgaaaggctgacttcaccctgcttggtgactgcatcaaca |
| SEQ ID NO: 37 | ctgcctcccgaatcacctccctttccgtcaagctcaaggtgccactgctgctctcatttgaggtgaggtgccttct |
| | gggtgatgaaatgcgagaggagcttgaaagtgctgggttgcacaaggtgaagggtcgtgacaagcccgtccaagtg |
| | taccaattcaatgcacctgagttggacagtgctgtggttcgcaccaaaattgagcagttcaacccaggaaggtatc |
| | gtgcactgtgtccagtcaaaccatatgacagcctgcaccctgcccaaagaccaccaatctttgatgacacaccacg |
| | tgacagccaacccaaggtgaaccaaatgcagcggcgtgacagtcttgtggatcgcctcagcatgattgcaaagctt |
| | gcttttccctcgagcatgatggtaggaggagagagtcagctcatcacattgacgtacatctcccaagcagcccacc |
| | ccatgtctcgcttggatttgtcttcaattcaaagagtttcattctcaaggaatgaatcatcaaatatcacaggctc |
| | cttgttgtatgtgaatggcttgtttgtacagacacttgaaggcccaaaaagtgctgttgtttccctgtacctcaag |
| | attcggcaggacaaacgccacaaagatgttgttgccgtgtttatggctcccattgaggagcgcgtgtatggcagcc |
| | ccttggacatgactgctgccacagaggagatgctggcaactttccctcccttgcaagatgtgctctcacagctggc |
| | caagtcattcatctcactggagacgtatgtgccctccactgtggtgcgctacttgactgcagggaacaatccccgg |
| | aacttgcaacccgtctcagtggaggttgtcatgctggccacggacatctgcagtttcaccccgctgtcagagagat |
| | gctctctgactgaggtgtggaccatctgcaacaccttcattgatgcctgcaccagtgcaatctgcaacgaaggtgg |
| | ggaagtcatcaaactgattggggattgtgtgacagcttacttccctccaacggctgccgataatgcagttcatgct |
| | tgccaggaaattgtcagtttctgtgctcagctgcgggatgcattccgagatgtactggactgccgctccgttgtgg |
| | cctgtggggtcggtctggatttcgggcaggtcatcatggcccagtgtgggagtttggggatgacagagtttgtcgt |
| | tgcgggggaggtcagtgcccgtgtgatggaggttgaggcactcacacgtgaagcagggcgtgccatcgtcatcacc |
| | gagcctgttgcagateggctctcccccaagttacgcgacactggcattgttccctgccaggagggtgttgatggag |
| | ttccctgctatgggattttgggtgtggaatgggagctggatgtggcaataatcaagaaaaatatctatggctttca |
| | tgaggcgcgtgcacaggcagccctcaagaaagtggacgatggcacaaatgctccgggccgtggtgcaccagctgct |
| | ggtgtaccgtcttcacccaaggctcgagccctgggccgcaccagttcagtttccagctacactcccgacctcaacg |
| | aaacactggacccccgcatggctgagtccgttttcaatgatttgtgcaaccagcgtggcgatgctcccaacaactc |
| | cattgctgcaaaattgcggcaagcagccaatgatgaccgattggacctgggccggatgctgcagggaccccatgag |
| | ctgatgccggtgctccaggcaatcaagcagctgaccaacctacggatgctcaacatgagtgacaacttcgttgatg |
| | acaacaatgtcggagaacttgcagagtcgtgcattcccatgcgatcattgcaggtgctggacctgtccaacaaccc |
| | tggcttaacaaaggtcatcgctctgaagcgcctcatcaagcacaacacccagattcgggaaattctgctgaacggc |
| | acgcgcattgcgccaatggagcagcgcaagctgcagtcctccatgaacgtcaaccggatgtgtgcctccactgact |
| | cgaaaagctcccacaaatatgatcactaatgacgatcgatgcacacacaatttatgtccattttgtttctccctct |
| | catgtgcacatttgatgtgaccaatgtgtgattgtggctgggaacaacttccggccacagcagtgggatttcacac |
| | acctgtccctccaccgt |
| Photoactivated | atgtacaaccgagtttataaggagcgtcaggagattcgcaccattcagggtctggaggcttcaggacagttccaga |
| adenylate | ccgccagcaacatcaccgatatccaaatctattctattaacgtgaccccaaccatgtccaagggggggagaccggg |
| cyclase subunit | gagactcagttgcgccggctgatgtatttatcggcaagcacggagcccgaaaagtgcaacgctgagtatctggcgg |
| alpha-like | acatggcccacgtggctactctccgtaataaggccatcggcgtcatccgcttcctgttgtacagcagtcccttctt |
| protein ST-pacA | tttccaggtcattgaaggcaccgacgaggatctagacttcctctttgccaaaatatccgccgaccctcgccacgag |
| Euglena gracilis | cgctgcatcgttctggccaacggcccttgtactggtcgtatgtatggcgagtggcacatgaaggactcccacatcg |
| mRNA | acaacattactaataacccggctatcaagacgatcctgttccagatcgctcgctctttcactagtatgtggtccta |
| SEQ ID NO: 38 | cctgcccaagaacgctgcgaacatgctgttgcttggaaagaaccccaacaagcagggtccagagccacgttcagtg |
| | gttctcacattcatctacctggtcgaattctctagtattctggcacatccgggtctgacagagcagtgcgcggaca |
| | tcttggcggccttcgtggaggcgtctgtgcggaactgcgaaggcacgtggggactggtcgcaaaatttatcaccgg |
| | tacgtgcgtcgcgtattggcctattaaccgggctgaggatgccctcgttggcctgcagcaattgtcagaggatctc |
| | gccgtgctgcgcagctaccaacccccgggttccgctctctcccgtatctactcgcgtcggggggtccactacggac |
| | gcgcgttgctgtgcaatgctgggttccggaaagcagacttcactttgctgggcgattgcattaacactgcatcgcg |
| | gattacttccctgtccgttaagttgaaggtgcccctcctgctcagcttcgaggtgcgctgcttgctcggagatgag |
| | atgcgcgaggaactggagcgctctgcgctgcataaggtcaaggggcgtgacaagcccgtgcaggtttaccagttca |
| | atgctcctgaattggatagtgcacttgtgcgtgctaagatcgagcaatttaaccctggtcgttaccgcgcgttgtg |
| | cccagtgaagccctacgagtcccttcacccagcccagcgtccacccatctttgatgacaccccaagagagaatcaa |
| | cctaagctttctcaggttcagcgccgggattcccttgttgacaggctgtccctgatcgcgaaactggctttcccct |
| | ctagcatgatggccggcggggagggccagttgattactttgacctacatctcgcaggccgcacaccccatgagccg |
| | cttcgacctcgcttcgattcagcggattgcctttgctcgcaacgagtcatctaatatcacgggatccttgctttac |
| | gtgtccggactgttcgtccagaccctggacgatccgaagggcgccgtcgagtccctgtacctgaagattcgacagg |
| | ataagcgccacaaggatgtcgtggcagtttttatggccccgattgacgaacgtgtgtacgggacacccctcgacat |
| | gacttcggcaacagaggaaatgcttgcaacgttcccgccactccaggatgtcctgtcacagttggctaagagcttc |
| | atcagcttggagacttacgtgccgagcacggtggttagatatctgacagccggcaacaatccccggaacttgcagc |
| | cagtgtctgtcgaggtcgttatgctggctacggacatctgctccttcactcccttgtccgagaagtgctctcttac |
| | agaggtttggaccatctgcaacaccttcattgatgcctgtacatccgcaatctgcaatgagggtggagaggtcatt |
| | aagcttatcggtgactgtgtgaccgcatatttcgcccctaccggggccgacaacgccgtgcatgcgtgtcaggaga |
| | tcgtgagtttctccgctcaactgagagacgcctttcatgacgtcttggattgccgctccgtggttgcatgcggtgt |
| | gggactggacttcggccaggtgatcatggcccagtgcgggagcttgaggctgacagagttcgtggtcgccggtgag |
| | gtgagcgcccgcgtaatggcagtggaggccttgacccgcgaagctggccgtgctatcgttatcaccgagcctgtgt |
| | ctgaccgcctgtccgccaagctccgcgacacaggtatcgtgccttgccaggaggggctgcacgccgtcttgctgtg |
| | gtacccggggcccagaatgggcattggttgcgccaaccaccaagaggatcacctgagggttcctcgccggccgggc |
| | ccaggtcggcacgaggaaggtggcaggcgacaccagcgccccggtcccggccgaccccgccggggccatccgttcc |
| | tgccccaggggagcaccacgcgacctcatcagttctctgtccagctgcaccctctgcccaagctgggcaccggctc |
| | tagtgacgggggaatcggggtcagcctccacgtctacagcgcaggcaggtaccggcagcaactgcaccggcgccag |
| | atcgctgccgggggccagcgccggtccgtgggccccggccaggacgttgctagggccacccgtgcccatgcctgcg |
| | acgctggacatcaggccccggatcaaccagccaacgcccagtacgagcgccagttccggggcgggcagcaacgatg |
| | gaggacctgcggcatcgtgcactcgaacgcctttgtgaccggtgtgggactggtgcagcaaccctggcttgatgag |
| | ggccatcgccctgaggcacctcaccagtccctccacccaggaccaggcgacccctcagagcagcatcctcaccgtc |
| | ccaaccgggcgacagaacccggcctgttggttgcgcgacagccggctgtgtgcctccaccgacccgaaggtttcgc |
| | ccagatttgtaccttctaa |
| Photoactivated | atgtacatcctggtgtggaagaaaggtcagcaaattaagactttccacaccctggacgaggccgctcagttcaagg |
| adenylate | cggcctctaacatcgacgaggcccagatgttcagcgtcactgtcgctcccgcgatctcggcgtccggcggttctaa |
| cyclase subunit | cgaggctaccaatctgcgccggttaatgtacctctccaagtccacaaaccctgaggaatgcaacccccagttcctg |
| beta-like protein | gccgagatggcacgtgttgccacgattcggaaccgggagatcggtgtgtccgggttcctcatgtactcttccccat |
| FB pacB | tctccttccaggtgattgaaggcacggacgaggaccttgactttctgttcgccaaaatcagcgctgatcctcgcca |
| Euglena gracilis | tgagcgatgcatcgtcttggccaacgggccctgtacaggaagaatgtacggcgattggcacatgaaggatagccac |
| mRNA | atggactcgatcacgacacacccggcaatgaagaccatcctttatcagattgcccggtcattcagctccatgtgga |
| SEQ ID NO: 39 | gttaccttcctaagtccgctgggaatatgttgctgcttggcaaagaccccgcggcccagccgccagagccgatgtc |
| | cgttgtcgtgacctttatttacctggtggagttcggcagcattctgtctaaccctaacctcacagaccaagcggcc |
| | gaggtgctgagtacctttgtggacgtctgcgtgaagaatgtggagggatcgggtggcaacattgctaagttcatca |
| | ccgggatttgcatggcttactggcccatcaaccgaaccgaagaggcactgaccgccatccagcaaatctcggagga |
| | tttggcacagctgcgatctcagcaagcacccggttccgccgtgagcttgatgtactcccaggccggggttcactac |
| | ggccgacccatgctgtgcaacgcagggcgtcgcaagagcgacttcaccctcctgggtgactgtatcaacacaactt |
| | ctcggatcgccaccttggccaagaaattcaagactcctcttttgttctcccaggaggtccgctgcctattgaggga |
| | tgagatgcgcgaggaaattgttggggccggtatgcaccaagtgaagggtcgcgacaagcccgtggttgtgtaccaa |
| | tttccaggcccagagctggacgtggagatggtgcgacagaagattgagcagtttacgccgggtagattccgctgcc |
| | aaatgccagttgtcgaatacgagggactgccgaactcccaacgccctccaattttcgatgacacccccaaggccaa |
| | caggcggcccaggacccctgggtacgggggtcggcagaggtccgattcccaggtggaccgccccatcatgattgca |
| | aagctcgcaggtcctagcgtgtccgcgacgggcgacactaccctgacgaccctgacctatatctcccaggctacga |
| | ggccaatgtctcggcaggatctgagtgccataatgcggaccgctactcgtcgcaatgcacagcaaagcatcactgg |
| | cacactcttgcatgtgaacggtctgttcgtccagactttggaaggccctaaggacgctgtcgccaatctgtacttg |
| | cgcgtgcggcaggatccgcgtcagactgacgtgaccactgtccacatggctccactgcaggagcgcgtttatccct |
| | ctgaatggactttgaccagtgcgacagccgaaatgctggcaacttttccgcccctgcaggatgtgctggcgcagct |
| | cgctaagtccttcacgtctttggagacttacgtcccgtccaccgtcgtgcgtcacctgacagcaggcaacaatcca |
| | cgcaacttgatgccagtgagttgtggggtcgttatgctggctaccgacatttgcagcttcacttcattgacagaga |
| | agagctcacttaccgaggtttggatgatctgcaacacgttcattgatgcctgcaccagcgccatctgccaggacgg |
| | cagagaagttatcaagctgatcggggactgtgtcacagcctattcgcctggaaacaacgccgacagcgcagttgct |
| | gcggcccaggagctgttcaccttctgccggcagctgcgagaggcattcgttgatgtgctcgatgtgcgcggatgcg |
| | tgtcttgcggggtcggcctggagtatggacaggttgtcatggcccagtgcggctccatgggacttaccgaatacgt |
| | ggttgccggagctgtctcagctcgtgtgatggaggtagaggcaatcacccgcgaggtgggctatgctatcgtcgtg |
| | acggagcccgttgccgaccggctgtcaccccagttgcgtgatcatggtatcgtgcccaccccccaggccatcgagg |
| | gcctcccttgctacggcatcgcgggcgaggaattcgagctcgacgttgactctatcaagcgcggcatcaaggcttt |
| | gcacgctgcccgctccggtgagaagccgctcacagagcccgaggcggccaagcccgattttcgtgtgtcgcccgga |
| | cgcgtacgtcatggagattctggccgccgttctaacagcgcccagggcaagcgctcaattcaggtccgctaa |
| Photoactivated | atgtacattctggtctggaaggaggggcaacagattcggacttttcaggacctggaagagtgcggccagttccaga |
| adenylate | cggcttccaacatcaccgatgggcagattttttctatcaacgtgacccccacgatgagcaagggcggggagactgg |
| cyclase subunit | tgagacccaactgcggcgcttgatgtacctctctgcgtctacagaacccgagaagtgctacgccgagtacctggct |
| alpha-like | gacatggcccacgtggccacactccgtaacaagcagatcggcgtgagcggcttcctgctttacagctcccccttct |
| protein FB pacA | ttttcgaggtcatcgagggtaccgatgaggaacttgacttcctgtttgcgaagatcagcgccgacccacggcatgt |
| Euglena gracilis | ccgttgcattgtgcttgctaacggtccgtgcaccggacgcttgtatggtgagtggcacatgaaagactcccacatc |
| mRNA | gacaatattacaaagcatcctgcgattaagacaatcttgttccaaatcgcccgcagtttcagctgcatgtggtctt |
| SEQ ID NO: 40 | atttgcctaagaacgccgcaaacatgctgcttttggggaaaaacccaaacaagcaggcgccagaaccaatgtctgt |
| | ggttgtgaccttcatctacttggtggagttttcctcgatcttggctcatcccgggcagaccgaacaatgcgccgac |
| | attctggcagccttcgtcgacgcctgtgttcgcaacgttgaagggacgggcgcccaggttgcgaagttcatcaccg |
| | gcatctgcattgcttactggccaattaaccgcgcagaggatgccctgatcggtttgcagcaattgtccgaggatct |
| | ggccgagctgcgcagccagcaacggcccggctccgcactctccctgatttactcccggtgcggtgtgcactacgga |
| | cgagctctcctgtgtaacgccgggttcagaaaggcagacttcaccctgcttggtgactgcatcaacaccgcgtcac |
| | ggatcacttccctgtcagttaagctgaaggtgcccctcttactgtcgtttgaggttcgctgtgtgctgggagacga |
| | gatgcgagaggtgctggagacatcaggtctccacaaggtcaagggccgcgaccagccggtccaggtgtaccagttc |
| | aacgcccgggagctcgacagcgctatggtgagagccaagattgagcagttgaatcccggctcctaccgcgcgcttt |
| | gccccgtgaagccttatgaatccctgttgccggcccagcggccccctatcttcgacgatacccctcgtgagaatca |
| | gcccaagctgagtcaggtccagcgacgggattcgctggtggaccgtctgtccctgatcgctaagttggcctttccg |
| | acatcgatgatggccggaggcgagggccagcttatcaccctcacatatatctcccaggcagctcacccgatgaccc |
| | gactcgatctggcgagcatccagaggattgctttcgcccgcaatgagtccagcaacatcactggatccctgttgca |
| | cgtctccggccttttcgttcagacgctggagggcccgaagggggctgtggtctcattgtatctcaagatccggcaa |
| | gacaaacggcacaaggatgttgtcgctgacttcatggctccttctgagggccgcgaatacggatccccactggata |
| | tgacgtctgcaacggaggaaatgttgacaagcttccctcccttgcaggagctgctaacgcacttggccaaaccgtt |
| | cattagcctggagacgtacgttcccaccactgttgtccgctacctgacagegggcaacaatcctcgaaacttgcaa |
| | cccgtgtcagtggaggtggttctcctggccaccgatatctgcagcttcacccctttgtcagagaagtgcagcctga |
| | ctgaggtgtggactatctgcaagacgatcttcgtggccttcattcgcgccattttcaacgagggtggcgaggtgat |
| | caagctcatcggcgactgcgtgaccgcatacttcccacccaccggtgctgataaggcagttcatgcttgccaggag |
| | atcgtaacattctgcgcccagttgggtgacgcctttcacgatgtgctggactgccgcagcgtcgttgcttgcggtg |
| | tgggtctggatttcggccaggttattatggctcaatgcggatccctgggtatgactgagttcgtggtcgcgggaga |
| | ggtgtccgcacgtgtcatggaagtcgtggccttgaccagagaagccggccgagcgattgtgattacagagccgctg |
| | ccagatcgcctcgcccctaagctgagggacactggtatcataccctgccaggaaggtgtggacggtgtgccatgtt |
| | atggcaacctgggccctgagtgggagctggatgtagctactatcaagaaaaatatctatggattccatgacgcgag |
| | ggcccttgctgcaatgaagaaagtggacgtggggactaacgcccccgggcgcggggccccagcagggggcattcca |
| | tcgtctcccaaggtccgccccccgggccgtactaactcggtttctagctacacccccgatcegaacgaggccctgg |
| | acccccctatggctgagtctgtgttcctggacatgtgccatcagcgtggggataccgcaaacaattccatcgcagt |
| | caagttgcgtcaggccgcgaatgacgatcgcctggacctcggacgcatgctgcagggcccccacgagttgatgccc |
| | gttatgcaggccatcaagcatttgactaatcttaggatgctgaacatgtccgataacttcgtcgtggataacaatg |
| | tcggagagttggtcgagagttgtatccccatgcggtctctgcaggttctcgacctgtctaacaacccaggcctcac |
| | caaggtcategccctcaagcgtttgatcaagcacaacacccaggttagggagtttctgcttaacggcacccgcatt |
| | gcacctacggaacagcgcaagctgcagagttccatgaacgtgaaccgtctttgcgcttctaccgacctcaagggga |
| | gtcacaagtacgagcactaa |
| Photoactivated | atgtatatcttggtgtggaaggaggggcagcaaatccgcactttccaggatctggaagagagcgtccagtttcgga |
| adenylate | cggcctctaacatcaccgatggccagattttcagcatcaacgtcaccccgactatgggcaagggaggtgagaccgg |
| cyclase subunit | agagacccagctgaggcggttgatgtacctgagcgcctcgacggagcctgaaaactgcaacgctgactacgtgggt |
| alpha-like | gacatggcccacttcgcaactctgagaaataagagtatcggggtcagcggtttctacatgtatagtgccccattct |
| protein 1224- | tgtttcaggtgatccagggaacagacgaggatctggatttcttgtgtgccaacggttccgccgatcctaggcacca |
| 5/1F pacA | gaggtgcattgagctggccgatgggcccaccacaggccggatgtacggttggtggcacttgaaggacagtcacatc |
| Euglena gracilis | gacaatatcacgaaggaccccgccattaagacgattttgatcgagatcgcgcgttccttctcctctatgtggtcat |
| mRNA | atttgcctcgcaacgccgctaacatggtgcttttgggaaagaaccccaacaaacaggcccccgagccaatgtctgt |
| SEQ ID NO: 41 | ggtcgtgaccgttatctacttggtcgaattctccgttattatggcccatccagggcttagcgagcagtgccctgac |
| | attttgccggcttttgttgacgcgtgcgtcccgcatgtcgatggcaccgggggcgaggtggccaagttcatcattg |
| | ccatatgcatggcttactggcccatcaactccgacgagcacgccttggtgggactgcagcaactttccgacgatct |
| | ggcggagttgcgctccctacaggaggatggcagcgcgagaagcctgatctactcccgttgtggactgcatttcggg |
| | cgagctatgctgtgcaacgcaggcttccgcaaggccgattttacacttctgggtgattggatcaactctgcgtctc |
| | gtatcacctccctgagtgtgaattccaaagttccactgttgcttagtttcctcgtgcgatgtctccttggtgatga |
| | gatgcgtgtcgaactggaacgttctggtttgcataaggtgggcggtcgcgtcaagccggttcaggtgtatcagttc |
| | aacgcccctgaattggacactgcaaccgtccgcggcgagatcaagcaattcaaccccggtcgtgagcgcgccctgt |
| | gccccgtgaagccatacgagtctttgcacccagcacatccggacccgatcttcgatgacactccacgcgagaacca |
| | gcccgagctctcacaggtgcagcgccgggattcgcttgtggatcgtctgtcccaaatcgctaagctggctttccct |
| | tcgtccatgatggctggtggcgagggccagttgattactctgacgtacatctctcaggctgcccatcccgtttcgc |
| | ggttggacttggcctccatccaaaggatcgctttcgcgcgcaacgagtccagtaacattaccaagtccctgttgta |
| | cgtgtcaggcctgttcgttcaaaccttggagggcaccaagggcgcggtggtttccctttacctcaagaaccgccag |
| | gacaagcgccacaaggacgtcgtggctgcgttcatggctcccatcgacgagggggtatacggctccccccttgaca |
| | tgacctctgcgaccgaggaaatgctggcaacgtttacgccactgcacgacgtgctctcacagctggccaagagctt |
| | catttcgctggagacctatgttccgtcgactgtggtccgccacttggccgcaggtaataacccacggaacctccaa |
| | cccgtgtctgttgaagtggttatgctggccacagccatctcctcttttactccactgtcggaaaagtgctcactga |
| | ccgaggtgtggaccatttgcaacacatttattgtggcatgcactagcgcaatctgcaatgagggcggggaggttat |
| | caagctgatcggagactgcgtgccggcttactttcctcccactaacgccgacaacgcggtccatgcatgtcaggaa |
| | attgtgagcttctgcgcccagctccgggcagctttccacgacgttctggattgtcgctccgtcgtggcctgcgggg |
| | ttggcctggactatgggcaggtgatcatggttcagtgcggtagcctgggcatgactgaattcgtggtcgctggcga |
| | ggtccgtgcacgagtgctggaggtcgaggctctcaccagggaagcagggcgcgcaatcgtcatcacggagcccgtg |
| | cccgagcgacagtcacccaagctgcgagacactggaattgtcccttgccaggagggggtggatggagcccgatgct |
| | acggaattctgggccccgagtgggagctcgacgtagccacaattaaaaagaatatcaatggattcctcaaggatgc |
| | tcgcgctctggccgcgaacaaaaaggtggacgatggtaccaatatctcttgccgcggtggcaacccccctgctggc |
| | gggatcccgacgagccctaaggtccgtcccccgggccggaccaattctgtttccagctatacacccgatcctaaac |
| | aggcccttgatccccgcatggcagagagcgtcttcttggacatgtgccaccagggtgacacagttaataacagcat |
| | tgccgtgaagcttggcctggccgcaaacgaggacggcttcgacgggggatggatcctgacctggtacgtcgagctg |
| | atgcccgtgaaacaagccattaaggtgctcacccagctgcggattggcaacatgtccgacaacttcgtggacgata |
| | acaatgtcggtgagctgctcgagaagtgcatcccaacccggtccctccaggtgctggacctctccaacaaccccgg |
| | cttaactaaggttatcgccctgaagcctaagcacaacacgcaggtgcgcgagatcctgctcaacgggacccgcatt |
| | gctcctacagagcagcggaagcaacagacaagcaagaacgttaaccgtctgtgcgcgtccaccgacctcaagggtt |
| | ctcacaagtacgagcactaa |
| Photoactivated | atgtatatcttggtttggaagaaaggacaacagattaagacctttcacaccttggatgaggccgctcaattcaagg |
| adenylate | ccgcgtcaaacattgacgaggcccagatgttctcggtgaccgtcgcctccgctatctctgcctccggcggttctac |
| cyclase subunit | cgaggccactaacctgcggaggcttatgtacctctctaagtctacgaaccctgaagagtgtaatccccaattcctg |
| beta-like | gcagaaatggctagggtcgcctttatcagaaatcgggagatcagggtgtccggattcttgatgtatagctcacctt |
| protein ST- | tctttatccaggtgatcgtggagaccgatgaggacttggatttcctgttcgccaagatctcagcggacccgcgcca |
| pacB | tgagatgtgcatcgtgctcgccaatgggccgggcaccgggaggatgtacggggactggcacatgaaagatagtcac |
| Euglena gracilis | ctggactctatcaccacgcacaccgccatgaagaccattctctatcagatcgctcgctcgttctcatccatgtgga |
| mRNA | tttatctgcagaagtccgccgcgaacatgcttatgctcggtaaggaccctgcggctcagcccccggagcgtcgctc |
| SEQ ID NO: 42 | cgttgtggttacgttcatctacctggtcgagttttgcagcatctttagtcatccgaacttgaccgagaagacggcc |
| | gacgtgctttctaccttcgtctacgtctgcgttaagaacgtggagggctctggtggcaacatcgcaaagtttatcc |
| | ccgccatttgcatgacatactggcctatcattcgaactaaggaagctttgaccgccatccagcaaatcagcgaaga |
| | cttggcccagctgcggagccaacaggctcaaggttcggccgtgagtctacgttattcgcaggcgggggtgcactac |
| | ggccgggggatgcagtgcaatgcaggctcccgtaagagcgacttcacattactgggagactgtattaatacaactt |
| | cccgtattgcgaccctggccaagaaactcaagacgccccttctgttcgggttcgaagtgaggtgcctgttgggcga |
| | cgagaagagagaggaaatcgagggcgcaggcattcaccaagtcaagggacgtgataagccagtcgtggtctaccag |
| | ttccccggtcccgatctcgacgttgccatggttcgccagaagatcgaacagttcactcccggacgtttccgctgcc |
| | agatgcctgtggttgagtacgaggcactccccatctctcagegcccgcccatctttgatgacacaccaaagtccaa |
| | cccccggccccgcaccccaggatacgggggccgccagcggagcgactctctggttgaccggctgattcgaatcgca |
| | aacttgtccggtttctccgagagcgctacgggcgataccactctgactaccctgacctacatatcccaggccacac |
| | gcccaatgagccgtctggatttgagtgcaatcatgccaactgccacccggcgcaacgcacagcaatccaatatcgg |
| | cacagtcttgcacgagaacggactgttcgtccagactctggagggccccaaggacgccgtggtcaacctgtacctg |
| | cgcatccgccaggatccacgccataccgatgtcacgaccgtccacatggcgcccctgcaggagcgtgtttacactt |
| | ccgagtggactcttaccctggctaccgaggaaatgctggcaactttcccccctgtccaggacgtgctgtcccagct |
| | cgctaagtcttacacttccctggagacgtacgtcccttcaacagtggtacgctacctcacggcaggcaacaacccg |
| | cgcaacctgatgccggtgtcttgcggggttgtcatgttggctacagatattagctccttcacatcccttactgaga |
| | tcagctcattcaccgaggtttggatgatctgcaacaccttcattgatgcatgtacaagcgctatctgccaggaagg |
| | tggggaggttattaaattgatcggggactgtgtcacaccatatttccccggtaacaacgctgatcttgccgtggcc |
| | gcggcccaggagttgttcattttctgccgccagttgcgagaggcctttgtggacgtactggatgtgcgcggctgcg |
| | tgtcctgcgctgtgggaatggactacggccaagttgtgatggcccagtgcggtctgctccgcttgtctgactacgt |
| | ggcagcgggcgctgtttctgccggggtcgtggaggtggaggctatcacgcgcgaggtcggataccagttcgtggtt |
| | accgtgccggatgcagaccgggtgcagccccagttccgagattatggcatcgagcctaccccgcaggctattggcg |
| | gactcccctgctacgggatcgtgcgggaggtttttgacctgccagtggagagcattaagcggggtattaaggctat |
| | gcatgcggcccgctccggtgagcagcctctgactgaacccgagcaggccaagccagacttccgggtttcgcctggt |
| | cgagaccgtcacggtgtgtccggtcgacgttcgaacagcagtcagggcaagggcagcattcaggtgggctaa |
| Putative alpha | tcaagacagtgccgcaagatgtacgtgcttgtttggaagaatggacagcaggtgaagacgtttgaaacgctcgatg |
| subunit of | gcgccaactcctataagactggttccaacattgccgatgatgactctaacatttttcatatcgatgtcatccccgc |
| photoactivated | ccagagtgccggcagcgccggaggaggcaacgtagacaagccgctccgtcgtctgatgtatttgtcgaagactaca |
| adenylyl | gaagcagaattgtgcactccggagtatttggctgatatggcccacgtggcaacgttgcgcaacatggagatcggag |
| cyclase, 4.6.1.1 | ttagtggattcttactttacagtgcaccgttcttcttccaggttatcgaaggtaccgacgaagatttggatttctt |
| Eutreptiella | gttcgccaagattggtgccgacccacgtcacgaaaggtgcattgtgttggccaacgggccttgcacaggacgtctt |
| gymnastica | tatggtgactggcatatgaaggattcgcacatcgagaatatcacaagtcatcctgccatcaaaaccattctgtacc |
| mRNA | agattgcgcgctcgttctccagtatgtggtcttaccttcccaagaacgctgccaacttgcttcttctaggtaaaaa |
| SEQ ID NO: 43 | cccaaacaaacagacccctgagcccactagtgtcgtcgttacgttcatttacttggtcgagttctcttcaattttg |
| | gcacaccctggtttgaccgagcaagttgcagacgttctctcccacttcgttgacatttgcgtgcgcaacatagaag |
| | gttccggcggtgcaattgccaagttcatcaccggtattaccatggcctattggcccataaacagagctgaggatgc |
| | cttgttggctctgcagcagatcaacgaagacctcgcccaattgcgttcgcagcaaccgccaggttcagctttgtct |
| | ttgatgtacggacgtgctggcgtgcactacggtcgtgcattgttgtgtaacgctggtgtccgcaagtctgatttca |
| | ctcttctcggagattgtatcaacactgcgtcgcgtgtcacttcgcttgccacgcagcttaagagtccactgctgtt |
| | ctccttcgaagtgcgttgcttgctgggagatgacatgagagacgaactcgaatctgctggtatgcacaaggtcaag |
| | gggcgagacaagccggtggccgtttaccagttcccttgcgagcctttggataatgatatggtgcgtgccaagattg |
| | aggctttcaacccggggagataccgagcccaattccccgtggttccatacgacagcctgcacggtgttcacaaacc |
| | acccatcttcgacgactcccccaaagaccctttggcaacgaggatcgcacaagctgccggcgagcgtaaggatagc |
| | ttgatcgataggctgatcatgatatctaacttggcaattccagcatccatggccggaggaggcaacaaccaactca |
| | tcacgttgacatacatatctcacgccgtgaagcctatgagccgtttggatttggccgccatcaacaggaacgctgt |
| | gcgtcggaacgcggcctccaaaatcacgggcactttgttgtacgtgaatggacttttcgttggtactttggaagga |
| | cccaaagatgccgtcctaacgttgtacaataaagttcgtcgtgacgcgcggcacgaggacgttgtgacggtgcact |
| | tggcgcctctagacgagcgtgtctatgacgtgccgttctctgtgactgctgctacggacagcatgttgtccaagtt |
| | tccgcctctgcaggatgtgttgtctcagttggctaagtctttcatttcccttgagacatacgttccgtccaccgtg |
| | gtgagatggttgacagccggcaacaacccccgtgggttgcacgccgtgagtaccgaggtggtcatgttcgcaaccg |
| | atatttgcagttttacgccactttccgaaaactgctcgttggtcgaagtttggttgatttgcaacactttcatcga |
| | tgcttgcacgcactccatttgcaacaagggtggagaagtcatcaagctgattggagattgtgtgacggcttacttc |
| | ccgcccgacggtgcggacgctgctgtgttggcttgccaagaggttgttaccttctgcgcacaacttagagaggcct |
| | tcgaggaagttcttgactgtcgatccgttgttgcctgcggtgtcggtctcgactatggtcaagtcgttatggccca |
| | atgtggaagtgaaggtatgaccgagtacgtggttgctggagaagtcagtgcacgtgttatggaagtggaggcactg |
| | actcgtgatgtggctcgggcaattgttgtgacggaaccggtggcagaccgtttgtcccctgccatgcgtgacaacg |
| | gaattatcccttgctcccaaggtgtcgatggtatcccttgctatgggatcttgggagaggagtgggagctagacat |
| | tccaacaatcaaggccaacattaagaggttccacgctactcgtgcggaaattgctaggcagaaggaagaggaggca |
| | gctgccgcggaggcaggaaagccaaaggccaaagcccgtgccgacagccgctttgggccacgcgggtctgttaata |
| | gttattgccccgatccgaacgaagttctcgaccctaggatggccgaggacgtctttgtggaggcttgtgcagcccg |
| | tggagacgcacctaatttggcccttaaagccaagatgaggcaagctgctaacgacgacaggctcgacttggcgagg |
| | gccctgcaaggacctcacgagttatctccgttgaccaccgctctcaagcaactctgccatttgagaagcctcaata |
| | tgagcgacaatttcattgacgacaacaatgttgtggaccttgtggaagcttgcctacctatggagaatttgcaggt |
| | gcttgatttgtccaacaaccctggcattacgaagattattgccttgaagcggttggtgaagcacaatgtccaaata |
| | cgggaaatcaacctcaatggcacccgcgtggccccaacggagcagcgcaagttgcaatcttccattaatgtcaacc |
| | gtttgtgtgcatccgagaagaggtaagcatatgacattggtaggggccccacatgctgtggcccatctaagcaaat |
| | gag |
| Putative alpha | ttttcccaaagacagaatgtacatccttgtttggaaagaaggtcagcaaatcaagactttccaagatcttgaagaa |
| subunit of | tgtggccagtttcaaactgcttctaacatcagcgatgggcaaaccttttccatcaatgtgagtcctgttatgagca |
| photoactivated | aaggaggagagacaggagagacacagctgcgacgattgatgtacctttctgcgtcaactgagcctgagcggtgtga |
| adenylyl | cggagaatatttggctgagatggcccatgtggccaccctgcggaatagtgagattggggtgagtggtttcctgctg |
| cyclase, 4.6.1.1 | tacagtgctccattcttctttcaagtaatcgagggtacagatgaagatctggacttcttgtttgcaaagattgggg |
| Colacium | cagatcctcgccatgagcggtgtattgtcctggctaacggaccatgcacaggtcgattgtatggggagtggcacat |
| sideropus | gaaggattctcacattgacaacatcacgaagcaccctgctatcaagaccatcctgtaccagattgcaaggtctttc |
| mRNA | tccagcatgtggtcctaccttccaaagaatgctgcaaacatgctgctcctgggaaagaaccccaataagcaagcgc |
| SEQ ID NO: 44 | ctgagcctatgagcgttgttgtcacttttatctatctggtggagttctccagcattctggctcatccaggtctgac |
| | cgagcagtgtgccgatgtccttgctgcatttgttgatgcttgtgttcgcaacgtggaaggcactggtggtcagatc |
| | gccaaattcatcactggcatttgtatggcatactggcccatcaaccgagccgaagatgccctcgttggattgcagc |
| | aactttctgaggatttggctgagttgcgaagccaacagcccccaggctctgcgctgtcgctggtgtactcccgctg |
| | cggggttcattatggccgagcactgctgtgcaatgctggaacccggaagtccgacttcacactgctgggagactgc |
| | atcaacacggcctcccgtatcacctcgttgtctgtcaagctgaatgtgcctctgctgctgtctttcgaggtgcgct |
| | gcctgcttggcgacgagatgcgagaggagctggagagtgccgggctgcacaaggtaaagggccgcgataagcctgt |
| | ccaggtttaccagttcaatgccccagagcttgacagcgcacaggtccgtgccaagattgagtcgttcaaccccggc |
| | cgcttccgccccatgtgccccgtaaagccctacgattcactgcaccccagccagagaccccccatcttcgacgaca |
| | ccccccgggagagcatgccgaagctgagcgtcgtccagcgccgtgacagcctcgtggaccggctgtcgctgatcgc |
| | taagctggcgttcccgccgaacatgatgaacgggggcgacaaccagctgatcacgcttacgtatatctcccaggca |
| | tcatacccgatgtcccggcttgacttggcggccatccagcgggttggatgccggcgcaacgagtcggcccagatca |
| | ccgggtcgctgatctacgtcaacgggctgttcgtgcagacgctcgagggccccaagtccgcggtcatcacgctgta |
| | cctcaagatccgccaggactcgcgccacaaggacgtcgtgatcgtgttcatggcgcccatcgaggaacgtgtgtac |
| | agcaccgcgctggacatgacgtccgcgacggaggagatgctcgccacgttcccccctttgcaggatgtgttgtcgc |
| | agctggccaagtctttcatctcgctggagacgtacgtgccatccacggtggtgcgctacctcaccgccgggaacaa |
| | cccgcgcagcctgcagcccgtctccgtggaggtcgtcatgctggccaccgacatttgcagcttcacgccgctgtcc |
| | gagaactgctcgctgacggaggtttggacgatctgcaacacgttcatcgacgcctgcacctctgccatctgcagtg |
| | aggggggtgaggtgatcaagctcattggggactgcgtcacggcctacttcccgccgacgtccgccgacaacgccgt |
| | ggccgcctgccaagagatcgtcaccttctgcacccagctgcgggaggcgttcagtgatgtgctggactgccggtcg |
| | gtcatcgcgtgcggggtgggcctggactacgggcaggtcgtcatggcgcagtgcgggagtctggggatgacagagt |
| | tcgtcgtcgccggggaggtcagcgcccgcgtcatggaggttgaggcgctgacgcgtgaggccgggttcaccatcgt |
| | ggtcacagagcccgtggcggaccgcctgtcccccaagctgcgggacaccggcatcatcccctgccacgagggggtg |
| | gacggcgtgccgtgctacgggattgccggtgacgagtgggtgctggatatcaagactatcaaggcgaacatctacg |
| | gcttccatgaggcccgcgcccaggctgccctgcggaaggtggatgatggcacccgtgccccaggccatgcggcccc |
| | tgcgtctcccaaggcacagaagggagcgggccgggccaactccgtcgccagctacacccccgacgtgaacgagctg |
| | ctggacccccggatggctgagtcggtgttccaggagctgtgccagcagcgtggggacgtcccgaatgtggggattt |
| | cagcaaagctgcggcaggcggcgcaggacgaccgcctggacctcggccgcacgctgcagggcccccatgagctcgg |
| | gccggtggtgcaggcggtaaagcagctcacaaacttgaagacgctgaacctgagcgacaactttgtggacgacggc |
| | agcgtcggggaaattgtggaggcctgcctccccatgcggaacctgcaggttctggacctctccaacaacccggggc |
| | tgacaaagatcatcgcattgaagcgcctcatcaaacacaacccggccgtgcgggagatcctcctgaacggcacccg |
| | cgtggccccgacggagcagcgaaagctgcagtcctccatcaacgtcaaccgcctctgctcctccaccgatgcgaag |
| | ggaggctcccacaagtatgagagccattaagggggcgccgccgtgccccacggccggctataaaaaccgaggcgtt |
| | gcgtgtggcggcaaaccgtcctgcttgcctgggcggttgtatcaacaaagggtgaggggtttggggtgcaggttgt |
| | aaaacgttggctgcactacctgttgtttttttttttttttttggagctggcccacaccaaaattggcctccagctc |
| | ccctttgtcaccagcgcatgatggcttcggccactgttccacaatccacggattccctgcttggtcgactctgtgc |
| | ccggcccctcccccaactgctgtcattcttgactgtcccatgaccccgccggcattgctactggcatcctgaactc |
| | agggcgatgaccttcatcgattcgcaaattcgcccgaagttgaaagtgatgtcttctccctcttcaatccgttgct |
| | gaccacagtgaccag |
| Putative alpha | cctgtcacgcccttcacctcgttgaggatgtacatccttgtttggaaagatggccaacaggttaaaaacttccaag |
| subunit of | atttggaagaatgcggcgcattccagacggctgctaacatcgtcgacggacagatcttcaatgtcggcgtcacctc |
| photoactivated | agcgatgaacaaaggaggagacactggtgaagtgcagcttcgccgcttgatgtacctttctgcctccacggagccg |
| adenylyl | gaggaatgcaccaccgagtatctcgctgaattggcacgtgcttcgtctctccggaacaaagagattggagtgagcg |
| cyclase, 4.6.1.1 | ggttcttgttgtacagtgcaccgttcttcttccaagtgattgaaggaaccgacgaagaccttgatttcctcttcgc |
| Euglena | caagatttcateggacccccgccacgagaagtgcatcgtcttggccaatgggccgtgcaccggacgcttgtacgga |
| quartana | gaatggcacatgaaggattcccacatcgacaacatcacaaagcaccctgcgatcaagaccatcctgttccaaatcg |
| mRNA | ccaaatcgttctccagcatgtggtcctacttgtcaaagaacacggcgaacatgcttctgttgggcaaacagcccaa |
| SEQ ID NO: 45 | caagcaagcaccagagcccatgagcgtggtcatatccttcatctatctggtggagttctcatccatcttggctcac |
| | cccgggttgacggagcagtgtgccgacgtcttggctgcatttgtggacgtttgcgtaaggaacatcgagggaactg |
| | gagggcatgtggccaagttcatcaccggaatctgcatggcttactggcccagcaacagggctgaagatgccctggt |
| | cggtcttcagcaactgtccgatgatctcgctgagcttcgaagccagcagcctccgggctctgcgctgtccctcgtc |
| | tactcgcggtgcggtgtgcactatggccgagcgctgctgtgcaacgccggtattaagaaggccgactttactctgc |
| | tcggcgactgcatcaacaccgcctcacgtatcacttcgttggcggggaagctgaaggtgcccctgttgctgtcttt |
| | tgaagtgcgttgcttgctgggagctgagatgcgggaagagctggaaagcgccgggatgcacaaggtgaaggggcgt |
| | gagcagccggtgcaggtgttttatttcaacgctgctgaggtggacagtaccgccgtgcgagcgaaaattgagcagt |
| | tcaaccccgggcgctaccgcgcgttgtgccccgtgaagccctacgacagcctgcacccctctcagaagccgccgat |
| | tttcgatgacactccgagagactccatgccgaggtccggtcatttgcaaaggcaagacagccttgtggaccggctc |
| | agcatgatcgccaagctggcgtttccgcccagtatggcagcgggcggagaaagtcagctcattggcctcacttaca |
| | tatcgcaagcgtcgcatgccatgtcgcgcctcgacctatccgacatccaaagggtcgcccaccggcgcaacgaaca |
| | ggtgaacatctccgcttgcctactctacatcaatgggcttttcgtgcaaacgctggaggggccgaagagcgctgtg |
| | gtgtctttgtacctcaagatccggcaagacccccggcacaaagacgtcgtggtggtccacatggcccctctcgagg |
| | agcgaatgtaccgcggcccgttggacacaacgtgtgctacggacgagatgctggccacatttccgcctttgcagga |
| | cgtactgtcccagctggccaagtccttcatctccctggagacgtatgtgccctccaccattgtgcgttacctcacc |
| | tccggcaacaacccgcgcaacctgcagcccgtgtcgctcgaggtggtcatgcttgccaccgacatctgcagcttca |
| | ctcctctgtcggagcactgctcattgacggagatatggaccatctgcaacacgttcatcgacgcctgcacctctgc |
| | catctgcagccagggcggcgaagtcatcaagctgatcggggactgcgtcacagcctacttccccccaacatctgca |
| | gacaatgcggtctatgcatgccaggatatcgtggccttctgcgcaaacctgcgcgaggcattccgcgatgtgttgg |
| | attgccgatcggtcgtggcttgcggtgtgggtcttgactttggacaggtcatcatggctcagtgcggcagctcggg |
| | gctgacagagttcgcggtggccggtgaggtcagcgctcgcgtgatggaggtggaggccctcacgcgtgaggcgggt |
| | cgcgcgatcgtactcacggagcctgtggcggatcacctttccccgaagctgcgtgataatggactggtaccttgcc |
| | aagaaggtgtggacggagtgccctgctatggcatcttgggagcggagtgggagctggacgtggtgaccatcaagag |
| | gaacatccaaaactttcacgccgcgcgcgctgtggctgccgtacagaaggtcgatgacggaatgaacgcccctggg |
| | cgtgccctgccagtcgcctccatgccagccatatcgtccccgtcccccaaggtgcgcactgcggggcgtgccgctt |
| | cggttgccagttacgctcccgatctcaacgatctgctggaccctcgcatggcagaggttgtgttccaggagctctg |
| | ccagcagcgcggagagatccccaacaacgccgtcctcctccggcttcggcaggcggcccacgaagagcgcgccgac |
| | ctcggccgctcgcttcagggaccgcacgagcttccccctctgacccaagccctgaagcagctcaccaacgtgaggg |
| | ctttgaacatgagcgacaacttcgtggacgatacctccatcggagacctcatcgaatcgtgtcttgcaatgcggtc |
| | cctgcagatgcttgacctgtccaacaaccccggactcaccaaagtcategccctcaagegcctcatcaaacacaac |
| | ccgcagctccgtgagatccacctcaacggcacgcgcategcccccacggagcaacgcaagctgcagtcctccctca |
| | atgtcaaccgcctctgcgcctccgctgaatccaagctgggttcgcacaagtacgaaggcgccgcccactagagccg |
| | cttttgctgct |
| Putative alpha | attcaagatgtacatcttggtttggaaagaggggcagcagatcaagacatttcaggatcttgaggaatgtggccaa |
| subunit of | ttccagacggcatgcaacatttcagatggccagatcttcagcgtgaacgttgctcccaccatgagcaagggtggag |
| photoactivated | aaagtggtgaacagcttttgcgtcgattgatgtacttatcaaagtcaacagagcctgaagcctgtaacgctgaata |
| adenylyl | cttggcacagatggcctttgtcgcaacatgcgtcaatagcacgattggagtcagtggtttcctgttgtacagcgca |
| cyclase, 4.6.1.1 | ccgttctttttccaagtcattgaaggaacggacgatgacctggactggctgtacgcgaagatctccagggacccac |
| Euglena stellata | ggcatgagcggtgcatcgtgctcgcgaatggtccttgcactggacgaatgtacggtgaatggcacatgaaggattc |
| mRNA | ccacattgataacatcaccaagcacccggccatcaagaccatcttgttccagatcgctcgctcgttctccagtatg |
| SEQ ID NO: 46 | tggtcgtacctccccaagaacgcagccaatatgcttttgcttggcaaggagcccaacaagcaagctccagagccaa |
| | tgagtgttgtgatcacgttcatttacctcgtggagttctcttctatccttgcacatcctggattgactgagcagtg |
| | cgccgatattctggaggcttttgttgatgcctgcgtgcgcaatatagaaggaaccgggggtcaggtcgccaagttc |
| | atcaccggtatttgtatggcgtactggccaatcaaccgcgctgaagatgccattgtggggctgcagcagctgtctg |
| | aagacctggcggaattgcgcagccagcagectcctgggtctgcattgtcactggtgtattcaagatgtggtgttca |
| | ctatggacgtgcattgctctgcaatgctgggttccggaaggctgacttcaccctcctgggtgattgcatcaacacc |
| | gcctcacgtatcacttcgctgtccgtcaagttaaaggttcccctgctcctctccttcgaggttcgctgcttgttgg |
| | gcgacgagatgcgtgaggagttggagaactctggcctgcataaggtgaagggtcgtgacaagcccgtgcaagttta |
| | ccagttcaacgctcctgagcttgacagtgccatggtacgagccaagatcgagcagttcaaccctggccgttaccgt |
| | gcgctgtgccccgtgaagccctatgacagcctgtctgctactcaaaagcctccaatcttcgacgacaccatgcgtg |
| | atggccacaataagcttagtgcggcacagcgccgggatagtcttgtggaccggctcagcctgatcgccaaattggc |
| | cttcccaccaagcatgctggccggtggagatggccagctcataacgctgacgtacatttcccaggcatcccgcccc |
| | atgtcgcggcttgacctgtcggcgatccaccgtgttgcaatgcgccgcaacgagtcctccaacatttcaggttgtt |
| | tgctgtatgtgaacggcttgtttgtgcagaccctggagggtcccaagagtgcggtggtttcgctgtatctgaagat |
| | ccgccaggatgggcgccacaaggatgtggtgacggtgtacatggcccccctagaggagcgcgtgtatgcgaccgcc |
| | ctggacatgactgccgctacggaggatatgctgtccacgttccctcccctgcaggacgtgctgtcccagctggcac |
| | ggtcgttcatctccctggagacgtatgtgccttctaccatcgtccgtttcctgacggccgggaacaacccccgcaa |
| | tctgcagccggtgtctgtggacgtcgtcatgctggccacggacatttgcagcttcactccattgtcagagcgctgc |
| | tctttgacagaagtgtggactatctgcaacacgttcattgatgcctgcacaagcgccatttgcaacgaaggcggag |
| | aagtcattaagctgattggggattgcgtgactgcctatttcccaccgcatgccgcagacaatgcggtcagcgcatg |
| | ccaggagatcgtaaccttctgcacccacctgcgcgaggcattcaaggatgtgctggactgccgttccgtgatcgcc |
| | tgtggtgtgggtctggacttcgggcaggtcattatggcccagtgcggtagcctgggaatgactgagtttgtggtgg |
| | ccggagaggtcagcgcccgagtcatggaggtggaagctttgacccgtgaggccgggcgtgccattgtggtcacaga |
| | gcctgtggccgatcgcctctcctcccggctgcgagacagcggcatcgtgccctgccaggagggtgtggacggggtg |
| | ccctgctacggtatcctcggaccggagtacgagctggacatcgtgaccgtcaaggccaacatctatggattccacg |
| | acgctcgtgcccaggcagctctgaggaaggctgacgatgggacgaacgctcctgggaagtcaggccctgcggtacc |
| | ttcatctccaaaggtccaccacgctcaaggccgtgccggctccgtggccagttacacccctgacccgaatgaggtc |
| | cttgaccctcgcatggcagaaatggtgttccaggatatgtgccagcaccgtggtgaccagcccagcaacgcgatcc |
| | ttatgaagttgcgaaacgccgcccaagatgatcggctggacctgggccgcaccttgcaggggccccatgagctggg |
| | acccctcacccaagccattaagcagctcaccaacctaaagatggtgaacatgagtgacaatttcattgatgacaac |
| | accattggggatttggtggaggcttgcttgtcaatgcgatctctccaggtccttgaccttagcaacaaccctggcc |
| | taaccaagactatcgccctgaaacgcctcatcaagcacaaccctcacgtccgtgaggtactcttgaatggtactcg |
| | catcgcgcccacggaacagcgtaagctgcagtcttccatgaatgtaaatcggctgactgcgtctgacaagggtggg |
| | tctcacaagtatgaccactagactatcgggtgccgataccgcgtgatgtaaagaatggtgcaccaaaacgatgcac |
| | tatt |
| Putative beta | tgcacgatgtacatcttggtttggaagaaaggccagcaaatcaaaacattccacacactcgatgaagcagcacaat |
| subunit of | ttaaagcagcctctaacatcgatgacgctcagatgttctcaattaacgttgctccagcagtcagtgcgtcaggggg |
| photoactivated | aagtaacgaggccactaacttgcgccgtctgatgtatctgtcgaagagcacagagccagaaaagtgcgatgtcccc |
| adenylyl | tatcttgccgagatggccggaatagccactctgaggaaccgtgaaattggagtcagtggtttccttatgtactctt |
| cyclase, 4.6.1.1 | ctccgttcttcttccaggtcattgaaggaacagatgaggatttggatttcctttttgcaaaaattggggctgatcc |
| Colacium | caggcacgaaaggtgcattgtgcttgcaaacgggccttgcacggggcgcatgtatggtgattggcacatgaaggac |
| sideropus mRNA | tctcacatcgacagcatcaccacccatcctgcgatgaagacaattttgtatcagattgcacgctccttctcaagta |
| SEQ ID NO: 47 | tgtggagctacttgccaaagagcgcaggaaacatgttgcttttgggcaaagatcctgcttcccaacctccggagcc |
| | aatgagtgtggtggtgacattcatttacctggtggagttcggatcaatcttgtcaaacccaaatttgactgaacaa |
| | gcagctgaagtgctgagcacctttgttgatgtttgtgtgaagaacgtggaaggcagtggtggaaacattgccaagt |
| | tcatcacaggaatttgcatggcgtattggcccatcaacagagctgaagatgcattgacatcaatccagcaaatcag |
| | tgaggatttggcccaactccgaagccaacaggcccccggctcagcagtttccctgatgtattcccaagccggggtg |
| | cactatggccgggccatgctctgcaatgccggaacccggaagtcggacttcaccctgctgggcgattgcatcaaca |
| | cgacgtcccgggtggccacattggccaagaagctgaagactccgttgctcttctccttcgaagtccgctgcctgct |
| | cggtgatgagatgcgtgaggagattgagggggctggcatgcatcaggtgaagggccgtgacaagcccgtggtggtg |
| | taccaattccccggagaggagctggacgtggatgaggtccgccacaagattgagaccttcaacgccggccgcttcc |
| | gctgccagatgccggtggtggagtacgacacgctccacccctcgcagcggcccccgatcttcgacgagaccccgaa |
| | ggggaacccccgcccccgcacccccggctacgggggacccagcgccgcgacagtctggtggaccgcctgatcatga |
| | tcgccaagctggccggtaccactgcttcgaccaccggagacacaacgctcatcacgctcacctacatcagccaggc |
| | gtcacgccccatgtcccggctggatttgtccgccatcatgcgcgcctccacgcgccgcaacacgcagcagaacatc |
| | acagggacactgctgcacgtgaacgggctgtttgtgcagaccctggagggcccccgggatgccgtgatgaacctat |
| | acctccgcatccgccaagacccccgacatactgacgtggtgaccgtccacatggcgaacttgcaggagcgtgtgta |
| | cccgccggagtggaccctcacttcggcctccgaagacatgctggcggcgttccctccactacaagacgtgatgtcc |
| | cagctggcgaagtcattcacttctttggagacctacgtgccatccaccgtcgtgcgttacctgactgctggcaaca |
| | accccaggaatttgatgcctatttcctgcggggtggtcatgttggccactgacatctgcagcttcaccagcctcac |
| | cgaaaagtcttctttgacggaggtctggatgatctgcaacacgttcatcgacgcgtgtacttcggccatttgccag |
| | gaggggggtgaggtcatcaagctgattggcgactgcgttaccgcctacttcccgggctctgctgccgacgctgctg |
| | tggccgctgcccaggagctgtacctgttctgcactcaactgcgtgaggccttcgtggatgtcctcgatgtgcgcgg |
| | ctgtgtgtcatgcggcgtggggcttgactacgggcaggttgtcatggcgcagtgcgggacccttgggctgacagag |
| | tacgttgtcgccggggccgtgagcgcccgcgtcatggaggtggaggccctgacccgagaggtggggcatgccattg |
| | tcgtcacagagccggtggcagaccgcctgtccccccagctgcgggactcgggcatcatcccgtgcccccaagccat |
| | cgagggcatcccgtgctacgggatcgccggggagcagttcgagctggacatcgactccatcaagcgtggcatcaag |
| | gcgttgcacgcggcgcgcagcggggagaaggtggcggtggcggagccggaggagaagggcggcgacttccgtgtct |
| | ccccgggccgcgtccgccgcagcgactccggccgccgctccaactccgcgcagggcaagcggggcacccctgtccg |
| | ctgatctttcgaaccaacagggtttgtaaacttcacagtgcctttcctttacctgctggctccactgtggtcgtga |
| | acttcattgggttgtactctataaaccttaccccttttttttctttcgagtttgtttgttcaccgtccgcgcttcg |
| | ttttggcggcacttcgtaacgatgaaccccttccgtgggatcattccgcaatgcatatccacttcgcttgacctga |
| | c |
| Putative beta | gggacgcatcagctgcaaacaccatgtttctcttggtttggaagaagggccagcaaatggcaaccttccacacgct |
| subunit of | ggaagaatccaacgagttcaaagccgcgtccaacattgaagattcacacgtcttccacttgaagctcacacccact |
| photoactivated | accagtggtgctggtggagccaacaatgccaccggatttcgccgtttgatgtacctgtcaaaatctacggaggctg |
| adenylyl | aggaagtgaacagcacttatttggccgagatggctggaatcgcaacgatccgtaaccaggagatcggcgttagtgg |
| cyclase, 4.6.1.1 | gttcctgatgtactctgaccctttcttcttccaggttatcgaagggactgacgaagacctagatttcctgtttgcc |
| Eutreptiella | aagataggagcggaccctcgtcacgaaaggtgcatcgtgctggccaacgggccttgcaccggacgcctgtacggag |
| gymnastica | actggcatatgaaggattctcacatcgatagcattacctogcaccccgccatgaagaccatcttgtatcagattgc |
| mRNA | cagatccttctctagtatgtggagctatttgcccaaaagtgcaggacacatgttgctgcttggtaaggaccccaac |
| SEQ ID NO: 48 | aaacagcctcctgagccgatgagtgtcgtcgtcaccttcatctacttggtggagttctcgtccattctctcacacc |
| | ccggcttgactgagcagtgcgctgaagttctgagcaccttcgtcgacgtgtgtgtgaggaacgttgaaggcagtgg |
| | aggaaatattgctaaattcatcaccggtatctgtatggcgtattggcctatcaacagggctgaagatgccttgaca |
| | gctctccagcagatcaacgaggatcttgctcagctccgtggagcgcaagcccctggctctgcgttgtccctgatgt |
| | attcccgttccggagtgcactacggtcgagctatgctgtgtaacgctgggactaggaagtctgacttcaccctcct |
| | cggagattgtatcaacactgcatcacgagttgcaagtttggctgttactctgaagtcccccctgttgttctcgttc |
| | gaagtccgctgtttgctcggcgacgagatgcgtgacgaaatcgaaagtgctggaatgcaccaggttaagggacgtg |
| | acaagcctgttgtggtttatcaattcccaagccctatcttggacagcgatgttgtgcgccagaagattgagcagtt |
| | caaccctggccgctacaggtgctccatgcctgtgcttgactatgaaaccatgaatcctgcaaagcgacccccaata |
| | tttgacaacgcccccaagggcaacccgaagattggcaagaagtacaacgctgctgcccaccgtgatagtttggttg |
| | acaggctcaccgagattgcccggctggcgttcccaggatcccaggctggcggtgattcgaccttgatcagcctgac |
| | ctatctttccaactccgttcgccctatgtcccggttggaccttgcagctatccagcgtactgcactccgccgtaat |
| | gcaagccagggcatcaccgggtgtttgctttatgtcaatggattgtttgtccagaccatcgagggcccaagagata |
| | atgtgatcaatctgtacttgaagattcgggaagatccccggcacaaggacgttgtcactgtctacatggtccccat |
| | ccaggaccgtgtgtaccaagaggcctggactatgaccactgccagtgaagacatgcttgccacgttcccgcctctg |
| | caagatgtgttgtcgcagttggccaagtcgttcacttctcttgagacctatgtgccatccactgtggtgagatatc |
| | tgactgctggaaacaatccaagagatctgatgccagtttctgtggagtgtgtcatgtttgccacagatatctgcag |
| | ctttactatgttgacagagaaatcatctctggaggaggtttggatgatctgcaacactttcattgatgcctgtacc |
| | tctgccatctgcgccgagggcggtgaggtcatcaagctgattggcgattgtgtcactgcctacttcccaagcgaga |
| | gggcagacaacgctgttgctgcttcacaggagctgtatatgttctgcgttcacatgagggaagctttccaggaaat |
| | tctcgatgtccgtgcctgcattgcttgtggggttggtttggactatggccaggtcgtcatggctcaggctggtagt |
| | ttggggttgactgaatatgctgtcgctggggctgtcagtgcccgcgtcatggaagtcgaggctctttcccgtgacg |
| | tcggccgtgccatcgtttgtaccgagcccgtggttgacagactgtcgccacacttgcgtgattctggtgtcattcc |
| | atgccctcaggcaattgagggaatcccttgctatggaattgctggtgaagattacgagctggatatccgatccatc |
| | aaggctggcattaagcagctgcatgctgcccgttctcttgaagatcaggctgctgctgcagaggaggaggctgcgc |
| | aggcccagcagcagttccgtgtgtccccagggcgtgtccgtcgcagtgatgctggccgccgctccaattctgccca |
| | aggccgtcgcatggccccagtccggtaaatattgcaaacatgccactcctgggttggttagatcagagggcaatgg |
| | ttggtggttggtctagcaggtgcctggaccgcatttgccatctccctttggttgccaatgatcggttcgattggat |
| | ggttgaagtttttttggtgctctgttttgcaatagttgcaaggccagccagtcatccactaattgcaaaaatgaaa |
| | ttcacttcgtatttggaggttgtcattcaggacgtgattgtaaagtagacctcttgtcaacgttctggattgtaaa |
| | gtagtagtagtatcatggaccataatattacaacggtattagagtatcagtgtcatgtcatgcaatcaaatcttca |
| | acattcatctgccatgccataccaagtttctgaccgttgtcagattgtttgattatcagaaaagagactgttcgtt |
| | ctatatcccagaaagtcatggcatctattggacctattttaccaggttgatggtgaaaataacatttttgcgcatc |
| | gataagattctgcttaaacatagtctcgtctctttgatgcgccatcctttctgcccatccattcatttcaatttgg |
| | attttaccaggtgtctggcattttgcagtgcagtgcagtgcagtggtgtttcccttcctacagtgcttctctggga |
| | tttcacggacaccgcacacagtatggcttataagagcacccatggtgcacacctcatctatccttgttggcttaca |
| | aaattagttttgtcccatggagtg |
| Putative beta | gccacagcatgtacatccttgtctggaagaaggggcagcaaatcaagtcattccatacgcttgatgaggctgcaga |
| subunit of | attcaaggcagcttcaaacatcgatgaggcacaaatgttcagcattacggttgctcctgcaatcagtgcctctgga |
| photoactivated | ggcaccaacgaggccacgaatctgcgacgtttgatgtacctttcaaagtccacaaatccagaagagtgcgacccag |
| adenylyl | cattcttggctgagatggcgggaattgcgaccattaggaataaggagatcggagtgagtggtttcctaatgtactc |
| cyclase, 4.6.1.1 | ctcccctttcttctttcaggtcattgaaggcacagatgaagaccttgatttcctctttgcaaagatctcagctgat |
| Euglena stellate | ccccgccatgaaaagtgcatcgttttggccaacggaccttgcactggtcgtatgtatggtgattggcacatgaaag |
| mRNA | actcgcacattgacagtatcaccacccatcctgcaatgaagaccatcttgtatcagatcgcgcgctcgttttcaag |
| SEQ ID NO: 49 | tatgtggagctatctgcccaagagcgctgggaacatgttgttgcttggaaaggaccctgcagcccaaccacctgag |
| | ccaatgagtgtgatcgtcactttcatctatttggtggaattcggctccatcctctcaaaccccgatctaaccgaac |
| | aagcggccgaagtgctgggaacctttgtggatgtctgtgtcaagaacgtggaaggcagcggaggtaatattgccaa |
| | gttcatcactggcatttgtatggcatattggcccattaatcgcgcggaggacgccctgacgtccatccagcagatt |
| | agcgaggatttggcccagctgcgtagccaacaggcccccggctctgccatcacgttgatgtactcccaggccggag |
| | tgcactatgggcgtgctatgctgtgtaacgctggtacacgcaagtccgacttcaccctcctgggcgactgcatcaa |
| | caccacgtcccgagtggccaccctggccaagaagctgaagaccccgctgctgttctccttcgaggtgcgctgcatg |
| | ttgggcgatgagatgcgggaggagatcgaaggcgctggcatgcatcaggtcaaaggacgcgtgaagcctgtggtgg |
| | tgtaccagttccctgggccagagctggatgtggaaatggtgcgacagaagattgaccagttcaaccctggtcgctt |
| | caggtgccagatgcctgtggtggattatgactcgctgcccgtttcccagcggccccccatcttcgatgacacgccg |
| | aagggcaacccccggccccgcactcccggctatggaggcacccagcgaagtgacagcctggtggaccgcctcatca |
| | tgattgccaagttggccggtccatccgcatcagccggaggggacacctccctgaccaccctgacgtacatctcgct |
| | ggccacccgccccatgtcccgcctggacctctccgccatcatgegcaccgccacccgccgcaactcgtcgcagggc |
| | atcacgggaaccctgctgtacatcaatgggctgtttgtgcagaccctggaggggcccaaggatgccgtggtcaacc |
| | tgtatttgaggatccgccaggacccacgccacacggcggtggtcacggtgcatcaggccgctctccaggagcgtgt |
| | ctatcctccggagtggactatgacgtctgccaccgaggagatgttggccacattcccgcccctccaagacgtgttg |
| | tcgcagctcgccaagtctttcacctccctggagacctacgtcccatccactgtggtgcgttacttgacagccggca |
| | ataaccccaggaacctcatgccagtgtcgtgttccgtggtcatgctggctaccgatatctgcagcttcaccagtct |
| | gacagagaagtcatccttgacagaggtgtggatgatctgcaacacgttcatcgatgcctgtacctccgccatctgc |
| | caagagggtggggaggtcatcaagctgattggggactgtgtgacggcctacttcccggggaatgccgccgacgctg |
| | ctatagctgctgctcaggaactgttcatgttctgcacgacgcttcgccaggcctttgtggatgtgctggacgtgcg |
| | cggctgtgtagcatgtggcgtaggactggactacgggcaggtagtcatggggcagtgcggtacgatgggtctcacg |
| | gagttcgtggtggccggggccgtcagcgctcgggtcatggaggtggaggcgctgacccgtgaagtgggccgggcca |
| | tcgtggtcacagagccagtggcagaccgcctgtccccccagatgcgtgacacgggtatcatcccctgccctcaagc |
| | catcgaggggttgccgtgctacgggatcgccggggatgactacgagctggacattgatgagatcaagcggtccatc |
| | aagcacctgcatgcggctcgcagcggggagaagatgccgtccgcggatcacgaggagaataagggcaacgatttcc |
| | gcgtgtcccccggccgggtgcgccgcagtgactcgggcaggaggtcgaactcggcccagggcaaacggggcacccc |
| | tgtgcggtgaactcaggtgggcgcataggtccgaggcagtcctctctctcatttgggcatggtacaccactttttt |
| | taattggccttggtactcgcattgggcataccctgagggatggtgccgctacattgtatggtcactttgct |
| Putative beta | taatcgtgctctcgcaaaatggttttcctgctggtttggaagaatggtacgcagctgaagtccttcctgaccctgg |
| subunit of | acgacgctactcaatacaaaacagcaagcagcattgatgatgctcagatctacaatgttgctttcacccctgctgt |
| photoactivated | cagtggagatggtgggtccaacgaagcaaccaatctccgacgtctcatgtatctgtcgaagagtacggagcctgaa |
| adenylyl | ctggtgaacacccaatatctcgccgaaatggctcacatcgcaacgctgcggaatcagtgcatcggcgtgagtggtt |
| cyclase, 4.6.1.1 | ttttgatgtactctcatcctttcttctttcaggtgatcgaagggactgatgaagatttggattttttgtttgccaa |
| Eutreptia viridis | gattggtatagacccacgccacgaaaggtgcattgtgttggcaaatggtccctgcactggaagactgtatggggat |
| mRNA | tggcacatgaaagattcgcacatcgacagcattacctcgcaccccgctatgaagacaattttgtatcagatcgcaa |
| SEQ ID NO: 50 | ggtctttctccagcatgtggagctatttgcccaagagtgccggcaacacactcctgctcggcaaggacccagctgc |
| | ccagccaccagagcccatgagtgtcgttgttacgttcatctacctcgttgaattctcgtccatcctctctcatccg |
| | ggattgacagagcaatgtgccgaagtgttgagtacctttgtagatgtttgcgtgaagaacgttgaaggaagcggtg |
| | gaaacattgcgaagttcatcacaggaatctgcatggcgtattggcccatcaacagggcagaagacgcgctgaccag |
| | cattgcgcagatcagtgaagacttgggacagctgcgaggccaacagcctccaggctctgctctatcgctcatgtac |
| | tctcgcgccggggtccactatgggcgtgccatgctgtgcaacgccggatcccgcaagtcggattttacactgctgg |
| | gggattgcatcaacacggcatcccgggtcgcgtcgttggccgtgactttgaagtccccgctgttgttctcattcga |
| | agtccgatgtcttctgggcgacgagatgcgagacgagatcgaaggggctggcatgcattcggtcaaggggaaagac |
| | aagcctgttgtggtgtaccagttcccagggcccgagctggatagcggtatggtgcgccagaaaattgaagagttca |
| | accctggtcggtacaggtgcggcatgcctgtcatggagtacgagtccctgcacgtgtctgagcagccccccatctt |
| | tgatgacacccccaaggggactcccaagccaggcacacgggcttggggaggaacgaaccgaaaggacagtctggcc |
| | gacaagctggccttgatttoccaattggccggccccctccagacatccggggacaccacgctgacaaccctcacct |
| | acatctcccactccacgcggccgatcacccgcatggatctggcctctatccaacgctgtgccctgcgccgcaacgc |
| | tcagcataacatcactggatgcctgttgtacgtcacgggcctgtttgtgcagaccctggagggccccaaggacgca |
| | gtcatcaacttgtaccacaagatccgcgctgacgcccgacacacggacgtcgtcaccgtacaccagggccccattc |
| | aggaccgtgtgtatgcctctgcctgggccctgacaaacaccagcgaggagatgctttccgctttccctccgttgca |
| | agacgtgctctcccagctggccaaatcttttacctcactggagacctacgtaccatcaaccattgtccgctacctg |
| | actgctggcaataaccccaggaatctgaggcctgtctccgtgggggttgtcatgttggcgactgacatatgtagtt |
| | tcacaatgcttactgagaagtcatcccttactgaggtctggatgatttgcaacacgtttattgatgcctgtaccaa |
| | tgccgtaattaccaatgggggagaggtgatcaagctcattggggactgtgtgaccgcctacttccctgggcatggt |
| | gctgacggtgctgtggccgctgcccaggagatcttttccttctgtacccaattgcgggaggcctttgctgacgcgc |
| | tggatgtgcgctccgccatttctgccggaatcggcctggactttgggcaggtggtgatggcccaggctggaagtct |
| | tggggtgacagaatttgctgtggcagggtctgtcagtgcccgggtcatggaggttgaggcgttgacccgtgatgtg |
| | gggcgcgccatcgtggtgacagaacctgtggcagaccgcctgtcccctcagttgcgtgaccgtggcctcattccat |
| | gcccccaagccatcgagggcattccgtgttacggccttgccggggatgactttgagctggacatcgcctccatcaa |
| | gggccgcatcagaaaccttcacgccatgcgggcaggggaaaagcccaacctcccagacgaccatgagcaagaggat |
| | aactacaagcaagcccagcaggacttcagagtgtcccctggacgagtgcgccgtgccgattctggccgccgctcca |
| | actcggcccagggccgccgcagcgcacctgtccgataaccgggcagggtacatcaccgcgcagtggccttgtaggg |
| | cccactcccgcagcatcgaaccttgcaaggggggggatggtaggcgcagcatctgcatgtgcaaggcccgaccctt |
| | cgacategggcccagcaaggggggggattggagtccaacctgccctcagccagggctttgtttcttatgcgtgatg |
| | gtggtgtgcaggcaatggacaaatcatggcgaatgctctctgccatgtaggtgctccccccgcccaattegaagta |
| | gtcccccagcctcttccaccctctgatgacaccctgctactgtactctattgtatgtttcttagggcccatcaatg |
| | actggggtgacagtctgcttgt |
| Putative alpha | gtcggacaacataaaaaatgtacgtgcttatttggaagtctgggcaacaaattcagactttccctgatctggaagc |
| subunit of | tgccagccagttcaaggctgctagcaacatcgaagattcgttcattttcagtgtcagcgtgacaccaacaatgctt |
| photoactivated | gcgggcggtgctgctggagagtcaggccttcgtcggctaatgtatctttccaaggccacagaacccgatgagtgta |
| adenylyl | cacccgagttccttgccgaactggcacacgttgcaaccctacggaaccaggagatcggagtcagcggtttcttgct |
| cyclase, 4.6.1.1 | gtacagegcacctttcttctttcaggttattgagggaactgacgaagacttggatttcctgttcggtaaaatcggt |
| Eutreptia viridis | ctcgacactcgtcatgaagcttgtattgtgttggccaatggcccctgctccggacgcatgtacggggagtggcaca |
| mRNA | tgaaggactcccatatcgacaacattactaagcaccctgcgatcaagaccattttgtaccagatcgcccgttcctt |
| SEQ ID NO: 51 | ctccagtatgtgggcttacttacccaaacaagctggaaacttgcttctactcggcaaagagcccaacaagcagcca |
| | cccgagcccatgagcgcagtggtcagcttcatttatctagttgagttctccaccatccttgccaatcccaatctga |
| | ctgagcaggtggccgacattctgattgcctttgtcgattcctgcgtgaagaacgtcgagggttcaggtggcagtgt |
| | tgcaaagttcatcaccgggatctgcatggcttactggcccattaaccgagccgaagatgcactgcttggtctccaa |
| | gctatctctgacgaccttgcagagttgagagcacatcaaccccccggttctgcgttgtctcttgtctactcccgag |
| | ccggggtgcattacggacgtgcactgttgtgcaacgccggaatgcagaagtctgacttcactcttcttggggactg |
| | catcaacactgcctcgcgggtcacctcactgtctgtgcaattgaaggtgcccctgctcttctcgttcgaagtgagg |
| | tgtttgcttggcgacgacatgcgtgaggaacttgaaagtctgggcatgcacaaggtgaagggtcgtgataaacccg |
| | tagctgtatatcagtttgctggggccccagagttagacagtgccatggtgaaggccaaggtcgagcaattcaaccc |
| | aggcaggtacagagcacagtgccccgtgcgagaactggaatcgctgcacccggacgttcgcccacctattttcgat |
| | gatactccccgcgaggcttcgatgccgaaggcaggcgcaggggaacgccgtgacagtcttgcggatcgtctgacaa |
| | tgattgccaagctggccttcccgacgategggggaggtactaattcgcagatgataacgttgacatacgtctcctt |
| | ttcctctcgacctatgtcgcggttggatcttgctgccatccaacgcgttggcctgcggcgcaatagccaagtggac |
| | atcaccggatctcttttgtacgtggcacctctgttcgtgcagacgctggaaggcccaaaggatgccgtgatctcag |
| | tctacatgaagattcgtgcagatccacgacaccgagatgtggtggcggtttacatggccccacaggaggagcgggt |
| | gtacaacagtgccttcgagctgaccactgcaacggaggagatgctgagtgctttcccccccttgcaggatgtgctt |
| | tcacagctggctaagtccttcatctccctggaaacctacgtgccctcgacagtcgttcgctatcttaccgctggca |
| | acaatcctcgcagtctccaagccatctccacgggggtggtgatgttcgccacggacatctgtagcttcacgcccct |
| | ctcagagaagtgcagcctgacggaggtgtggctgatctgcaacaccttcatcgacgcctgcaccgccgccatcgtc |
| | caggagcgcggggaggtcatcaagctgatcggcgattgtgtcaccgcctacttcccccccaccaatgccgatggcg |
| | ccgtgatggcttgccaggagatcgtcgccttctgcaccggactgcgggcggcgttcagcgacgtgctggacgtgcg |
| | gcaggtggtggcgtgcggggggggctggactacggggacgtggtgatggcgcagtgcggcagcatggggatgacgg |
| | agtttgtggtggcgggggaggtgtcggcgcgcgtcatggaggtcgaggccctcacccgcgaggtcggccgcgccat |
| | cgtcatcaccgagcccgtcgccgacegcctctcccccaagatgegcgacaccggcategccccctgccccgagggc |
| | gtcgacggcatcccctgctacggcattgttggcgacgactgggatctggacattgtcaccatcaaaaagaacatct |
| | acagcttccatgaccgccgcgatgccgtggcccacgctaaggccgcggcagcccaggaggccctggacgctgcagc |
| | ccccagccccaagaaagggggccgtggctctgggaggtccagctcagtgtccagctacgccgcagatcccaacgag |
| | gtgctggacccccgtatggccgaggcggcgtatacgcagggctgccagcagcgcggcgaacagcccacgatggcca |
| | ttgtgaacaagctaaggggcgccggcaatgacacccggatggatttgggccgtgcactccagggtccgcatgagct |
| | catggcgctgtgcacggccctcaagcacctcacgcacctgcgcctgctcaacatgtccgacaacttcgtggacgac |
| | aacagcattgccgagatcgtggaggcctgcctgcccatgaagtccttggcggtgctggacctctccaacaacccgg |
| | ggctgaccaagatcctggccctgaagcggctggtcaagcacaaccccggcattaaggagatcatcctgcagggcac |
| | ccgcatcgccccccccgagcagcgcaagctgcagtccagcatcaacgtcaaccgtctgtgtgcctcctcccccaac |
| | ggcgcggagagcaaggcgggccacaagtacgaccagagcatgtccagtagccaccactgatgcctcccgacgtgcc |
| | gccgccagcagggggagcaggcgccattgtcccgttgttccctgtgtacagtaaccegccccctcccccaaccccg |
| | gcctctcgcccccttttcatcctcagcaaagccgcacgtccccatcgctggtcacagacaatcacagccggtcaca |
| | gacaaggcgaatggaatggacagccaactcccaaacaccccagcccttccccccgccaccagcatgccagacattt |
| | ttccaccgggcgctgggggggcctaggctttatctacgtaagggagacgtgcatacactttgtcacaggtgcagta |
| | ttgcaggaggtatgttaaaccaaggctaaatgcaacccccccccttggggtcggccgggccgggaccctatcaaac |
| | ttgacccccccccccgggtgttccgggggttgggcgtcccccccgccctgtggtccgcggacaagcacaacccttg |
| | ttcacgcctgcgcttgcccctcgaaagctgcgctcacaatccccgtgccatgggcacacegttgggccagccattg |
| | cgtccctgcctgaccttgggacacggcagatcgcctcag |
| Photoactivated | atgtacattttggtgtggaagaaaggtcagcaaatcaagactttccatacgctggatgaggctgcgcagtttaagg |
| adenylate | cggctagtaacatcgacgaggcccaggtcttctctgtgaccgacgctcctgacatctccgcctccggtggctcaaa |
| cyclase subunit | cgaagcccgtaaccgtcgccgtctgatgtacctgaccaactcaacaaacccagaagagtgcaatccccagttcgtg |
| beta-like protein | ggagagatggcgagggtcgcaaccattcagaatagagagatcggcgctcgtggcttcctgatgtactcttgccgct |
| 1224-5/1F | ttctgcttcaggtgattgaagagacggtggaagaccccgactcattgttcgcgaagttcagcccagagccccgaca |
| pacB | tgagcgctggattgagctcgataacggaccttgcaccggccgcatgtacggggactggaacatgaaggacagccac |
| Euglena gracilis | ctcgattcgatcaccacacacccagccatgttgacgatccttttccagatggcgcggagcatttcttgcctgtggt |
| mRNA | cctatttgcagaagagcgagagaatcatgcttctcttcggtaaggatccgggtgcccagcggcccgaacccatgcg |
| SEQ ID NO: 52 | tgttgtggttaccttcacatacttggtcgatttcggcagcatgctcagcatccccaatctcaccgagcaggcagcc |
| | gaggttttgacgactttcgtggatggctgcgttaagaacgtggaggggtccgggggttctattgccaagttcacaa |
| | ccggcatttgcatggctcagtggccgattaaccgcacagtggaagccctgacagcgatccagcaaatctcggagga |
| | tttggcccagctgcgcagccagcaagcccctggctccgccgtttctctgatgtactcccaagccggattgcactac |
| | ggacggggcatgctgtgtaacgccgggagtcgcaagagcgacttcaccctcttggattttatcaacaccacgagcc |
| | gcatagctaccctggcaaacaagctgaagaccccactgttgttccactccttcgatgtgaggtgtttactgggcga |
| | tgaaatgcgcgaggaaatcgagggagcaggaatgcaccaagtgaagggtagggacaagcccgtcggagtctatcag |
| | tttcccgggccggagttggatgtggaaatggttttgcggcagaaagtggagcaattcaccccaggccggttccgct |
| | gccaaatgcctgtggttgaatatgaggcactgccaatctggcagcgacccccgatcgttgatgacacacccaaggg |
| | ttatccacggcctcggacgatgcctggctacggcgggcgccagagatctgactctctcgtggatcggctgattatg |
| | atcgctaagctggcagggccttctgtgtctgcgactggggacaccacgcttactacgctcacctacatttcccagg |
| | caacccgcaagccgatgtcacggctgcacctgtccgctctgatgggcacggccacccgcaggaatgctcaacagtc |
| | tatcaccgggactctgcttgacgttaacggcctgttcggtcagaccctggagggtccgaaagtcgcagtcgtaatg |
| | tacctctccatccggcaggatgcccgccagacagacgtaactaccgtccatatggcacctttgcaggagcgtgttt |
| | gcccgagtgagtggactctgacctccgctactgaggaaatgttggccactttcccccctctgcaggacgtgttgtc |
| | ccagttggccaagtcttgcacatccctggagacctacgtgcgctccactgtggggtatctgactgccggcaacaat |
| | cgtcgaaacctcatgcccgtttcgtgcggagtggtcatgcttgccaccgacatcggtagtttcacctccctgacag |
| | aggaatcctcgctcacagaggtttggatgatctgcaacacttttattgacgcctgcacgtctgctgtctgccagga |
| | gggcggggaggtgattaagcgcatcggcgactgcgttactgcctactttcctggcaacaatgccgagtccgctgtc |
| | gctgcggctcaggagttgttcaccttttgccgccagctgcgcgaggctttcgtcgacgtcgtggatgtgcgtggct |
| | gcgtctcctgcggggtgggactagactattcccaggtggtcatggcacagtgtggatcgctgggtcttacggagta |
| | cgttgtcgcaggtgtgagcgcccgggtgatggaggtcgatgcgatgactcgggaggttggttacgctatcggtgtc |
| | accgagccctacgtgaccgaccgcttgagcccccagtcccgcgaccatggaatcgtgcccactccacaggcaatcg |
| | agctgccgtgttacggcattgccggcgagcagctggagctcgacgttgacagcatcaagcggggcatcaaggccct |
| | tcacgcggctaggagcggcgagatgccagtgaccgagcccgaggaggccctgcgtctgttccgagtctcgcccggt |
| | cgagtgcgtcacggggacacaggtcgtcgatcaaacagcgcccagggcaagcgcagtattcaagtgcg |
| | ctaa |
| Photoactivated | atgtatattttggtctggaagaaaggccagcaaattaagaccttccacaccctggacgaggctgcccagttcaagg |
| adenylate | ccgcgtccaacttcgatgaagcaccaatgttctcggttactgtggcccccgcaatctccgcctccgggggatcaaa |
| cyclase subunit | cgaggccaccaacctgaggcgcctgatgtacctgtcaaagggtaccaacccggaggaatgcaatccccagtttgtg |
| beta-like protein | gccgagctggctcgggttgctactattcgcaaccgcgagattggtttatctggattcttgatgtcgagctctcctt |
| 1224-5/9F | tcttgttccaggcgatcgaggggaccgatcaggatctggatttcttgttcgccaagatttctgcagacccgaggca |
| pacB | cgagcgctgcatcgttctggcaaaccgtccctgcaccggaaggatgagtggagtttggcacatgaaggactcacac |
| Euglena gracilis | atggactctatcacgacccatgacgccatgatgacaatcctttaccaaatcgcccggtcattttccagcatgctga |
| mRNA | cttacctgccgaagcgggccgggaacatgctgttgagcggcctcgaccccgcagcgcagccacctgagccgaagtc |
| SEQ ID NO: 53 | ggtcgtagtgacattcatctacctggttgcctatggtagcttgctgtctaatccgtactctaccgaacagccggcg |
| | gaggtgctgtccacgttcgtcgacgtttgcgtcaagaacctggagggcagcgggggcatcattactaagttcatca |
| | ccgggatctgcatggcttcctggccgtttaaccgcaccgaggaagctttgacagccatccagcaaatctcggagga |
| | tttggcacagctgccctcccagcaagcgcctggctcctctccgagtcttatgtatagccaggccggtgtgcattac |
| | ggccgggctatgctttgtaacgcaggttctggcaaatccgacttcacgctgttgggcgactggattaacactacgt |
| | ctcggatcgcaaccctcgctaaaaagctgacatccccagttctgttcagcttccaggtccgatgtctgcttggcga |
| | cgaaatgcgcgaggaaatcgagggtgccgttatggatcaggtgaagggacgtgacaagccagtcgttgagtaccag |
| | ttccctggccccgagcgtgatgtggaggtggtcgggcaaaaaattgagcagttcacccctgggcgattccgttgcc |
| | agatgcgagtggggcagtatgaggctctgcctatctcacagcgtccacctatcttcgacgatacgcccaagtgcaa |
| | tccccgcccacgcacccccgggtgcgagggccggcaacgctctcacaccctagtggacccgcacattatgatggcc |
| | aagctccccggtccctccgtcagcgccactggcgacaccacagttcctacgttgacatacattagccatgccaccc |
| | gcccgatgagtagactcgatctctccgctattatgggaactgctactcgcatcaatgctcttcagcgaatcactgg |
| | gaccttgctccatggcaacggcctgtttgtccagacccaagagggcccaaaggaagcggagatcaacctccttctc |
| | cggattcgtcaggacacccgtcgcagcgacgtggctaccgaccacatgccccagccccaggaacgcgtctacccca |
| | gcgaatggacactcacctccggcaaggaggaaatcttggctatctttccacctttgcaggatgtgctgtcgcgtct |
| | ggcgaagtcattcactgcccaggagacctacgtgccttcccgcgtggtctcgtatctgacagcaggtaataaccct |
| | cgcgagttgatgcctgtctcctgcggtgttgtgatgaccgacatctgcagcctgacgtccttgacggagaactcct |
| | gcttgtctgaggtgtggatgatatgcaacacattcatcgacgcctgtccctccgctatttgccaggaaggcgcgga |
| | ggtgatcaagctgatcgggaactgcgtgaccgcttactttcccggaaacaatgcggacagtgccgtggcctccgcg |
| | caggagctgtttacgttctgtcgcgagcacagacaggcaatcgtcgatgtgctcgatgtgcgcggttgcgtgtctt |
| | acggcgttgctctggacaacggtcaggtggtcatggcacagtgcggttccgagggcctcaccgagtacgtcgttgc |
| | tggcgccgtgtctgcacgggtgatggaggtggaggccatcacacggggcacgtgcccccggtgggccactcccctg |
| | tggagcgccgaccgactgagcccacagcttagagatcacggtattgtgcccactccccaagcaattgaaggcctgc |
| | cctgctacgggatcgccggagaggagttcgagctggacgtcgattctattaagttgggaatcaaggcccttcacgc |
| | tgcccgtagcggcgagaagcccctgactgagccagaggaggcgaagctggatttctcgccaggacgcgttcgccat |
| | ggtgactccgggcggcgttccaactccgcccaaggtaagctcagtatccaggtgaggtaa |
| Putative | ccaatgaggttttctgtctacagcctatatgccctcgcctgcctcctcgccgttggaggggccattctcaccggca |
| membrane- | tcatacttctcgacacttttgacggccagcagcggaacagtgagaacgcacagttcacggccggcaaggggcaggt |
| bound adenylyl | ccagtccatgcagggggtcatcgctaaccgcatgacccgcatgcagaccatcaccgaggagcatagccggcagatc |
| cyclase, 4.6.1.1 | atgatgttcttgcagagcctccccagcaatgccattcagcctgataacgtggtgcaggctgtaaaccattccatct |
| Euglena gracilis | ttgacagctgggtcccctccgtcaaggcgaacaactctttgaatggttacggcttgacattcacgtacaccaacga |
| mRNA | gacgactggcaacagtttcgaccgcaccttctgggtgtattgggacctgatgacgaccggggtgtacgactacatc |
| SEQ ID NO: 54 | tacgcctacaccaacatgagcgatgggtatatccacgcttacgagacactgtggccgaatttctcgaccccacagc |
| | tgggactggacatgtaccgcttcaggagtactgtcttggcagccagcatcaaggacgatgatttcttcaccaccgc |
| | ccgtgtttgggtctccgcagacggcaactcctactggtacttcacccacatgcgcaccttccagttccagggagtg |
| | tacgccaatgttcagtcatgggacgtggccatcagctggctggacttgatgcgaactggtctgacaccggaggcaa |
| | acgtcgtggcctttgacagccagggctatgtgatggccgccaccaacaaggacgagctggcgaggctctcacagtg |
| | ccgcggatcgttcaacagcgggaatttggcggccacttgtatcagttattttgcccgaaatcatcccatcctcgag |
| | atccgcaacctgtacaacgcgctgcacaccccggtgtgggacgacctcggtgctgcgccgattcccctcgagcagg |
| | ccacgctgtccctctcagggaagaggtacatggccatcattgccactttgtactccaaagccgatttacggacaac |
| | cgtcgtgtggtaccaaccgtggttcgtcttacagacggacacagtcagtgtcacggccctcgtttgcatcctgacc |
| | atgctatccacatttgtactcacgctgctgggggtcttcggggtgctgcggccactgcaggccctcggggctgcaa |
| | tgcgagcggtggctggcacgctgaaggaaggggatggcagcaaggagacagttctggagccccgcaaacctaacgt |
| | gttccgggaggtggacgacattggggccgatttcgagaccatcgtggtggacttcctcggcttctccaacactaac |
| | gctcgagacaacaagtacgccccgaaggacccaaatgcgcctttcgccgtcgtcttcaccgacatccagtccagca |
| | cggcactgtggggcaagaaccccgccgagatgtctcgctgtgtccaggcccaccacgaactcattcgggagctcat |
| | caagcagcaccggctgtacgaggtgaagacggtgggggactcattcatggtcaccacgacctccgcccaggccgcc |
| | cttcgcttcgcgctggacgtccagaccacgcttttcgagcatgactgggactgggatggggcggacgagctctacc |
| | agcacacaggcgtcgtgttcaccaaggtgtgcgacgggccgttccggtccgagagcgagtacggagagctgtggaa |
| | tggccttcgcgtccgcatcgggatccatttcgggatgggagacgtactgtacgatgaggtgtccaagggctacgac |
| | tactacggcaacgtggtcaacgccgccgcccgaatcgaggccctcgcccacggcgggcaggtcgtcgtcaccgagg |
| | ccctgctggcggccctgccggcaaaactggactccagcgtcggcctcaccaggttgctcggcaagttccccctgcg |
| | cggggggcggagcccccggcactggtggaggtgacgcccaccgctctgcggggccgtacgtatccgctgctgcgca |
| | ccgaactcaccaccaccgagtcgcccgtagtggggcaggaaatccagagcgtcagcagcgacacccccaaaagcac |
| | cgaccgtcgctccagcggcccgacgaccgccagccatgcctccgaccggtgccccctccagcagctggcggaggac |
| | ttcgcccgcacccacaccctggtccggagcagcgtgctcccggcggaggttgtggcccagcagttgctgacactgt |
| | acagcatcgtggaggaccttctgatgcccctcgcccccgcgcagtgcactgcggtgctgaaggcgcttgccaaggg |
| | ctggggcttcactggccccatgccaaagaccgatgccgcccaccgtatgcggctcgtgcatcggatgtcggagacc |
| | acccaggtgtttacacggctcggcaacgtgacaaggtcccacactccaatgcacgcaggatgcaccccgagccact |
| | caggaggagggttcattcatgtagaacttgtctaactgcaactccattgcctagcccgcacagtgtattcatatgt |
| | ggctttgatggcatccactgaccatcagaaccaacccttcatcagcctgtgccaaatagcgttttctcaatcccat |
| | tccttcaatcaactgatttcacattccctcgcgcaccttgctcactccctctctcgatcggtttcccctcatgttg |
| | atggttgcagcttctgtgacatatccctccttcaccgattgttgctccgtcatttgtgcttccatatcgaactctg |
| | actccagaccctttgccatgttgctgtcttgcttttcctatgcttccaccaaagctgatgttgttccacctcatct |
| | tttggcagtccaggctgagtttgatgccccctgggatttttcagtttgggcagacaagtcagtgccacgtgtccat |
| | tgttaatactcaaagggcctgccaattatacaatctccaaaccacagtcagttcaagcgcgggctggtcctgtttg |
| | accggttgaccgaagccagctggccaactaccctcgcgactagagcggactggacgtcgcttctttgccccacatc |
| | ccaaccccctgttgcgacccatgctgcggccctggaccctcggcgacctatgcgggccaggatgccagccagcgtc |
| | gccccgaccggagcaggattggacgtcgcttctcgcccaactcaccccccccccaaccctgtogtaaccttgtggc |
| | agccttcccgtatccccaatattacccttgctggagcacgattggacagaattccctcgctcattttgcccccact |
| | ccctctctctatcgagaccactacatccccctcacgttgacggatgcagattctgtgacgtatccccctcctcccc |
| | ccttggccctgcccaccccagtatagaacgcactcacctgttggtgcagttccgcagctgcactgacatccactcc |
| | acattattatgcattctgatttcttttccatcgaacgttttagcgttaatccagagttttgtgtggattgctgctc |
| | catcattgggcttccatatccaactatctcctcctgcacccttgctatatcgctgcctttcttttcttgtgcatcc |
| | acatcatttaccaggcacagccacgtgcccatcgttccagattgcgaaagttttgctcttccactgcttgccattt |
| | atacagtctccaaaccgcagtcagttcaagtgcgggctggtcctgtttgaccgttggcagtcaccgaggccagctg |
| | tccaactaccctcgcgactagagcagactggacgtcgcttctttgcctcacattccaacccccctgccgcgaccca |
| | tgctgcggccctggaccctoggcgacctatgtgggccaggatgccagccagcgtcgccccgattcagctaactccc |
| | cttcacgcctggagcaggattggacgtcccttcctcgcccagctcacccccccccccaaaacctgtcacgagccat |
| | gtggcgactttcaccctcccctaactgcctccacaacgctggcaggcaaaggggaagggcaaagtagccgcctgtg |
| | gatccggagccagccagtatggccacccaaataagtttttgtagtggtgcgtagcaggtgcaccgacaaacgtcgc |
| | aaacgctgtgtttgagcccgtgacccttccgcttttagcttgcactgttgcaaaaaaaaaaaaaaaaaaaa |
| Photoactivated | atggattcaatgcaaagtaatctgcctcaccgagagatggcgagaaataattttggtatcaatacagatggtattc |
| adenylyl cyclase | ttacaaagaagactactactaactacctcctgaatggtggaggtgaacaagatagagcaatttctgttccgactag |
| Naegleria | tgttgttcaacacaatgaaatcaatccttctgaagctgcaaaaacagtaacggaattatcattcagtggtaataaa |
| australiensis | agtccttctaccttgacacaagctcgtatgatgaaaagatgttcaaaacaacaattactaagcaagcttccgaaga |
| mRNA | agcctttcctaagaattcaatatgcttctcattgttgttctgggaaaatatcagagtcggttaagaatgaaattct |
| SEQ ID NO: 55 | cagtgttgctcaaagagaaaacgagattagaggtattactggagttttgatttgcgtcaacgatatgttctttcaa |
| | atcattgagggtcatacacaacatgtcgaaactctctatcacaacattttaaaggataaaagacattatgatgtgg |
| | tgtgtgttgacaagatgtactttgattcagaaagtgagagatatttccctgattggtgtatgcaatcacttgacat |
| | ggagagtttatttgatcactgtcttgatccatcactctccactgctgttggtgctgcttttagagctttcaaaaca |
| | ttattagtatctctattgaatactcagcaagtgtttagaaactatgctcaacctgtcatctctaaatatttaagtg |
| | aaggagaaaatccaatgattaaggaatccatcgagactgataaagttatattattcattgatttggtcaattatac |
| | cagtataaccgaaaagttgtcaaaaacaagaaagccatcacaagtggtcgaaattttaaacaaattctttggttgt |
| | gttatggaaatagtggaagactattgtggcgagataactaaattaattggagactgtgtaatggtatattttgatg |
| | gtagtgaatgccaagaagcagttaattgctcaattgaaatattagagagattggagattttaaggcaaacatcaga |
| | agatccttttgtgaaaatgatttactgtaccattggaataagcagaggttctgttatctttggaaacgttggaagt |
| | gttggtagaaagttggattttacattaattggagattcagttaatattgcatcaagacttcaatcatttgcctcca |
| | tttcaccttatttcctaatatttgatgagagtgtaaaagggagattggataaagactcatgtgatataatagaagt |
| | gggcccaatagaactcaaaggcaaggaaaaaactgttaatacttatactgtcgaacacgaacttaacagaaaacct |
| | ccaaagaaggaaatggaccgaataatgtctctagcctcattaagtgaatattgttga |
| Photoactivated | atgtcatcacaaactccaccctacataagaatgcaatatgtttccctgtccaccgagggtaacatttctgaatcaa |
| adenylyl cyclase | caaagggaaagatactggcaacggcagcaagggaaaatccacgttataatattacaggtgtattgatatgcgtgag |
| Naegleria | ggatatgtttttccaaattattgaaggcaggattgaggatatagaccgactctatgtaaatattaagaaagacaaa |
| australiensis | agacacaacaatatcgcatgcatagacaagacatactttcatagagaggacgagagaatgttcccctcgtgggata |
| mRNA | tgaaatccatagaagtagatagcttgtttccaacaaagaaagaggttgtgatttgtggtgaatattgtaaccctct |
| SEQ ID NO: 56 | ggatccatcacatcaaaaggtcattgagacggcattctctgcttttactacaatgatgggaagcatgtttagagca |
| | caacaagtttttaagaattatgcacagccaagtgttagtagatttgttagggatggattgaatccgcttgctattg |
| | aatctgtagaaactgaaaaggttgtgctatttattgatttggtcaattatacaaccctgaccgaaattatctctca |
| | atcaagaaaaacatcagaggttgtccgaattttgaatgttttcttttcgtatgtgatggaatgtgttgagaatttt |
| | ggtggtgatatcactaaactaataggcgattgcgtaatgtgccactttgatggaaatgattgtcaaaaagcagttg |
| | attgttccctgaaaatactggaatccttagataaccttagaaaagaaaccaatgacccatttgaaaagctcatctt |
| | ttgtacaataggtatttctaaaggtaatgtaatcctgggcaatgttggaacagttggtagaaagctagactataca |
| | gtgattggtgattcggtaaatgtttctgcgagattacaatcgtttgcctcctcaacagagtactatctcatttttg |
| | atggaaatgtaaagaatggtttggatcgagttgactctgtggtatcatgtggctcaatagaactgaaaggaaaaaa |
| | gaatgctgtaaatgcgtacacaatagaaaatgtatataatagaagagtattggcagaggatctgaaagccatgtac |
| | tgcactctgaattaa |
| Photoactivated | atgtcatcatcacaaactcctccctacataagaatgcaatatgtttccctgtccaccgagggtatcatttctgaat |
| adenylyl cyclase | caacaaaggcaaagatactggcaacagcagcaagggaaaatccacgttataatattacaggtgtattgatatgtgt |
| Naegleria | gagggatatgtttttccaaattattgaaggcaggattgaggatatagaccgactttatgtaaatattaagaaagac |
| australiensis | aataggcacaacaatatcgcatgcatagacaagacatattttcatagagaggacgagagaatgtttccctcgtggg |
| mRNA | atatgaagtccatagaagtagacagcttgtttccaacaaagaaagaggttgtgatttgtggtgaatattgtaaccc |
| SEQ ID NO: 57 | tctggacccatcacatcaaaaggtcattgagacagcattctctgcttttactacactgatgggaagcatgtttaga |
| | gcacaacaagtttttaagaattatgctcagccaagtgttagtagatttgttagggacggattgaatccaattgcta |
| | ttgaatctgtagaaactgaaaaggttgtgctatttattgatttggtcaattatacaaccctgaccgaaattatctc |
| | tcaatcaagaaaaacatcagaggttgtccgaattttgaacgttttcttttcgtgtgtgatggaatgtgtcgaggat |
| | tttggtggtgatatcactaaactgataggtgattgtgtaatgtgccactttgatggaaatgattgtcaaacagcag |
| | ttgattgttccctgaaaatactggaatccttagataaccttagaaaagaaaccaatgacccatttgaaaagctcat |
| | cttttgtacaataggtatttctaaagggaatgtaatcctgggcaatgttggaacagttggtagaaagctagactat |
| | acagtgattggtgattcggtaaatgtttctgcgagattacaatcgtttgcctcctcaacagagtactatctcattt |
| | ttgatgaaaatgtaaagaatggtttggatcgagttgactctgtggtatcatgtggctcaatagaactgaaaggaaa |
| | aaagaatgctgtaaatgcgtacacaatagaaaatgtatataatagaagagtattggcagaggatctgaaagcaatg |
| | tactgcactctgaattaa |
| Putative beta | ggaagatgcacgtcctcatttggaaaaaaggccagcagatcaagacattccacacgcttgatgaggcggctcagtt |
| subunit of | caaagccgcttcaaatattgatgacgctcaaatatttaatgtgtctgtgacgcccgccatcagcgcttcgggcggc |
| photoactivated | agcaatgaagccaccaacctacggcggctgatgtacctttccaagtcgacaaatcctgaggagtgcactccgcagt |
| adenylyl | ttcttgccgagctcggacggatatcgagcattcgcaacaaggagatcggagtcagcggattcctgatgtactccgc |
| cyclase, 4.6.1.1 | tcccttctttttccaggttattgaaggcaccgacgaagacctcgatttccttttctcgaaaatttccatggacgac |
| Euglena | cggcatgacaggtgcattgtgctggccaatgggccgtgcacgggaagaatgtatggtgactggcacatgaaggact |
| quartana | ctcacatcgacagcatcaccacacaccctgcgatgaaaaccatcctttaccagatcgcccgatctttctccagtat |
| mRNA | gtggagctaccttcccaagagcaccggcaacatgttgctgctcggcaaggatcctgccgctcaaccgcccgtaccc |
| SEQ ID NO: 58 | atgagcgtcgtggttactttcctttatttggttgagttcggcaccatcctgtccaagcccaacctcacggagcaag |
| | cggctgaagtcttgagcaccttcgtcgatgtttgtgtgaagaatgtggagggcagtggtggcaacattgccaagtt |
| | cattactggagtctgtatggcgtattggccaatcaacagggcggaggacgctattcaggccatccagcagatcagc |
| | gacgatttggctcagcttcgcagccaacagccgccgggatcagcgatttcgttgatgtattctcaggctggcgtgc |
| | actacggccgagctttgctgtgcaacgccgggacgcgtaagtccgatttcacgctgctcggtgattgcattaacac |
| | cacatcccgcatcgccacgctggcgaagaagctgaagacaccactgctgttctcctttgaagtgcgctgtctgctg |
| | ggggacgagatgcgcgaggagatcgagagcgctggcatgcaccaggtaaagggacgggacaaacccgtcgttgtgt |
| | accaatttcccggagccacgctggatgtggaggttgtaaagcagaagatcgagcagttcaaccctggtcgattccg |
| | gtgccagatgcccgtcgtggactacgacactttgcttccttctcagcgcccacccatttttgatgagacacagagg |
| | gacgtcatcaagccgcgagtgcctgggatgcagaggcgggacagcctcgccgaccgtttggcgatgatcgccaaac |
| | tggctggcacgactggtggagcagcgggggctggtgatagtactctggtcactctgacgtacatctcccagaccgt |
| | acgaccaatgtcacggttggacctcgcggccatcatgcgtgtggccacccgccgcaacgcccagcagggcatcaca |
| | ggcactttgctgcacgtcagcggattgtttgtgcagacgctggagggcccgaggagtgctgttttgggcctctata |
| | tgcgcattcggcaagacccccgccacaaggatgtggtcactgtccacttggctccgatttcggaacgcgtgtaccc |
| | gccagactggacactgaccactgccacggaacagatgctgtegacctttccccctctccaagacgtgctgtcccaa |
| | ttggccaagtccttcacgtcgttagagacctacgtgccgtcgaccgtggtccgctacctgaccgccggcaacaacc |
| | caaggaatctagttcccgtgtcgtgcggcgtcgtgatgctggccacagatatctgctccttcaccagcctgaccga |
| | gaagtcttcgctgacggaggtttggctgatttgcaacacattcatcgacgcctgcaccaacgccatctgccaagcc |
| | ggtggagaggtcatcaagctgattggtgactgcgtgaccgcttattttcctgggactgccgctgacaatgccgtgt |
| | cggcggcgcaggagctgatcaccttctgtcgtgagctgcgagaggccttcgttgacgtgctggacgtccgcggctg |
| | catctattgcggcgtcgggctggatttcggccaggtggtgatggcacagtgtggcacccttggtctcagcgagtac |
| | gccgtggcgggtgccgtcagcgcgcgtgtgatggaggtggaggccctgacgcgcgaggtcggtcgcgccattgttc |
| | taaccgagcccctggccgaccggctgtgccctcagatgcaggagatgggcattgttcccagtccacaagccatcga |
| | tgggttgccatgctatggagtggcgggcgacgagttcgtgctcgatgttgatactatcaagcgcagcattaagaac |
| | ttccacgctgcacgctccggtgaaaagctcaatgtgtccgggcttgcgaatggcgatctggaggagagcatgacgg |
| | gctcgcctcttcgagccacaatcaacgacttccgcgtgtccccgggccgtgtgcgccgcggcgactctgtgcgtcg |
| | gtcaccgtccatcgcgggccggtatggcgtggcaacccgctgagaaatgggtctaggcttgggcaacctcgtggac |
| | agccacattttgccacctcgtttgggcagcctcgtggatgccgcagcagccctcacgcgggcgatgtcgtcatgta |
| | cgatggacg |
| Beta subunit of | atgaaaaggctaacttatattagtagcttctcgcgatcgcttagcaaaagagagattgaagccattggacatattt |
| photoactivated | ctcagactaataattctagggaaggaattacgggggttttattaagctgcaacggcattttctttcaaattttaga |
| adenylyl cyclase | aggcgatgaagaccgcattgatcggctgtacgagaagattttacgagatgatcgtcatagcaatattttatgcttg |
| ABRG53 0929 | aaatctgaattaaatgttttagcccgtaagtttcctgattggtcaatggaagtcatcgttcttgatgaaaataatg |
| Pseudanabaena | acattctcttgcaaccaattaaaattcttttgcaaactctaggcgaatctcaaaccattctcgctaactataccca |
| sp. ABRG5-3 | gtcaggagttttgcaagcaattgagaatgggattaatcccctcaccctcaaaccactctatagegataaaatcatc |
| mRNA | ttatttgctgatattatctccttttctggcattactgaaaaattgcaagttgctgaagtagtagatttgctcaacc |
| SEQ ID NO: 59 | agttctttaccatttgcacgcgagcgattactgcaagaggtggtgaagttacgaaatttattggcgattgcgtaat |
| | ggcatattttgatggcgatcgcgcagatgattcgctacaggcaagcctagatattttggcggaactagagctttta |
| | cgcaatcaagcccctgccaatagtccattacgagttctctacattggcattggtctatctaaaggaattgtgttag |
| | agggaaatatcggttcgcaaacaaagaaggaatatacagtgattggtgatgccgtcaatgttgccgccagaatcga |
| | agctctcaccagacagttaaatcgatttctcgtattttctgcttctgtacgtgcaggattaagcgatcgctggaag |
| | cctgttaggcttggcgattttcataccaagggaagagaaagtatcgtcgaagtctattctgttgacgatgtattaa |
| | ccttccaaaaatcagattttcataaattaaaacaagagatcaagcaacacctagagctaattagctgccaagcata |
| | a |
| Beta subunit of | atgaaaaggctaacctatattagtaacttctcgcgatcattgaccagaaaggaaatagaaaatattgagattattt |
| photoactivated | cacaaaaaaataattttagggaagggattacaggaatattactgagttgcagtggtattttctttcaaattttgga |
| adenylyl cyclase | aggggatgatcatcgagtagattcattgtacgaaaaaattttaaaagatgatcgtcaccatcaagttttatgtctt |
| ABRG53 0928 | aagtcagaagcaaacattactgagagattctttcctgactggtcgatgaaagtaattacaatagatgaaaacagcg |
| Pseudanabaena | agattttattaaaaccaattaaagtattgctacaaagcttgggagagtcaaaaatggctttggcaaactatacgca |
| sp. ABRG5-3 | ttcaggggttttacaagccatcgaaaacggaatcaatcctcttaccctgaccccacgctataccgaaaaaattatt |
| mRNA | ttctttgcggatattatgggctttgccagtatgactgagaaactccaagttaccgaagttgcagagttactcaatc |
| SEQ ID NO: 60 | aattttttacaatttgcacttcagaaatcgcagctaaaggcggagaagtcacgaagtttatcggcgattgcgtgat |
| | ggcatatttcgatggtgatcgtgcaaatgaagctttacaagcaagtctagacattctttcagaattagagatttta |
| | cgagatcgatcgcctgttaatagcccgttgcgagttctttatgtggggattgggctatctaagggaacagttttag |
| | aaggaaatatcggttctcaaaccaaaaaagagtatgcaatcatcggcgatgcagttaatgttgccgcaagaattga |
| | atcgctaacgcgaaagttaaatagatttttagtattttcggcagcagtacgagagggactcagtgatcgctggaaa |
| | ttgttaaaaatgggggatttctatgctaaaggaagagaagcgatcgtggaagtatattcaattgataatcctttgg |
| | catctcaaaaatcagatttccaccatctaaaacaagagattaacgagcagcttgaactcataggaaagcaaactct |
| | agtgtcttacaaacaacgcaaaacactatttaagctttacgatcgctaa |
| Putative | actgcaaaaaaatgcgaatttctgtatatgtgctgtacataggcgcttgccttctggcaattggagctgccatttt |
| membrane- | gagtggggttatcctacttaacacgttcgcgggacagcagcaacagagcgaggatgcgcagtacaccaccggccgg |
| bound adenylyl | ggccaggtgcaggccatccagggcgtcatcgccaaccaaatgtcccgcatggtggcctccgtggacgaccgtagtc |
| cyclase, 4.6.1.1 | ggcagctcctcatgaccctgaaggagctgcccgacgacgcccttgagccccagaacgccgtggcctcgctgaacca |
| Euglena gracilis | cagcatcttcacgacctgggttccttctatcatggcaaacgatactgtgctcgggaacggcttgaccatcatgtac |
| mRNA | gtgaacgagagcaatgggcagctgtacgaccgcacgttttccgtgtaccgggagctgctgaagagcgaccagtgga |
| SEQ ID NO: 61 | attacttgtacggctacacctttcctgatgagttgtcccatgttttcttcatcaactggctggattattcaactcc |
| | gcgacttgggaaggacgtgtaccaagtcaatttcacaacggtcaaggccccagtctatcagaaggacaacttcttt |
| | aaccacgtgatgccatggggttcgactgacggcaacactttctggtatttcacgtacatgaggtcgttttacaccc |
| | atggcgtgtggctcaatatgcagtgctgggatgtcacggtcaactggctgtcgctgatgcagagcggcttgacggc |
| | cggggccagcgtggcctcctacgacagccaggggtatgtggtcaccgccaccaacaaggacgagctgcggcggctg |
| | gcccagtgcattgagactggcgatgtgggctcgtcctgtatctctgaaggaggctgcaactgcctcaccactttcg |
| | caaagtttcatccgattgcggagctccgagacttgtatctcgcattgcacacccccttgtgggacgacctggacgc |
| | accaccaattccgctccagcagcaggacattcagctctctggtcagcggtacatggccatcgtggccacactgttc |
| | gctaaaagcggcttccgggtcaccgtcgtgtggtaccagccgtgggtcctcaccgacacctccgccgtgggcgtga |
| | ccgctctgatatgcgtcctgaccatgctgtcaacgttcctactcacgctgcttggcatcttcggcgtgctgcggcc |
| | actgatggccctgggcagtgccatgcgagtggtcgcccacacgctgcggaagggcgatgccgagagggaggcggtg |
| | ctgcagcctcggaggccgaacattttccgcgaggtggacaccatcgggcaggattttgagaccatcgtggtggact |
| | tcctcggcttctccagtacaaacgcgcgggacaacaagcacgcccccaaggatcccaacaagcctttcgtcgtcat |
| | cttcaccgacatccagtcgagtacggggctgtggggccgggaccccgcccagatgtcccgctgcgtgcaggtccac |
| | cacgagataatccggacgctcatcaaggatcaccacctctacgaagtgaaaactgcgggtgactcgttcatgatca |
| | ccaccgccagcccgcccgacgccctgcggttcgccctggacctgcagcaggcgctgtacgactacgactgggactg |
| | ggacgagtgcgacgacttctaccgcgagaccaccctggcgctgtggccggcggctctgggcgccaagtacgccgac |
| | atgtggaacgggcttcgggtccgcgttgggatccattttgggttgggcgatgttgtgtacgacgaggtctcgaagg |
| | gctacgactattacggcaacgtggtcaacaccgcggcccgcattgaggccctcgcccacgggggcagatcgtggtg |
| | acggagtccctgctggccgccctgcccaccccgctggaccccagggttagccgcacggtgtcgctcggcactgtcc |
| | ccttgcgcggcgtgacggcgccgccggcattggtggaggtccagatggcctccctggcaacccgcacttttccgcc |
| | gctgcgggtggagcacgctaatgaagcggcgaagcaggagtgcattaatgtgcccgacctcctcagtgacaaggct |
| | ggttctgatggcctgctgaccggtcggctggggtttcagaccccaagtctgcaatccgattccctggtgcaggtgg |
| | cggaggacttggccaagacccacgcgatggtgcggcggggctatttctcggcggaggcggtgacccaacacctgtt |
| | ggtcctctaccgcattgtggaggacttgttgacgcccctcgccccgtcacagttcgccacggtgacgaaggcgctg |
| | gcccggggctggggcgtggcgccgcccaagtcgaaggcggagttcggcccggtcgggctgcggctgatgcagcgca |
| | tgtcggagacaaccaccgtcctgaagaacttagcggcgccgcaccacagagctcagtccgccgggccggacgcgcg |
| | gctgctggacatggaagccacggcgtagctgccagggccagctttgcaaggttcctggtcgcccagtcgagtcgtg |
| | tctagtcgtatcgtctcctacccctacgcctgcctttegggaaccgtccccagtcccccgccccccaactccccac |
| | acggggtttctgcacaaccactgtccagctgttctttccttgcgtgtaccgcgcaccccctgcaaccctagtgctt |
| | ctgaacctgcccttttaaaattcggcccgagcccgagtcgtcgtcaggaaccgtcattgcatgtgtatcagtactg |
| | tgttctcttaaccctaagccgcggggcaagggagagagttctcctccctctcctcgcaacttgacacactccgtct |
| | cacccagtcacacaggtatacccacaacccccgaactgatagagcttaggcccactggcttagcatggtcactgcc |
| | ttctcatgccttcgggtcttttcatgtcatttcgcttccgtggccgcacccattgageggccctgaaagtttgagg |
| | cacactgccgcatctccagcccgcaatcctcagcctgacctccctccggcccattgcaagcactgctgcctcctcc |
| | ttgccaggtgttgtgctccccatgcaacggactgcccatcagctttggtgtccatggccgcgctaccactggtgca |
| | atgctgccagtgctcatcctgccagcatggcactctcccgtcgctggctgccactcctcggttgccccgacctgag |
| | aaacacctattggactcattgttcttcgctcagtctcgtgacaccggctattgtgtcccatattgattttcatggg |
| | ccagccgcacgcctcatctcgcataacgcgcacgcatccactgacccagttgtcccttctccgcataagtcagcct |
| | cctcactccagttctgcctgcctttatatcgatgcgtggttgtgttccttttctttttctttgcaaaattttttcc |
| | acacttccgatttacccgcccttgcaaccctctcaaaaaaaaaaaaaaaa |
| Beta subunit of | atgatgaaaagattagtttatattagcaaaatttcaggtcatttatcccttgaagaaatccaacgaattggtaaag |
| photoactivated | tatccattaaaaataatcaacgggataacattaccggcgtattactgtatttacaaggactatttttccaaatatt |
| adenylyl cyclase | ggagggagaaaatgagaaagtcgacaaattgtataaaaaaattttggttgatgatcgacataccaatattctatgt |
| BGP 1043 | ttaaaaacggaatatgatatcactgataggatgtttcctaattgggcgatgaaaacgattaatctgaatgaaaata |
| Beggiatoa sp. | gcgaattgatgattcaaccgattaaaagtttattacaaaccatcacacaatcgcatgtgtgttagaaaaatatatg |
| PS | ccggctcgtgttatttacctcattaatcaaggtattaatccactcaccgtcgaaccacaattggttgaaaaaatca |
| mRNA | tttttttcagtgatattctagctttttccaccttgacagaaaaactcccggtgaacgaagttgtcatcctcgttaa |
| SEQ ID NO: 62 | tcgttattttagtatttgcacgcgaattatttcagcttatggtggcgaagtgaccaaatttattggcgattgtgtc |
| | atggcatcatttaccaaagaacagggagatgctgctatccgtaccagtttagacattatcagcgaattgaaacaat |
| | tgcgtcatcatgttgaagcgactaatccattacatttattgtataccggcattgggctatcttacgggcatgtgat |
| | tgaaggcaatatgggttcttcattaaaaatggatcataccttgttaggtgatgcggtgaatgtggctgcacgttta |
| | gaggctttaacgcgacaattgccgtatgctttggcattcacagccggtgttaaaaaatgttgtcaagcacaatgga |
| | cttttatcaatctaggtgcccatcaagtcaaaggcaaacaagaagcgattgaagtgtataccgttaatgaagcaca |
| | aaaatattacgatactctgcaaatcacacaactgattaggcaaactcttgaaaatgataaatga |

TABLE 4
Plasmid

| Description | |
|---|---|
| SEQ ID NO | Sequence |
| Plasmid | agcttggcactggccgtcgttttacaacgtcgtgactgggaaaaccctggcgttacccaacttaatcgccttgcagcac |
| bPAC S27A_T | atccccctttcgccagctggcgtaatagcgaagaggcccgcaccgatcgcccttcccaacagttgcgcagcctgaatgg |
| 2A_mRFP_PG | cgaatggcgcctgatgcggtattttctccttacgcatctgtgcggtatttcacaccgcatatggtgcactctcagtaca |
| K_Hygro | atctgctctgatgccgcatagttaagccagccccgacacccgccaacacccgctgacgcgccctgacgggcttgtctgc |
| SEQ ID NO: 63 | tcccggcatccgcttacagacaagctgtgaccgtctccgggagctgcatgtgtcagaggttttcaccgtcatcaccgaa |
| | acgcgcgagacgaaagggcctcgtgatacgcctatttttataggttaatgtcatgataataatggtttcttagacgtca |
| | ggtggcacttttcggggaaatgtgcgcggaacccctatttgtttatttttctaaatacattcaaatatgtatccgctca |
| | tgagacaataaccctgataaatgcttcaataatattgaaaaaggaagagtatgagtattcaacatttccgtgtcgccct |
| | tattcccttttttgcggcattttgccttcctgtttttgctcacccagaaacgctggtgaaagtaaaagatgctgaagat |
| | cagttgggtgcacgagtgggttacatcgaactggatctcaacagcggtaagatccttgagagttttcgccccgaagaac |
| | gttttccaatgatgagcacttttaaagttctgctatgtggcgcggtattatcccgtattgacgccgggcaagagcaact |
| | cggtcgccgcatacactattctcagaatgacttggttgagtactcaccagtcacagaaaagcatcttacggatggcatg |
| | acagtaagagaattatgcagtgctgccataaccatgagtgataacactgcggccaacttacttctgacaacgatcggag |
| | gaccgaaggagctaaccgcttttttgcacaacatgggggatcatgtaactcgccttgatcgttgggaaccggagctgaa |
| | tgaagccataccaaacgacgagcgtgacaccacgatgcctgtagcaatggcaacaacgttgcgcaaactattaactggc |
| | gaactacttactctagcttcccggcaacaattaatagactggatggaggcggataaagttgcaggaccacttctgcgct |
| | cggcccttccggctggctggtttattgctgataaatctggagccggtgagcgtgggtctcgcggtatcattgcagcact |
| | ggggccagatggtaagccctcccgtatcgtagttatctacacgacggggagtcaggcaactatggatgaacgaaataga |
| | cagatcgctgagataggtgcctcactgattaagcattggtaactgtcagaccaagtttactcatatatactttagattg |
| | atttaaaacttcatttttaatttaaaaggatctaggtgaagatcctttttgataatctcatgaccaaaatcccttaacg |
| | tgagttttcgttccactgagcgtcagaccccgtagaaaagatcaaaggatcttcttgagatcctttttttctgcgcgta |
| | atctgctgcttgcaaacaaaaaaaccaccgctaccagcggtggtttgtttgccggatcaagagctaccaactctttttc |
| | cgaaggtaactggcttcagcagagcgcagataccaaatactgtccttctagtgtagccgtagttaggccaccacttcaa |
| | gaactctgtagcaccgcctacatacctcgctctgctaatcctgttaccagtggctgctgccagtggcgataagtcgtgt |
| | cttaccgggttggactcaagacgatagttaccggataaggcgcagcggtcgggctgaacggggggttcgtgcacacagc |
| | ccagcttggagcgaacgacctacaccgaactgagatacctacagcgtgagctatgagaaagcgccacgcttcccgaagg |
| | gagaaaggcggacaggtatccggtaagcggcagggtcggaacaggagagcgcacgagggagcttccagggggaaacgcc |
| | tggtatctttatagtcctgtcgggtttcgccacctctgacttgagcgtcgatttttgtgatgctcgtcaggggggcgga |
| | gcctatggaaaaacgccagcaacgcggcctttttacggttcctggccttttgctggccttttgctcacatgttctttcc |
| | tgcgttatcccctgattctgtggataaccgtattaccgcctttgagtgagctgataccgctcgccgcagccgaacgacc |
| | gagcgcagcgagtcagtgagcgaggaagcggaagagcgcccaatacgcaaaccgcctctccccgcgcgttggccgattc |
| | attaatgcagctggcacgacaggtttcccgactggaaagcgggcagtgagcgcaacgcaattaatgtgagttagctcac |
| | tcattaggcaccccaggctttacactttatgcttccggctcgtatgttgtgtggaattgtgagcggataacaatttcac |
| | acaggaaacagctatgacatgattacgaatttggtctctgcaaaagtctaggcctccaaaaaagcctcctcactacttc |
| | tggaatagctcagaggccgaggcggcctcggcctctgcataaataaaaaaaattagtcagccatggggcggagaatggg |
| | cggaactgggcggagttaggggcgggatgggcggagttaggggcgggatagctagagccagacatgataagatacattg |
| | atgagtttggacaaaccacaactagaatgcagtgaaaaaaatgctttatttgtgaaatttgtgatgctattgctttatt |
| | tgtaaccattataagctgcaataaacaagttcctctcactctctgatattcatttctttgcaagttataaatactgaat |
| | aataagatgacatgaactactactgctagagattttccacactgactaaaagggtctgagggatctctagttaccagag |
| | tcacacaacagacgggcacacactacttgaagcactcaaggcaagctttattgaggcttaagcagtgggttccctagtt |
| | agccagagagctcccaggctcagatctggtctaaccagagagacccagtacaagcaaaaagcagatcttatcttcgttg |
| | ggagtgaattagcccttccagtccccccttttcttttaaaaagtggctaagatctacagctgccttgtaagtcattggt |
| | cttaaaggtaccaggggggaggcggcccaaagggagatccgactcgtctgagggcgaaggcgaagacgcggaagaggcc |
| | gcagagccggcagcaggccgcgggaaggaaggtccgctggattgagggccgaagggacgtagcagaaggacgtcccgcg |
| | cagaatccaggtggcaacacaggcgagcagccaaggaaaggacgatgatttccccgacaacaccacggaattgtcagtg |
| | cccaacagccgagcccctgtccagcagcgggcaaggcaggcggcgatgagttccgccgtggcaataggggggggaaagc |
| | gaaagtcccggaaaggagctgacaggtggtggcaatgccccaaccagtgggggttgcgtcagcaaacacagtgcacacc |
| | acgccacgttgcctgacaacgggccacaactcctcataaagagacagcaaccaggatttatacaaggaggagaaaatga |
| | aagccatacgggaagcaatagcatgatacaaaggcattaaagcagcgtatccacatagcgtaaaaggagcaacatagtt |
| | aagaataccagtcaatctttcacaaattttgtaatccagaggttgattgtcgacttatccagcgcctgtgctatgtctg |
| | ccctcagctctctcatattgttccacgatggtgtagtccccattatggctgataatgtcgagtttaatatcagtcatgt |
| | aggcgccaggcagctgcacttgtttcttggccttgtaggttgttttgacctcggcatcgtagtgtcctccgtctttgag |
| | tttcagtctcattttaatttcgcctttcagagcgccatcctcaggatacattctctcggtggaggcttcccatcccatt |
| | gtttttttttgcatgacagggccatcggaggggaagttggttcctctgagtttcaccttataaataaactctccgtcct |
| | ggagggtgctatcttgtgtgactgtcaccacgcctccgtcctcgaagttcatgaatctctcccacttgaagccttcagg |
| | gaaggagagcttcagatagtcagggatgtcggcagggtgtttaacataggctttgcttccgtactggaactgagggctc |
| | agaatatcccaggagaaggggaggggtcctccttttgtgaccttgagcttagcggtttgtgtgccctcgtaaggccggc |
| | cttctccttctccctcaatctcgaactcgtgtccgttgacgcttccctccatcttgaccttaaatctcataaactcttt |
| | gatgacatcctcggagctggccattccggaagggccgggattctcctccacgtcaccgcatgttagaagacttcctctg |
| | ccctcagcggccaacaggtcctcctccgagatcagcttttgctcgtacgtccgcggcttgtcgttttccagggtctgcc |
| | ggatcagctgggtgatctgcagggtgtcgtagtacttctgggcctcattcacggtgtagacctcgatggcttcctgctt |
| | gcccttgacctggtgggctcccaggttgatgaaggtccactgggcctggcagcacttcttcacgccggctgtaaaggcc |
| | agggcgtagggcagctgtcttgtcagggcttccagcctggcggccacgttcacggcgtcccccagcagggtgtggtcca |
| | tcttcaggctgctgcccatgttgccctcgatcacgtggccgtagctcaggccgatgccggtgtacagcaggtgcagggg |
| | gttggtggcctccacgtggtgccgcagctgcttcagctcggagatgatgtccaggctggtccggatggcggcgtcgccc |
| | tgctctttggtgaagctggccatcacgcagtcgccgatgaacttggtcacttcgccgccgtaggcgctgatgatccggg |
| | tgcagatgctgaagtaccggttgaccaggatgaccacctcgttcacgggcagcttctcggtcagggtgctgaaggccag |
| | gatgtcgctgaagaagatgatcttctcgaccagctggggctccacggtcagggggttgatgccctggttgatcaggtag |
| | atcactctggcgggcatgtacttttccagcacccggtggctctgggtgatggtctgcagcaggctcttgatgggctgga |
| | tcatcagctcgctgttctcgttcaggttgatggttttcatggcccagttggggaacatccggtcggtgatgtcgtactc |
| | ggttttcaggcacaggatgttggtgtgccggtcgtccaccaggattttcttgtacagcttgtccaccttctcgttctcg |
| | ccttccaggatctggaagaacaggccctgcaggtacagcagcacgccggtgatgttgtcccgctggttgttcttgatgg |
| | ccaccttgccgatccgctggatctcttccaggctcaggtggccgctgatcttgctgatgtacaccagccgcttcatcat |
| | ggccgcggatccgatttaaattcgaattcgctagctctagatcacgacacctgaaatggaagaaaaaaactttgaacca |
| | ctgtctgaggcttgagaatgaaccaagatccaaactcaaaaagggcaaattccaaggagaattacatcaagtgccaagc |
| | tggcctaacttcagtctccacccactcagtgtggggaaactccatcgcataaaacccctccccccaacctaaagacgac |
| | gtactccaaaaggtcgagaactaatcgaggtgcctggacggcgcccggtactccgtggagtcacatgaagcgacggctg |
| | aggacggaaaggcccttttcctttgtgtgggtgactcacccgcccgctctcccgagcgccgcgtcctccattttgagct |
| | ccctgcagcagggccgggaagcggccatctttccgctcacgcaactggtgccgaccgggccagccttgccgcccagggc |
| | ggggcgatacacggcggcgcgaggccaggcaccagagcaggccggccagcttgagactacccccgtccgattctcggtg |
| | gccgcgctcgcaggccccgcctcgccgaacatgtgcgctgggacgcacgggccccgtcgccgcccgcggccccaaaaac |
| | cgaaataccagtgtgcagatcttggcccgcatttacaagactatcttgccagaaaaaaagcgtcgcagcaggtcatcaa |
| | aaattttaaatggctagagacttatcgaaagcagcgagacaggcgcgaaggtgccaccagattcgcacgcggcggcccc |
| | agcgcccaggccaggcctcaactcaagcacgaggcgaaggggctccttaagcgcaaggcctcgaactctcccacccact |
| | tccaacccgaagctcgggatcaagaatcacgtactgcagccaggtggaagtaattcaaggcacgcaagggccataaccc |
| | gtaaagaggccaggcccgcgggaaccacacacggcacttacctgtgttctggcggcaaacccgttgcgaaaaagaacgt |
| | tcacggcgactactgcacttatatacggttctcccccaccctcgggaaaaaggcggagccagtacacgacatcactttc |
| | ccagtttaccccgcgccaccttctctaggcaccggttcaattgccgacccctccccacaacttctcggggactgtgggc |
| | gatgtgcgctctgcccactgacgggcaccggagccgctagcttctaccgggtaggggaggcgcttttcccaaggcagtc |
| | tggagcatgcgctttagcagccccgctgggcacttggcgctacacaagtggcctctggcctcgcacacattccacatcc |
| | accggtaggcgccaaccggctccgttctttggtggccccttcgcgccaccttctactcctcccctagtcaggaagttcc |
| | cccccgccccgcagctcgcgtcgtgcaggacgtgacaaatggaagtagcacgtctcactagtctcgtgcagatggacag |
| | caccgctgagcaatggaagcgggtaggcctttggggcagcggccaatagcagctttgctccttcgctttctgggctcag |
| | aggctgggaaggggtgggtccggggggggctcaggggcgggctcaggggggggcgggcgcccgaaggtcctccggaggc |
| | ccggcattctgcacgcttcaaaagcgcacgtctgccgcgctgttctcctcttcctcatctccgggcctttcgacctgca |
| | gcccaagctttaattaaatgaagaagcccgaactcaccgctaccagcgttgaaaaatttctcatcgagaagttcgacag |
| | tgtgagcgacctgatgcagttgtcggagggcgaagagagccgagccttcagcttcgatgtcggcggacgcggctatgta |
| | ctgcgggtgaatagctgcgctgatggcttctacaaagaccgctacgtgtaccgccacttcgccagcgctgcactaccca |
| | tccccgaagtgttggacatcggcgagttcagcgagagcctgacatactgcatcagtagacgcgcccaaggcgttactct |
| | ccaagacctccccgaaacagagctgcctgctgtgttacagcctgtcgccgaagctatggatgctattgccgccgccgac |
| | ctcagtcaaaccagcggcttcggcccattcgggccccaaggcatcggccagtacacaacctggcgggatttcatttgcg |
| | ccattgctgatccccatgtctaccactggcagaccgtgatggacgacaccgtgtccgccagcgtagctcaagccctgga |
| | cgaactgatgctgtgggccgaagactgtcccgaggtgcgccacctcgtccatgccgacttcggcagcaacaacgtcctg |
| | accgacaacggccgcatcaccgccgtaatcgactggtccgaagctatgttcggggacagtcagtacgaggtggccaaca |
| | tcttcttctggcggccctggctggcttgcatggagcagcagactcgctacttcgagcgccggcatcccgagctggccgg |
| | cagccctcgtctgcgagcctacatgctgcgcatcggcctggatcagctctaccagagcctcgtggacggcaacttcgac |
| | gatgctgcctgggctcaaggccgctgcgatgccatcgtccgcagcggggccggcaccgtcggtcgcacacaaatcgctc |
| | gccggagcgcagccgtatggaccgacggctgcgtcgaggtgctggccgacagcggcaaccgccggcccagtacacgacc |
| | gcgcgctaaggaggtaggtcgagtttaagtttaaactcgagcgactgtgccttctagttgccagccatctgttgtttgc |
| | ccctcccccgtgccttccttgaccctggaaggtgccactcccactgtcctttcctaataaaatgaggaaattgcatcgc |
| | attgtctgagtaggtgtcattctattctggggggtggggtggggcaggacagcaagggggaggattgggaagacaatag |
| | caggcatgctggggatgcggtgggctctatggctcgagataaaattttgaattttgtaatttgtttttgtaattcttta |
| | gtttgtatgtctgttgctattatgtctactattctttcccctgcactgtaccccccaatccccccttttttttaaaagt |
| | taaccgataccgtcgagatccgttcactaatcgaatggatctgtctctgtctctctctccaccttcttcttctattcct |
| | tcgggcctgtcgggtcccctcggggttgggaggtgggtctgaaacgataatggtgaatatccctgcctaactctattca |
| | ctatagaaagtacagcaaaaactattcttaaacctaccaagcctcctactatcattatgaataattttatataccacag |
| | ccaatttgttatgttaaaccaattccacaaacttgcccatttatctaattccaataattcttgttcattcttttcttgc |
| | tggtttgcgattcttcaattaaggagtgtattaagcttgtgtaattgttaatttctctgtcccactccatccaggtcgt |
| | gtgattccaaatctgttccagagatttattactccaactagcattccaaggcacagcagtggtgcaaatgagttttcca |
| | gagcaaccccaaatccccaggagctgttgatcctttaggtatctttccacagccaggattcttgcctggagctgcttga |
| | tgccccagactgtgagttgcaacagatgctgttgcgcctcaatagccctcagcaaattgttctgctgctgcactatacc |
| | agacaataattgtctggcctgtaccgtcagcgtcattgacgctgcgcccatagtgcttcctgctgctcccaagaaccca |
| | aggaacaaagctcctattcccactgctcttttttctctctgcaccactcttctctttgccttggtgggtgctactccta |
| | atggttcaatttttactactttatatttatataattcacttctccaattgtccctcatatctcctcctccaggtctgaa |
| | gatcagtggccgcttgctgtgcggtggtcttacttttgttttgctcttcctctatcttgtctaaagcttccttggtgtc |
| | ttttatctctatcctttgatgcacacaatagagggttgctactgtattatataatgatctaagttcttctgatcctgtc |
| | tgaagggatggttgtagctgtcccagtatttgtctacagccttctgatgtttctaacaggccaggattaactgcgaatc |
| | gttctagctccctgcttgcccatactatatgttttaatttatattttttctttccccctggccttaaccgaattttttc |
| | ccatcgcgatctaattctcccccgcttaatactgacgctctcgcacccatctctctccttctagcctccgctagtcaaa |
| | atttttggcgtactcaccagtcgccgcccctcgcctcttgccgtgcgcgcttcagcaagccgagtcctgcgtcgagaga |
| | gctcctctggtttccctttcgctttcaagtccctgttcgggcgccactgctagagattttccacactgactaaaagggt |
| | ctgagggatctctagttaccagagtcacacaacagacgggcacacactacttgaagcactcaaggcaagctttattgag |
| | gcttaagcagtgggttccctagttagccagagagctcccaggctcagatctggtctaaccagagagacccgtttatgta |
| | tcgagctaggcacttaaatacaatatctctgcaatgcggcaattcagtggttcgtccaatccatgtcagacccgtctgt |
| | tgccttcctaataaggcacgatcgtaccaccttacttccaccaatcggcatgcacggtgctttttctctccttgtaagg |
| | catgttgctaactcatcgttaccatgttgcaagactacaagagtattgcataagactacacgcgt |

TABLE 5
Bluf domain

| Description | |
|---|---|
| SEQ ID NO | Sequence |
| Bluf Domain | mmkrlvyiskisghlsleeiqrigkvsiknnqrdnitgvllylqglffqilegenekvdklykkilvddrhtnilc |
| (aa) | lkteyditdrmfpnwamktinlnenselmiqpiksllqtitqshrvlekymparviylinqginpltvepqlveki |
| SEQ ID NO: 64 | iffsdilafstlteklpvnevvilvnryfsictriisayggevtkfigdcvmasftkeqgdaairtsldiiselkq |
| | lrhhveatnplhllytgiglsyghviegnmgsslkmdhtllgdavnvaarlealtrqlpyalaftagvkkccqaqw |
| | tfinlgahqvkgkqeaievytvneaqkyydtlqitqlirqtlendk |
| Bluf Domain | atgatgaagcggctggtgtacatcagcaagatcagcggccacctgagcctggaagagatccagcggatcggcaagg |
| (nt) | tgtccatcaagaacaaccagcgggacaacatcaccggcgtgctgctgtacctgcagggcctgttcttccagatcct |
| SEQ ID NO: 65 | ggaaggcgagaacgagaaggtggacaagctgtacaagaaaatcctggtggacgaccggcacaccaacatcctgtgc |
| | ctgaaaaccgagtacgacatcaccgaccggatgttccccaactgggccatgaaaaccatcaacctgaacgagaaca |
| | gcgagctgatgatccagcccatcaagagcctgctgcagaccatcacccagagccaccgggtgctggaaaagtacat |
| | gcccgccagagtgatctacctgatcaaccagggcatcaaccccctgaccgtggagccccagctggtcgagaagatc |
| | atcttcttcagcgacatcctggccttcagcaccctgaccgagaagctgcccgtgaacgaggtggtcatcctggtca |
| | accggtacttcagcatctgcacccggatcatcagcgcctacggcggcgaagtgaccaagttcatcggcgactgcgt |
| | gatggccagcttcaccaaagagcagggcgacgccgccatccggaccagcctggacatcatctccgagctgaagcag |
| | ctgcggcaccacgtggaggccaccaaccccctgcacctgctgtacaccggcatcggcctgagctacggccacgtga |
| | tcgagggcaacatgggcagcagcctgaagatggaccacaccctgctgggggacgccgtgaacgtggccgccaggct |
| | ggaagccctgacaagacagctgccctacgccctggcctttacagccggcgtgaagaagtgctgccaggcccagtgg |
| | accttcatcaacctgggagcccaccaggtcaagggcaagcaggaagccatcgaggtctacaccgtgaatgaggccc |
| | agaagtactacgacaccctgcagatcacccagctgatccggcagaccctggaaaacgacaag |

### REFERENCES

1. Lumeng, C. N., and Saltiel, A. R. (2011). Inflammatory links between obesity and metabolic disease. J Clin Invest 121, 2111-2117. 10.1172/JCI57132.
2. Harms, M., and Seale, P. (2013). Brown and beige fat: development, function and therapeutic potential. Nat Med 19, 1252-1263. 10.1038/nm.3361.
3. Cypess, A. M., Lehman, S., Williams, G., Tal, I., Rodman, D., Goldfine, A. B., Kuo, F. C., Palmer, E. L., Tseng, Y. H., Doria, A., et al. (2009). Identification and importance of brown adipose tissue in adult humans. N Engl J Med 360, 1509-1517. 10.1056/NEJMoa0810780.
4. Wu, J., Cohen, P., and Spiegelman, B. M. (2013). Adaptive thermogenesis in adipocytes: is beige the new brown? Genes Dev 27, 234-250. 10.1101/gad.211649.112.
5. Becher, T., Palanisamy, S., Kramer, D. J., Eljalby, M., Marx, S. J., Wibmer, A. G., Butler, S. D., Jiang, C. S., Vaughan, R., Schoder, H., et al. (2021). Brown adipose tissue is associated with cardiometabolic health. Nat Med 27, 58-65. 10.1038/s41591-020-1126-7.
6. Cannon, B., and Nedergaard, J. (2004). Brown adipose tissue: function and physiological significance. Physiol Rev 84, 277-359. 10.1152/physrev.00015.2003.
7. Bachman, E. S., Dhillon, H., Zhang, C. Y., Cinti, S., Bianco, A. C., Kobilka, B. K., and Lowell, B. B. (2002). betaAR signaling required for diet-induced thermogenesis and obesity resistance. Science 297, 843-845. 10.1126/science.1073160.
8. Wade, G., McGahee, A., Ntambi, J. M., and Simcox, J. (2021). Lipid Transport in Brown Adipocyte Thermogenesis. Front Physiol 12, 787535. 10.3389/fphys.2021.787535.
9. Fenzl, A., and Kiefer, F. W. (2014). Brown adipose tissue and thermogenesis. Horm Mol Biol Clin Investig 19, 25-37. 10.1515/hmbci-2014-0022.
10. Auger, C., and Kajimura, S. (2021). Detouring adrenergic stimulation to induce adipose thermogenesis. Nat Rev Endocrinol 17, 579-580. 10.1038/s41574-021-00546-6.
11. Vosselman, M. J., van der Lans, A. A., Brans, B., Wierts, R., van Baak, M. A., Schrauwen, P., and van Marken Lichtenbelt, W. D. (2012). Systemic beta-adrenergic stimulation of thermogenesis is not accompanied by brown adipose tissue activity in humans. Diabetes 61, 3106-3113. 10.2337/db12-0288.
12. Cypess, A. M., Weiner, L. S., Roberts-Toler, C., Franquet Elia, E., Kessler, S. H., Kahn, P. A., English, J., Chatman, K., Trauger, S. A., Doria, A., and Kolodny, G. M. (2015). Activation of human brown adipose tissue by a beta3-adrenergic receptor agonist. Cell Metab 21, 33-38. 10.1016/j.cmet.2014.12.009.
13. Sui, W., Li, H., Yang, Y., Jing, X., Xue, F., Cheng, J., Dong, M., Zhang, M., Pan, H., Chen, Y., et al. (2019). Bladder drug mirabegron exacerbates atherosclerosis through activation of brown fat-mediated lipolysis. Proc Natl Acad Sci USA 116, 10937-10942. 10.1073/pnas. 1901655116.
14. Oeckl, J., Bast-Habersbrunner, A., Fromme, T., Klingenspor, M., and Li, Y. (2020). Isolation, Culture, and Functional Analysis of Murine Thermogenic Adipocytes. STAR Protoc 1, 100118. 10.1016/j.xpro.2020.100118.
15. Li, Y., and Fromme, T. (2022). Uncoupling Protein 1 Does Not Produce Heat without Activation. Int J Mol Sci 23. 10.3390/ijms23052406.
16. Dickson, L. M., Gandhi, S., Layden, B. T., Cohen, R. N., and Wicksteed, B. (2016). Protein kinase A induces UCP1 expression in specific adipose depots to increase energy expenditure and improve metabolic health. Am J Physiol Regul Integr Comp Physiol 311, R79-88. 10.1152/ajpregu.00114.2016.
17. Tong, T., Shen, Y., Lee, H. W., Yu, R., and Park, T. (2016). Adenylyl cyclase 3 haploinsufficiency confers susceptibility to diet-induced obesity and insulin resistance in mice. Sci Rep 6, 34179. 10.1038/srep34179.
18. Evans, B. A., Merlin, J., Bengtsson, T., and Hutchinson, D. S. (2019). Adrenoceptors in white, brown, and brite adipocytes. Br J Pharmacol 176, 2416-2432. 10.1111/bph.14631.
19. Wang, L., Pydi, S. P., Cui, Y., Zhu, L., Meister, J., Gavrilova, O., Berdeaux, R., Fortin, J. P., Bence, K. K., Vernochet, C., and Wess, J. (2019). Selective activation of G(s) signaling in adipocytes causes striking metabolic improvements in mice. Mol Metab 27, 83-91. 10.1016/j.molmet.2019.06.018.
20. Caron, A., Reynolds, R. P., Castorena, C. M., Michael, N. J., Lee, C. E., Lee, S., Berdeaux, R., Scherer, P. E., and Elmquist, J. K. (2019). Adipocyte Gₛ but not Gi signaling regulates whole-body glucose homeostasis. Mol Metab 27, 11-21. 10.1016/j.molmet.2019.06.019.
21. Goutaudier, R., Coizet, V., Carcenac, C., and Carnicella, S. (2019). DREADDs: The Power of the Lock, the Weakness of the Key. Favoring the Pursuit of Specific Conditions Rather than Specific Ligands. eNeuro 6. 10.1523/ENEURO.0171-19.2019.
22. Lyons, C. E., Razzoli, M., Larson, E., Svedberg, D., Frontini, A., Cinti, S., Vulchanova, L., Sanders, M., Thomas, M., and Bartolomucci, A. (2020). Optogenetic-induced sympathetic neuromodulation of brown adipose tissue thermogenesis. FASEB J 34, 2765-2773. 10.1096/fj.201901361RR.
23. Stierl, M., Stumpf, P., Udwari, D., Gueta, R., Hagedorn, R., Losi, A., Gartner, W., Petereit, L., Efetova, M., Schwarzel, M., et al. (2011). Light modulation of cellular cAMP by a small bacterial photoactivated adenylyl cyclase, bPAC, of the soil bacterium Beggiatoa. J Biol Chem 286, 1181-1188. 10.1074/jbc.M110.185496.
24. Jansen, V., Alvarez, L., Balbach, M., Strunker, T., Hegemann, P., Kaupp, U. B., and Wachten, D. (2015). Controlling fertilization and cAMP signaling in sperm by optogenetics. Elife 4. 10.7554/eLife.05161.
25. Tanwar, M., Khera, L., Haokip, N., Kaul, R., Naorem, A., and Kateriya, S. (2017). Modulation of cyclic nucleotide-mediated cellular signaling and gene expression using photoactivated adenylyl cyclase as an optogenetic tool. Sci Rep 7, 12048. 10.1038/s41598-017-12162-4.
26. Galmozzi, A., Sonne, S. B., Altshuler-Keylin, S., Hasegawa, Y., Shinoda, K., Luijten, I. H. N., Chang, J. W., Sharp, L. Z., Cravatt, B. F., Saez, E., and Kajimura, S. (2014). ThermoMouse: an in vivo model to identify modulators of UCP1 expression in brown adipose tissue. Cell Rep 9, 1584-1593. 10.1016/j.celrep.2014.10.066.
27. Katz, S., Backeris, P., Merck, C., Suprun, M., D'Souza, S., Bishop, D. F., Desnick, R. J., Moore, K., Ubarretxena-Belandia, I., and Lemischka, I. R. (2018). Design and validation of an open-source modular Microplate Photoirradiation System for high-throughput photobiology experiments.

PLOS One 13, e0203597. 10.1371/journal.pone.0203597.
28. Zhang, X., Odom, D. T., Koo, S. H., Conkright, M. D., Canettieri, G., Best, J., Chen, H., Jenner, R., Herbolsheimer, E., Jacobsen, E., et al. (2005). Genome-wide analysis of cAMP-response element binding protein occupancy, phosphorylation, and target gene activation in human tissues. Proc Natl Acad Sci USA 102, 4459-4464. 10.1073/pnas.0501076102.
29. Olsen, J. M., Sato, M., Dallner, O. S., Sandstrom, A. L., Pisani, D. F., Chambard, J. C., Amri, E. Z., Hutchinson, D. S., and Bengtsson, T. (2014). Glucose uptake in brown fat cells is dependent on mTOR complex 2-promoted GLUT1 translocation. J Cell Biol 207, 365-374. 10.1083/jcb.201403080.
30. Bokhari, M. H., Halleskog, C., Aslund, A., Boulet, N., Casadesus Rendos, E., de Jong, J. M. A., Csikasz, R., Amri, E. Z., Shabalina, I., and Bengtsson, T. (2021). Isothermal microcalorimetry measures UCP1-mediated thermogenesis in mature brite adipocytes. Commun Biol 4, 1108. 10.1038/s42003-021-02639-4
31. Keidar, A. (2011). Bariatric surgery for type 2 diabetes reversal: the risks. Diabetes Care 34 Suppl 2, S361-266. 10.2337/dc11-s254.
32. Kozak, L. P., and Anunciado-Koza, R. (2008). UCP1: its involvement and utility in obesity. Int J Obes (Lond) 32 Suppl 7, S32-38. 10.1038/ijo.2008.236.
33. Davies, V. S., Lindsund, E., Petrovic, N., Cannon, B., and Nedergaard, J. (2023). Repeated short excursions from thermoneutrality suffice to restructure brown adipose tissue. Biochimie. 10.1016/j.biochi.2023.01.006.
34. Yang, S., Constantin, O. M., Sachidanandan, D., Hofmann, H., Kunz, T. C., Kozjak-Pavlovic, V., Oertner, T. G., Nagel, G., Kittel, R. J., Gee, C. E., and Gao, S. (2021). PACmn for improved optogenetic control of intracellular cAMP. BMC Biol 19, 227. 10.1186/s12915-021-01151-9.
35. Ikeda, K., Kang, Q., Yoneshiro, T., Camporez, J. P., Maki, H., Homma, M., Shinoda, K., Chen, Y., Lu, X., Maretich, P., et al. (2017). UCP1-independent signaling involving SERCA2b-mediated calcium cycling regulates beige fat thermogenesis and systemic glucose homeostasis. Nat Med 23, 1454-1465. 10.1038/nm.4429.
36. Kazak, L., Chouchani, E. T., Jedrychowski, M. P., Erickson, B. K., Shinoda, K., Cohen, P., Vetrivelan, R., Lu, G. Z., Laznik-Bogoslavski, D., Hasenfuss, S. C., et al. (2015). A creatine-driven substrate cycle enhances energy expenditure and thermogenesis in beige fat. Cell 163, 643-655. 10.1016/j.cell.2015.09.035.
37. Rahbani, J. F., Scholtes, C., Lagarde, D. M., Hussain, M. F., Roesler, A., Dykstra, C. B., Bunk, J., Samborska, B., O'Brien, S. L., Tripp, E., et al. (2022). ADRAIA-Galpha (q) signalling potentiates adipocyte thermogenesis through CKB and TNAP. Nat Metab 4, 1459-1473. 10.1038/s42255-022-00667-w.
38. Nayak, G., Zhang, K. X., Vemaraju, S., Odaka, Y., Buhr, E. D., Holt-Jones, A., Kernodle, S., Smith, A. N., Upton, B. A., D'Souza, S., et al. (2020). Adaptive Thermogenesis in Mice Is Enhanced by Opsin 3-Dependent Adipocyte Light Sensing. Cell Rep 30, 672-686 e678. 10.1016/j.celrep.2019.12.043.
39. Sato, M., Tsuji, T., Yang, K., Ren, X., Dreyfuss, J. M., Huang, T. L., Wang, C. H., Shamsi, F., Leiria, L. O., Lynes, M. D., et al. (2020). Cell-autonomous light sensitivity via Opsin3 regulates fuel utilization in brown adipocytes. PLOS Biol 18, e3000630. 10.1371/journal.pbio.3000630.
40. Wang, C. H., Lundh, M., Fu, A., Kriszt, R., Huang, T. L., Lynes, M. D., Leiria, L. O., Shamsi, F., Darcy, J., Greenwood, B. P., et al. (2020). CRISPR-engineered human brown-like adipocytes prevent diet-induced obesity and ameliorate metabolic syndrome in mice. Sci Transl Med 12. 10.1126/scitranslmed.aaz8664.
41. Tajima, K., Ikeda, K., Tanabe, Y., Thomson, E. A., Yoneshiro, T., Oguri, Y., Ferro, M. D., Poon, A. S. Y., and Kajimura, S. (2020). Wireless optogenetics protects against obesity via stimulation of non-canonical fat thermogenesis. Nat Commun 11, 1730. 10.1038/s41467-020-15589-y.
42. Stierl, M., Penzkofer, A., Kennis, J. T., Hegemann, P., and Mathes, T. (2014). Key residues for the light regulation of the blue light-activated adenylyl cyclase from Beggiatoa sp. Biochemistry 53, 5121-5130. 10.1021/bi500479v.
43. Su, S., Guntur, A. R., Nguyen, D. C., Fakory, S. S., Doucette, C. C., Leech, C., Lotana, H., Kelley, M., Kohli, J., Martino, J., et al. (2018). A Renewable Source of Human Beige Adipocytes for Development of Therapies to Treat Metabolic Syndrome. Cell Rep 25, 3215-3228 e3219. 10.1016/j.celrep.2018.11.037.

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

What is claimed is:

1. A cell comprising at least one non-native gene or gene cassette encoding one or more adenylyl cyclase enzymes, wherein the adenylyl cyclase enzyme comprises an optogenetic moiety.

2. The cell of claim 1, wherein the gene or gene cassette is operably linked to a promoter that is not associated with the adenylyl cyclase enzyme in nature.

3. The cell of claim 2, wherein the promoter is inducible.

4. The cell of claim 2 or 3, wherein the promoter is induced in adipocytes.

5. The cell of any one of claims 2-4, wherein the adipocyte-specific promoter is AP2 or AdipoQ.

6. The cell of claim 2 or 3, wherein the promoter is selected from the group consisting of EF1α promoter, CAG promoter, and CMV promoter.

7. The cell of any one of the previous claims, wherein the adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa).

8. The cell of claim 5, wherein the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007.

9. The cell of claim 7 or 8, wherein the adenylyl cyclase enzyme is from Beggiatoa sp. PS.

10. The cell of any one of the previous claims, wherein the amino acid sequence of the adenylyl cyclase enzyme comprises one or more amino acid substitutions relative to a wild type adenylyl cyclase sequence.

11. The cell of claim 10, wherein the one or more amino acid substitutions are at positions corresponding to position 27, 123, 198, 266, 267, 278, 197, and/or 201 in the wild type adenylyl cyclase enzyme sequence.

12. The cell of claim 10 or 11, wherein the wild type adenylyl cyclase enzyme amino acid sequence comprises SEQ ID NO: 1 or SEQ ID NO: 31.

13. The cell of any one of claims 10-12, wherein the amino acid sequence of the adenylyl cyclase enzyme comprises an alanine (A) at a position corresponding to position 27 in the amino acid sequence of SEQ ID NO: 1 or SEQ ID NO: 31.

14. The cell of any one of claims 1-12, wherein the amino acid sequence of the adenylyl cyclase enzyme comprises SEQ ID NO: 2.

15. The cell of any one of the previous claims, wherein the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of any one of SEQ ID NOs: 32, 33, or 62.

16. The cell of any one of the previous claims, wherein the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of SEQ ID NOs: 1, 2, or 31.

17. The cell of any one of claims 1-16, wherein the optogenetic moiety and the adenylyl cyclase enzyme are found in the same polypeptide in nature.

18. The cell of any one of claims 1-16, wherein the optogenetic moiety is fused to the adenylyl cyclase enzyme, and is not associated with the adenylyl cyclase enzyme in nature.

19. The cell of claim 18, wherein the optogenetic moiety and the adenylyl cyclase enzyme are derived from different organisms.

20. The cell of any one of the previous claims, wherein the gene or gene cassette encoding the adenylyl cyclase enzyme comprising the optogenetic moiety is codon optimized for expression in a mammalian cell.

21. The cell of any one of the previous claims, wherein the cell is a mammalian cell.

22. The cell of claim 21, wherein the mammalian cell is a human cell.

23. The cell of any one of the previous claims, wherein the cell is a pluripotent stem cell.

24. The cell of claim 23, wherein the pluripotent stem cell is a preadipocyte.

25. The cell of claim 23 or 24, wherein the pluripotent stem cell or preadipocyte is capable of differentiating into an adipocyte.

26. The cell of claim 25, wherein the adipocyte is a brown adipocyte, white adipocyte, or beige adipocyte.

27. A method of generating the cell of any one of claims 1-26, comprising:
a) providing a cell which is capable of differentiation into an adipocyte;
b) transfecting the cell with a vector comprising the one or more gene cassettes encoding one or more adenylyl cyclase enzymes comprising the optogenetic moiety;
thereby generating the cell of any one of claims 1-25.

28. The method of claim 27, wherein the transfected cell is differentiated into an adipocyte, wherein the adipocyte is a brown adipocyte, white adipocyte, or beige adipocyte.

29. The method of claim 27 or 28, wherein the transfected cell, adipocyte, brown adipocyte, white adipocyte, or beige adipocyte is expanded in culture.

30. The method of any one of claims 27-29, wherein the vector is a plasmid, a lentivirus vector or an AAV vector.

31. The method of any one of claims 27-30, wherein the vector further comprises a gene encoding a polypeptide to induce integration of the gene or gene cassette encoding the one or more adenylyl cyclase enzymes comprising the optogenetic moiety into the genome of the cell.

32. The method of claim 31, wherein the gene encoding a polypeptide to induce integration of the gene or gene cassette into the genome is a DNA endonuclease.

33. The method of claim 32, wherein the DNA endonuclease is a Cas endonuclease.

34. The method of claim 33, wherein the Cas endonuclease is a Cas9 endonuclease.

35. A system for activating uncoupling protein-1 (UCP-1) in a cell, the system comprising:
a) a delivery vector comprising a non-native nucleotide sequence encoding one or more adenylyl cyclase enzymes, wherein the adenylyl cyclase enzyme comprises an optogenetic moiety, wherein the delivery vector provides for delivery of the one or more adenylyl cyclase enzymes comprising the optogenetic moiety to a cell;
b) a light source; and
c) a control device.

36. A system for increasing the levels of uncoupling protein-1 (UCP-1) in a cell, the system comprising:
a) a delivery vector comprising a non-native nucleotide sequence encoding one or more adenylyl cyclase enzymes, wherein the adenylyl cyclase enzyme comprises an optogenetic moiety, wherein the delivery vector provides for delivery of the one or more adenylyl cyclase enzymes comprising the optogenetic moiety to a cell;
b) a light source; and
c) a control device.

37. The system of claim 35 or 36, wherein the delivery vector is a plasmid, a lentivirus vector or an AAV vector.

38. The system of any one of claims 35-37, wherein the non-native nucleotide sequence is operably linked to a promoter that is not associated with the adenylyl cyclase enzyme in nature.

39. The system of claim 38, wherein the promoter is inducible.

40. The system of claim 38 or 39, wherein the promoter is induced in adipocytes.

41. The system of any one of claim 40, wherein the adipocyte-specific promoter is AP2 or AdipoQ.

42. The system of claim 38 or 39, wherein the promoter is selected from the group consisting of EF1α promoter, CAG promoter, and CMV promoter.

43. The system of any one of claims 35-42, wherein the adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa).

44. The system of claim 43, wherein the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007.

45. The system of claim 43 or 44, wherein the adenylyl cyclase enzyme is from Beggiatoa sp. PS.

46. The system of any one of claims 35-45, wherein the amino acid sequence of the adenylyl cyclase enzyme comprises one or more amino acid substitutions relative to a wild type adenylyl cyclase sequence.

47. The system of claim 46, wherein the one or more amino acid substitutions are at positions corresponding to position 27, 123, 198, 266, 267, 278, 197, and/or 201 in the wild type adenylyl cyclase enzyme amino acid sequence.

48. The system of claim 46 or 47, wherein the wild type adenylyl cyclase enzyme amino acid sequence comprises SEQ ID NO: 1 or SEQ ID NO: 30.

49. The system of any one of claims 46-48, wherein the amino acid sequence of the adenylyl cyclase enzyme sequence comprises an alanine (A) at a position corresponding to position 27 in the sequence of SEQ ID NO: 1 or SEQ ID NO: 31.

50. The system of any one of claims 35-49, wherein the amino acid sequence of the adenylyl cyclase enzyme comprises SEQ ID NO: 2.

51. The system of any one of claims 35-50, wherein the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of any one of SEQ ID NOs: 32, 33, or 62.

52. The system of any one of claims 35-51, wherein the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of SEQ ID NOs: 1, 2, or 31.

53. The system of any one of claims 35-52, wherein the optogenetic moiety and the adenylyl cyclase enzyme are found in the same polypeptide in nature.

54. The system of any one of claims 35-53, wherein the optogenetic moiety is fused to the adenylyl cyclase enzyme, and is not associated with the adenylyl cyclase enzyme in nature.

55. The system of claim 54, wherein the optogenetic moiety and the adenylyl cyclase enzyme are derived from different organisms

56. The system of any one of claims 35-55, wherein the gene or gene cassette encoding the adenylyl cyclase enzyme comprising the optogenetic moiety is codon optimized for expression in a mammalian cell.

57. The system of any one of claims 35-56, wherein the cell is a pluripotent cell or a preadipocyte.

58. The system of claim 57, wherein the pluripotent stem cell or the preadipocyte is capable of differentiating into an adipocyte.

59. The system of any one of claims 35-58, wherein the light source is a light-emitting diode or a laser.

60. The system of any one of claims 35-59, wherein the light source produces transient blue light, continuous blue light, or oscillating pulses of blue light.

61. The system of any one of claims 35-60, wherein the radiation intensity of the blue light produced by the light source is at least 0.05 mW/cm², at least 0.1 mW/cm², at least 1 mW/cm², at least 2 mW/cm², at least 5 mW/cm², or at least 10 mW/cm²,

62. The system of any one of claims 35-61, wherein the control device is implantable.

63. The system of any one of the claims 35-62, further wherein the system activates UCP-1 in a subject.

64. A method for activating uncoupling protein-1 (UCP-1) in a brown adipocyte of a subject, the method comprising:
a) administering to the subject the cell of any one of claims 1-26, or the cell generated by the method of any one of claims 27-33;
b) activating the optogenetic moiety with a light source, wherein the adenylyl cyclase enzyme is activated, and wherein the light source is controlled by a control device;
thereby activating UCP-1 in the cell of the subject.

65. A method for increasing the levels of uncoupling protein-1 (UCP-1) in a brown adipocyte of a subject, the method comprising:
a) administering to the subject the cell of any one of claims 1-26, or the cell generated by the method of any one of claims 27-33;
b) activating the optogenetic moiety with a light source, wherein the adenylyl cyclase enzyme is activated, and wherein the light source is controlled by a control device;
thereby increasing the levels of UCP-1 in the cell of the subject.

66. A method for promoting the transcription of uncoupling protein-1 (UCP-1) in a brown adipocyte of a subject, the method comprising:
a) administering to the subject the cell of any one of claims 1-26, or the cell generated by the method of any one of claims 27-33;
b) activating the optogenetic moiety with a light source, wherein the adenylyl cyclase enzyme is activated, and wherein the light source is controlled by a control device;
thereby promoting the transcription of UCP-1 in the cell of the subject.

67. The method of any one of claims 64-66, wherein activating the optogenetic moiety with a light source modulates the expression of at least one gene with a mitochondrial function.

68. The method of claim 67, wherein the at least one gene has a function in mitochondrial biogenesis; ATP synthesis, electron transport, or mitophagy.

69. The method of claim 67 or 68, wherein the at least one gene is selected from the group consisting of: Pgc1a, Tfam, Atp5g1, Atp5g3, Ndufs4, Ndufb8/complex I, Sdhb/complex II, Uqcr2/complex III, Cox412/complex IV, Bnip3, Bnip31, Pink1, and Mff.

70. The method of any of claims 64-69, wherein activating the optogenetic moiety with a light source modulates fatty acid uptake and insulin-independent glucose uptake in the cell.

71. The method of any of claims 64-70, wherein activating the optogenetic moiety with a light source, results in increased thermogenesis in the cells compared to cells that are not treated with a light source.

72. A method of treating a metabolic disease or disorder in a subject, the method comprising:
a) administering to the subject the cell of any one of claims 1-6, or the cell generated by the method of any one of claims 27-33;
b) activating the optogenetic moiety with a light source, wherein the adenylyl cyclase enzyme is activated, and wherein the light source is controlled by a control device;
thereby treating the subject.

73. The method of claim 72, wherein the metabolic disease or disorder is selected from obesity, diabetes or hyperlipidemia.

74. The method of claim 73, wherein the subject is obese and in need of treatment.

75. The method of any one of claims 64-74, wherein the cell of any one of claims 1-26 or the cell generated by the method of any one of claims 27-33 are derived from the subject or a donor.

76. The method of any one of claims 64-75, wherein the optogenetic moiety is activated by transient blue light, continuous blue light, or oscillating pulses of blue light.

77. The method of any one of claims 64-76, wherein the administered cells are treated with the light source for at least 10 minutes, at least 30 minutes, at least 1 hour, at least 2 hours, at least 3 hours, at least 4 hours, at least 5 hours, or at least 6 hours.

78. The method of any one of claims 63-76, wherein the administered cells are treated with the light source for about 10 milliseconds to about 500 milliseconds every second for at least 1 hour, at least 2 hours, at least 3 hours, at least 4 hours, or at least 5 hours.

79. The method of any one of claims 64-76, wherein the administered cells are treated with the light source for between about 10 minutes and 6 hours, about 30 minutes and about 6 hours, about 1 hour to about 6 hours, about 2 hours to about 6 hours, about 3 hours to about 6 hours, about 3 hours to about 6 hours, about 4 hours to about 6 hours, about 5 hours to about 6 hours.

80. The method of any one of claims 64-79, wherein the radiation intensity of the light source is at least 0.05 mW/cm², at least 0.1 mW/cm², at least 1 mW/cm², at least 2 mW/cm², at least 5 mW/cm², at least 10 mW/cm²,

81. The method of any one of claims 64-80, wherein the treatment with the light source is administered once a day, twice a day, three times a day, once a week, twice a week, three times a week, 4 times a week, 5 times a week, 6 times a week, or every day for at least 1 month, at least 2 months, at least 3 months, at least 4 months, at least 5 months, at least 6 months, at least 7 months, at least 8 months, at least 9 months, at least 10 months, at least 11 months, or at least 1 year.

82. The method of any one of claims 64-81, further wherein a cell of any one of claims 1-26, or a cell generated by the method of any one of claims 27-33 are grown on a scaffold prior to being administered to the subject.

83. A method of treating a subject in need thereof, the method comprising:
a) administering to a cell of the subject a nucleic acid encoding one or more adenylyl cyclase enzymes, wherein the adenylyl cyclase enzyme comprises an optogenetic moiety;
b) activating the optogenetic moiety with a light source, wherein the adenylyl cyclase enzyme is activated, and wherein the light source is controlled by a control device;
thereby treating the subject.

84. The method of claim 83, wherein the delivery vector is a plasmid, a lentivirus vector or an AAV vector.

85. The method of claim 83 or 84, wherein the nucleotide sequence encoding the one or more adenylyl cyclase enzymes is operably linked to a promoter that is not associated with the adenylyl cyclase enzyme in nature.

86. The method of claim 85, wherein the promoter is inducible.

87. The method of claims 85 and 86, wherein the promoter is induced in adipocytes.

88. The method of any one of claims 85-87, wherein the adipocyte-specific promoter is AP2 or AdipoQ.

89. The method of claim 85 or 86, wherein the promoter is selected from the group consisting of EF1α promoter, CAG promoter, and CMV promoter.

90. The method of any one of claims 83-88, wherein the adenylyl cyclase enzyme is from a strain of Naegleria australiensis, Pseudanabaena, Pseudanabaena yagii, Beggiatoa, Planktothrix, Pseudanabaena, Oxynema aestuarii, Cyanobacteria bacterium, Eutreptiella gymnastica, Eutreptia viridis, Euglena stellate, Colacium sideropus, Euglena quartana, Euglena gracilis, Oscillatoria acuminate and Euglena longa (Euglenophycean alga) (Astasia longa).

91. The method of claim 90, wherein the adenylyl cyclase enzyme is from Beggiatoa sp. PS., Pseudanabaena sp. ABRG5-3, Planktothrix sp. FACHB-1375, Pseudanabaena sp. FACHB-1277, Oxynema aestuarii AP17, or Cyanobacteria bacterium J007.

92. The method of claim 90 or 91, wherein the adenylyl cyclase enzyme is from Beggiatoa sp. PS.

93. The method of any one of claims 90-92, wherein the amino acid sequence of the adenylyl cyclase enzyme comprises one or more amino acid substitutions relative to a wild type adenylyl cyclase sequence.

94. The method of claim 93, wherein the one or more amino acid substitutions are at positions corresponding to position 27, 123, 198, 266, 267, 278, 197, and/or 201 in the wild type adenylyl cyclase enzyme amino acid sequence.

95. The method of claim 93 or 94, wherein the wild type adenylyl cyclase enzyme amino acid sequence comprises SEQ ID NO: 1 or SEQ ID NO: 30.

96. The method of any one of claims 93-95, wherein the amino acid sequence of the adenylyl cyclase enzyme comprises an alanine (A) at a position corresponding to position 27 in the sequence of SEQ ID NO: 1 or SEQ ID NO: 31.

97. The method of any one of claims 83-96, wherein the amino acid sequence of the adenylyl cyclase enzyme comprises SEQ ID NO: 2.

98. The method of any one of claims 83-96, wherein the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of any one of SEQ ID NOs: 32, 33, or 62.

99. The method of any one of claims 83-96, wherein the gene or gene cassette encoding the adenylyl cyclase enzyme comprises an amino acid sequence that has at least 90%, 91%, 92%, 93%, 94%, 95%, 96%, 97%, 98%, or 99% identity to, comprises, or consists of SEQ ID NOs: 1, 2, or 31.

100. The method of any one of claims 83-99, wherein the optogenetic moiety and the adenylyl cyclase enzyme are found in the same polypeptide in nature.

101. The method of any one of claims 83-99, wherein the optogenetic moiety is fused to the adenylyl cyclase enzyme, and is not associated with the adenylyl cyclase enzyme in nature.

102. The method of claim 101, wherein the optogenetic moiety and the adenylyl cyclase enzyme are from different organisms.

103. The method of any one of claims 83-102, wherein the gene or gene cassette encoding the adenylyl cyclase enzyme comprising the optogenetic moiety is codon optimized for expression in a mammalian cell.

104. The method of any one of claims 83-103, wherein the cell is a pluripotent cell or a preadipocyte.

105. The method of any one of claims 83-104, wherein the light source is a light-emitting diode or a laser.

106. The method of any one of claims 83-105, wherein the control device is implantable.

107. The method of any one of claims 83-106, wherein the optogenetic moiety is activated by transient blue light, continuous blue light, or oscillating pulses of blue light.

108. The method of any one of claims 83-107, wherein the cells are treated with the light source for at least 10 minutes, at least 30 minutes, at least 1 hour, at least 2 hours, at least 3 hours, at least 4 hours, at least 5 hours, or at least 6 hours.

109. The method of any one of claims 83-108, wherein the cells are treated with the light source for about 10 milliseconds to about 500 milliseconds every second for at least 1 hour, at least 2 hours, at least 3 hours, at least 4 hours, or at least 5 hours.

110. The method of any one of claims 83-109, wherein the cells are treated with the light source for between about 10 minutes and 6 hours, about 30 minutes and about 6 hours, about 1 hour to about 6 hours, about 2 hours to about 6 hours, about 3 hours to about 6 hours, about 3 hours to about 6 hours, about 4 hours to about 6 hours, about 5 hours to about 6 hours.

111. The method of any one of claims 83-110, wherein the treatment with the light source is administered once a day, twice a day, three times a day, once a week, twice a week, three times a week, 4 times a week, 5 times a week, 6 times a week, or every day for at least 1 month, at least 2 months, at least 3 months, at least 4 months, at least 5 months, at least 6 months, at least 7 months, at least 8 months, at least 9 months, at least 10 months, at least 11 months, or at least 1 year.

112. The method of any one of claims 83-111, wherein the radiation intensity of the light source is at least 0.05 mW/cm², at least 0.1 mW/cm², at least 1 mW/cm², at least 2 mW/cm², at least 5 mW/cm², at least 10 mW/cm².


SEQUENCE LISTING (97 sequences)

SEQ ID NO: 1 - protein, 353 aa, unidentified
  REGION 1..353: note = Beggiatoa sp.
  REGION 1..353: note = misc_feature - PAC wt (aa) Codon optimized
MKRLVYISKISGHLSLEEIQRIGKVSIKNNQRDNITGVLLYLQGLFFQILEGENEKVDKLYKKILVDDRHTNILCLKTEYDITDRMFPNWAMKTINLNENSELMIQPIKSLLQTITQSHRVLEKYMPARVIYLINQGINPLTVEPQLVEKIIFFSDILAFSTLTEKLPVNEVVILVNRYFSICTRIISAYGGEVTKFIGDCVMASFTKEQGDAAIRTSLDIISELKQLRHHVEATNPLHLLYTGIGLSYGHVIEGNMGSSLKMDHTLLGDAVNVAARLEALTRQLPYALAFTAGVKKCCQAQWTFINLGAHQVKGKQEAIEVYTVNEAQKYYDTLQITQLIRQTLENDKPRTY

SEQ ID NO: 2 - protein, 354 aa, unidentified
  REGION 1..354: note = Beggiatoa sp.
  REGION 1..354: note = misc_feature - PAC (S27A) (aa) Codon optimized
MMKRLVYISKISGHLSLEEIQRIGKVAIKNNQRDNITGVLLYLQGLFFQILEGENEKVDKLYKKILVDDRHTNILCLKTEYDITDRMFPNWAMKTINLNENSELMIQPIKSLLQTITQSHRVLEKYMPARVIYLINQGINPLTVEPQLVEKIIFFSDILAFSTLTEKLPVNEVVILVNRYFSICTRIISAYGGEVTKFIGDCVMASFTKEQGDAAIRTSLDIISELKQLRHHVEATNPLHLLYTGIGLSYGHVIEGNMGSSLKMDHTLLGDAVNVAARLEALTRQLPYALAFTAGVKKCCQAQWTFINLGAHQVKGKQEAIEVYTVNEAQKYYDTLQITQLIRQTLENDKPRTY

SEQ ID NO: 3 - protein, 859 aa, unidentified
  REGION 1..859: note = Euglena gracilis
  REGION 1..859: note = misc_feature - Photoactivated adenylate cyclase subunit beta, 4.6.1.1, Photoactivated adenylyl cyclase subunit betapacB
MYILVWKKGQQIKTFHTLDEAAQFKAASNIDEAQMFSVTVAPAISASGGSNEATNLRRLMYLSKSTNPEECNPQFLAEMARVATIRNREIGVSGFLMYSSPFFFQVIEGTDEDLDFLFAKISADPRHERCIVLANGPCTGRMYGDWHMKDSHMDSITTHPAMKTILYQIARSFSSMWSYLPKSAGNMLLLGKDPAAQPPEPMSVVVTFIYLVEFGSILSNPNLTEQAAEVLSTFVDVCVKNVEGSGGNIAKFITGICMAYWPINRTEEALTAIQQISEDLAQLRSQQAPGSAVSLMYSQAGVHYGRAMLCNAGSRKSDFTLLGDCINTTSRIATLAKKLKTPLLFSFEVRCLLGDEMREEIEGAGMHQVKGRDKPVVVYQFPGPELDVEMVRQKIEQFTPGRFRCQMPVVEYEALPISQRPPIFDDTPKGNPRPRTPGYGGRQRSDSLVDRLIMIAKLAGPSVSATGDTTLTTLTYISQATRPMSRLDLSAIMRTATRRNAQQSITGTLLHVNGLFVQTLEGPKDAVVNLYLRIRQDPRHTDVTTVHMAPLQERVYPSEWTLTSATEEMLATFPPLQDVLSQLAKSFTSLETYVPSTVVRYLTAGNNPRNLMPVSCGVVMLATDICSFTSLTEKSSLTEVWMICNTFIDACTSAICQEGGEVIKLIGDCVTAYFPGNNADSAVAAAQELFTFCRQLREAFVDVLDVRGCVSCGVGLDYGQVVMAQCGSLGLTEYVVAGAVSARVMEVEAITREVGYAIVVTEPVADRLSPQLRDHGIVPTPQAIEGLPCYGIAGEEFELDVDSIKRGIKALHAARSGEKPLTEPEEAKPDFRVSPGRVRHGDSGRRSNSAQGKRSIQVR

SEQ ID NO: 4 - protein, 1019 aa, unidentified
  REGION 1..1019: note = Euglena gracilis
  REGION 1..1019: note = misc_feature - Photoactivated adenylate cyclase subunit alpha, 4.6.1.1, Photoactivated adenylyl cyclase subunit alphapacA
MYILVWKEGQQIRTFQDLEECGQFQTASNITDGQIFSINVTPTMSKGGETGETQLRRLMYLSASTEPEKCNAEYLADMAHVATLRNKQIGVSGFLLYSSPFFFQVIEGTDEDLDFLFAKISADPRHERCIVLANGPCTGRMYGEWHMKDSHIDNITKHPAIKTILFQIARSFSSMWSYLPKNAANMLLLGKNPNKQAPEPMSVVVTFIYLVEFSSILAHPGLTEQCADILAAFVDACVRNVEGTGGQVAKFITGICMAYWPINRAEDALVGLQQLSEDLAELRSQQPPGSALSLIYSRCGVHYGRALLCNAGFRKADFTLLGDCINTASRITSLSVKLKVPLLLSFEVRCLLGDEMREELESSGLHKVKGRDKPVQVYQFNAPELDSAMVRAKIEQFNPGRYRALCPVKPYESLHPAQRPPIFDDTPRENQPKLSQVQRRDSLVDRLSLIAKLAFPSSMMAGGEGQLITLTYISQAAHPMSRLDLASIQRIAFARNESSNITGSLLYVSGLFVQTLEGPKGAVVSLYLKIRQDKRHKDVVAVFMAPIDERVYGSPLDMTSATEEMLATFPPLQDVLSQLAKSFISLETYVPSTVVRYLTAGNNPRNLQPVSVEVVMLATDICSFTPLSEKCSLTEVWTICNTFIDACTSAICNEGGEVIKLIGDCVTAYFPPTGADNAVHACQEIVSFCAQLRDAFHDVLDCRSVVACGVGLDFGQVIMAQCGSLGMTEFVVAGEVSARVMEVEALTREAGRAIVITEPVADRLSPKLRDTGIVPCQEGVDGVPCYGILGPEWELDVATIKKNIYGFHDARALAAMKKVDDGTNAPGRGAPAGGIPSSPKVRPPGRTNSVSSYTPDPNEALDPRMAESVFLDMCHQRGDTANNSIAVKLRQAANDDRLDLGRMLQGPHELMPVMQAIKHLTNLRMLNMSDNFVDDNNVGELVESCIPMRSLQVLDLSNNPGLTKVIALKRLIKHNTQVREILLNGTRIAPTEQRKLQSSMNVNRLCASTDLKGSHKYEH

SEQ ID NO: 5 - protein, 860 aa, unidentified
  REGION 1..860: note = Euglena longa
  REGION 1..860: note = misc_feature - Photoactivated adenylate cyclase subunit beta, 4.6.1.1, Photoactivated adenylyl cyclase subunit betapacB (Euglenophycean alga) (Astasia longa)
MYILVWKRGQQIKTFHTLDEAANFKAASNIDEAQMFSITVAPAISASGGSNEATNLRRLMYLSKSTNPEECNPQFLAEMARVATLRNREIGVSGFLMYSSPFFFQVIEGTDEDLDFLFAKISADPRHERCIVLANGPCTGRMYGDWHMKDSHIDSITTHPAMKTILYQIARSFSSMWSYLPKSAGNMLLLGKDPAAQPPEPMSVVVTFIYLVEFGSILSNPNLTEQAAEVLSTFVDVCVKNVEGSGGNIAKFITGICMAYWPINRTEDALNAIQQISEDLAQLRSQQAPGSAISLMYSQAGVHYGRAMLCNAGSRKSDFTLLGDCINTTSRIATLAKKLKTPLLFSFEVRCLLGDEMREEVEGAGMHQVKGRDKPVVVYQFPGPELDVEMVRQKIEHFTPGRFRCQMPVVEYESLPISQRPPIFDDTPKGKPRPRTPGYGGRQRSDSLVDRLIMIAKLAGPSISATGDSTLTTLTYISQATRPMSRLDLSAIMRTATRRNAQLSITGTLLYVNGLFVQTLEGPKDAMVNLYLKIRQDPRHTDVVTVHMAPIQERAYPAEWTLTSATEEMLATFPPLQDVLSQLAKSFTSLETYVPSTVVRYLTAGNNPRNLMPVSCGVVMLATDICSFTSLTEKSSLTEVWMICNTFIDACTSAICQEGGEVIKLIGDCVTAYFPGNGADSAVAAAQELFTFCRQLREAFVDVLDVRGCVSCGVGLDYGQVVMAQCGSLGLTEYVVAGAVSARVMEVESITREVGYAIVITEPVADRLSPQLRDHGIIPTPQAIEGLPCYGIAGEEFELDVDSIKRGIKALHAARSGEKPLALEPEEAKQDYRVSPGRMRHGDSGRRSNSAQGKRSTQVR

SEQ ID NO: 6 - protein, 1019 aa, unidentified
  REGION 1..1019: note = Euglena longa
  REGION 1..1019: note = misc_feature - Photoactivated adenylate cyclase subunit alpha, 4.6.1.1, Photoactivated adenylyl cyclase subunit alphapacA (Euglenophycean alga) (Astasia longa)
MYILVWKEGQQIKTFQDLEECGQFQIASNITDGQIFSVSVTPTMSKGGETGETQLRRLMYLSASTEPEKCNAEYLADMAHVATLRNKQIGVSGFLLYSSPFFFQVIEGTDEDLDFLFAKISSDPRHERCIVLANGPCTGRMYGEWHMKDSHIDNITKHPAIKTILFQIARSFSSMWSYLPKNAANMLLLGKNPNKQAPEPMSVVVTFIYLVEFSSILAHPGLTEQCADILAAFVDACVRNVEGTGGQVAKFITGICMAYWPINRAEDALVGLQQLSEDLAELRSQQPPGSALSLIYSRCGVHYGRALLCNAGFRKADFTLLGDCINTASRITSLSVKLKVPLLLSFEVRCLLGDEMREELESAGLHKVKGRDKPVQVYQFNAPELDSAVVRTKIEQFNPGRYRALCPVKPYDSLHPAQRPPIFDDTPRDSQPKVNQMQRRDSLVDRLSMIAKLAFPSSMMVGGESQLITLTYISQAAHPMSRLDLSSIQRVSFSRNESSNITGSLLYVNGLFVQTLEGPKSAVVSLYLKIRQDKRHKDVVAVFMAPIEERVYGSPLDMTAATEEMLATFPPLQDVLSQLAKSFISLETYVPSTVVRYLTAGNNPRNLQPVSVEVVMLATDICSFTPLSERCSLTEVWTICNTFIDACTSAICNEGGEVIKLIGDCVTAYFPPTAADNAVHACQEIVSFCAQLRDAFRDVLDCRSVVACGVGLDFGQVIMAQCGSLGMTEFVVAGEVSARVMEVEALTREAGRAIVITEPVADRLSPKLRDTGIVPCQEGVDGVPCYGILGVEWELDVAIIKKNIYGFHEARAQAALKKVDDGTNAPGRGAPAAGVPSSPKARALGRTSSVSSYTPDLNETLDPRMAESVFNDLCNQRGDAPNNSIAAKLRQAANDDRLDLGRMLQGPHELMPVLQAIKQLTNLRMLNMSDNFVDDNNVGELAESCIPMRSLQVLDLSNNPGLTKVIALKRLIKHNTQIREILLNGTRIAPMEQRKLQSSMNVNRMCASTDSKSSHKYDH

SEQ ID NO: 7 - protein, 1019 aa, unidentified
  REGION 1..1019: note = Euglena gracilis
  REGION 1..1019: note = misc_feature - Photoactivated adenylate cyclase subunit alpha-like protein ST-pacA
MYNRVYKERQEIRTIQGLEASGQFQTASNITDIQIYSINVTPTMSKGGETGETQLRRLMYLSASTEPEKCNAEYLADMAHVATLRNKAIGVIRFLLYSSPFFFQVIEGTDEDLDFLFAKISADPRHERCIVLANGPCTGRMYGEWHMKDSHIDNITNNPAIKTILFQIARSFTSMWSYLPKNAANMLLLGKNPNKQGPEPRSVVLTFIYLVEFSSILAHPGLTEQCADILAAFVEASVRNCEGTWGLVAKFITGTCVAYWPINRAEDALVGLQQLSEDLAVLRSYQPPGSALSRIYSRRGVHYGRALLCNAGFRKADFTLLGDCINTASRITSLSVKLKVPLLLSFEVRCLLGDEMREELERSALHKVKGRDKPVQVYQFNAPELDSALVRAKIEQFNPGRYRALCPVKPYESLHPAQRPPIFDDTPRENQPKLSQVQRRDSLVDRLSLIAKLAFPSSMMAGGEGQLITLTYISQAAHPMSRFDLASIQRIAFARNESSNITGSLLYVSGLFVQTLDDPKGAVESLYLKIRQDKRHKDVVAVFMAPIDERVYGTPLDMTSATEEMLATFPPLQDVLSQLAKSFISLETYVPSTVVRYLTAGNNPRNLQPVSVEVVMLATDICSFTPLSEKCSLTEVWTICNTFIDACTSAICNEGGEVIKLIGDCVTAYFAPTGADNAVHACQEIVSFSAQLRDAFHDVLDCRSVVACGVGLDFGQVIMAQCGSLRLTEFVVAGEVSARVMAVEALTREAGRAIVITEPVSDRLSAKLRDTGIVPCQEGLHAVLLWYPGPRMGIGCANHQEDHLRVPRRPGPGRHEEGGRRHQRPGPGRPRRGHPFLPQGSTTRPHQFSVQLHPLPKLGTGSSDGGIGVSLHVYSAGRYRQQLHRRQIAAGGQRRSVGPGQDVARATRAHACDAGHQAPDQPANAQYERQFRGGQQRWRTCGIVHSNAFVTGVGLVQQPWLDEGHRPEAPHQSLHPGPGDPSEQHPHRPNRATEPGLLVARQPAVCLHRPEGFAQICTF

SEQ ID NO: 8 - protein, 859 aa, unidentified
  REGION 1..859: note = Euglena gracilis
  REGION 1..859: note = misc_feature - Photoactivated adenylate cyclase subunit beta-like protein FBpacB
MYILVWKKGQQIKTFHTLDEAAQFKAASNIDEAQMFSVTVAPAISASGGSNEATNLRRLMYLSKSTNPEECNPQFLAEMARVATIRNREIGVSGFLMYSSPFSFQVIEGTDEDLDFLFAKISADPRHERCIVLANGPCTGRMYGDWHMKDSHMDSITTHPAMKTILYQIARSFSSMWSYLPKSAGNMLLLGKDPAAQPPEPMSVVVTFIYLVEFGSILSNPNLTDQAAEVLSTFVDVCVKNVEGSGGNIAKFITGICMAYWPINRTEEALTAIQQISEDLAQLRSQQAPGSAVSLMYSQAGVHYGRPMLCNAGRRKSDFTLLGDCINTTSRIATLAKKFKTPLLFSQEVRCLLRDEMREEIVGAGMHQVKGRDKPVVVYQFPGPELDVEMVRQKIEQFTPGRFRCQMPVVEYEGLPNSQRPPIFDDTPKANRRPRTPGYGGRQRSDSQVDRPIMIAKLAGPSVSATGDTTLTTLTYISQATRPMSRQDLSAIMRTATRRNAQQSITGTLLHVNGLFVQTLEGPKDAVANLYLRVRQDPRQTDVTTVHMAPLQERVYPSEWTLTSATAEMLATFPPLQDVLAQLAKSFTSLETYVPSTVVRHLTAGNNPRNLMPVSCGVVMLATDICSFTSLTEKSSLTEVWMICNTFIDACTSAICQDGREVIKLIGDCVTAYSPGNNADSAVAAAQELFTFCRQLREAFVDVLDVRGCVSCGVGLEYGQVVMAQCGSMGLTEYVVAGAVSARVMEVEAITREVGYAIVVTEPVADRLSPQLRDHGIVPTPQAIEGLPCYGIAGEEFELDVDSIKRGIKALHAARSGEKPLTEPEAAKPDFRVSPGRVRHGDSGRRSNSAQGKRSIQVR

SEQ ID NO: 9 - protein, 1019 aa, unidentified
  REGION 1..1019: note = Euglena gracilis
  REGION 1..1019: note = misc_feature - Photoactivated adenylate cyclase subunit alpha-like protein FBpacA
MYILVWKEGQQIRTFQDLEECGQFQTASNITDGQIFSINVTPTMSKGGETGETQLRRLMYLSASTEPEKCYAEYLADMAHVATLRNKQIGVSGFLLYSSPFFFEVIEGTDEELDFLFAKISADPRHVRCIVLANGPCTGRLYGEWHMKDSHIDNITKHPAIKTILFQIARSFSCMWSYLPKNAANMLLLGKNPNKQAPEPMSVVVTFIYLVEFSSILAHPGQTEQCADILAAFVDACVRNVEGTGAQVAKFITGICIAYWPINRAEDALIGLQQLSEDLAELRSQQRPGSALSLIYSRCGVHYGRALLCNAGFRKADFTLLGDCINTASRITSLSVKLKVPLLLSFEVRCVLGDEMREVLETSGLHKVKGRDQPVQVYQFNARELDSAMVRAKIEQLNPGSYRALCPVKPYESLLPAQRPPIFDDTPRENQPKLSQVQRRDSLVDRLSLIAKLAFPTSMMAGGEGQLITLTYISQAAHPMTRLDLASIQRIAFARNESSNITGSLLHVSGLFVQTLEGPKGAVVSLYLKIRQDKRHKDVVADFMAPSEGREYGSPLDMTSATEEMLTSFPPLQELLTHLAKPFISLETYVPTTVVRYLTAGNNPRNLQPVSVEVVLLATDICSFTPLSEKCSLTEVWTICKTIFVAFIRAIFNEGGEVIKLIGDCVTAYFPPTGADKAVHACQEIVTFCAQLGDAFHDVLDCRSVVACGVGLDFGQVIMAQCGSLGMTEFVVAGEVSARVMEVVALTREAGRAIVITEPLPDRLAPKLRDTGIIPCQEGVDGVPCYGNLGPEWELDVATIKKNIYGFHDARALAAMKKVDVGTNAPGRGAPAGGIPSSPKVRPPGRTNSVSSYTPDPNEALDPPMAESVFLDMCHQRGDTANNSIAVKLRQAANDDRLDLGRMLQGPHELMPVMQAIKHLTNLRMLNMSDNFVVDNNVGELVESCIPMRSLQVLDLSNNPGLTKVIALKRLIKHNTQVREFLLNGTRIAPTEQRKLQSSMNVNRLCASTDLKGSHKYEH

SEQ ID NO: 10 - protein, 1019 aa, unidentified
  REGION 1..1019: note = Euglena gracilis
  REGION 1..1019: note = misc_feature - Photoactivated adenylate cyclase subunit alpha-like protein 1224-5/1FpacA
MYILVWKEGQQIRTFQDLEESVQFRTASNITDGQIFSINVTPTMGKGGETGETQLRRLMYLSASTEPENCNADYVGDMAHFATLRNKSIGVSGFYMYSAPFLFQVIQGTDEDLDFLCANGSADPRHQRCIELADGPTTGRMYGWWHLKDSHIDNITKDPAIKTILIEIARSFSSMWSYLPRNAANMVLLGKNPNKQAPEPMSVVVTVIYLVEFSVIMAHPGLSEQCPDILPAFVDACVPHVDGTGGEVAKFIIAICMAYWPINSDEHALVGLQQLSDDLAELRSLQEDGSARSLIYSRCGLHFGRAMLCNAGFRKADFTLLGDWINSASRITSLSVNSKVPLLLSFLVRCLLGDEMRVELERSGLHKVGGRVKPVQVYQFNAPELDTATVRGEIKQFNPGRERALCPVKPYESLHPAHPDPIFDDTPRENQPELSQVQRRDSLVDRLSQIAKLAFPSSMMAGGEGQLITLTYISQAAHPVSRLDLASIQRIAFARNESSNITKSLLYVSGLFVQTLEGTKGAVVSLYLKNRQDKRHKDVVAAFMAPIDEGVYGSPLDMTSATEEMLATFTPLHDVLSQLAKSFISLETYVPSTVVRHLAAGNNPRNLQPVSVEVVMLATAISSFTPLSEKCSLTEVWTICNTFIVACTSAICNEGGEVIKLIGDCVPAYFPPTNADNAVHACQEIVSFCAQLRAAFHDVLDCRSVVACGVGLDYGQVIMVQCGSLGMTEFVVAGEVRARVLEVEALTREAGRAIVITEPVPERQSPKLRDTGIVPCQEGVDGARCYGILGPEWELDVATIKKNINGFLKDARALAANKKVDDGTNISCRGGNPPAGGIPTSPKVRPPGRTNSVSSYTPDPKQALDPRMAESVFLDMCHQGDTVNNSIAVKLGLAANEDGFDGGWILTWYVELMPVKQAIKVLTQLRIGNMSDNFVDDNNVGELLEKCIPTRSLQVLDLSNNPGLTKVIALKPKHNTQVREILLNGTRIAPTEQRKQQTSKNVNRLCASTDLKGSHKYEH

SEQ ID NO: 11 - protein, 859 aa, unidentified
  REGION 1..859: note = Euglena gracilis
  REGION 1..859: note = misc_feature - Photoactivated adenylate cyclase subunit beta-like protein ST-pacB
MYILVWKKGQQIKTFHTLDEAAQFKAASNIDEAQMFSVTVASAISASGGSTEATNLRRLMYLSKSTNPEECNPQFLAEMARVAFIRNREIRVSGFLMYSSPFFIQVIVETDEDLDFLFAKISADPRHEMCIVLANGPGTGRMYGDWHMKDSHLDSITTHTAMKTILYQIARSFSSMWIYLQKSAANMLMLGKDPAAQPPERRSVVVTFIYLVEFCSIFSHPNLTEKTADVLSTFVYVCVKNVEGSGGNIAKFIPAICMTYWPIIRTKEALTAIQQISEDLAQLRSQQAQGSAVSLRYSQAGVHYGRGMQCNAGSRKSDFTLLGDCINTTSRIATLAKKLKTPLLFGFEVRCLLGDEKREEIEGAGIHQVKGRDKPVVVYQFPGPDLDVAMVRQKIEQFTPGRFRCQMPVVEYEALPISQRPPIFDDTPKSNPRPRTPGYGGRQRSDSLVDRLIRIANLSGFSESATGDTTLTTLTYISQATRPMSRLDLSAIMPTATRRNAQQSNIGTVLHENGLFVQTLEGPKDAVVNLYLRIRQDPRHTDVTTVHMAPLQERVYTSEWTLTLATEEMLATFPPVQDVLSQLAKSYTSLETYVPSTVVRYLTAGNNPRNLMPVSCGVVMLATDISSFTSLTEISSFTEVWMICNTFIDACTSAICQEGGEVIKLIGDCVTPYFPGNNADLAVAAAQELFIFCRQLREAFVDVLDVRGCVSCAVGMDYGQVVMAQCGLLRLSDYVAAGAVSAGVVEVEAITREVGYQFVVTVPDADRVQPQFRDYGIEPTPQAIGGLPCYGIVREVFDLPVESIKRGIKAMHAARSGEQPLTEPEQAKPDFRVSPGRDRHGVSGRRSNSSQGKGSIQVG

SEQ ID NO: 12 - protein, 1015 aa, unidentified
  REGION 1..1015: note = Eutreptiella gymnastica
  REGION 1..1015: note = misc_feature - Putative alpha subunit of photoactivated adenylyl cyclase, 4.6.1.1
MYVLVWKNGQQVKTFETLDGANSYKTGSNIADDDSNIFHIDVIPAQSAGSAGGGNVDKPLRRLMYLSKTTEAELCTPEYLADMAHVATLRNMEIGVSGFLLYSAPFFFQVIEGTDEDLDFLFAKIGADPRHERCIVLANGPCTGRLYGDWHMKDSHIENITSHPAIKTILYQIARSFSSMWSYLPKNAANLLLLGKNPNKQTPEPTSVVVTFIYLVEFSSILAHPGLTEQVADVLSHFVDICVRNIEGSGGAIAKFITGITMAYWPINRAEDALLALQQINEDLAQLRSQQPPGSALSLMYGRAGVHYGRALLCNAGVRKSDFTLLGDCINTASRVTSLATQLKSPLLFSFEVRCLLGDDMRDELESAGMHKVKGRDKPVAVYQFPCEPLDNDMVRAKIEAFNPGRYRAQFPVVPYDSLHGVHKPPIFDDSPKDPLATRIAQAAGERKDSLIDRLIMISNLAIPASMAGGGNNQLITLTYISHAVKPMSRLDLAAINRNAVRRNAASKITGTLLYVNGLFVGTLEGPKDAVLTLYNKVRRDARHEDVVTVHLAPLDERVYDVPFSVTAATDSMLSKFPPLQDVLSQLAKSFISLETYVPSTVVRWLTAGNNPRGLHAVSTEVVMFATDICSFTPLSENCSLVEVWLICNTFIDACTHSICNKGGEVIKLIGDCVTAYFPPDGADAAVLACQEVVTFCAQLREAFEEVLDCRSVVACGVGLDYGQVVMAQCGSEGMTEYVVAGEVSARVMEVEALTRDVARAIVVTEPVADRLSPAMRDNGIIPCSQGVDGIPCYGILGEEWELDIPTIKANIKRFHATRAEIARQKEEEAAAAEAGKPKAKARADSRFGPRGSVNSYCPDPNEVLDPRMAEDVFVEACAARGDAPNLALKAKMRQAANDDRLDLARALQGPHELSPLTTALKQLCHLRSLNMSDNFIDDNNVVDLVEACLPMENLQVLDLSNNPGITKIIALKRLVKHNVQIREINLNGTRVAPTEQRKLQSSINVNRLCASEKR

SEQ ID NO: 13 - protein, 1017 aa, unidentified
  REGION 1..1017: note = Colacium sideropus
  REGION 1..1017: note = misc_feature - Putative alpha subunit of photoactivated adenylyl cyclase, 4.6.1.1
MYILVWKEGQQIKTFQDLEECGQFQTASNISDGQTFSINVSPVMSKGGETGETQLRRLMYLSASTEPERCDGEYLAEMAHVATLRNSEIGVSGFLLYSAPFFFQVIEGTDEDLDFLFAKIGADPRHERCIVLANGPCTGRLYGEWHMKDSHIDNITKHPAIKTILYQIARSFSSMWSYLPKNAANMLLLGKNPNKQAPEPMSVVVTFIYLVEFSSILAHPGLTEQCADVLAAFVDACVRNVEGTGGQIAKFITGICMAYWPINRAEDALVGLQQLSEDLAELRSQQPPGSALSLVYSRCGVHYGRALLCNAGTRKSDFTLLGDCINTASRITSLSVKLNVPLLLSFEVRCLLGDEMREELESAGLHKVKGRDKPVQVYQFNAPELDSAQVRAKIESFNPGRFRPMCPVKPYDSLHPSQRPPIFDDTPRESMPKLSVVQRRDSLVDRLSLIAKLAFPPNMMNGGDNQLITLTYISQASYPMSRLDLAAIQRVGCRRNESAQITGSLIYVNGLFVQTLEGPKSAVITLYLKIRQDSRHKDVVIVFMAPIEERVYSTALDMTSATEEMLATFPPLQDVLSQLAKSFISLETYVPSTVVRYLTAGNNPRSLQPVSVEVVMLATDICSFTPLSENCSLTEVWTICNTFIDACTSAICSEGGEVIKLIGDCVTAYFPPTSADNAVAACQEIVTFCTQLREAFSDVLDCRSVIACGVGLDYGQVVMAQCGSLGMTEFVVAGEVSARVMEVEALTREAGFTIVVTEPVADRLSPKLRDTGIIPCHEGVDGVPCYGIAGDEWVLDIKTIKANIYGFHEARAQAALRKVDDGTRAPGHAAPASPKAQKGAGRANSVASYTPDVNELLDPRMAESVFQELCQQRGDVPNVGISAKLRQAAQDDRLDLGRTLQGPHELGPVVQAVKQLTNLKTLNLSDNFVDDGSVGEIVEACLPMRNLQVLDLSNNPGLTKIIALKRLIKHNPAVREILLNGTRVAPTEQRKLQSSINVNRLCSSTDAKGGSHKYESH

SEQ ID NO: 14 - protein, 1027 aa, unidentified
  REGION 1..1027: note = Euglena quartana
  REGION 1..1027: note = misc_feature - Putative alpha subunit of photoactivated adenylyl cyclase, 4.6.1.1
MYILVWKDGQQVKNFQDLEECGAFQTAANIVDGQIFNVGVTSAMNKGGDTGEVQLRRLMYLSASTEPEECTTEYLAELARASSLRNKEIGVSGFLLYSAPFFFQVIEGTDEDLDFLFAKISSDPRHEKCIVLANGPCTGRLYGEWHMKDSHIDNITKHPAIKTILFQIAKSFSSMWSYLSKNTANMLLLGKQPNKQAPEPMSVVISFIYLVEFSSILAHPGLTEQCADVLAAFVDVCVRNIEGTGGHVAKFITGICMAYWPSNRAEDALVGLQQLSDDLAELRSQQPPGSALSLVYSRCGVHYGRALLCNAGIKKADFTLLGDCINTASRITSLAGKLKVPLLLSFEVRCLLGAEMREELESAGMHKVKGREQPVQVFYFNAAEVDSTAVRAKIEQFNPGRYRALCPVKPYDSLHPSQKPPIFDDTPRDSMPRSGHLQRQDSLVDRLSMIAKLAFPPSMAAGGESQLIGLTYISQASHAMSRLDLSDIQRVAHRRNEQVNISACLLYINGLFVQTLEGPKSAVVSLYLKIRQDPRHKDVVVVHMAPLEERMYRGPLDTTCATDEMLATFPPLQDVLSQLAKSFISLETYVPSTIVRYLTSGNNPRNLQPVSLEVVMLATDICSFTPLSEHCSLTEIWTICNTFIDACTSAICSQGGEVIKLIGDCVTAYFPPTSADNAVYACQDIVAFCANLREAFRDVLDCRSVVACGVGLDFGQVIMAQCGSSGLTEFAVAGEVSARVMEVEALTREAGRAIVLTEPVADHLSPKLRDNGLVPCQEGVDGVPCYGILGAEWELDVVTIKRNIQNFHAARAVAAVQKVDDGMNAPGRALPVASMPAISSPSPKVRTAGRAASVASYAPDLNDLLDPRMAEVVFQELCQQRGEIPNNAVLLRLRQAAHEERADLGRSLQGPHELPPLTQALKQLTNVRALNMSDNFVDDTSIGDLIESCLAMRSLQMLDLSNNPGLTKVIALKRLIKHNPQLREIHLNGTRIAPTEQRKLQSSLNVNRLCASAESKLGSHKYEGAAH

SEQ ID NO: 15 - protein, 1017 aa, unidentified
  REGION 1..1017: note = Euglena stellata
  REGION 1..1017: note = misc_feature - Putative alpha subunit of photoactivated adenylyl cyclase, 4.6.1.1
MYILVWKEGQQIKTFQDLEECGQFQTACNISDGQIFSVNVAPTMSKGGESGEQLLRRLMYLSKSTEPEACNAEYLAQMAFVATCVNSTIGVSGFLLYSAPFFFQVIEGTDDDLDWLYAKISRDPRHERCIVLANGPCTGRMYGEWHMKDSHIDNITKHPAIKTILFQIARSFSSMWSYLPKNAANMLLLGKEPNKQAPEPMSVVITFIYLVEFSSILAHPGLTEQCADILEAFVDACVRNIEGTGGQVAKFITGICMAYWPINRAEDAIVGLQQLSEDLAELRSQQPPGSALSLVYSRCGVHYGRALLCNAGFRKADFTLLGDCINTASRITSLSVKLKVPLLLSFEVRCLLGDEMREELENSGLHKVKGRDKPVQVYQFNAPELDSAMVRAKIEQFNPGRYRALCPVKPYDSLSATQKPPIFDDTMRDGHNKLSAAQRRDSLVDRLSLIAKLAFPPSMLAGGDGQLITLTYISQASRPMSRLDLSAIHRVAMRRNESSNISGCLLYVNGLFVQTLEGPKSAVVSLYLKIRQDGRHKDVVTVYMAPLEERVYATALDMTAATEDMLSTFPPLQDVLSQLARSFISLETYVPSTIVRFLTAGNNPRNLQPVSVDVVMLATDICSFTPLSERCSLTEVWTICNTFIDACTSAICNEGGEVIKLIGDCVTAYFPPHAADNAVSACQEIVTFCTHLREAFKDVLDCRSVIACGVGLDFGQVIMAQCGSLGMTEFVVAGEVSARVMEVEALTREAGRAIVVTEPVADRLSSRLRDSGIVPCQEGVDGVPCYGILGPEYELDIVTVKANIYGFHDARAQAALRKADDGTNAPGKSGPAVPSSPKVHHAQGRAGSVASYTPDPNEVLDPRMAEMVFQDMCQHRGDQPSNAILMKLRNAAQDDRLDLGRTLQGPHELGPLTQAIKQLTNLKMVNMSDNFIDDNTIGDLVEACLSMRSLQVLDLSNNPGLTKTIALKRLIKHNPHVREVLLNGTRIAPTEQRKLQSSMNVNRLTASDKGGSHKYDH

SEQ ID NO: 16 - protein, 860 aa, unidentified
  REGION 1..860: note = Colacium sideropus
  REGION 1..860: note = misc_feature - Putative beta subunit of photoactivated adenylyl cyclase, 4.6.1.1
MYILVWKKGQQIKTFHTLDEAAQFKAASNIDDAQMFSINVAPAVSASGGSNEATNLRRLMYLSKSTEPEKCDVPYLAEMAGIATLRNREIGVSGFLMYSSPFFFQVIEGTDEDLDFLFAKIGADPRHERCIVLANGPCTGRMYGDWHMKDSHIDSITTHPAMKTILYQIARSFSSMWSYLPKSAGNMLLLGKDPASQPPEPMSVVVTFIYLVEFGSILSNPNLTEQAAEVLSTFVDVCVKNVEGSGGNIAKFITGICMAYWPINRAEDALTSIQQISEDLAQLRSQQAPGSAVSLMYSQAGVHYGRAMLCNAGTRKSDFTLLGDCINTTSRVATLAKKLKTPLLFSFEVRCLLGDEMREEIEGAGMHQVKGRDKPVVVYQFPGEELDVDEVRHKIETFNAGRFRCQMPVVEYDTLHPSQRPPIFDETPKGNPRPRTPGYGGTQRRDSLVDRLIMIAKLAGTTASTTGDTTLITLTYISQASRPMSRLDLSAIMRASTRRNTQQNITGTLLHVNGLFVQTLEGPRDAVMNLYLRIRQDPRHTDVVTVHMANLQERVYPPEWTLTSASEDMLAAFPPLQDVMSQLAKSFTSLETYVPSTVVRYLTAGNNPRNLMPISCGVVMLATDICSFTSLTEKSSLTEVWMICNTFIDACTSAICQEGGEVIKLIGDCVTAYFPGSAADAAVAAAQELYLFCTQLREAFVDVLDVRGCVSCGVGLDYGQVVMAQCGTLGLTEYVVAGAVSARVMEVEALTREVGHAIVVTEPVADRLSPQLRDSGIIPCPQAIEGIPCYGIAGEQFELDIDSIKRGIKALHAARSGEKVAVAEPEEKGGDFRVSPGRVRRSDSGRRSNSAQGKRGTPVR

SEQ ID NO: 17 - protein, 862 aa, unidentified
  REGION 1..862: note = Eutreptiella gymnastica
  REGION 1..862: note = misc_feature - Putative beta subunit of photoactivated adenylyl cyclase, 4.6.1.1
MFLLVWKKGQQMATFHTLEESNEFKAASNIEDSHVFHLKLTPTTSGAGGANNATGFRRLMYLSKSTEAEEVNSTYLAEMAGIATIRNQEIGVSGFLMYSDPFFFQVIEGTDEDLDFLFAKIGADPRHERCIVLANGPCTGRLYGDWHMKDSHIDSITSHPAMKTILYQIARSFSSMWSYLPKSAGHMLLLGKDPNKQPPEPMSVVVTFIYLVEFSSILSHPGLTEQCAEVLSTFVDVCVRNVEGSGGNIAKFITGICMAYWPINRAEDALTALQQINEDLAQLRGAQAPGSALSLMYSRSGVHYGRAMLCNAGTRKSDFTLLGDCINTASRVASLAVTLKSPLLFSFEVRCLLGDEMRDEIESAGMHQVKGRDKPVVVYQFPSPILDSDVVRQKIEQFNPGRYRCSMPVLDYETMNPAKRPPIFDNAPKGNPKIGKKYNAAAHRDSLVDRLTEIARLAFPGSQAGGDSTLISLTYLSNSVRPMSRLDLAAIQRTALRRNASQGITGCLLYVNGLFVQTIEGPRDNVINLYLKIREDPRHKDVVTVYMVPIQDRVYQEAWTMTTASEDMLATFPPLQDVLSQLAKSFTSLETYVPSTVVRYLTAGNNPRDLMPVSVECVMFATDICSFTMLTEKSSLEEVWMICNTFIDACTSAICAEGGEVIKLIGDCVTAYFPSERADNAVAASQELYMFCVHMREAFQEILDVRACIACGVGLDYGQVVMAQAGSLGLTEYAVAGAVSARVMEVEALSRDVGRAIVCTEPVVDRLSPHLRDSGVIPCPQAIEGIPCYGIAGEDYELDIRSIKAGIKQLHAARSLEDQAAAAEEEAAQAQQQFRVSPGRVRRSDAGRRSNSAQGRRMAPVR

SEQ ID NO: 18 - protein, 861 aa, unidentified
  REGION 1..861: note = Euglena stellate
  REGION 1..861: note = misc_feature - Putative beta subunit of photoactivated adenylyl cyclase, 4.6.1.1
MYILVWKKGQQIKSFHTLDEAAEFKAASNIDEAQMFSITVAPAISASGGTNEATNLRRLMYLSKSTNPEECDPAFLAEMAGIATIRNKEIGVSGFLMYSSPFFFQVIEGTDEDLDFLFAKISADPRHEKCIVLANGPCTGRMYGDWHMKDSHIDSITTHPAMKTILYQIARSFSSMWSYLPKSAGNMLLLGKDPAAQPPEPMSVIVTFIYLVEFGSILSNPDLTEQAAEVLGTFVDVCVKNVEGSGGNIAKFITGICMAYWPINRAEDALTSIQQISEDLAQLRSQQAPGSAITLMYSQAGVHYGRAMLCNAGTRKSDFTLLGDCINTTSRVATLAKKLKTPLLFSFEVRCMLGDEMREEIEGAGMHQVKGRVKPVVVYQFPGPELDVEMVRQKIDQFNPGRFRCQMPVVDYDSLPVSQRPPIFDDTPKGNPRPRTPGYGGTQRSDSLVDRLIMIAKLAGPSASAGGDTSLTTLTYISLATRPMSRLDLSAIMRTATRRNSSQGITGTLLYINGLFVQTLEGPKDAVVNLYLRIRQDPRHTAVVTVHQAALQERVYPPEWTMTSATEEMLATFPPLQDVLSQLAKSFTSLETYVPSTVVRYLTAGNNPRNLMPVSCSVVMLATDICSFTSLTEKSSLTEVWMICNTFIDACTSAICQEGGEVIKLIGDCVTAYFPGNAADAAIAAAQELFMFCTTLRQAFVDVLDVRGCVACGVGLDYGQVVMGQCGTMGLTEFVVAGAVSARVMEVEALTREVGRAIVVTEPVADRLSPQMRDTGIIPCPQAIEGLPCYGIAGDDYELDIDEIKRSIKHLHAARSGEKMPSADHEENKGNDFRVSPGRVRRSDSGRRSNSAQGKRGTPVR

SEQ ID NO: 19 - protein, 867 aa, unidentified
  REGION 1..867: note = Eutreptia viridis
  REGION 1..867: note = misc_feature - Putative beta subunit of photoactivated adenylyl cyclase, 4.6.1.1
MVFLLVWKNGTQLKSFLTLDDATQYKTASSIDDAQIYNVAFTPAVSGDGGSNEATNLRRLMYLSKSTEPELVNTQYLAEMAHIATLRNQCIGVSGFLMYSHPFFFQVIEGTDEDLDFLFAKIGIDPRHERCIVLANGPCTGRLYGDWHMKDSHIDSITSHPAMKTILYQIARSFSSMWSYLPKSAGNTLLLGKDPAAQPPEPMSVVVTFIYLVEFSSILSHPGLTEQCAEVLSTFVDVCVKNVEGSGGNIAKFITGICMAYWPINRAEDALTSIAQISEDLGQLRGQQPPGSALSLMYSRAGVHYGRAMLCNAGSRKSDFTLLGDCINTASRVASLAVTLKSPLLFSFEVRCLLGDEMRDEIEGAGMHSVKGKDKPVVVYQFPGPELDSGMVRQKIEEFNPGRYRCGMPVMEYESLHVSEQPPIFDDTPKGTPKPGTRAWGGTNRKDSLADKLALISQLAGPLQTSGDTTLTTLTYISHSTRPITRMDLASIQRCALRRNAQHNITGCLLYVTGLFVQTLEGPKDAVINLYHKIRADARHTDVVTVHQGPIQDRVYASAWALTNTSEEMLSAFPPLQDVLSQLAKSFTSLETYVPSTIVRYLTAGNNPRNLRPVSVGVVMLATDICSFTMLTEKSSLTEVWMICNTFIDACTNAVITNGGEVIKLIGDCVTAYFPGHGADGAVAAAQEIFSFCTQLREAFADALDVRSAISAGIGLDFGQVVMAQAGSLGVTEFAVAGSVSARVMEVEALTRDVGRAIVVTEPVADRLSPQLRDRGLIPCPQAIEGIPCYGLAGDDFELDIASIKGRIRNLHAMRAGEKPNLPDDHEQEDNYKQAQQDFRVSPGRVRRADSGRRSNSAQGRRSAPVR

SEQ ID NO: 20 - protein, 1027 aa, unidentified
  REGION 1..1027: note = Eutreptia viridis
  REGION 1..1027: note = misc_feature - Putative alpha subunit of photoactivated adenylyl cyclase, 4.6.1.1
MYVLIWKSGQQIQTFPDLEAASQFKAASNIEDSFIFSVSVTPTMLAGGAAGESGLRRLMYLSKATEPDECTPEFLAELAHVATLRNQEIGVSGFLLYSAPFFFQVIEGTDEDLDFLFGKIGLDTRHEACIVLANGPCSGRMYGEWHMKDSHIDNITKHPAIKTILYQIARSFSSMWAYLPKQAGNLLLLGKEPNKQPPEPMSAVVSFIYLVEFSTILANPNLTEQVADILIAFVDSCVKNVEGSGGSVAKFITGICMAYWPINRAEDALLGLQAISDDLAELRAHQPPGSALSLVYSRAGVHYGRALLCNAGMQKSDFTLLGDCINTASRVTSLSVQLKVPLLFSFEVRCLLGDDMREELESLGMHKVKGRDKPVAVYQFAGAPELDSAMVKAKVEQFNPGRYRAQCPVRELESLHPDVRPPIFDDTPREASMPKAGAGERRDSLADRLTMIAKLAFPTIGGGTNSQMITLTYVSFSSRPMSRLDLAAIQRVGLRRNSQVDITGSLLYVAPLFVQTLEGPKDAVISVYMKIRADPRHRDVVAVYMAPQEERVYNSAFELTTATEEMLSAFPPLQDVLSQLAKSFISLETYVPSTVVRYLTAGNNPRSLQAISTGVVMFATDICSFTPLSEKCSLTEVWLICNTFIDACTAAIVQERGEVIKLIGDCVTAYFPPTNADGAVMACQEIVAFCTGLRAAFSDVLDVRQVVACGVGLDYGDVVMAQCGSMGMTEFVVAGEVSARVMEVEALTREVGRAIVITEPVADRLSPKMRDTGIAPCPEGVDGIPCYGIVGDDWDLDIVTIKKNIYSFHDRRDAVAHAKAAAAQEALDAAAPSPKKGGRGSGRSSSVSSYAADPNEVLDPRMAEAAYTQGCQQRGEQPTMAIVNKLRGAGNDTRMDLGRALQGPHELMALCTALKHLTHLRLLNMSDNFVDDNSIAEIVEACLPMKSLAVLDLSNNPGLTKILALKRLVKHNPGIKEIILQGTRIAPPEQRKLQSSINVNRLCASSPNGAESKAGHKYDQSMSSSHH

SEQ ID NO: 21 - protein, 859 aa, unidentified
  REGION 1..859: note = Euglena gracilis
  REGION 1..859: note = misc_feature - Photoactivated adenylate cyclase subunit beta-like protein 1224-5/1FpacB
MYILVWKKGQQIKTFHTLDEAAQFKAASNIDEAQVFSVTDAPDISASGGSNEARNRRRLMYLTNSTNPEECNPQFVGEMARVATIQNREIGARGFLMYSCRFLLQVIEETVEDPDSLFAKFSPEPRHERWIELDNGPCTGRMYGDWNMKDSHLDSITTHPAMLTILFQMARSISCLWSYLQKSERIMLLFGKDPGAQRPEPMRVVVTFTYLVDFGSMLSIPNLTEQAAEVLTTFVDGCVKNVEGSGGSIAKFTTGICMAQWPINRTVEALTAIQQISEDLAQLRSQQAPGSAVSLMYSQAGLHYGRGMLCNAGSRKSDFTLLDFINTTSRIATLANKLKTPLLFHSFDVRCLLGDEMREEIEGAGMHQVKGRDKPVGVYQFPGPELDVEMVLRQKVEQFTPGRFRCQMPVVEYEALPIWQRPPIVDDTPKGYPRPRTMPGYGGRQRSDSLVDRLIMIAKLAGPSVSATGDTTLTTLTYISQATRKPMSRLHLSALMGTATRRNAQQSITGTLLDVNGLFGQTLEGPKVAVVMYLSIRQDARQTDVTTVHMAPLQERVCPSEWTLTSATEEMLATFPPLQDVLSQLAKSCTSLETYVRSTVGYLTAGNNRRNLMPVSCGVVMLATDIGSFTSLTEESSLTEVWMICNTFIDACTSAVCQEGGEVIKRIGDCVTAYFPGNNAESAVAAAQELFTFCRQLREAFVDVVDVRGCVSCGVGLDYSQVVMAQCGSLGLTEYVVAGVSARVMEVDAMTREVGYAIGVTEPYVTDRLSPQSRDHGIVPTPQAIELPCYGIAGEQLELDVDSIKRGIKALHAARSGEMPVTEPEEALRLFRVSPGRVRHGDTGRRSNSAQGKRSIQVR

SEQ ID NO: 22 - protein, 855 aa, unidentified
  REGION 1..855: note = Euglena gracilis
  REGION 1..855: note = misc_feature - Photoactivated adenylate cyclase subunit beta-like protein 1224-5/9FpacB
MYILVWKKGQQIKTFHTLDEAAQFKAASNFDEAPMFSVTVAPAISASGGSNEATNLRRLMYLSKGTNPEECNPQFVAELARVATIRNREIGLSGFLMSSSPFLFQAIEGTDQDLDFLFAKISADPRHERCIVLANRPCTGRMSGVWHMKDSHMDSITTHDAMMTILYQIARSFSSMLTYLPKRAGNMLLSGLDPAAQPPEPKSVVVTFIYLVAYGSLLSNPYSTEQPAEVLSTFVDVCVKNLEGSGGIITKFITGICMASWPFNRTEEALTAIQQISEDLAQLPSQQAPGSSPSLMYSQAGVHYGRAMLCNAGSGKSDFTLLGDWINTTSRIATLAKKLTSPVLFSFQVRCLLGDEMREEIEGAVMDQVKGRDKPVVEYQFPGPERDVEVVGQKIEQFTPGRFRCQMRVGQYEALPISQRPPIFDDTPKCNPRPRTPGCEGRQRSHTLVDPHIMMAKLPGPSVSATGDTTVPTLTYISHATRPMSRLDLSAIMGTATRINALQRITGTLLHGNGLFVQTQEGPKEAEINLLLRIRQDTRRSDVATDHMPQPQERVYPSEWTLTSGKEEILAIFPPLQDVLSRLAKSFTAQETYVPSRVVSYLTAGNNPRELMPVSCGVVMTDICSLTSLTENSCLSEVWMICNTFIDACPSAICQEGAEVIKLIGNCVTAYFPGNNADSAVASAQELFTFCREHRQAIVDVLDVRGCVSYGVALDNGQVVMAQCGSEGLTEYVVAGAVSARVMEVEAITRGTCPRWATPLWSADRLSPQLRDHGIVPTPQAIEGLPCYGIAGEEFELDVDSIKLGIKALHAARSGEKPLTEPEEAKLDFSPGRVRHGDSGRRSNSAQGKLSIQVR

SEQ ID NO: 23 - protein, 846 aa, unidentified
  REGION 1..846: note = Euglena gracilis
  REGION 1..846: note = misc_feature - Putative membrane-bound adenylyl cyclase, 4.6.1.1
MRFSVYSLYALACLLAVGGAILTGIILLDTFDGQQRNSENAQFTAGKGQVQSMQGVIANRMTRMQTITEEHSRQIMMFLQSLPSNAIQPDNVVQAVNHSIFDSWVPSVKANNSLNGYGLTFTYTNETTGNSFDRTFWVYWDLMTTGVYDYIYAYTNMSDGYIHAYETLWPNFSTPQLGLDMYRFRSTVLAASIKDDDFFTTARVWVSADGNSYWYFTHMRTFQFQGVYANVQSWDVAISWLDLMRTGLTPEANVVAFDSQGYVMAATNKDELARLSQCRGSFNSGNLAATCISYFARNHPILEIRNLYNALHTPVWDDLGAAPIPLEQATLSLSGKRYMAIIATLYSKADLRTTVVWYQPWFVLQTDTVSVTALVCILTMLSTFVLTLLGVFGVLRPLQALGAAMRAVAGTLKEGDGSKETVLEPRKPNVFREVDDIGADFETIVVDFLGFSNTNARDNKYAPKDPNAPFAVVFTDIQSSTALWGKNPAEMSRCVQAHHELIRELIKQHRLYEVKTVGDSFMVTTTSAQAALRFALDVQTTLFEHDWDWDGADELYQHTGVVFTKVCDGPFRSESEYGELWNGLRVRIGIHFGMGDVLYDEVSKGYDYYGNVVNAAARIEALAHGGQVVVTEALLAALPAKLDSSVGLTRLLGKFPLRGVAEPPALVEVTPTALRGRTYPLLRTELTTTESPVVGQEIQSVSSDTPKSTDRRSSGPTTASHASDRCPLQQLAEDFARTHTLVRSSVLPAEVVAQQLLTLYSIVEDLLMPLAPAQCTAVLKALAKGWGFTGPMPKTDAAHRMRLVHRMSETTQVFTRLGNVTRSHTPMHAGCTPSHSGGGFIHVELV

SEQ ID NO: 24 - protein, 474 aa, unidentified
  REGION 1..474: note = Naegleria australiensis
  REGION 1..474: note = misc_feature - Photoactivated adenylyl cyclase
MDSMQSNLPHREMARNNFGINTDGILTKKTTTNYLLNGGGEQDRAISVPTSVVQHNEINPSEAAKTVTELSFSGNKSPSTLTQARMMKRCSKQQLLSKLPKKPFLRIQYASHCCSGKISESVKNEILSVAQRENEIRGITGVLICVNDMFFQIIEGHTQHVETLYHNILKDKRHYDVVCVDKMYFDSESERYFPDWCMQSLDMESLFDHCLDPSLSTAVGAAFRAFKTLLVSLLNTQQVFRNYAQPVISKYLSEGENPMIKESIETDKVILFIDLVNYTSITEKLSKTRKPSQVVEILNKFFGCVMEIVEDYCGEITKLIGDCVMVYFDGSECQEAVNCSIEILERLEILRQTSEDPFVKMIYCTIGISRGSVIFGNVGSVGRKLDFTLIGDSVNIASRLQSFASISPYFLIFDESVKGRLDKDSCDIIEVGPIELKGKEKTVNTYTVEHELNRKPPKKEMDRIMSLASLSEYC

SEQ ID NO: 25 - protein, 384 aa, unidentified
  REGION 1..384: note = Naegleria australiensis
  REGION 1..384: note = misc_feature - Photoactivated adenylyl cyclase
MSSQTPPYIRMQYVSLSTEGNISESTKGKILATAARENPRYNITGVLICVRDMFFQIIEGRIEDIDRLYVNIKKDKRHNNIACIDKTYFHREDERMFPSWDMKSIEVDSLFPTKKEVVICGEYCNPLDPSHQKVIETAFSAFTTMMGSMFRAQQVFKNYAQPSVSRFVRDGLNPLAIESVETEKVVLFIDLVNYTTLTEIISQSRKTSEVVRILNVFFSYVMECVENFGGDITKLIGDCVMCHFDGNDCQKAVDCSLKILESLDNLRKETNDPFEKLIFCTIGISKGNVILGNVGTVGRKLDYTVIGDSVNVSARLQSFASSTEYYLIFDGNVKNGLDRVDSVVSCGSIELKGKKNAVNAYTIENVYNRRVLAEDLKAMYCTLN

SEQ ID NO: 26 - protein, 385 aa, unidentified
  REGION 1..385: note = Naegleria australiensis
  REGION 1..385: note = misc_feature - Photoactivated adenylyl cyclase
MSSSQTPPYIRMQYVSLSTEGIISESTKAKILATAARENPRYNITGVLICVRDMFFQIIEGRIEDIDRLYVNIKKDNRHNNIACIDKTYFHREDERMFPSWDMKSIEVDSLFPTKKEVVICGEYCNPLDPSHQKVIETAFSAFTTLMGSMFRAQQVFKNYAQPSVSRFVRDGLNPIAIESVETEKVVLFIDLVNYTTLTEIISQSRKTSEVVRILNVFFSCVMECVEDFGGDITKLIGDCVMCHFDGNDCQTAVDCSLKILESLDNLRKETNDPFEKLIFCTIGISKGNVILGNVGTVGRKLDYTVIGDSVNVSARLQSFASSTEYYLIFDENVKNGLDRVDSVVSCGSIELKGKKNAVNAYTIENVYNRRVLAEDLKAMYCTLN

SEQ ID NO: 27 - protein, 873 aa, unidentified
  REGION 1..873: note = Euglena quartana
  REGION 1..873: note = misc_feature - Putative beta subunit of photoactivated adenylyl cyclase, 4.6.1.1
MHVLIWKKGQQIKTFHTLDEAAQFKAASNIDDAQIFNVSVTPAISASGGSNEATNLRRLMYLSKSTNPEECTPQFLAELGRISSIRNKEIGVSGFLMYSAPFFFQVIEGTDEDLDFLFSKISMDDRHDRCIVLANGPCTGRMYGDWHMKDSHIDSITTHPAMKTILYQIARSFSSMWSYLPKSTGNMLLLGKDPAAQPPVPMSVVVTFLYLVEFGTILSKPNLTEQAAEVLSTFVDVCVKNVEGSGGNIAKFITGVCMAYWPINRAEDAIQAIQQISDDLAQLRSQQPPGSAISLMYSQAGVHYGRALLCNAGTRKSDFTLLGDCINTTSRIATLAKKLKTPLLFSFEVRCLLGDEMREEIESAGMHQVKGRDKPVVVYQFPGATLDVEVVKQKIEQFNPGRFRCQMPVVDYDTLLPSQRPPIFDETQRDVIKPRVPGMQRRDSLADRLAMIAKLAGTTGGAAGAGDSTLVTLTYISQTVRPMSRLDLAAIMRVATRRNAQQGITGTLLHVSGLFVQTLEGPRSAVLGLYMRIRQDPRHKDVVTVHLAPISERVYPPDWTLTTATEQMLSTFPPLQDVLSQLAKSFTSLETYVPSTVVRYLTAGNNPRNLVPVSCGVVMLATDICSFTSLTEKSSLTEVWLICNTFIDACTNAICQAGGEVIKLIGDCVTAYFPGTAADNAVSAAQELITFCRELREAFVDVLDVRGCIYCGVGLDFGQVVMAQCGTLGLSEYAVAGAVSARVMEVEALTREVGRAIVLTEPLADRLCPQMQEMGIVPSPQAIDGLPCYGVAGDEFVLDVDTIKRSIKNFHAARSGEKLNVSGLANGDLEESMTGSPLRATINDFRVSPGRVRRGDSVRRSPSIAGRYGVATR

SEQ ID NO: 28 - protein, 354 aa, unidentified
  REGION 1..354: note = Pseudanabaena sp.
  REGION 1..354: note = misc_feature - Beta subunit of photoactivated adenylyl cyclase ABRG53_0929, Pseudanabaena sp. ABRG5-3
MKRLTYISSFSRSLSKREIEAIGHISQTNNSREGITGVLLSCNGIFFQILEGDEDRIDRLYEKILRDDRHSNILCLKSELNVLARKFPDWSMEVIVLDENNDILLQPIKILLQTLGESQTILANYTQSGVLQAIENGINPLTLKPLYSDKIILFADIISFSGITEKLQVAEVVDLLNQFFTICTRAITARGGEVTKFIGDCVMAYFDGDRADDSLQASLDILAELELLRNQAPANSPLRVLYIGIGLSKGIVLEGNIGSQTKKEYTVIGDAVNVAARIEALTRQLNRFLVFSASVRAGLSDRWKPVRLGDFHTKGRESIVEVYSVDDVLTFQKSDFHKLKQEIKQHLELISCQA

SEQ ID NO: 29 - protein, 370 aa, unidentified
  REGION 1..370: note = Pseudanabaena sp.
  REGION 1..370: note = misc_feature - Beta subunit of photoactivated adenylyl cyclase ABRG53_0928, Pseudanabaena sp. ABRG5-3
MKRLTYISNFSRSLTRKEIENIEIISQKNNFREGITGILLSCSGIFFQILEGDDHRVDSLYEKILKDDRHHQVLCLKSEANITERFFPDWSMKVITIDENSEILLKPIKVLLQSLGESKMALANYTHSGVLQAIENGINPLTLTPRYTEKIIFFADIMGFASMTEKLQVTEVAELLNQFFTICTSEIAAKGGEVTKFIGDCVMAYFDGDRANEALQASLDILSELEILRDRSPVNSPLRVLYVGIGLSKGTVLEGNIGSQTKKEYAIIGDAVNVAARIESLTRKLNRFLVFSAAVREGLSDRWKLLKMGDFYAKGREAIVEVYSIDNPLASQKSDFHHLKQEINEQLELIGKQTLVSYKQRKTLFKLYDR

SEQ ID NO: 30 - protein, 841 aa, unidentified
  REGION 1..841: note = Euglena gracilis
  REGION 1..841: note = misc_feature - Putative membrane-bound adenylyl cyclase, 4.6.1.1
MRISVYVLYIGACLLAIGAAILSGVILLNTFAGQQQQSEDAQYTTGRGQVQAIQGVIANQMSRMVASVDDRSRQLLMTLKELPDDALEPQNAVASLNHSIFTTWVPSIMANDTVLGNGLTIMYVNESNGQLYDRTFSVYRELLKSDQWNYLYGYTFPDELSHVFFINWLDYSTPRLGKDVYQVNFTTVKAPVYQKDNFFNHVMPWGSTDGNTFWYFTYMRSFYTHGVWLNMQCWDVTVNWLSLMQSGLTAGASVASYDSQGYVVTATNKDELRRLAQCIETGDVGSSCISEGGCNCLTTFAKFHPIAELRDLYLALHTPLWDDLDAPPIPLQQQDIQLSGQRYMAIVATLFAKSGFRVTVVWYQPWVLTDTSAVGVTALICVLTMLSTFLLTLLGIFGVLRPLMALGSAMRVVAHTLRKGDAEREAVLQPRRPNIFREVDTIGQDFETIVVDFLGFSSTNARDNKHAPKDPNKPFVVIFTDIQSSTGLWGRDPAQMSRCVQVHHEIIRTLIKDHHLYEVKTAGDSFMITTASPPDALRFALDLQQALYDYDWDWDECDDFYRETTLALWPAALGAKYADMWNGLRVRVGIHFGLGDVVYDEVSKGYDYYGNVVNTAARIEALAHGGQIVVTESLLAALPTPLDPRVSRTVSLGTVPLRGVTAPPALVEVQMASLATRTFPPLRVEHANEAAKQECINVPDLLSDKAGSDGLLTGRLGFQTPSLQSDSLVQVAEDLAKTHAMVRRGYFSAEAVTQHLLVLYRIVEDLLTPLAPSQFATVTKALARGWGVAPPKSKAEFGPVGLRLMQRMSETTTVLKNLAAPHHRAQSAGPDARLLDMEATA

SEQ ID NO: 31 - protein, 350 aa, unidentified
  REGION 1..350: note = Beggiatoa sp.
  REGION 1..350: note = misc_feature - Beta subunit of photoactivated adenylyl cyclase BGP_1043
MMKRLVYISKISGHLSLEEIQRIGKVSIKNNQRDNITGVLLYLQGLFFQILEGENEKVDKLYKKILVDDRHTNILCLKTEYDITDRMFPNWAMKTINLNENSELMIQPIKSLLQTITQSHRVLEKYMPARVIYLINQGINPLTVEPQLVEKIIFFSDILAFSTLTEKLPVNEVVILVNRYFSICTRIISAYGGEVTKFIGDCVMASFTKEQGDAAIRTSLDIISELKQLRHHVEATNPLHLLYTGIGLSYGHVIEGNMGSSLKMDHTLLGDAVNVAARLEALTRQLPYALAFTAGVKKCCQAQWTFINLGAHQVKGKQEAIEVYTVNEAQKYYDTLQITQLIRQTLENDK

SEQ ID NO: 32 - DNA, 1062 bp, unidentified
  misc_feature 1..1062: note = Beggiatoa sp.
  misc_feature 1..1062: note = PAC wt (nt) Codon optimized
  source 1..1062: mol_type = genomic DNA
atgatgaagcggctggtgtacatcagcaagatcagcggccacctgagcctggaagagatccagcggatcggcaaggtgtccatcaagaacaaccagcgggacaacatcaccggcgtgctgctgtacctgcagggcctgttcttccagatcctggaaggcgagaacgagaaggtggacaagctgtacaagaaaatcctggtggacgaccggcacaccaacatcctgtgcctgaaaaccgagtacgacatcaccgaccggatgttccccaactgggccatgaaaaccatcaacctgaacgagaacagcgagctgatgatccagcccatcaagagcctgctgcagaccatcacccagagccaccgggtgctggaaaagtacatgcccgccagagtgatctacctgatcaaccagggcatcaaccccctgaccgtggagccccagctggtcgagaagatcatcttcttcagcgacatcctggccttcagcaccctgaccgagaagctgcccgtgaacgaggtggtcatcctggtcaaccggtacttcagcatctgcacccggatcatcagcgcctacggcggcgaagtgaccaagttcatcggcgactgcgtgatggccagcttcaccaaagagcagggcgacgccgccatccggaccagcctggacatcatctccgagctgaagcagctgcggcaccacgtggaggccaccaaccccctgcacctgctgtacaccggcatcggcctgagctacggccacgtgatcgagggcaacatgggcagcagcctgaagatggaccacaccctgctgggggacgccgtgaacgtggccgccaggctggaagccctgacaagacagctgccctacgccctggcctttacagccggcgtgaagaagtgctgccaggcccagtggaccttcatcaacctgggagcccaccaggtcaagggcaagcaggaagccatcgaggtctacaccgtgaatgaggcccagaagtactacgacaccctgcagatcacccagctgatccggcagaccctggaaaacgacaagccgcggacgtac

SEQ ID NO: 33 - DNA, 1062 bp, unidentified
  misc_feature 1..1062: note = Beggiatoa sp.
  misc_feature 1..1062: note = PAC (S27A) (nt) Codon optimized
  source 1..1062: mol_type = genomic DNA
atgatgaagcggctggtgtacatcagcaagatcagcggccacctgagcctggaagagatccagcggatcggcaaggtggccatcaagaacaaccagcgggacaacatcaccggcgtgctgctgtacctgcagggcctgttcttccagatcctggaaggcgagaacgagaaggtggacaagctgtacaagaaaatcctggtggacgaccggcacaccaacatcctgtgcctgaaaaccgagtacgacatcaccgaccggatgttccccaactgggccatgaaaaccatcaacctgaacgagaacagcgagctgatgatccagcccatcaagagcctgctgcagaccatcacccagagccaccgggtgctggaaaagtacatgcccgccagagtgatctacctgatcaaccagggcatcaaccccctgaccgtggagccccagctggtcgagaagatcatcttcttcagcgacatcctggccttcagcaccctgaccgagaagctgcccgtgaacgaggtggtcatcctggtcaaccggtacttcagcatctgcacccggatcatcagcgcctacggcggcgaagtgaccaagttcatcggcgactgcgtgatggccagcttcaccaaagagcagggcgacgccgccatccggaccagcctggacatcatctccgagctgaagcagctgcggcaccacgtggaggccaccaaccccctgcacctgctgtacaccggcatcggcctgagctacggccacgtgatcgagggcaacatgggcagcagcctgaagatggaccacaccctgctgggggacgccgtgaacgtggccgccaggctggaagccctgacaagacagctgccctacgccctggcctttacagccggcgtgaagaagtgctgccaggcccagtggaccttcatcaacctgggagcccaccaggtcaagggcaagcaggaagccatcgaggtctacaccgtgaatgaggcccagaagtactacgacaccctgcagatcacccagctgatccggcagaccctggaaaacgacaagccgcggacgtac

SEQ ID NO: 34 - DNA, 2818 bp, unidentified
  misc_feature 1..2818: note = Euglena gracilis
  misc_feature 1..2818: note = Photoactivated adenylate cyclase subunit beta, 4.6.1.1, Photoactivated adenylyl cyclase subunit betapacB
  source 1..2818: mol_type = genomic DNA
cactttctgagtgtctattttttttcgcttcaaagcaatgtacattcttgtctggaagaagggccagcaaatcaaaacttttcatacgcttgatgaggcagctcagttcaaggcagcttcaaacattgatgaagcacaaatgttcagtgtgactgttgcaccagccatcagcgcctcagggggaagcaatgaagcaacaaacctgcggaggctgatgtatttatcgaagtcaacaaaccctgaagagtgcaacccacaattccttgcagaaatggcccgagttgcaaccattcgaaacagagaaattggagtcagtggtttcctcatgtactcctctcccttctttttccaggtcattgaagggactgatgaggaccttgatttcttatttgcgaagatttctgctgacccacgacatgaaaggtgcattgtccttgctaacgggccatgtactgggcgcatgtatggggactggcatatgaaagactcccacatggacagtatcacgacgcatcctgcaatgaagacaattttgtatcagattgcccgatctttctcaagtatgtggagctatctcccgaagagtgcaggcaacatgcttctgttgggcaaagacccagctgcgcaaccaccagaacccatgagtgtggtggtcaccttcatctaccttgtggagtttggctctatcttgtcgaaccccaatttgacagagcaggcagctgaagtgctgagcacttttgttgatgtctgtgtgaagaatgtggagggcagtggtggcaacatcgccaagttcatcacagggatttgcatggcatactggccgatcaacagaacggaagaagccctgaccgccatccagcagatcagtgaagacctggcccagctccgcagccagcaggcaccggggtcggccgtctcgctgatgtactcgcaggccggggtgcactatggccgggccatgctgtgcaacgccggaagccggaagtcggactttactttgcttggcgattgcatcaacaccacgtcacgtattgcaacgctggccaaaaagttaaagaccccactgctgttttcgtttgaagttcgatgcctattgggtgacgagatgcgggaagagattgagggtgctgggatgcatcaggtcaaaggaagggataagccagtggttgtttaccaattccctgggcctgagctggatgtcgagatggtgcgccagaagatcgagcagttcacccctggtcgcttccggtgccaaatgcccgtggtcgagtacgaggctttgccgatttcgcagcgcccccccatcttcgacgacacccccaagggcaacccccggccacggacccccggctatgggggccggcagcgcagtgacagcctggtggaccgcctcattatgattgccaaactggccggcccctctgtgtcggccacaggggacaccacgctgaccaccctgacgtacatctcgcaagccactcggccaatgtcgcggctggacctgtccgccatcatgcgcaccgccacccgccgcaacgcccagcagagcatcaccgggaccctgctgcacgtgaacggcctgttcgtccagacgctggagggcccgaaggatgcggtggtcaatttgtacctcaggatccgccaggaccccaggcacaccgacgtcacgaccgtccacatggccccacttcaggagcgggtgtacccgtcggagtggaccctcacctcagccacagaggagatgctggcaacattcccacccctgcaagatgtgttgtcccagttggcgaagtcgttcacctcgctggagacttacgtgccttccacggtggtgaggtatttgacagctgggaacaacccaaggaatctgatgcctgtttcatgtggcgttgtcatgttggccaccgacatttgcagcttcacaagcttgacagagaaatcctccttgacagaggtttggatgatctgcaacacatttattgatgcgtgcacctccgccatctgccaggaaggaggagaggtgatcaaactgattggtgactgtgtgacagcatatttccccggaaacaatgcagattcggctgtggctgcagcccaggagctgttcactttctgccgccagctccgagaagcgtttgtggatgtcctcgatgtgcggggatgtgtctcgtgcggcgtcggcctggattacggccaggtggtcatggcccaatgcggttcattgggactgacagagtatgtggtggccggggccgtcagtgcccgggtgatggaggtggaggccattacccgcgaggtgggctacgccattgtggtcacggaacctgtcgccgaccggctctccccacagttgcgggaccacggcatcgtcccgacgccccaggccatcgaggggctgccctgctatggcatcgccggtgaggagttcgagcttgacgtcgattccatcaagcggggcatcaaggcgctgcatgcggcgcgcagcggggagaagcccctgaccgagcccgaggaggcgaagccggacttccgggtctccccggggcgggtgcgccacggcgactcgggccggcgctccaactccgcccaggggaagcgcagcatccaagtccgctgaccgacgtgcccgtgacatgaccggatctgtgcatttggcccttttgaatacatcaggggcacagtgcactgagaatggctgctcagtgtttcccctccacgcttcagcgatcacccgacgcgcacgtgagtggttcctcccccctggaacaacatacttttgtgttgtcgtgtgctttgcaactccaaaaaccaacctcct

SEQ ID NO: 35 - DNA, 3298 bp, unidentified
  misc_feature 1..3298: note = Euglena gracilis
  misc_feature 1..3298: note = Photoactivated adenylate cyclase subunit alpha, 4.6.1.1, Photoactivated adenylyl cyclase subunit alphapacA
  source 1..3298: mol_type = genomic DNA
cactttctgagtgtctattttttttcggtttgcggcaacatgtacatccttgtttggaaagaaggccagcagatcaggactttccaagatttggaggagtgtgggcagttccaaactgcatccaatataacagatggtcagatattcagtatcaatgtcacacccaccatgagcaagggaggagaaaccggagagacgcaactccggcggctcatgtatctctccgcatcaactgagccagaaaaatgcaatgctgagtaccttgctgatatggcgcacgttgcaactctcaggaacaaacaaattggtgtcagcggcttcttgttgtacagttcccctttctttttccaagttattgaaggaactgacgaagacctggacttccttttcgcaaaaatctctgctgatccgagacatgagagatgtattgtgcttgcaaatggtccctgtacagggcgcatgtatggggagtggcacatgaaagactcccacattgacaacataacgaagcacccagccattaagaccatcctcttccaaattgcaaggtcattctccagcatgtggtcctacctgccaaagaatgcggcgaacatgttgcttctcgggaagaacccaaacaagcaagcccctgaacccatgagtgtggtggtcactttcatctatctggtggagttctcttccattttggcccaccctggactgactgagcagtgtgcagacatccttgctgcgtttgtggacgcctgtgttcgcaacgtcgaaggtacagggggccaagtggcaaagttcatcactgggatttgcatggcgtattggccaataaacagggccgaagatgctctggtcgggttgcagcaactgtcggaggacttggccgagctgcgcagccagcaacctcctggatctgcgctgtccctgatttactcccggtgtggagtgcattacggtcgagctttgctgtgcaatgctggtttccgaaaggctgatttcaccctccttggcgactgcatcaacactgcctctcggatcacctcgttgtccgtcaaactcaaggtgccacttctcctttctttcgaagtgaggtgccttctgggtgatgaaatgcgagaggagcttgaaagttctgggctgcacaaggtgaaaggccgtgacaagcctgtgcaagtgtaccaattcaatgctccagagctggacagcgcgatggttcgtgcgaagattgagcagttcaaccctggaaggtacagggcattgtgcccagtcaagccgtatgagagtttgcacccagctcagagaccaccaatctttgatgacaccccacgagagaaccagccaaaactcagccaagttcagcgacgcgacagccttgtggatcgcctcagcttgattgcaaagcttgctttcccttcgagcatgatggcaggtggtgaaggccagctcatcacattgacatacatttcccaagcagcccatcccatgtcccgtttggatttggcatcaatccagaggattgcatttgctcggaatgaatcctcaaatatcacaggctcattgctttatgtgagcggtttgtttgtacagacattggaaggtcccaagggcgctgtggtttccctgtatctcaagatcaggcaagacaagcgacacaaagatgtggttgcggtcttcatggcccccatcgatgaacgggtgtatggcagtcccttggatatgacctccgccactgaggaaatgttggccaccttcccacccctgcaggacgtgctctcgcagctggccaagtcgttcatctccctcgagacctatgtcccctccacggtggtgcgctacctcaccgccggcaacaatccccgcaatctgcaacctgtttcggtggaagttgtcatgcttgccactgacatctgcagcttcactccactgtccgagaagtgttccctgacagaggtgtggaccatttgcaacaccttcattgacgcctgcaccagtgccatctgcaatgaaggtggggaggtcattaaattgattggtgattgtgtgacagcatatttccccccgacaggtgccgacaacgctgtccatgcatgccaggagatcgtcagcttctgtgctcagctgcgagatgccttccacgacgtcttggactgccggtctgtggtggcttgtggggtggggctggacttcgggcaggtcatcatggcccagtgcggcagtttggggatgacagagtttgtggtggctggggaggtcagcgctcgtgtgatggaggttgaagccctcacgcgcgaggcagggcgtgcgatcgtcatcaccgaaccagttgcggatcgactgtctcccaaactgcgtgacacgggcatcgtcccctgccaggaaggagtggacggcgtcccttgctatggaatcctgggccctgaatgggaattggatgtggcaaccatcaagaagaacatttacgggttccacgacgcccgggccctggccgccatgaagaaggtggacgacggcaccaacgccccgggccggggcgcccccgccgggggcatcccctcctcccccaaggttcgaccaccaggccgcaccaattcagtgtccagctacacccctgaccccaatgaggcactggatcctcggatggcggaatcggtgtttcttgacatgtgtcatcagcggggagataccgccaacaactccatcgccgtcaaattgcggcaggcggccaacgacgatcggttggacctgggcaggatgttgcaagggccacacgagctcatgcctgtgatgcaggccatcaagcacctgaccaacctgcgaatgctcaatatgagcgacaatttcgtggatgacaacaacgttggagaacttgtggaatcgtgcattccaatgcgttcgttacaggtgttggacttgtccaacaaccctggcttgacgaaggtcatcgccctgaagcgcctcatcaagcataacacccaggtccgggagatccttctgaacggcacccgcatcgccccaaccgagcaacgcaagctgcagtcctcgatgaacgtcaaccggctgtgtgcctccaccgacctgaagggttcgcacaaatatgaacattaacgatacggcgctgtttgtacgtctggcctagcacatcacgacgtcactgctttcgcttgccccccccctccgtgtgctggtttacatgttgtgagttgcgggggagtgggattacaactcccagcatcgcagcggcattcgcacgtatatgccctgtgactggattccatttccaccgtttcgtttgtgacaccgtaac

SEQ ID NO: 36 - DNA, 2799 bp, unidentified
  misc_feature 1..2799: note = Euglena longa
  misc_feature 1..2799: note = Photoactivated adenylate cyclase subunit beta, 4.6.1.1, Photoactivated adenylyl cyclase subunit betapacB (Euglenophycean alga) (Astasia longa)
  source 1..2799: mol_type = genomic DNA
tccaaaaatgtacatccttgtgtggaaaaggggtcaacaaatcaaaactttccacacacttgatgaagcagcgaatttcaaggcagcttcaaacattgatgaggcgcagatgttcagcataactgttgcaccagccatcagtgcttcgggcggaagcaatgaagcaaccaatctgcgaaggctcatgtacctctcgaagtcaacaaatcctgaagagtgcaatccacaattccttgcagaaatggcccgagtggccaccctcagaaacagagaaattggagtcagcggattcctcatgtattcctctcccttctttttccaggtgattgaagggacggatgaggacctcgattttctgtttgcaaagatatctgcagaccccagacatgaaaggtgcattgttcttgcaaatgggccctgcactggtcgcatgtatggtgactggcatatgaaagactcccatattgacagtatcacaacacatcctgcaatgaagacaatcttatatcagatcgcccgctccttctcaagtatgtggagctatttgccaaagagcgcaggcaacatgcttctgttgggaaaagacccagctgcacagccaccagaaccgatgagtgtggttgtcacctttatctaccttgtggaatttggttccatcttgtccaatcccaatttgacagagcaagcagctgaggtgctcagcacgtttgttgacgtttgtgtcaagaacgtggagggcagcggtggcaatatcgcaaaattcatcactggcatctgtatggcatactggccaatcaacaggacagaggatgccctgaatgcaatacagcagattagtgaagaccttgcacaacttcgcagccagcaggctcctggatctgcaatctcactcatgtattcccaggctggtgttcattatgggcgagccatgttgtgcaacgcagggagccgaaagtctgacttcaccttgcttggagattgcatcaacaccacatcacggattgcaacactggcaaagaagctgaagaccccgttgctcttttcatttgaagttcgatgtttgctgggtgatgagatgcgtgaagaagtcgagggtgctgggatgcatcaagtgaaaggaagggacaagccggttgttgtttaccagttccctgggcctgagctggatgttgagatggtgcgacaaaagattgagcatttcaccccaggtcgcttccgctgccagatgcctgtggtggagtacgaatcattgccaatttcacagaggccaccaatctttgatgacaccccaaagggcaagcctaggccccggaccccaggctatggtggccggcaacggagtgacagccttgtggaccgcctcattatgattgccaagctggctggcccctctatctcggccacaggagacagcacgttgacaaccttgacatacatctcgcaagccacgcgacccatgtcacgacttgacctgtcagccattatgcggactgccacccgccgcaatgcccagctgagcatcactggaacacttctctatgtaaatggcttgtttgtgcaaacactggaggggccgaaggatgccatggtgaatttgtatttgaaaatccgccaggaccccaggcacactgacgtggtcaccgttcacatggccccaattcaggagcgggcatatccagcagaatggacactcacctctgccactgaagagatgctggccaccttcccacccctgcaagatgtgctttcccagctggccaagtcttttacctcgttggagacctatgtgccctcaaccgttgtaaggtatttgacagctgggaacaacccaagaaacctgatgcctgtctcatgcggagttgtcatgttggccactgacatctgcagcttcaccagcttgacggagaagtcatccctcacagaggtgtggatgatctgcaacacattcattgatgcgtgcacttcagctatctgtcaggaaggtggagaggtgattaagttgattggtgattgtgtgacagcatatttccctggaaatggagctgattctgctgtggctgctgcccaggagctgttcactttctgccgccagctcagagaggcatttgtggatgtcctcgatgtgcgggggtgtgtgtcatgtggcgttgggcttgattatggccaggtggtgatggcccagtgtggctcactggggctgacagagtatgttgtggctggggccgtcagtgcccgggtcatggaggtggagtccatcacccgcgaggtgggctatgccatcgtcatcacagagccagtggctgaccggctttcaccacagttgcgggaccatggtatcatccccacaccccaagctatcgaaggtctcccctgctatggcattgcgggtgaggagttcgagcttgatgtcgattccatcaagcgaggaatcaaagccctgcacgcagcaaggagcggggagaagccattggctttggagcctgaggaggcgaagcaggactaccgggtgtcccccggtcggatgcgccatggtgactctggacgacgttcaaactcagcccaggggaagcggagcacccaagtgcggtaagtgcggtgagcccatggaacaagtgcatagaaagagggcccaaggaccttcccttgaagtgcatctgggttcaggtgctgtacattgaacacctccagtgacgtggttttctttcaggcctccgaatgcccaaatgcggaaacgttgtaccagtgtgtatttgtgcctctcccatgctgcaacccccagtccggtgagtcccaccattg

SEQ ID NO: 37 - DNA, 3209 bp, unidentified
  misc_feature 1..3209: note = Euglena longa
  misc_feature 1..3209: note = Photoactivated adenylate cyclase subunit alpha, 4.6.1.1, Photoactivated adenylyl cyclase subunit alphapacA (Euglenophycean alga) (Astasia longa)
  source 1..3209: mol_type = genomic DNA
gcagggaaaatgtacattctcgtttggaaggagggccagcagatcaagacctttcaagatttggaagagtgtgggcagtttcagattgcctcaaatatcaccgatggtcagatattcagcgtcagtgtcacaccaaccatgagcaaaggaggcgaaacaggtgagacacaactacgtcggctgatgtatctgtcagcatcaacagaaccagagaaatgcaacgctgagtaccttgctgacatggcacatgtggcaacacttcgcaacaaacagattggagtcagcggtttcttgctctacagctcaccattcttctttcaagtcattgaaggaactgatgaagacttggacttcctgtttgccaaaatctcttctgatccaaggcatgaaaggtgcattgtgcttgcaaatggtccttgcacaggccgaatgtatggggaatggcacatgaaagactctcacattgacaacatcacgaaacaccctgcaataaagaccattctctttcaaattgcaaggtcattttcaagcatgtggtcatacctgccgaagaatgcagcaaatatgctgctattggggaagaatcccaacaagcaagccccagagccaatgagtgtggtggtcactttcatatatctggttgaattctcctccatcttggcacaccctggactgacagagcagtgtgctgacatccttgctgcttttgtggatgcatgcgttcgcaacgttgaagggactggtggccaagttgcaaagttcataacgggaatttgcatggcatattggccgatcaacagagcagaggatgcattggtgggcttgcagcagttgtcggaggatttggcagagctgcgtagccagcagcctcctggctctgccctgtccctcatttactcacggtgcggagttcattatggccgggctttgctgtgcaatgcaggattccgaaaggctgacttcaccctgcttggtgactgcatcaacactgcctcccgaatcacctccctttccgtcaagctcaaggtgccactgctgctctcatttgaggtgaggtgccttctgggtgatgaaatgcgagaggagcttgaaagtgctgggttgcacaaggtgaagggtcgtgacaagcccgtccaagtgtaccaattcaatgcacctgagttggacagtgctgtggttcgcaccaaaattgagcagttcaacccaggaaggtatcgtgcactgtgtccagtcaaaccatatgacagcctgcaccctgcccaaagaccaccaatctttgatgacacaccacgtgacagccaacccaaggtgaaccaaatgcagcggcgtgacagtcttgtggatcgcctcagcatgattgcaaagcttgcttttccctcgagcatgatggtaggaggagagagtcagctcatcacattgacgtacatctcccaagcagcccaccccatgtctcgcttggatttgtcttcaattcaaagagtttcattctcaaggaatgaatcatcaaatatcacaggctccttgttgtatgtgaatggcttgtttgtacagacacttgaaggcccaaaaagtgctgttgtttccctgtacctcaagattcggcaggacaaacgccacaaagatgttgttgccgtgtttatggctcccattgaggagcgcgtgtatggcagccccttggacatgactgctgccacagaggagatgctggcaactttccctcccttgcaagatgtgctctcacagctggccaagtcattcatctcactggagacgtatgtgccctccactgtggtgcgctacttgactgcagggaacaatccccggaacttgcaacccgtctcagtggaggttgtcatgctggccacggacatctgcagtttcaccccgctgtcagagagatgctctctgactgaggtgtggaccatctgcaacaccttcattgatgcctgcaccagtgcaatctgcaacgaaggtggggaagtcatcaaactgattggggattgtgtgacagcttacttccctccaacggctgccgataatgcagttcatgcttgccaggaaattgtcagtttctgtgctcagctgcgggatgcattccgagatgtactggactgccgctccgttgtggcctgtggggtcggtctggatttcgggcaggtcatcatggcccagtgtgggagtttggggatgacagagtttgtcgttgcgggggaggtcagtgcccgtgtgatggaggttgaggcactcacacgtgaagcagggcgtgccatcgtcatcaccgagcctgttgcagatcggctctcccccaagttacgcgacactggcattgttccctgccaggagggtgttgatggagttccctgctatgggattttgggtgtggaatgggagctggatgtggcaataatcaagaaaaatatctatggctttcatgaggcgcgtgcacaggcagccctcaagaaagtggacgatggcacaaatgctccgggccgtggtgcaccagctgctggtgtaccgtcttcacccaaggctcgagccctgggccgcaccagttcagtttccagctacactcccgacctcaacgaaacactggacccccgcatggctgagtccgttttcaatgatttgtgcaaccagcgtggcgatgctcccaacaactccattgctgcaaaattgcggcaagcagccaatgatgaccgattggacctgggccggatgctgcagggaccccatgagctgatgccggtgctccaggcaatcaagcagctgaccaacctacggatgctcaacatgagtgacaacttcgttgatgacaacaatgtcggagaacttgcagagtcgtgcattcccatgcgatcattgcaggtgctggacctgtccaacaaccctggcttaacaaaggtcatcgctctgaagcgcctcatcaagcacaacacccagattcgggaaattctgctgaacggcacgcgcattgcgccaatggagcagcgcaagctgcagtcctccatgaacgtcaaccggatgtgtgcctccactgactcgaaaagctcccacaaatatgatcactaatgacgatcgatgcacacacaatttatgtccattttgtttctccctctcatgtgcacatttgatgtgaccaatgtgtgattgtggctgggaacaacttccggccacagcagtgggatttcacacacctgtccctccaccgt

SEQ ID NO: 38 - DNA, 3060 bp, unidentified
  misc_feature 1..3060: note = Euglena gracilis
  misc_feature 1..3060: note = Photoactivated adenylate cyclase subunit alpha-like protein ST-pacA
  source 1..3060: mol_type = genomic DNA
atgtacaaccgagtttataaggagcgtcaggagattcgcaccattcagggtctggaggcttcaggacagttccagaccgccagcaacatcaccgatatccaaatctattctattaacgtgaccccaaccatgtccaagggtggggagaccggggagactcagttgcgccggctgatgtatttatcggcaagcacggagcccgaaaagtgcaacgctgagtatctggcggacatggcccacgtggctactctccgtaataaggccatcggcgtcatccgcttcctgttgtacagcagtcccttctttttccaggtcattgaaggcaccgacgaggatctagacttcctctttgccaaaatatccgccgaccctcgccacgagcgctgcatcgttctggccaacggcccttgtactggtcgtatgtatggcgagtggcacatgaaggactcccacatcgacaacattactaataacccggctatcaagacgatcctgttccagatcgctcgctctttcactagtatgtggtcctacctgcccaagaacgctgcgaacatgctgttgcttggaaagaaccccaacaagcagggtccagagccacgttcagtggttctcacattcatctacctggtcgaattctctagtattctggcacatccgggtctgacagagcagtgcgcggacatcttggcggccttcgtggaggcgtctgtgcggaactgcgaaggcacgtggggactggtcgcaaaatttatcaccggtacgtgcgtcgcgtattggcctattaaccgggctgaggatgccctcgttggcctgcagcaattgtcagaggatctcgccgtgctgcgcagctaccaacccccgggttccgctctctcccgtatctactcgcgtcggggggtccactacggacgcgcgttgctgtgcaatgctgggttccggaaagcagacttcactttgctgggcgattgcattaacactgcatcgcggattacttccctgtccgttaagttgaaggtgcccctcctgctcagcttcgaggtgcgctgcttgctcggagatgagatgcgcgaggaactggagcgctctgcgctgcataaggtcaaggggcgtgacaagcccgtgcaggtttaccagttcaatgctcctgaattggatagtgcacttgtgcgtgctaagatcgagcaatttaaccctggtcgttaccgcgcgttgtgcccagtgaagccctacgagtcccttcacccagcccagcgtccacccatctttgatgacaccccaagagagaatcaacctaagctttctcaggttcagcgccgggattcccttgttgacaggctgtccctgatcgcgaaactggctttcccctctagcatgatggccggcggggagggccagttgattactttgacctacatctcgcaggccgcacaccccatgagccgcttcgacctcgcttcgattcagcggattgcctttgctcgcaacgagtcatctaatatcacgggatccttgctttacgtgtccggactgttcgtccagaccctggacgatccgaagggcgccgtcgagtccctgtacctgaagattcgacaggataagcgccacaaggatgtcgtggcagtttttatggccccgattgacgaacgtgtgtacgggacacccctcgacatgacttcggcaacagaggaaatgcttgcaacgttcccgccactccaggatgtcctgtcacagttggctaagagcttcatcagcttggagacttacgtgccgagcacggtggttagatatctgacagccggcaacaatccccggaacttgcagccagtgtctgtcgaggtcgttatgctggctacggacatctgctccttcactcccttgtccgagaagtgctctcttacagaggtttggaccatctgcaacaccttcattgatgcctgtacatccgcaatctgcaatgagggtggagaggtcattaagcttatcggtgactgtgtgaccgcatatttcgcccctaccggggccgacaacgccgtgcatgcgtgtcaggagatcgtgagtttctccgctcaactgagagacgcctttcatgacgtcttggattgccgctccgtggttgcatgcggtgtgggactggacttcggccaggtgatcatggcccagtgcgggagcttgaggctgacagagttcgtggtcgccggtgaggtgagcgcccgcgtaatggcagtggaggccttgacccgcgaagctggccgtgctatcgttatcaccgagcctgtgtctgaccgcctgtccgccaagctccgcgacacaggtatcgtgccttgccaggaggggctgcacgccgtcttgctgtggtacccggggcccagaatgggcattggttgcgccaaccaccaagaggatcacctgagggttcctcgccggccgggcccaggtcggcacgaggaaggtggcaggcgacaccagcgccccggtcccggccgaccccgccggggccatccgttcctgccccaggggagcaccacgcgacctcatcagttctctgtccagctgcaccctctgcccaagctgggcaccggctctagtgacgggggaatcggggtcagcctccacgtctacagcgcaggcaggtaccggcagcaactgcaccggcgccagatcgctgccgggggccagcgccggtccgtgggccccggccaggacgttgctagggccacccgtgcccatgcctgcgacgctggacatcaggccccggatcaaccagccaacgcccagtacgagcgccagttccggggcgggcagcaacgatggaggacctgcggcatcgtgcactcgaacgcctttgtgaccggtgtgggactggtgcagcaaccctggcttgatgagggccatcgccctgaggcacctcaccagtccctccacccaggaccaggcgacccctcagagcagcatcctcaccgtcccaaccgggcgacagaacccggcctgttggttgcgcgacagccggctgtgtgcctccaccgacccgaaggtttcgcccagatttgtaccttctaa

SEQ ID NO: 39 - DNA, 2580 bp, unidentified
  misc_feature 1..2580: note = Euglena gracilis
  misc_feature 1..2580: note = Photoactivated adenylate cyclase subunit beta-like protein FBpacB
  source 1..2580: mol_type = genomic DNA
atgtacatcctggtgtggaagaaaggtcagcaaattaagactttccacaccctggacgaggccgctcagttcaaggcggcctctaacatcgacgaggcccagatgttcagcgtcactgtcgctcccgcgatctcggcgtccggcggttctaacgaggctaccaatctgcgccggttaatgtacctctccaagtccacaaaccctgaggaatgcaacccccagttcctggccgagatggcacgtgttgccacgattcggaaccgggagatcggtgtgtccgggttcctcatgtactcttccccattctccttccaggtgattgaaggcacggacgaggaccttgactttctgttcgccaaaatcagcgctgatcctcgccatgagcgatgcatcgtcttggccaacgggccctgtacaggaagaatgtacggcgattggcacatgaaggatagccacatggactcgatcacgacacacccggcaatgaagaccatcctttatcagattgcccggtcattcagctccatgtggagttaccttcctaagtccgctgggaatatgttgctgcttggcaaagaccccgcggcccagccgccagagccgatgtccgttgtcgtgacctttatttacctggtggagttcggcagcattctgtctaaccctaacctcacagaccaagcggccgaggtgctgagtacctttgtggacgtctgcgtgaagaatgtggagggatcgggtggcaacattgctaagttcatcaccgggatttgcatggcttactggcccatcaaccgaaccgaagaggcactgaccgccatccagcaaatctcggaggatttggcacagctgcgatctcagcaagcacccggttccgccgtgagcttgatgtactcccaggccggggttcactacggccgacccatgctgtgcaacgcagggcgtcgcaagagcgacttcaccctcctgggtgactgtatcaacacaacttctcggatcgccaccttggccaagaaattcaagactcctcttttgttctcccaggaggtccgctgcctattgagggatgagatgcgcgaggaaattgttggggccggtatgcaccaagtgaagggtcgcgacaagcccgtggttgtgtaccaatttccaggcccagagctggacgtggagatggtgcgacagaagattgagcagtttacgccgggtagattccgctgccaaatgccagttgtcgaatacgagggactgccgaactcccaacgccctccaattttcgatgacacccccaaggccaacaggcggcccaggacccctgggtacgggggtcggcagaggtccgattcccaggtggaccgccccatcatgattgcaaagctcgcaggtcctagcgtgtccgcgacgggcgacactaccctgacgaccctgacctatatctcccaggctacgaggccaatgtctcggcaggatctgagtgccataatgcggaccgctactcgtcgcaatgcacagcaaagcatcactggcacactcttgcatgtgaacggtctgttcgtccagactttggaaggccctaaggacgctgtcgccaatctgtacttgcgcgtgcggcaggatccgcgtcagactgacgtgaccactgtccacatggctccactgcaggagcgcgtttatccctctgaatggactttgaccagtgcgacagccgaaatgctggcaacttttccgcccctgcaggatgtgctggcgcagctcgctaagtccttcacgtctttggagacttacgtcccgtccaccgtcgtgcgtcacctgacagcaggcaacaatccacgcaacttgatgccagtgagttgtggggtcgttatgctggctaccgacatttgcagcttcacttcattgacagagaagagctcacttaccgaggtttggatgatctgcaacacgttcattgatgcctgcaccagcgccatctgccaggacggcagagaagttatcaagctgatcggggactgtgtcacagcctattcgcctggaaacaacgccgacagcgcagttgctgcggcccaggagctgttcaccttctgccggcagctgcgagaggcattcgttgatgtgctcgatgtgcgcggatgcgtgtcttgcggggtcggcctggagtatggacaggttgtcatggcccagtgcggctccatgggacttaccgaatacgtggttgccggagctgtctcagctcgtgtgatggaggtagaggcaatcacccgcgaggtgggctatgctatcgtcgtgacggagcccgttgccgaccggctgtcaccccagttgcgtgatcatggtatcgtgcccaccccccaggccatcgagggcctcccttgctacggcatcgcgggcgaggaattcgagctcgacgttgactctatcaagcgcggcatcaaggctttgcacgctgcccgctccggtgagaagccgctcacagagcccgaggcggccaagcccgattttcgtgtgtcgcccggacgcgtacgtcatggagattctggccgccgttctaacagcgcccagggcaagcgctcaattcaggtccgctaa

SEQ ID NO: 40 - DNA, 3060 bp, unidentified
  misc_feature 1..3060: note = Euglena gracilis
  misc_feature 1..3060: note = Photoactivated adenylate cyclase subunit alpha-like protein FBpacA
  source 1..3060: mol_type = genomic DNA
atgtacattctggtctggaaggaggggcaacagattcggacttttcaggacctggaagagtgcggccagttccagacggcttccaacatcaccgatgggcagattttttctatcaacgtgacccccacgatgagcaagggcggggagactggtgagacccaactgcggcgcttgatgtacctctctgcgtctacagaacccgagaagtgctacgccgagtacctggctgacatggcccacgtggccacactccgtaacaagcagatcggcgtgagcggcttcctgctttacagctcccccttctttttcgaggtcatcgagggtaccgatgaggaacttgacttcctgtttgcgaagatcagcgccgacccacggcatgtccgttgcattgtgcttgctaacggtccgtgcaccggacgcttgtatggtgagtggcacatgaaagactcccacatcgacaatattacaaagcatcctgcgattaagacaatcttgttccaaatcgcccgcagtttcagctgcatgtggtcttatttgcctaagaacgccgcaaacatgctgcttttggggaaaaacccaaacaagcaggcgccagaaccaatgtctgtggttgtgaccttcatctacttggtggagttttcctcgatcttggctcatcccgggcagaccgaacaatgcgccgacattctggcagccttcgtcgacgcctgtgttcgcaacgttgaagggacgggcgcccaggttgcgaagttcatcaccggcatctgcattgcttactggccaattaaccgcgcagaggatgccctgatcggtttgcagcaattgtccgaggatctggccgagctgcgcagccagcaacggcccggctccgcactctccctgatttactcccggtgcggtgtgcactacggacgagctctcctgtgtaacgccgggttcagaaaggcagacttcaccctgcttggtgactgcatcaacaccgcgtcacggatcacttccctgtcagttaagctgaaggtgcccctcttactgtcgtttgaggttcgctgtgtgctgggagacgagatgcgagaggtgctggagacatcaggtctccacaaggtcaagggccgcgaccagccggtccaggtgtaccagttcaacgcccgggagctcgacagcgctatggtgagagccaagattgagcagttgaatcccggctcctaccgcgcgctttgccccgtgaagccttatgaatccctgttgccggcccagcggccccctatcttcgacgatacccctcgtgagaatcagcccaagctgagtcaggtccagcgacgggattcgctggtggaccgtctgtccctgatcgctaagttggcctttccgacatcgatgatggccggaggcgagggccagcttatcaccctcacatatatctcccaggcagctcacccgatgacccgactcgatctggcgagcatccagaggattgctttcgcccgcaatgagtccagcaacatcactggatccctgttgcacgtctccggccttttcgttcagacgctggagggcccgaagggggctgtggtctcattgtatctcaagatccggcaagacaaacggcacaaggatgttgtcgctgacttcatggctccttctgagggccgcgaatacggatccccactggatatgacgtctgcaacggaggaaatgttgacaagcttccctcccttgcaggagctgctaacgcacttggccaaaccgttcattagcctggagacgtacgttcccaccactgttgtccgctacctgacagcgggcaacaatcctcgaaacttgcaacccgtgtcagtggaggtggttctcctggccaccgatatctgcagcttcacccctttgtcagagaagtgcagcctgactgaggtgtggactatctgcaagacgatcttcgtggccttcattcgcgccattttcaacgagggtggcgaggtgatcaagctcatcggcgactgcgtgaccgcatacttcccacccaccggtgctgataaggcagttcatgcttgccaggagatcgtaacattctgcgcccagttgggtgacgcctttcacgatgtgctggactgccgcagcgtcgttgcttgcggtgtgggtctggatttcggccaggttattatggctcaatgcggatccctgggtatgactgagttcgtggtcgcgggagaggtgtccgcacgtgtcatggaagtcgtggccttgaccagagaagccggccgagcgattgtgattacagagccgctgccagatcgcctcgcccctaagctgagggacactggtatcataccctgccaggaaggtgtggacggtgtgccatgttatggcaacctgggccctgagtgggagctggatgtagctactatcaagaaaaatatctatggattccatgacgcgagggcccttgctgcaatgaagaaagtggacgtggggactaacgcccccgggcgcggggccccagcagggggcattccatcgtctcccaaggtccgccccccgggccgtactaactcggtttctagctacacccccgatccgaacgaggccctggacccccctatggctgagtctgtgttcctggacatgtgccatcagcgtggggataccgcaaacaattccatcgcagtcaagttgcgtcaggccgcgaatgacgatcgcctggacctcggacgcatgctgcagggcccccacgagttgatgcccgttatgcaggccatcaagcatttgactaatcttaggatgctgaacatgtccgataacttcgtcgtggataacaatgtcggagagttggtcgagagttgtatccccatgcggtctctgcaggttctcgacctgtctaacaacccaggcctcaccaaggtcatcgccctcaagcgtttgatcaagcacaacacccaggttagggagtttctgcttaacggcacccgcattgcacctacggaacagcgcaagctgcagagttccatgaacgtgaaccgtctttgcgcttctaccgacctcaaggggagtcacaagtacgagcactaa

SEQ ID NO: 41 - DNA, 3060 bp, unidentified
  misc_feature 1..3060: note = Euglena gracilis
  misc_feature 1..3060: note = Photoactivated adenylate cyclase subunit alpha-like protein 1224-5/1FpacA
  source 1..3060: mol_type = genomic DNA
atgtatatcttggtgtggaaggaggggcagcaaatccgcactttccaggatctggaagagagcgtccagtttcggacggcctctaacatcaccgatggccagattttcagcatcaacgtcaccccgactatgggcaagggaggtgagaccggagagacccagctgaggcggttgatgtacctgagcgcctcgacggagcctgaaaactgcaacgctgactacgtgggtgacatggcccacttcgcaactctgagaaataagagtatcggggtcagcggtttctacatgtatagtgccccattcttgtttcaggtgatccagggaacagacgaggatctggatttcttgtgtgccaacggttccgccgatcctaggcaccagaggtgcattgagctggccgatgggcccaccacaggccggatgtacggttggtggcacttgaaggacagtcacatcgacaatatcacgaaggaccccgccattaagacgattttgatcgagatcgcgcgttccttctcctctatgtggtcatatttgcctcgcaacgccgctaacatggtgcttttgggaaagaaccccaacaaacaggcccccgagccaatgtctgtggtcgtgaccgttatctacttggtcgaattctccgttattatggcccatccagggcttagcgagcagtgccctgacattttgccggcttttgttgacgcgtgcgtcccgcatgtcgatggcaccgggggcgaggtggccaagttcatcattgccatatgcatggcttactggcccatcaactccgacgagcacgccttggtgggactgcagcaactttccgacgatctggcggagttgcgctccctacaggaggatggcagcgcgagaagcctgatctactcccgttgtggactgcatttcgggcgagctatgctgtgcaacgcaggcttccgcaaggccgattttacacttctgggtgattggatcaactctgcgtctcgtatcacctccctgagtgtgaattccaaagttccactgttgcttagtttcctcgtgcgatgtctccttggtgatgagatgcgtgtcgaactggaacgttctggtttgcataaggtgggcggtcgcgtcaagccggttcaggtgtatcagttcaacgcccctgaattggacactgcaaccgtccgcggcgagatcaagcaattcaaccccggtcgtgagcgcgccctgtgccccgtgaagccatacgagtctttgcacccagcacatccggacccgatcttcgatgacactccacgcgagaaccagcccgagctctcacaggtgcagcgccgggattcgcttgtggatcgtctgtcccaaatcgctaagctggctttcccttcgtccatgatggctggtggcgagggccagttgattactctgacgtacatctctcaggctgcccatcccgtttcgcggttggacttggcctccatccaaaggatcgctttcgcgcgcaacgagtccagtaacattaccaagtccctgttgtacgtgtcaggcctgttcgttcaaaccttggagggcaccaagggcgcggtggtttccctttacctcaagaaccgccaggacaagcgccacaaggacgtcgtggctgcgttcatggctcccatcgacgagggggtatacggctccccccttgacatgacctctgcgaccgaggaaatgctggcaacgtttacgccactgcacgacgtgctctcacagctggccaagagcttcatttcgctggagacctatgttccgtcgactgtggtccgccacttggccgcaggtaataacccacggaacctccaacccgtgtctgttgaagtggttatgctggccacagccatctcctcttttactccactgtcggaaaagtgctcactgaccgaggtgtggaccatttgcaacacatttattgtggcatgcactagcgcaatctgcaatgagggcggggaggttatcaagctgatcggagactgcgtgccggcttactttcctcccactaacgccgacaacgcggtccatgcatgtcaggaaattgtgagcttctgcgcccagctccgggcagctttccacgacgttctggattgtcgctccgtcgtggcctgcggggttggcctggactatgggcaggtgatcatggttcagtgcggtagcctgggcatgactgaattcgtggtcgctggcgaggtccgtgcacgagtgctggaggtcgaggctctcaccagggaagcagggcgcgcaatcgtcatcacggagcccgtgcccgagcgacagtcacccaagctgcgagacactggaattgtcccttgccaggagggggtggatggagcccgatgctacggaattctgggccccgagtgggagctcgacgtagccacaattaaaaagaatatcaatggattcctcaaggatgctcgcgctctggccgcgaacaaaaaggtggacgatggtaccaatatctcttgccgcggtggcaacccccctgctggcgggatcccgacgagccctaaggtccgtcccccgggccggaccaattctgtttccagctatacacccgatcctaaacaggcccttgatccccgcatggcagagagcgtcttcttggacatgtgccaccagggtgacacagttaataacagcattgccgtgaagcttggcctggccgcaaacgaggacggcttcgacgggggatggatcctgacctggtacgtcgagctgatgcccgtgaaacaagccattaaggtgctcacccagctgcggattggcaacatgtccgacaacttcgtggacgataacaatgtcggtgagctgctcgagaagtgcatcccaacccggtccctccaggtgctggacctctccaacaaccccggcttaactaaggttatcgccctgaagcctaagcacaacacgcaggtgcgcgagatcctgctcaacgggacccgcattgctcctacagagcagcggaagcaacagacaagcaagaacgttaaccgtctgtgcgcgtccaccgacctcaagggttctcacaagtacgagcactaa

SEQ ID NO: 42 - DNA, 2580 bp, unidentified
  misc_feature 1..2580: note = Euglena gracilis
  misc_feature 1..2580: note = Photoactivated adenylate cyclase subunit beta-like protein ST-pacB
  source 1..2580: mol_type = genomic DNA
atgtatatcttggtttggaagaaaggacaacagattaagacctttcacaccttggatgaggccgctcaattcaaggccgcgtcaaacattgacgaggcccagatgttctcggtgaccgtcgcctccgctatctctgcctccggcggttctaccgaggccactaacctgcggaggcttatgtacctctctaagtctacgaaccctgaagagtgtaatccccaattcctggcagaaatggctagggtcgcctttatcagaaatcgggagatcagggtgtccggattcttgatgtatagctcacctttctttatccaggtgatcgtggagaccgatgaggacttggatttcctgttcgccaagatctcagcggacccgcgccatgagatgtgcatcgtgctcgccaatgggccgggcaccgggaggatgtacggggactggcacatgaaagatagtcacctggactctatcaccacgcacaccgccatgaagaccattctctatcagatcgctcgctcgttctcatccatgtggatttatctgcagaagtccgccgcgaacatgcttatgctcggtaaggaccctgcggctcagcccccggagcgtcgctccgttgtggttacgttcatctacctggtcgagttttgcagcatctttagtcatccgaacttgaccgagaagacggccgacgtgctttctaccttcgtctacgtctgcgttaagaacgtggagggctctggtggcaacatcgcaaagtttatccccgccatttgcatgacatactggcctatcattcgaactaaggaagctttgaccgccatccagcaaatcagcgaagacttggcccagctgcggagccaacaggctcaaggttcggccgtgagtctacgttattcgcaggcgggggtgcactacggccgggggatgcagtgcaatgcaggctcccgtaagagcgacttcacattactgggagactgtattaatacaacttcccgtattgcgaccctggccaagaaactcaagacgccccttctgttcgggttcgaagtgaggtgcctgttgggcgacgagaagagagaggaaatcgagggcgcaggcattcaccaagtcaagggacgtgataagccagtcgtggtctaccagttccccggtcccgatctcgacgttgccatggttcgccagaagatcgaacagttcactcccggacgtttccgctgccagatgcctgtggttgagtacgaggcactccccatctctcagcgcccgcccatctttgatgacacaccaaagtccaacccccggccccgcaccccaggatacgggggccgccagcggagcgactctctggttgaccggctgattcgaatcgcaaacttgtccggtttctccgagagcgctacgggcgataccactctgactaccctgacctacatatcccaggccacacgcccaatgagccgtctggatttgagtgcaatcatgccaactgccacccggcgcaacgcacagcaatccaatatcggcacagtcttgcacgagaacggactgttcgtccagactctggagggccccaaggacgccgtggtcaacctgtacctgcgcatccgccaggatccacgccataccgatgtcacgaccgtccacatggcgcccctgcaggagcgtgtttacacttccgagtggactcttaccctggctaccgaggaaatgctggcaactttcccccctgtccaggacgtgctgtcccagctcgctaagtcttacacttccctggagacgtacgtcccttcaacagtggtacgctacctcacggcaggcaacaacccgcgcaacctgatgccggtgtcttgcggggttgtcatgttggctacagatattagctccttcacatcccttactgagatcagctcattcaccgaggtttggatgatctgcaacaccttcattgatgcatgtacaagcgctatctgccaggaaggtggggaggttattaaattgatcggggactgtgtcacaccatatttccccggtaacaacgctgatcttgccgtggccgcggcccaggagttgttcattttctgccgccagttgcgagaggcctttgtggacgtactggatgtgcgcggctgcgtgtcctgcgctgtgggaatggactacggccaagttgtgatggcccagtgcggtctgctccgcttgtctgactacgtggcagcgggcgctgtttctgccggggtcgtggaggtggaggctatcacgcgcgaggtcggataccagttcgtggttaccgtgccggatgcagaccgggtgcagccccagttccgagattatggcatcgagcctaccccgcaggctattggcggactcccctgctacgggatcgtgcgggaggtttttgacctgccagtggagagcattaagcggggtattaaggctatgcatgcggcccgctccggtgagcagcctctgactgaacccgagcaggccaagccagacttccgggtttcgcctggtcgagaccgtcacggtgtgtccggtcgacgttcgaacagcagtcagggcaagggcagcattcaggtgggctaa

SEQ ID NO: 43 - DNA, 3119 bp, unidentified
  misc_feature 1..3119: note = Eutreptiella gymnastica
  misc_feature 1..3119: note = Putative alpha subunit of photoactivated adenylyl cyclase, 4.6.1.1
  source 1..3119: mol_type = genomic DNA
tcaagacagtgccgcaagatgtacgtgcttgtttggaagaatggacagcaggtgaagacgtttgaaacgctcgatggcgccaactcctataagactggttccaacattgccgatgatgactctaacatttttcatatcgatgtcatccccgcccagagtgccggcagcgccggaggaggcaacgtagacaagccgctccgtcgtctgatgtatttgtcgaagactacagaagcagaattgtgcactccggagtatttggctgatatggcccacgtggcaacgttgcgcaacatggagatcggagttagtggattcttactttacagtgcaccgttcttcttccaggttatcgaaggtaccgacgaagatttggatttcttgttcgccaagattggtgccgacccacgtcacgaaaggtgcattgtgttggccaacgggccttgcacaggacgtctttatggtgactggcatatgaaggattcgcacatcgagaatatcacaagtcatcctgccatcaaaaccattctgtaccagattgcgcgctcgttctccagtatgtggtcttaccttcccaagaacgctgccaacttgcttcttctaggtaaaaacccaaacaaacagacccctgagcccactagtgtcgtcgttacgttcatttacttggtcgagttctcttcaattttggcacaccctggtttgaccgagcaagttgcagacgttctctcccacttcgttgacatttgcgtgcgcaacatagaaggttccggcggtgcaattgccaagttcatcaccggtattaccatggcctattggcccataaacagagctgaggatgccttgttggctctgcagcagatcaacgaagacctcgcccaattgcgttcgcagcaaccgccaggttcagctttgtctttgatgtacggacgtgctggcgtgcactacggtcgtgcattgttgtgtaacgctggtgtccgcaagtctgatttcactcttctcggagattgtatcaacactgcgtcgcgtgtcacttcgcttgccacgcagcttaagagtccactgctgttctccttcgaagtgcgttgcttgctgggagatgacatgagagacgaactcgaatctgctggtatgcacaaggtcaaggggcgagacaagccggtggccgtttaccagttcccttgcgagcctttggataatgatatggtgcgtgccaagattgaggctttcaacccggggagataccgagcccaattccccgtggttccatacgacagcctgcacggtgttcacaaaccacccatcttcgacgactcccccaaagaccctttggcaacgaggatcgcacaagctgccggcgagcgtaaggatagcttgatcgataggctgatcatgatatctaacttggcaattccagcatccatggccggaggaggcaacaaccaactcatcacgttgacatacatatctcacgccgtgaagcctatgagccgtttggatttggccgccatcaacaggaacgctgtgcgtcggaacgcggcctccaaaatcacgggcactttgttgtacgtgaatggacttttcgttggtactttggaaggacccaaagatgccgtcctaacgttgtacaataaagttcgtcgtgacgcgcggcacgaggacgttgtgacggtgcacttggcgcctctagacgagcgtgtctatgacgtgccgttctctgtgactgctgctacggacagcatgttgtccaagtttccgcctctgcaggatgtgttgtctcagttggctaagtctttcatttcccttgagacatacgttccgtccaccgtggtgagatggttgacagccggcaacaacccccgtgggttgcacgccgtgagtaccgaggtggtcatgttcgcaaccgatatttgcagttttacgccactttccgaaaactgctcgttggtcgaagtttggttgatttgcaacactttcatcgatgcttgcacgcactccatttgcaacaagggtggagaagtcatcaagctgattggagattgtgtgacggcttacttcccgcccgacggtgcggacgctgctgtgttggcttgccaagaggttgttaccttctgcgcacaacttagagaggccttcgaggaagttcttgactgtcgatccgttgttgcctgcggtgtcggtctcgactatggtcaagtcgttatggcccaatgtggaagtgaaggtatgaccgagtacgtggttgctggagaagtcagtgcacgtgttatggaagtggaggcactgactcgtgatgtggctcgggcaattgttgtgacggaaccggtggcagaccgtttgtcccctgccatgcgtgacaacggaattatcccttgctcccaaggtgtcgatggtatcccttgctatgggatcttgggagaggagtgggagctagacattccaacaatcaaggccaacattaagaggttccacgctactcgtgcggaaattgctaggcagaaggaagaggaggcagctgccgcggaggcaggaaagccaaaggccaaagcccgtgccgacagccgctttgggccacgcgggtctgttaatagttattgccccgatccgaacgaagttctcgaccctaggatggccgaggacgtctttgtggaggcttgtgcagcccgtggagacgcacctaatttggcccttaaagccaagatgaggcaagctgctaacgacgacaggctcgacttggcgagggccctgcaaggacctcacgagttatctccgttgaccaccgctctcaagcaactctgccatttgagaagcctcaatatgagcgacaatttcattgacgacaacaatgttgtggaccttgtggaagcttgcctacctatggagaatttgcaggtgcttgatttgtccaacaaccctggcattacgaagattattgccttgaagcggttggtgaagcacaatgtccaaatacgggaaatcaacctcaatggcacccgcgtggccccaacggagcagcgcaagttgcaatcttccattaatgtcaaccgtttgtgtgcatccgagaagaggtaagcatatgacattggtaggggccccacatgctgtggcccatctaagcaaatgag

SEQ ID NO: 44 - DNA, 3511 bp, unidentified
  misc_feature 1..3511: note = Colacium sideropus
  misc_feature 1..3511: note = Putative alpha subunit of photoactivated adenylyl cyclase, 4.6.1.1
  source 1..3511: mol_type = genomic DNA
ttttcccaaagacagaatgtacatccttgtttggaaagaaggtcagcaaatcaagactttccaagatcttgaagaatgtggccagtttcaaactgcttctaacatcagcgatgggcaaaccttttccatcaatgtgagtcctgttatgagcaaaggaggagagacaggagagacacagctgcgacgattgatgtacctttctgcgtcaactgagcctgagcggtgtgacggagaatatttggctgagatggcccatgtggccaccctgcggaatagtgagattggggtgagtggtttcctgctgtacagtgctccattcttctttcaagtaatcgagggtacagatgaagatctggacttcttgtttgcaaagattggggcagatcctcgccatgagcggtgtattgtcctggctaacggaccatgcacaggtcgattgtatggggagtggcacatgaaggattctcacattgacaacatcacgaagcaccctgctatcaagaccatcctgtaccagattgcaaggtctttctccagcatgtggtcctaccttccaaagaatgctgcaaacatgctgctcctgggaaagaaccccaataagcaagcgcctgagcctatgagcgttgttgtcacttttatctatctggtggagttctccagcattctggctcatccaggtctgaccgagcagtgtgccgatgtccttgctgcatttgttgatgcttgtgttcgcaacgtggaaggcactggtggtcagatcgccaaattcatcactggcatttgtatggcatactggcccatcaaccgagccgaagatgccctcgttggattgcagcaactttctgaggatttggctgagttgcgaagccaacagcccccaggctctgcgctgtcgctggtgtactcccgctgcggggttcattatggccgagcactgctgtgcaatgctggaacccggaagtccgacttcacactgctgggagactgcatcaacacggcctcccgtatcacctcgttgtctgtcaagctgaatgtgcctctgctgctgtctttcgaggtgcgctgcctgcttggcgacgagatgcgagaggagctggagagtgccgggctgcacaaggtaaagggccgcgataagcctgtccaggtttaccagttcaatgccccagagcttgacagcgcacaggtccgtgccaagattgagtcgttcaaccccggccgcttccgccccatgtgccccgtaaagccctacgattcactgcaccccagccagagaccccccatcttcgacgacaccccccgggagagcatgccgaagctgagcgtcgtccagcgccgtgacagcctcgtggaccggctgtcgctgatcgctaagctggcgttcccgccgaacatgatgaacgggggcgacaaccagctgatcacgcttacgtatatctcccaggcatcatacccgatgtcccggcttgacttggcggccatccagcgggttggatgccggcgcaacgagtcggcccagatcaccgggtcgctgatctacgtcaacgggctgttcgtgcagacgctcgagggccccaagtccgcggtcatcacgctgtacctcaagatccgccaggactcgcgccacaaggacgtcgtgatcgtgttcatggcgcccatcgaggaacgtgtgtacagcaccgcgctggacatgacgtccgcgacggaggagatgctcgccacgttcccccctttgcaggatgtgttgtcgcagctggccaagtctttcatctcgctggagacgtacgtgccatccacggtggtgcgctacctcaccgccgggaacaacccgcgcagcctgcagcccgtctccgtggaggtcgtcatgctggccaccgacatttgcagcttcacgccgctgtccgagaactgctcgctgacggaggtttggacgatctgcaacacgttcatcgacgcctgcacctctgccatctgcagtgaggggggtgaggtgatcaagctcattggggactgcgtcacggcctacttcccgccgacgtccgccgacaacgccgtggccgcctgccaagagatcgtcaccttctgcacccagctgcgggaggcgttcagtgatgtgctggactgccggtcggtcatcgcgtgcggggtgggcctggactacgggcaggtcgtcatggcgcagtgcgggagtctggggatgacagagttcgtcgtcgccggggaggtcagcgcccgcgtcatggaggttgaggcgctgacgcgtgaggccgggttcaccatcgtggtcacagagcccgtggcggaccgcctgtcccccaagctgcgggacaccggcatcatcccctgccacgagggggtggacggcgtgccgtgctacgggattgccggtgacgagtgggtgctggatatcaagactatcaaggcgaacatctacggcttccatgaggcccgcgcccaggctgccctgcggaaggtggatgatggcacccgtgccccaggccatgcggcccctgcgtctcccaaggcacagaagggagcgggccgggccaactccgtcgccagctacacccccgacgtgaacgagctgctggacccccggatggctgagtcggtgttccaggagctgtgccagcagcgtggggacgtcccgaatgtggggatttcagcaaagctgcggcaggcggcgcaggacgaccgcctggacctcggccgcacgctgcagggcccccatgagctcgggccggtggtgcaggcggtaaagcagctcacaaacttgaagacgctgaacctgagcgacaactttgtggacgacggcagcgtcggggaaattgtggaggcctgcctccccatgcggaacctgcaggttctggacctctccaacaacccggggctgacaaagatcatcgcattgaagcgcctcatcaaacacaacccggccgtgcgggagatcctcctgaacggcacccgcgtggccccgacggagcagcgaaagctgcagtcctccatcaacgtcaaccgcctctgctcctccaccgatgcgaagggaggctcccacaagtatgagagccattaagggggcgccgccgtgccccacggccggctataaaaaccgaggcgttgcgtgtggcggcaaaccgtcctgcttgcctgggcggttgtatcaacaaagggtgaggggtttggggtgcaggttgtaaaacgttggctgcactacctgttgtttttttttttttttttggagctggcccacaccaaaattggcctccagctcccctttgtcaccagcgcatgatggcttcggccactgttccacaatccacggattccctgcttggtcgactctgtgcccggcccctcccccaactgctgtcattcttgactgtcccatgaccccgccggcattgctactggcatcctgaactcagggcgatgaccttcatcgattcgcaaattcgcccgaagttgaaagtgatgtcttctccctcttcaatccgttgctgaccacagtgaccag

SEQ ID NO: 45 - DNA, 3127 bp, unidentified
  misc_feature 1..3127: note = Euglena quartana
  misc_feature 1..3127: note = Putative alpha subunit of photoactivated adenylyl cyclase, 4.6.1.1
  source 1..3127: mol_type = genomic DNA
cctgtcacgcccttcacctcgttgaggatgtacatccttgtttggaaagatggccaacaggttaaaaacttccaagatttggaagaatgcggcgcattccagacggctgctaacatcgtcgacggacagatcttcaatgtcggcgtcacctcagcgatgaacaaaggaggagacactggtgaagtgcagcttcgccgcttgatgtacctttctgcctccacggagccggaggaatgcaccaccgagtatctcgctgaattggcacgtgcttcgtctctccggaacaaagagattggagtgagcgggttcttgttgtacagtgcaccgttcttcttccaagtgattgaaggaaccgacgaagaccttgatttcctcttcgccaagatttcatcggacccccgccacgagaagtgcatcgtcttggccaatgggccgtgcaccggacgcttgtacggagaatggcacatgaaggattcccacatcgacaacatcacaaagcaccctgcgatcaagaccatcctgttccaaatcgccaaatcgttctccagcatgtggtcctacttgtcaaagaacacggcgaacatgcttctgttgggcaaacagcccaacaagcaagcaccagagcccatgagcgtggtcatatccttcatctatctggtggagttctcatccatcttggctcaccccgggttgacggagcagtgtgccgacgtcttggctgcatttgtggacgtttgcgtaaggaacatcgagggaactggagggcatgtggccaagttcatcaccggaatctgcatggcttactggcccagcaacagggctgaagatgccctggtcggtcttcagcaactgtccgatgatctcgctgagcttcgaagccagcagcctccgggctctgcgctgtccctcgtctactcgcggtgcggtgtgcactatggccgagcgctgctgtgcaacgccggtattaagaaggccgactttactctgctcggcgactgcatcaacaccgcctcacgtatcacttcgttggcggggaagctgaaggtgcccctgttgctgtcttttgaagtgcgttgcttgctgggagctgagatgcgggaagagctggaaagcgccgggatgcacaaggtgaaggggcgtgagcagccggtgcaggtgttttatttcaacgctgctgaggtggacagtaccgccgtgcgagcgaaaattgagcagttcaaccccgggcgctaccgcgcgttgtgccccgtgaagccctacgacagcctgcacccctctcagaagccgccgattttcgatgacactccgagagactccatgccgaggtccggtcatttgcaaaggcaagacagccttgtggaccggctcagcatgatcgccaagctggcgtttccgcccagtatggcagcgggcggagaaagtcagctcattggcctcacttacatatcgcaagcgtcgcatgccatgtcgcgcctcgacctatccgacatccaaagggtcgcccaccggcgcaacgaacaggtgaacatctccgcttgcctactctacatcaatgggcttttcgtgcaaacgctggaggggccgaagagcgctgtggtgtctttgtacctcaagatccggcaagacccccggcacaaagacgtcgtggtggtccacatggcccctctcgaggagcgaatgtaccgcggcccgttggacacaacgtgtgctacggacgagatgctggccacatttccgcctttgcaggacgtactgtcccagctggccaagtccttcatctccctggagacgtatgtgccctccaccattgtgcgttacctcacctccggcaacaacccgcgcaacctgcagcccgtgtcgctcgaggtggtcatgcttgccaccgacatctgcagcttcactcctctgtcggagcactgctcattgacggagatatggaccatctgcaacacgttcatcgacgcctgcacctctgccatctgcagccagggcggcgaagtcatcaagctgatcggggactgcgtcacagcctacttccccccaacatctgcagacaatgcggtctatgcatgccaggatatcgtggccttctgcgcaaacctgcgcgaggcattccgcgatgtgttggattgccgatcggtcgtggcttgcggtgtgggtcttgactttggacaggtcatcatggctcagtgcggcagctcggggctgacagagttcgcggtggccggtgaggtcagcgctcgcgtgatggaggtggaggccctcacgcgtgaggcgggtcgcgcgatcgtactcacggagcctgtggcggatcacctttccccgaagctgcgtgataatggactggtaccttgccaagaaggtgtggacggagtgccctgctatggcatcttgggagcggagtgggagctggacgtggtgaccatcaagaggaacatccaaaactttcacgccgcgcgcgctgtggctgccgtacagaaggtcgatgacggaatgaacgcccctgggcgtgccctgccagtcgcctccatgccagccatatcgtccccgtcccccaaggtgcgcactgcggggcgtgccgcttcggttgccagttacgctcccgatctcaacgatctgctggaccctcgcatggcagaggttgtgttccaggagctctgccagcagcgcggagagatccccaacaacgccgtcctcctccggcttcggcaggcggcccacgaagagcgcgccgacctcggccgctcgcttcagggaccgcacgagcttccccctctgacccaagccctgaagcagctcaccaacgtgagggctttgaacatgagcgacaacttcgtggacgatacctccatcggagacctcatcgaatcgtgtcttgcaatgcggtccctgcagatgcttgacctgtccaacaaccccggactcaccaaagtcatcgccctcaagcgcctcatcaaacacaacccgcagctccgtgagatccacctcaacggcacgcgcatcgcccccacggagcaacgcaagctgcagtcctccctcaatgtcaaccgcctctgcgcctccgctgaatccaagctgggttcgcacaagtacgaaggcgccgcccactagagccgcttttgctgct

SEQ ID NO: 46 - DNA, 3120 bp, unidentified
  misc_feature 1..3120: note = Euglena stellata
  misc_feature 1..3120: note = Putative alpha subunit of photoactivated adenylyl cyclase, 4.6.1.1
  source 1..3120: mol_type = genomic DNA
attcaagatgtacatcttggtttggaaagaggggcagcagatcaagacatttcaggatcttgaggaatgtggccaattccagacggcatgcaacatttcagatggccagatcttcagcgtgaacgttgctcccaccatgagcaagggtggagaaagtggtgaacagcttttgcgtcgattgatgtacttatcaaagtcaacagagcctgaagcctgtaacgctgaatacttggcacagatggcctttgtcgcaacatgcgtcaatagcacgattggagtcagtggtttcctgttgtacagcgcaccgttctttttccaagtcattgaaggaacggacgatgacctggactggctgtacgcgaagatctccagggacccacggcatgagcggtgcatcgtgctcgcgaatggtccttgcactggacgaatgtacggtgaatggcacatgaaggattcccacattgataacatcaccaagcacccggccatcaagaccatcttgttccagatcgctcgctcgttctccagtatgtggtcgtacctccccaagaacgcagccaatatgcttttgcttggcaaggagcccaacaagcaagctccagagccaatgagtgttgtgatcacgttcatttacctcgtggagttctcttctatccttgcacatcctggattgactgagcagtgcgccgatattctggaggcttttgttgatgcctgcgtgcgcaatatagaaggaaccgggggtcaggtcgccaagttcatcaccggtatttgtatggcgtactggccaatcaaccgcgctgaagatgccattgtggggctgcagcagctgtctgaagacctggcggaattgcgcagccagcagcctcctgggtctgcattgtcactggtgtattcaagatgtggtgttcactatggacgtgcattgctctgcaatgctgggttccggaaggctgacttcaccctcctgggtgattgcatcaacaccgcctcacgtatcacttcgctgtccgtcaagttaaaggttcccctgctcctctccttcgaggttcgctgcttgttgggcgacgagatgcgtgaggagttggagaactctggcctgcataaggtgaagggtcgtgacaagcccgtgcaagtttaccagttcaacgctcctgagcttgacagtgccatggtacgagccaagatcgagcagttcaaccctggccgttaccgtgcgctgtgccccgtgaagccctatgacagcctgtctgctactcaaaagcctccaatcttcgacgacaccatgcgtgatggccacaataagcttagtgcggcacagcgccgggatagtcttgtggaccggctcagcctgatcgccaaattggccttcccaccaagcatgctggccggtggagatggccagctcataacgctgacgtacatttcccaggcatcccgccccatgtcgcggcttgacctgtcggcgatccaccgtgttgcaatgcgccgcaacgagtcctccaacatttcaggttgtttgctgtatgtgaacggcttgtttgtgcagaccctggagggtcccaagagtgcggtggtttcgctgtatctgaagatccgccaggatgggcgccacaaggatgtggtgacggtgtacatggcccccctagaggagcgcgtgtatgcgaccgccctggacatgactgccgctacggaggatatgctgtccacgttccctcccctgcaggacgtgctgtcccagctggcacggtcgttcatctccctggagacgtatgtgccttctaccatcgtccgtttcctgacggccgggaacaacccccgcaatctgcagccggtgtctgtggacgtcgtcatgctggccacggacatttgcagcttcactccattgtcagagcgctgctctttgacagaagtgtggactatctgcaacacgttcattgatgcctgcacaagcgccatttgcaacgaaggcggagaagtcattaagctgattggggattgcgtgactgcctatttcccaccgcatgccgcagacaatgcggtcagcgcatgccaggagatcgtaaccttctgcacccacctgcgcgaggcattcaaggatgtgctggactgccgttccgtgatcgcctgtggtgtgggtctggacttcgggcaggtcattatggcccagtgcggtagcctgggaatgactgagtttgtggtggccggagaggtcagcgcccgagtcatggaggtggaagctttgacccgtgaggccgggcgtgccattgtggtcacagagcctgtggccgatcgcctctcctcccggctgcgagacagcggcatcgtgccctgccaggagggtgtggacggggtgccctgctacggtatcctcggaccggagtacgagctggacatcgtgaccgtcaaggccaacatctatggattccacgacgctcgtgcccaggcagctctgaggaaggctgacgatgggacgaacgctcctgggaagtcaggccctgcggtaccttcatctccaaaggtccaccacgctcaaggccgtgccggctccgtggccagttacacccctgacccgaatgaggtccttgaccctcgcatggcagaaatggtgttccaggatatgtgccagcaccgtggtgaccagcccagcaacgcgatccttatgaagttgcgaaacgccgcccaagatgatcggctggacctgggccgcaccttgcaggggccccatgagctgggacccctcacccaagccattaagcagctcaccaacctaaagatggtgaacatgagtgacaatttcattgatgacaacaccattggggatttggtggaggcttgcttgtcaatgcgatctctccaggtccttgaccttagcaacaaccctggcctaaccaagactatcgccctgaaacgcctcatcaagcacaaccctcacgtccgtgaggtactcttgaatggtactcgcatcgcgcccacggaacagcgtaagctgcagtcttccatgaatgtaaatcggctgactgcgtctgacaagggtgggtctcacaagtatgaccactagactatcgggtgccgataccgcgtgatgtaaagaatggtgcaccaaaacgatgcactatt

SEQ ID NO: 47 - DNA, 2814 bp, unidentified
  misc_feature 1..2814: note = Colacium sideropus
  misc_feature 1..2814: note = Putative beta subunit of photoactivated adenylyl cyclase, 4.6.1.1
  source 1..2814: mol_type = genomic DNA
tgcacgatgtacatcttggtttggaagaaaggccagcaaatcaaaacattccacacactcgatgaagcagcacaatttaaagcagcctctaacatcgatgacgctcagatgttctcaattaacgttgctccagcagtcagtgcgtcagggggaagtaacgaggccactaacttgcgccgtctgatgtatctgtcgaagagcacagagccagaaaagtgcgatgtcccctatcttgccgagatggccggaatagccactctgaggaaccgtgaaattggagtcagtggtttccttatgtactcttctccgttcttcttccaggtcattgaaggaacagatgaggatttggatttcctttttgcaaaaattggggctgatcccaggcacgaaaggtgcattgtgcttgcaaacgggccttgcacggggcgcatgtatggtgattggcacatgaaggactctcacatcgacagcatcaccacccatcctgcgatgaagacaattttgtatcagattgcacgctccttctcaagtatgtggagctacttgccaaagagcgcaggaaacatgttgcttttgggcaaagatcctgcttcccaacctccggagccaatgagtgtggtggtgacattcatttacctggtggagttcggatcaatcttgtcaaacccaaatttgactgaacaagcagctgaagtgctgagcacctttgttgatgtttgtgtgaagaacgtggaaggcagtggtggaaacattgccaagttcatcacaggaatttgcatggcgtattggcccatcaacagagctgaagatgcattgacatcaatccagcaaatcagtgaggatttggcccaactccgaagccaacaggcccccggctcagcagtttccctgatgtattcccaagccggggtgcactatggccgggccatgctctgcaatgccggaacccggaagtcggacttcaccctgctgggcgattgcatcaacacgacgtcccgggtggccacattggccaagaagctgaagactccgttgctcttctccttcgaagtccgctgcctgctcggtgatgagatgcgtgaggagattgagggggctggcatgcatcaggtgaagggccgtgacaagcccgtggtggtgtaccaattccccggagaggagctggacgtggatgaggtccgccacaagattgagaccttcaacgccggccgcttccgctgccagatgccggtggtggagtacgacacgctccacccctcgcagcggcccccgatcttcgacgagaccccgaaggggaacccccgcccccgcacccccggctacggcgggacccagcgccgcgacagtctggtggaccgcctgatcatgatcgccaagctggccggtaccactgcttcgaccaccggagacacaacgctcatcacgctcacctacatcagccaggcgtcacgccccatgtcccggctggatttgtccgccatcatgcgcgcctccacgcgccgcaacacgcagcagaacatcacagggacactgctgcacgtgaacgggctgtttgtgcagaccctggagggcccccgggatgccgtgatgaacctatacctccgcatccgccaagacccccgacatactgacgtggtgaccgtccacatggcgaacttgcaggagcgtgtgtacccgccggagtggaccctcacttcggcctccgaagacatgctggcggcgttccctccactacaagacgtgatgtcccagctggcgaagtcattcacttctttggagacctacgtgccatccaccgtcgtgcgttacctgactgctggcaacaaccccaggaatttgatgcctatttcctgcggggtggtcatgttggccactgacatctgcagcttcaccagcctcaccgaaaagtcttctttgacggaggtctggatgatctgcaacacgttcatcgacgcgtgtacttcggccatttgccaggaggggggtgaggtcatcaagctgattggcgactgcgttaccgcctacttcccgggctctgctgccgacgctgctgtggccgctgcccaggagctgtacctgttctgcactcaactgcgtgaggccttcgtggatgtcctcgatgtgcgcggctgtgtgtcatgcggcgtggggcttgactacgggcaggttgtcatggcgcagtgcgggacccttgggctgacagagtacgttgtcgccggggccgtgagcgcccgcgtcatggaggtggaggccctgacccgagaggtggggcatgccattgtcgtcacagagccggtggcagaccgcctgtccccccagctgcgggactcgggcatcatcccgtgcccccaagccatcgagggcatcccgtgctacgggatcgccggggagcagttcgagctggacatcgactccatcaagcgtggcatcaaggcgttgcacgcggcgcgcagcggggagaaggtggcggtggcggagccggaggagaagggcggcgacttccgtgtctccccgggccgcgtccgccgcagcgactccggccgccgctccaactccgcgcagggcaagcggggcacccctgtccgctgatctttcgaaccaacagggtttgtaaacttcacagtgcctttcctttacctgctggctccactgtggtcgtgaacttcattgggttgtactctataaaccttaccccttttttttctttcgagtttgtttgttcaccgtccgcgcttcgttttggcggcacttcgtaacgatgaaccccttccgtgggatcattccgcaatgcatatccacttcgcttgacctgac

SEQ ID NO: 48 - DNA, 3368 bp, unidentified
  misc_feature 1..3368: note = Eutreptiella gymnastica
  misc_feature 1..3368: note = Putative beta subunit of photoactivated adenylyl cyclase, 4.6.1.1
  source 1..3368: mol_type = genomic DNA
gggacgcatcagctgcaaacaccatgtttctcttggtttggaagaagggccagcaaatggcaaccttccacacgctggaagaatccaacgagttcaaagccgcgtccaacattgaagattcacacgtcttccacttgaagctcacacccactaccagtggtgctggtggagccaacaatgccaccggatttcgccgtttgatgtacctgtcaaaatctacggaggctgaggaagtgaacagcacttatttggccgagatggctggaatcgcaacgatccgtaaccaggagatcggcgttagtgggttcctgatgtactctgaccctttcttcttccaggttatcgaagggactgacgaagacctagatttcctgtttgccaagataggagcggaccctcgtcacgaaaggtgcatcgtgctggccaacgggccttgcaccggacgcctgtacggagactggcatatgaaggattctcacatcgatagcattacctcgcaccccgccatgaagaccatcttgtatcagattgccagatccttctctagtatgtggagctatttgcccaaaagtgcaggacacatgttgctgcttggtaaggaccccaacaaacagcctcctgagccgatgagtgtcgtcgtcaccttcatctacttggtggagttctcgtccattctctcacaccccggcttgactgagcagtgcgctgaagttctgagcaccttcgtcgacgtgtgtgtgaggaacgttgaaggcagtggaggaaatattgctaaattcatcaccggtatctgtatggcgtattggcctatcaacagggctgaagatgccttgacagctctccagcagatcaacgaggatcttgctcagctccgtggagcgcaagcccctggctctgcgttgtccctgatgtattcccgttccggagtgcactacggtcgagctatgctgtgtaacgctgggactaggaagtctgacttcaccctcctcggagattgtatcaacactgcatcacgagttgcaagtttggctgttactctgaagtcccccctgttgttctcgttcgaagtccgctgtttgctcggcgacgagatgcgtgacgaaatcgaaagtgctggaatgcaccaggttaagggacgtgacaagcctgttgtggtttatcaattcccaagccctatcttggacagcgatgttgtgcgccagaagattgagcagttcaaccctggccgctacaggtgctccatgcctgtgcttgactatgaaaccatgaatcctgcaaagcgacccccaatatttgacaacgcccccaagggcaacccgaagattggcaagaagtacaacgctgctgcccaccgtgatagtttggttgacaggctcaccgagattgcccggctggcgttcccaggatcccaggctggcggtgattcgaccttgatcagcctgacctatctttccaactccgttcgccctatgtcccggttggaccttgcagctatccagcgtactgcactccgccgtaatgcaagccagggcatcaccgggtgtttgctttatgtcaatggattgtttgtccagaccatcgagggcccaagagataatgtgatcaatctgtacttgaagattcgggaagatccccggcacaaggacgttgtcactgtctacatggtccccatccaggaccgtgtgtaccaagaggcctggactatgaccactgccagtgaagacatgcttgccacgttcccgcctctgcaagatgtgttgtcgcagttggccaagtcgttcacttctcttgagacctatgtgccatccactgtggtgagatatctgactgctggaaacaatccaagagatctgatgccagtttctgtggagtgtgtcatgtttgccacagatatctgcagctttactatgttgacagagaaatcatctctggaggaggtttggatgatctgcaacactttcattgatgcctgtacctctgccatctgcgccgagggcggtgaggtcatcaagctgattggcgattgtgtcactgcctacttcccaagcgagagggcagacaacgctgttgctgcttcacaggagctgtatatgttctgcgttcacatgagggaagctttccaggaaattctcgatgtccgtgcctgcattgcttgtggggttggtttggactatggccaggtcgtcatggctcaggctggtagtttggggttgactgaatatgctgtcgctggggctgtcagtgcccgcgtcatggaagtcgaggctctttcccgtgacgtcggccgtgccatcgtttgtaccgagcccgtggttgacagactgtcgccacacttgcgtgattctggtgtcattccatgccctcaggcaattgagggaatcccttgctatggaattgctggtgaagattacgagctggatatccgatccatcaaggctggcattaagcagctgcatgctgcccgttctcttgaagatcaggctgctgctgcagaggaggaggctgcgcaggcccagcagcagttccgtgtgtccccagggcgtgtccgtcgcagtgatgctggccgccgctccaattctgcccaaggccgtcgcatggccccagtccggtaaatattgcaaacatgccactcctgggttggttagatcagagggcaatggttggtggttggtctagcaggtgcctggaccgcatttgccatctccctttggttgccaatgatcggttcgattggatggttgaagtttttttggtgctctgttttgcaatagttgcaaggccagccagtcatccactaattgcaaaaatgaaattcacttcgtatttggaggttgtcattcaggacgtgattgtaaagtagacctcttgtcaacgttctggattgtaaagtagtagtagtatcatggaccataatattacaacggtattagagtatcagtgtcatgtcatgcaatcaaatcttcaacattcatctgccatgccataccaagtttctgaccgttgtcagattgtttgattatcagaaaagagactgttcgttctatatcccagaaagtcatggcatctattggacctattttaccaggttgatggtgaaaataacatttttgcgcatcgataagattctgcttaaacatagtctcgtctctttgatgcgccatcctttctgcccatccattcatttcaatttggattttaccaggtgtctggcattttgcagtgcagtgcagtgcagtggtgtttcccttcctacagtgcttctctgggatttcacggacaccgcacacagtatggcttataagagcacccatggtgcacacctcatctatccttgttggcttacaaaattagttttgtcccatggagtg

SEQ ID NO: 49 - DNA, 2731 bp, unidentified
  misc_feature 1..2731: note = Euglena stellate
  misc_feature 1..2731: note = Putative beta subunit of photoactivated adenylyl cyclase, 4.6.1.1
  source 1..2731: mol_type = genomic DNA
gccacagcatgtacatccttgtctggaagaaggggcagcaaatcaagtcattccatacgcttgatgaggctgcagaattcaaggcagcttcaaacatcgatgaggcacaaatgttcagcattacggttgctcctgcaatcagtgcctctggaggcaccaacgaggccacgaatctgcgacgtttgatgtacctttcaaagtccacaaatccagaagagtgcgacccagcattcttggctgagatggcgggaattgcgaccattaggaataaggagatcggagtgagtggtttcctaatgtactcctcccctttcttctttcaggtcattgaaggcacagatgaagaccttgatttcctctttgcaaagatctcagctgatccccgccatgaaaagtgcatcgttttggccaacggaccttgcactggtcgtatgtatggtgattggcacatgaaagactcgcacattgacagtatcaccacccatcctgcaatgaagaccatcttgtatcagatcgcgcgctcgttttcaagtatgtggagctatctgcccaagagcgctgggaacatgttgttgcttggaaaggaccctgcagcccaaccacctgagccaatgagtgtgatcgtcactttcatctatttggtggaattcggctccatcctctcaaaccccgatctaaccgaacaagcggccgaagtgctgggaacctttgtggatgtctgtgtcaagaacgtggaaggcagcggaggtaatattgccaagttcatcactggcatttgtatggcatattggcccattaatcgcgcggaggacgccctgacgtccatccagcagattagcgaggatttggcccagctgcgtagccaacaggcccccggctctgccatcacgttgatgtactcccaggccggagtgcactatgggcgtgctatgctgtgtaacgctggtacacgcaagtccgacttcaccctcctgggcgactgcatcaacaccacgtcccgagtggccaccctggccaagaagctgaagaccccgctgctgttctccttcgaggtgcgctgcatgttgggcgatgagatgcgggaggagatcgaaggcgctggcatgcatcaggtcaaaggacgcgtgaagcctgtggtggtgtaccagttccctgggccagagctggatgtggaaatggtgcgacagaagattgaccagttcaaccctggtcgcttcaggtgccagatgcctgtggtggattatgactcgctgcccgtttcccagcggccccccatcttcgatgacacgccgaagggcaacccccggccccgcactcccggctatggaggcacccagcgaagtgacagcctggtggaccgcctcatcatgattgccaagttggccggtccatccgcatcagccggaggggacacctccctgaccaccctgacgtacatctcgctggccacccgccccatgtcccgcctggacctctccgccatcatgcgcaccgccacccgccgcaactcgtcgcagggcatcacgggaaccctgctgtacatcaatgggctgtttgtgcagaccctggaggggcccaaggatgccgtggtcaacctgtatttgaggatccgccaggacccacgccacacggcggtggtcacggtgcatcaggccgctctccaggagcgtgtctatcctccggagtggactatgacgtctgccaccgaggagatgttggccacattcccgcccctccaagacgtgttgtcgcagctcgccaagtctttcacctccctggagacctacgtcccatccactgtggtgcgttacttgacagccggcaataaccccaggaacctcatgccagtgtcgtgttccgtggtcatgctggctaccgatatctgcagcttcaccagtctgacagagaagtcatccttgacagaggtgtggatgatctgcaacacgttcatcgatgcctgtacctccgccatctgccaagagggtggggaggtcatcaagctgattggggactgtgtgacggcctacttcccggggaatgccgccgacgctgctatagctgctgctcaggaactgttcatgttctgcacgacgcttcgccaggcctttgtggatgtgctggacgtgcgcggctgtgtagcatgtggcgtaggactggactacgggcaggtagtcatggggcagtgcggtacgatgggtctcacggagttcgtggtggccggggccgtcagcgctcgggtcatggaggtggaggcgctgacccgtgaagtgggccgggccatcgtggtcacagagccagtggcagaccgcctgtccccccagatgcgtgacacgggtatcatcccctgccctcaagccatcgaggggttgccgtgctacgggatcgccggggatgactacgagctggacattgatgagatcaagcggtccatcaagcacctgcatgcggctcgcagcggggagaagatgccgtccgcggatcacgaggagaataagggcaacgatttccgcgtgtcccccggccgggtgcgccgcagtgactcgggcaggaggtcgaactcggcccagggcaaacggggcacccctgtgcggtgaactcaggtgggcgcataggtccgaggcagtcctctctctcatttgggcatggtacaccactttttttaattggccttggtactcgcattgggcataccctgagggatggtgccgctacattgtatggtcactttgct

SEQ ID NO: 50 - DNA, 2988 bp, unidentified
  misc_feature 1..2988: note = Eutreptia viridis
  misc_feature 1..2988: note = Putative beta subunit of photoactivated adenylyl cyclase, 4.6.1.1
  source 1..2988: mol_type = genomic DNA
taatcgtgctctcgcaaaatggttttcctgctggtttggaagaatggtacgcagctgaagtccttcctgaccctggacgacgctactcaatacaaaacagcaagcagcattgatgatgctcagatctacaatgttgctttcacccctgctgtcagtggagatggtgggtccaacgaagcaaccaatctccgacgtctcatgtatctgtcgaagagtacggagcctgaactggtgaacacccaatatctcgccgaaatggctcacatcgcaacgctgcggaatcagtgcatcggcgtgagtggttttttgatgtactctcatcctttcttctttcaggtgatcgaagggactgatgaagatttggattttttgtttgccaagattggtatagacccacgccacgaaaggtgcattgtgttggcaaatggtccctgcactggaagactgtatggggattggcacatgaaagattcgcacatcgacagcattacctcgcaccccgctatgaagacaattttgtatcagatcgcaaggtctttctccagcatgtggagctatttgcccaagagtgccggcaacacactcctgctcggcaaggacccagctgcccagccaccagagcccatgagtgtcgttgttacgttcatctacctcgttgaattctcgtccatcctctctcatccgggattgacagagcaatgtgccgaagtgttgagtacctttgtagatgtttgcgtgaagaacgttgaaggaagcggtggaaacattgcgaagttcatcacaggaatctgcatggcgtattggcccatcaacagggcagaagacgcgctgaccagcattgcgcagatcagtgaagacttgggacagctgcgaggccaacagcctccaggctctgctctatcgctcatgtactctcgcgccggggtccactatgggcgtgccatgctgtgcaacgccggatcccgcaagtcggattttacactgctgggggattgcatcaacacggcatcccgggtcgcgtcgttggccgtgactttgaagtccccgctgttgttctcattcgaagtccgatgtcttctgggcgacgagatgcgagacgagatcgaaggggctggcatgcattcggtcaaggggaaagacaagcctgttgtggtgtaccagttcccagggcccgagctggatagcggtatggtgcgccagaaaattgaagagttcaaccctggtcggtacaggtgcggcatgcctgtcatggagtacgagtccctgcacgtgtctgagcagccccccatctttgatgacacccccaaggggactcccaagccaggcacacgggcttggggaggaacgaaccgaaaggacagtctggccgacaagctggccttgatttcccaattggccggccccctccagacatccggggacaccacgctgacaaccctcacctacatctcccactccacgcggccgatcacccgcatggatctggcctctatccaacgctgtgccctgcgccgcaacgctcagcataacatcactggatgcctgttgtacgtcacgggcctgtttgtgcagaccctggagggccccaaggacgcagtcatcaacttgtaccacaagatccgcgctgacgcccgacacacggacgtcgtcaccgtacaccagggccccattcaggaccgtgtgtatgcctctgcctgggccctgacaaacaccagcgaggagatgctttccgctttccctccgttgcaagacgtgctctcccagctggccaaatcttttacctcactggagacctacgtaccatcaaccattgtccgctacctgactgctggcaataaccccaggaatctgaggcctgtctccgtgggggttgtcatgttggcgactgacatatgtagtttcacaatgcttactgagaagtcatcccttactgaggtctggatgatttgcaacacgtttattgatgcctgtaccaatgccgtaattaccaatgggggagaggtgatcaagctcattggggactgtgtgaccgcctacttccctgggcatggtgctgacggtgctgtggccgctgcccaggagatcttttccttctgtacccaattgcgggaggcctttgctgacgcgctggatgtgcgctccgccatttctgccggaatcggcctggactttgggcaggtggtgatggcccaggctggaagtcttggggtgacagaatttgctgtggcagggtctgtcagtgcccgggtcatggaggttgaggcgttgacccgtgatgtggggcgcgccatcgtggtgacagaacctgtggcagaccgcctgtcccctcagttgcgtgaccgtggcctcattccatgcccccaagccatcgagggcattccgtgttacggccttgccggggatgactttgagctggacatcgcctccatcaagggccgcatcagaaaccttcacgccatgcgggcaggggaaaagcccaacctcccagacgaccatgagcaagaggataactacaagcaagcccagcaggacttcagagtgtcccctggacgagtgcgccgtgccgattctggccgccgctccaactcggcccagggccgccgcagcgcacctgtccgataaccgggcagggtacatcaccgcgcagtggccttgtagggcccactcccgcagcatcgaaccttgcaaggggtggggatggtaggcgcagcatctgcatgtgcaaggcccgacccttcgacatcgggcccagcaaggggtggggattggagtccaacctgccctcagccagggctttgtttcttatgcgtgatggtggtgtgcaggcaatggacaaatcatggcgaatgctctctgccatgtaggtgctccccccgcccaattcgaagtagtcccccagcctcttccaccctctgatgacaccctgctactgtactctattgtatgtttcttagggcccatcaatgactggggtgacagtctgcttgt

SEQ ID NO: 51 - DNA, 3689 bp, unidentified
  misc_feature 1..3689: note = Eutreptia viridis
  misc_feature 1..3689: note = Putative alpha subunit of photoactivated adenylyl cyclase, 4.6.1.1
  source 1..3689: mol_type = genomic DNA
gtcggacaacataaaaaatgtacgtgcttatttggaagtctgggcaacaaattcagactttccctgatctggaagctgccagccagttcaaggctgctagcaacatcgaagattcgttcattttcagtgtcagcgtgacaccaacaatgcttgcgggcggtgctgctggagagtcaggccttcgtcggctaatgtatctttccaaggccacagaacccgatgagtgtacacccgagttccttgccgaactggcacacgttgcaaccctacggaaccaggagatcggagtcagcggtttcttgctgtacagcgcacctttcttctttcaggttattgagggaactgacgaagacttggatttcctgttcggtaaaatcggtctcgacactcgtcatgaagcttgtattgtgttggccaatggcccctgctccggacgcatgtacggggagtggcacatgaaggactcccatatcgacaacattactaagcaccctgcgatcaagaccattttgtaccagatcgcccgttccttctccagtatgtgggcttacttacccaaacaagctggaaacttgcttctactcggcaaagagcccaacaagcagccacccgagcccatgagcgcagtggtcagcttcatttatctagttgagttctccaccatccttgccaatcccaatctgactgagcaggtggccgacattctgattgcctttgtcgattcctgcgtgaagaacgtcgagggttcaggtggcagtgttgcaaagttcatcaccgggatctgcatggcttactggcccattaaccgagccgaagatgcactgcttggtctccaagctatctctgacgaccttgcagagttgagagcacatcaaccccccggttctgcgttgtctcttgtctactcccgagccggggtgcattacggacgtgcactgttgtgcaacgccggaatgcagaagtctgacttcactcttcttggggactgcatcaacactgcctcgcgggtcacctcactgtctgtgcaattgaaggtgcccctgctcttctcgttcgaagtgaggtgtttgcttggcgacgacatgcgtgaggaacttgaaagtctgggcatgcacaaggtgaagggtcgtgataaacccgtagctgtatatcagtttgctggggccccagagttagacagtgccatggtgaaggccaaggtcgagcaattcaacccaggcaggtacagagcacagtgccccgtgcgagaactggaatcgctgcacccggacgttcgcccacctattttcgatgatactccccgcgaggcttcgatgccgaaggcaggcgcaggggaacgccgtgacagtcttgcggatcgtctgacaatgattgccaagctggccttcccgacgatcgggggaggtactaattcgcagatgataacgttgacatacgtctccttttcctctcgacctatgtcgcggttggatcttgctgccatccaacgcgttggcctgcggcgcaatagccaagtggacatcaccggatctcttttgtacgtggcacctctgttcgtgcagacgctggaaggcccaaaggatgccgtgatctcagtctacatgaagattcgtgcagatccacgacaccgagatgtggtggcggtttacatggccccacaggaggagcgggtgtacaacagtgccttcgagctgaccactgcaacggaggagatgctgagtgctttcccccccttgcaggatgtgctttcacagctggctaagtccttcatctccctggaaacctacgtgccctcgacagtcgttcgctatcttaccgctggcaacaatcctcgcagtctccaagccatctccacgggggtggtgatgttcgccacggacatctgtagcttcacgcccctctcagagaagtgcagcctgacggaggtgtggctgatctgcaacaccttcatcgacgcctgcaccgccgccatcgtccaggagcgcggggaggtcatcaagctgatcggcgattgtgtcaccgcctacttcccccccaccaatgccgatggcgccgtgatggcttgccaggagatcgtcgccttctgcaccggactgcgggcggcgttcagcgacgtgctggacgtgcggcaggtggtggcgtgcggggtggggctggactacggggacgtggtgatggcgcagtgcggcagcatggggatgacggagtttgtggtggcgggggaggtgtcggcgcgcgtcatggaggtcgaggccctcacccgcgaggtcggccgcgccatcgtcatcaccgagcccgtcgccgaccgcctctcccccaagatgcgcgacaccggcatcgccccctgccccgagggcgtcgacggcatcccctgctacggcattgttggcgacgactgggatctggacattgtcaccatcaaaaagaacatctacagcttccatgaccgccgcgatgccgtggcccacgctaaggccgcggcagcccaggaggccctggacgctgcagcccccagccccaagaaagggggccgtggctctgggaggtccagctcagtgtccagctacgccgcagatcccaacgaggtgctggacccccgtatggccgaggcggcgtatacgcagggctgccagcagcgcggcgaacagcccacgatggccattgtgaacaagctaaggggcgccggcaatgacacccggatggatttgggccgtgcactccagggtccgcatgagctcatggcgctgtgcacggccctcaagcacctcacgcacctgcgcctgctcaacatgtccgacaacttcgtggacgacaacagcattgccgagatcgtggaggcctgcctgcccatgaagtccttggcggtgctggacctctccaacaacccggggctgaccaagatcctggccctgaagcggctggtcaagcacaaccccggcattaaggagatcatcctgcagggcacccgcatcgccccccccgagcagcgcaagctgcagtccagcatcaacgtcaaccgtctgtgtgcctcctcccccaacggcgcggagagcaaggcgggccacaagtacgaccagagcatgtccagtagccaccactgatgcctcccgacgtgccgccgccagcagggggagcaggcgccattgtcccgttgttccctgtgtacagtaacccgccccctcccccaaccccggcctctcgcccccttttcatcctcagcaaagccgcacgtccccatcgctggtcacagacaatcacagccggtcacagacaaggcgaatggaatggacagccaactcccaaacaccccagcccttccccccgccaccagcatgccagacatttttccaccgggcgctggggtgggcctaggctttatctacgtaagggagacgtgcatacactttgtcacaggtgcagtattgcaggaggtatgttaaaccaaggctaaatgcaacccccccccttggggtcggccgggccgggaccctatcaaacttgacccccccccccgggtgttccgggggttgggcgtcccccccgccctgtggtccgcggacaagcacaacccttgttcacgcctgcgcttgcccctcgaaagctgcgctcacaatccccgtgccatgggcacaccgttgggccagccattgcgtccctgcctgaccttgggacacggcagatcgcctcag

SEQ ID NO: 52 - DNA, 2580 bp, unidentified
  misc_feature 1..2580: note = Euglena gracilis
  misc_feature 1..2580: note = Photoactivated adenylate cyclase subunit beta-like protein 1224-5/1FpacB
  source 1..2580: mol_type = genomic DNA
atgtacattttggtgtggaagaaaggtcagcaaatcaagactttccatacgctggatgaggctgcgcagtttaaggcggctagtaacatcgacgaggcccaggtcttctctgtgaccgacgctcctgacatctccgcctccggtggctcaaacgaagcccgtaaccgtcgccgtctgatgtacctgaccaactcaacaaacccagaagagtgcaatccccagttcgtgggagagatggcgagggtcgcaaccattcagaatagagagatcggcgctcgtggcttcctgatgtactcttgccgctttctgcttcaggtgattgaagagacggtggaagaccccgactcattgttcgcgaagttcagcccagagccccgacatgagcgctggattgagctcgataacggaccttgcaccggccgcatgtacggggactggaacatgaaggacagccacctcgattcgatcaccacacacccagccatgttgacgatccttttccagatggcgcggagcatttcttgcctgtggtcctatttgcagaagagcgagagaatcatgcttctcttcggtaaggatccgggtgcccagcggcccgaacccatgcgtgttgtggttaccttcacatacttggtcgatttcggcagcatgctcagcatccccaatctcaccgagcaggcagccgaggttttgacgactttcgtggatggctgcgttaagaacgtggaggggtccgggggttctattgccaagttcacaaccggcatttgcatggctcagtggccgattaaccgcacagtggaagccctgacagcgatccagcaaatctcggaggatttggcccagctgcgcagccagcaagcccctggctccgccgtttctctgatgtactcccaagccggattgcactacggacggggcatgctgtgtaacgccgggagtcgcaagagcgacttcaccctcttggattttatcaacaccacgagccgcatagctaccctggcaaacaagctgaagaccccactgttgttccactccttcgatgtgaggtgtttactgggcgatgaaatgcgcgaggaaatcgagggagcaggaatgcaccaagtgaagggtagggacaagcccgtcggagtctatcagtttcccgggccggagttggatgtggaaatggttttgcggcagaaagtggagcaattcaccccaggccggttccgctgccaaatgcctgtggttgaatatgaggcactgccaatctggcagcgacccccgatcgttgatgacacacccaagggttatccacggcctcggacgatgcctggctacggcgggcgccagagatctgactctctcgtggatcggctgattatgatcgctaagctggcagggccttctgtgtctgcgactggggacaccacgcttactacgctcacctacatttcccaggcaacccgcaagccgatgtcacggctgcacctgtccgctctgatgggcacggccacccgcaggaatgctcaacagtctatcaccgggactctgcttgacgttaacggcctgttcggtcagaccctggagggtccgaaagtcgcagtcgtaatgtacctctccatccggcaggatgcccgccagacagacgtaactaccgtccatatggcacctttgcaggagcgtgtttgcccgagtgagtggactctgacctccgctactgaggaaatgttggccactttcccccctctgcaggacgtgttgtcccagttggccaagtcttgcacatccctggagacctacgtgcgctccactgtggggtatctgactgccggcaacaatcgtcgaaacctcatgcccgtttcgtgcggagtggtcatgcttgccaccgacatcggtagtttcacctccctgacagaggaatcctcgctcacagaggtttggatgatctgcaacacttttattgacgcctgcacgtctgctgtctgccaggagggcggggaggtgattaagcgcatcggcgactgcgttactgcctactttcctggcaacaatgccgagtccgctgtcgctgcggctcaggagttgttcaccttttgccgccagctgcgcgaggctttcgtcgacgtcgtggatgtgcgtggctgcgtctcctgcggggtgggactagactattcccaggtggtcatggcacagtgtggatcgctgggtcttacggagtacgttgtcgcaggtgtgagcgcccgggtgatggaggtcgatgcgatgactcgggaggttggttacgctatcggtgtcaccgagccctacgtgaccgaccgcttgagcccccagtcccgcgaccatggaatcgtgcccactccacaggcaatcgagctgccgtgttacggcattgccggcgagcagctggagctcgacgttgacagcatcaagcggggcatcaaggcccttcacgcggctaggagcggcgagatgccagtgaccgagcccgaggaggccctgcgtctgttccgagtctcgcccggtcgagtgcgtcacggggacacaggtcgtcgatcaaacagcgcccagggcaagcgcagtattcaagtgcgctaa

SEQ ID NO: 53 - DNA, 2568 bp, unidentified
  misc_feature 1..2568: note = Euglena gracilis
  misc_feature 1..2568: note = Photoactivated adenylate cyclase subunit beta-like protein 1224-5/9FpacB
  source 1..2568: mol_type = genomic DNA
atgtatattttggtctggaagaaaggccagcaaattaagaccttccacaccctggacgaggctgcccagttcaaggccgcgtccaacttcgatgaagcaccaatgttctcggttactgtggcccccgcaatctccgcctccgggggatcaaacgaggccaccaacctgaggcgcctgatgtacctgtcaaagggtaccaacccggaggaatgcaatccccagtttgtggccgagctggctcgggttgctactattcgcaaccgcgagattggtttatctggattcttgatgtcgagctctcctttcttgttccaggcgatcgaggggaccgatcaggatctggatttcttgttcgccaagatttctgcagacccgaggcacgagcgctgcatcgttctggcaaaccgtccctgcaccggaaggatgagtggagtttggcacatgaaggactcacacatggactctatcacgacccatgacgccatgatgacaatcctttaccaaatcgcccggtcattttccagcatgctgacttacctgccgaagcgggccgggaacatgctgttgagcggcctcgaccccgcagcgcagccacctgagccgaagtcggtcgtagtgacattcatctacctggttgcctatggtagcttgctgtctaatccgtactctaccgaacagccggcggaggtgctgtccacgttcgtcgacgtttgcgtcaagaacctggagggcagcgggggcatcattactaagttcatcaccgggatctgcatggcttcctggccgtttaaccgcaccgaggaagctttgacagccatccagcaaatctcggaggatttggcacagctgccctcccagcaagcgcctggctcctctccgagtcttatgtatagccaggccggtgtgcattacggccgggctatgctttgtaacgcaggttctggcaaatccgacttcacgctgttgggcgactggattaacactacgtctcggatcgcaaccctcgctaaaaagctgacatccccagttctgttcagcttccaggtccgatgtctgcttggcgacgaaatgcgcgaggaaatcgagggtgccgttatggatcaggtgaagggacgtgacaagccagtcgttgagtaccagttccctggccccgagcgtgatgtggaggtggtcgggcaaaaaattgagcagttcacccctgggcgattccgttgccagatgcgagtggggcagtatgaggctctgcctatctcacagcgtccacctatcttcgacgatacgcccaagtgcaatccccgcccacgcacccccgggtgcgagggccggcaacgctctcacaccctagtggacccgcacattatgatggccaagctccccggtccctccgtcagcgccactggcgacaccacagttcctacgttgacatacattagccatgccacccgcccgatgagtagactcgatctctccgctattatgggaactgctactcgcatcaatgctcttcagcgaatcactgggaccttgctccatggcaacggcctgtttgtccagacccaagagggcccaaaggaagcggagatcaacctccttctccggattcgtcaggacacccgtcgcagcgacgtggctaccgaccacatgccccagccccaggaacgcgtctaccccagcgaatggacactcacctccggcaaggaggaaatcttggctatctttccacctttgcaggatgtgctgtcgcgtctggcgaagtcattcactgcccaggagacctacgtgccttcccgcgtggtctcgtatctgacagcaggtaataaccctcgcgagttgatgcctgtctcctgcggtgttgtgatgaccgacatctgcagcctgacgtccttgacggagaactcctgcttgtctgaggtgtggatgatatgcaacacattcatcgacgcctgtccctccgctatttgccaggaaggcgcggaggtgatcaagctgatcgggaactgcgtgaccgcttactttcccggaaacaatgcggacagtgccgtggcctccgcgcaggagctgtttacgttctgtcgcgagcacagacaggcaatcgtcgatgtgctcgatgtgcgcggttgcgtgtcttacggcgttgctctggacaacggtcaggtggtcatggcacagtgcggttccgagggcctcaccgagtacgtcgttgctggcgccgtgtctgcacgggtgatggaggtggaggccatcacacggggcacgtgcccccggtgggccactcccctgtggagcgccgaccgactgagcccacagcttagagatcacggtattgtgcccactccccaagcaattgaaggcctgccctgctacgggatcgccggagaggagttcgagctggacgtcgattctattaagttgggaatcaaggcccttcacgctgcccgtagcggcgagaagcccctgactgagccagaggaggcgaagctggatttctcgccaggacgcgttcgccatggtgactccgggcggcgttccaactccgcccaaggtaagctcagtatccaggtgaggtaa

SEQ ID NO: 54 - DNA, 4252 bp, unidentified
  misc_feature 1..4252: note = Euglena gracilis
  misc_feature 1..4252: note = Putative membrane-bound adenylyl cyclase, 4.6.1.1
  source 1..4252: mol_type = genomic DNA
ccaatgaggttttctgtctacagcctatatgccctcgcctgcctcctcgccgttggaggggccattctcaccggcatcatacttctcgacacttttgacggccagcagcggaacagtgagaacgcacagttcacggccggcaaggggcaggtccagtccatgcagggggtcatcgctaaccgcatgacccgcatgcagaccatcaccgaggagcatagccggcagatcatgatgttcttgcagagcctccccagcaatgccattcagcctgataacgtggtgcaggctgtaaaccattccatctttgacagctgggtcccctccgtcaaggcgaacaactctttgaatggttacggcttgacattcacgtacaccaacgagacgactggcaacagtttcgaccgcaccttctgggtgtattgggacctgatgacgaccggggtgtacgactacatctacgcctacaccaacatgagcgatgggtatatccacgcttacgagacactgtggccgaatttctcgaccccacagctgggactggacatgtaccgcttcaggagtactgtcttggcagccagcatcaaggacgatgatttcttcaccaccgcccgtgtttgggtctccgcagacggcaactcctactggtacttcacccacatgcgcaccttccagttccagggagtgtacgccaatgttcagtcatgggacgtggccatcagctggctggacttgatgcgaactggtctgacaccggaggcaaacgtcgtggcctttgacagccagggctatgtgatggccgccaccaacaaggacgagctggcgaggctctcacagtgccgcggatcgttcaacagcgggaatttggcggccacttgtatcagttattttgcccgaaatcatcccatcctcgagatccgcaacctgtacaacgcgctgcacaccccggtgtgggacgacctcggtgctgcgccgattcccctcgagcaggccacgctgtccctctcagggaagaggtacatggccatcattgccactttgtactccaaagccgatttacggacaaccgtcgtgtggtaccaaccgtggttcgtcttacagacggacacagtcagtgtcacggccctcgtttgcatcctgaccatgctatccacatttgtactcacgctgctgggggtcttcggggtgctgcggccactgcaggccctcggggctgcaatgcgagcggtggctggcacgctgaaggaaggggatggcagcaaggagacagttctggagccccgcaaacctaacgtgttccgggaggtggacgacattggggccgatttcgagaccatcgtggtggacttcctcggcttctccaacactaacgctcgagacaacaagtacgccccgaaggacccaaatgcgcctttcgccgtcgtcttcaccgacatccagtccagcacggcactgtggggcaagaaccccgccgagatgtctcgctgtgtccaggcccaccacgaactcattcgggagctcatcaagcagcaccggctgtacgaggtgaagacggtgggggactcattcatggtcaccacgacctccgcccaggccgcccttcgcttcgcgctggacgtccagaccacgcttttcgagcatgactgggactgggatggggcggacgagctctaccagcacacaggcgtcgtgttcaccaaggtgtgcgacgggccgttccggtccgagagcgagtacggagagctgtggaatggccttcgcgtccgcatcgggatccatttcgggatgggagacgtactgtacgatgaggtgtccaagggctacgactactacggcaacgtggtcaacgccgccgcccgaatcgaggccctcgcccacggcgggcaggtcgtcgtcaccgaggccctgctggcggccctgccggcaaaactggactccagcgtcggcctcaccaggttgctcggcaagttccccctgcgcggggtggcggagcccccggcactggtggaggtgacgcccaccgctctgcggggccgtacgtatccgctgctgcgcaccgaactcaccaccaccgagtcgcccgtagtggggcaggaaatccagagcgtcagcagcgacacccccaaaagcaccgaccgtcgctccagcggcccgacgaccgccagccatgcctccgaccggtgccccctccagcagctggcggaggacttcgcccgcacccacaccctggtccggagcagcgtgctcccggcggaggttgtggcccagcagttgctgacactgtacagcatcgtggaggaccttctgatgcccctcgcccccgcgcagtgcactgcggtgctgaaggcgcttgccaagggctggggcttcactggccccatgccaaagaccgatgccgcccaccgtatgcggctcgtgcatcggatgtcggagaccacccaggtgtttacacggctcggcaacgtgacaaggtcccacactccaatgcacgcaggatgcaccccgagccactcaggaggagggttcattcatgtagaacttgtctaactgcaactccattgcctagcccgcacagtgtattcatatgtggctttgatggcatccactgaccatcagaaccaacccttcatcagcctgtgccaaatagcgttttctcaatcccattccttcaatcaactgatttcacattccctcgcgcaccttgctcactccctctctcgatcggtttcccctcatgttgatggttgcagcttctgtgacatatccctccttcaccgattgttgctccgtcatttgtgcttccatatcgaactctgactccagaccctttgccatgttgctgtcttgcttttcctatgcttccaccaaagctgatgttgttccacctcatcttttggcagtccaggctgagtttgatgccccctgggatttttcagtttgggcagacaagtcagtgccacgtgtccattgttaatactcaaagggcctgccaattatacaatctccaaaccacagtcagttcaagcgcgggctggtcctgtttgaccggttgaccgaagccagctggccaactaccctcgcgactagagcggactggacgtcgcttctttgccccacatcccaaccccctgttgcgacccatgctgcggccctggaccctcggcgacctatgcgggccaggatgccagccagcgtcgccccgaccggagcaggattggacgtcgcttctcgcccaactcaccccccccccaaccctgtcgtaaccttgtggcagccttcccgtatccccaatattacccttgctggagcacgattggacagaattccctcgctcattttgcccccactccctctctctatcgagaccactacatccccctcacgttgacggatgcagattctgtgacgtatccccctcctccccccttggccctgcccaccccagtatagaacgcactcacctgttggtgcagttccgcagctgcactgacatccactccacattattatgcattctgatttcttttccatcgaacgttttagcgttaatccagagttttgtgtggattgctgctccatcattgggcttccatatccaactatctcctcctgcacccttgctatatcgctgcctttcttttcttgtgcatccacatcatttaccaggcacagccacgtgcccatcgttccagattgcgaaagttttgctcttccactgcttgccatttatacagtctccaaaccgcagtcagttcaagtgcgggctggtcctgtttgaccgttggcagtcaccgaggccagctgtccaactaccctcgcgactagagcagactggacgtcgcttctttgcctcacattccaacccccctgccgcgacccatgctgcggccctggaccctcggcgacctatgtgggccaggatgccagccagcgtcgccccgattcagctaactccccttcacgcctggagcaggattggacgtcccttcctcgcccagctcacccccccccccaaaacctgtcacgagccatgtggcgactttcaccctcccctaactgcctccacaacgctggcaggcaaaggggaagggcaaagtagccgcctgtggatccggagccagccagtatggccacccaaataagtttttgtagtggtgcgtagcaggtgcaccgacaaacgtcgcaaacgctgtgtttgagcccgtgacccttccgcttttagcttgcactgttgcaaaaaaaaaaaaaaaaaaaa

SEQ ID NO: 55 - DNA, 1425 bp, unidentified
  misc_feature 1..1425: note = Naegleria australiensis
  misc_feature 1..1425: note = Photoactivated adenylyl cyclase
  source 1..1425: mol_type = genomic DNA
atggattcaatgcaaagtaatctgcctcaccgagagatggcgagaaataattttggtatcaatacagatggtattcttacaaagaagactactactaactacctcctgaatggtggaggtgaacaagatagagcaatttctgttccgactagtgttgttcaacacaatgaaatcaatccttctgaagctgcaaaaacagtaacggaattatcattcagtggtaataaaagtccttctaccttgacacaagctcgtatgatgaaaagatgttcaaaacaacaattactaagcaagcttccgaagaagcctttcctaagaattcaatatgcttctcattgttgttctgggaaaatatcagagtcggttaagaatgaaattctcagtgttgctcaaagagaaaacgagattagaggtattactggagttttgatttgcgtcaacgatatgttctttcaaatcattgagggtcatacacaacatgtcgaaactctctatcacaacattttaaaggataaaagacattatgatgtggtgtgtgttgacaagatgtactttgattcagaaagtgagagatatttccctgattggtgtatgcaatcacttgacatggagagtttatttgatcactgtcttgatccatcactctccactgctgttggtgctgcttttagagctttcaaaacattattagtatctctattgaatactcagcaagtgtttagaaactatgctcaacctgtcatctctaaatatttaagtgaaggagaaaatccaatgattaaggaatccatcgagactgataaagttatattattcattgatttggtcaattataccagtataaccgaaaagttgtcaaaaacaagaaagccatcacaagtggtcgaaattttaaacaaattctttggttgtgttatggaaatagtggaagactattgtggcgagataactaaattaattggagactgtgtaatggtatattttgatggtagtgaatgccaagaagcagttaattgctcaattgaaatattagagagattggagattttaaggcaaacatcagaagatccttttgtgaaaatgatttactgtaccattggaataagcagaggttctgttatctttggaaacgttggaagtgttggtagaaagttggattttacattaattggagattcagttaatattgcatcaagacttcaatcatttgcctccatttcaccttatttcctaatatttgatgagagtgtaaaagggagattggataaagactcatgtgatataatagaagtgggcccaatagaactcaaaggcaaggaaaaaactgttaatacttatactgtcgaacacgaacttaacagaaaacctccaaagaaggaaatggaccgaataatgtctctagcctcattaagtgaatattgttga

SEQ ID NO: 56 - DNA, 1155 bp, unidentified
  misc_feature 1..1155: note = Naegleria australiensis
  misc_feature 1..1155: note = Photoactivated adenylyl cyclase
  source 1..1155: mol_type = genomic DNA
atgtcatcacaaactccaccctacataagaatgcaatatgtttccctgtccaccgagggtaacatttctgaatcaacaaagggaaagatactggcaacggcagcaagggaaaatccacgttataatattacaggtgtattgatatgcgtgagggatatgtttttccaaattattgaaggcaggattgaggatatagaccgactctatgtaaatattaagaaagacaaaagacacaacaatatcgcatgcatagacaagacatactttcatagagaggacgagagaatgttcccctcgtgggatatgaaatccatagaagtagatagcttgtttccaacaaagaaagaggttgtgatttgtggtgaatattgtaaccctctggatccatcacatcaaaaggtcattgagacggcattctctgcttttactacaatgatgggaagcatgtttagagcacaacaagtttttaagaattatgcacagccaagtgttagtagatttgttagggatggattgaatccgcttgctattgaatctgtagaaactgaaaaggttgtgctatttattgatttggtcaattatacaaccctgaccgaaattatctctcaatcaagaaaaacatcagaggttgtccgaattttgaatgttttcttttcgtatgtgatggaatgtgttgagaattttggtggtgatatcactaaactaataggcgattgcgtaatgtgccactttgatggaaatgattgtcaaaaagcagttgattgttccctgaaaatactggaatccttagataaccttagaaaagaaaccaatgacccatttgaaaagctcatcttttgtacaataggtatttctaaaggtaatgtaatcctgggcaatgttggaacagttggtagaaagctagactatacagtgattggtgattcggtaaatgtttctgcgagattacaatcgtttgcctcctcaacagagtactatctcatttttgatggaaatgtaaagaatggtttggatcgagttgactctgtggtatcatgtggctcaatagaactgaaaggaaaaaagaatgctgtaaatgcgtacacaatagaaaatgtatataatagaagagtattggcagaggatctgaaagccatgtactgcactctgaattaa

SEQ ID NO: 57 - DNA, 1158 bp, unidentified
  misc_feature 1..1158: note = Naegleria australiensis
  misc_feature 1..1158: note = Photoactivated adenylyl cyclase
  source 1..1158: mol_type = genomic DNA
atgtcatcatcacaaactcctccctacataagaatgcaatatgtttccctgtccaccgagggtatcatttctgaatcaacaaaggcaaagatactggcaacagcagcaagggaaaatccacgttataatattacaggtgtattgatatgtgtgagggatatgtttttccaaattattgaaggcaggattgaggatatagaccgactttatgtaaatattaagaaagacaataggcacaacaatatcgcatgcatagacaagacatattttcatagagaggacgagagaatgtttccctcgtgggatatgaagtccatagaagtagacagcttgtttccaacaaagaaagaggttgtgatttgtggtgaatattgtaaccctctggacccatcacatcaaaaggtcattgagacagcattctctgcttttactacactgatgggaagcatgtttagagcacaacaagtttttaagaattatgctcagccaagtgttagtagatttgttagggacggattgaatccaattgctattgaatctgtagaaactgaaaaggttgtgctatttattgatttggtcaattatacaaccctgaccgaaattatctctcaatcaagaaaaacatcagaggttgtccgaattttgaacgttttcttttcgtgtgtgatggaatgtgtcgaggattttggtggtgatatcactaaactgataggtgattgtgtaatgtgccactttgatggaaatgattgtcaaacagcagttgattgttccctgaaaatactggaatccttagataaccttagaaaagaaaccaatgacccatttgaaaagctcatcttttgtacaataggtatttctaaagggaatgtaatcctgggcaatgttggaacagttggtagaaagctagactatacagtgattggtgattcggtaaatgtttctgcgagattacaatcgtttgcctcctcaacagagtactatctcatttttgatgaaaatgtaaagaatggtttggatcgagttgactctgtggtatcatgtggctcaatagaactgaaaggaaaaaagaatgctgtaaatgcgtacacaatagaaaatgtatataatagaagagtattggcagaggatctgaaagcaatgtactgcactctgaattaa

SEQ ID NO: 58 - DNA, 2745 bp, unidentified
  misc_feature 1..2745: note = Euglena quartana
  misc_feature 1..2745: note = Putative beta subunit of photoactivated adenylyl cyclase, 4.6.1.1
  source 1..2745: mol_type = genomic DNA
ggaagatgcacgtcctcatttggaaaaaaggccagcagatcaagacattccacacgcttgatgaggcggctcagttcaaagccgcttcaaatattgatgacgctcaaatatttaatgtgtctgtgacgcccgccatcagcgcttcgggcggcagcaatgaagccaccaacctacggcggctgatgtacctttccaagtcgacaaatcctgaggagtgcactccgcagtttcttgccgagctcggacggatatcgagcattcgcaacaaggagatcggagtcagcggattcctgatgtactccgctcccttctttttccaggttattgaaggcaccgacgaagacctcgatttccttttctcgaaaatttccatggacgaccggcatgacaggtgcattgtgctggccaatgggccgtgcacgggaagaatgtatggtgactggcacatgaaggactctcacatcgacagcatcaccacacaccctgcgatgaaaaccatcctttaccagatcgcccgatctttctccagtatgtggagctaccttcccaagagcaccggcaacatgttgctgctcggcaaggatcctgccgctcaaccgcccgtacccatgagcgtcgtggttactttcctttatttggttgagttcggcaccatcctgtccaagcccaacctcacggagcaagcggctgaagtcttgagcaccttcgtcgatgtttgtgtgaagaatgtggagggcagtggtggcaacattgccaagttcattactggagtctgtatggcgtattggccaatcaacagggcggaggacgctattcaggccatccagcagatcagcgacgatttggctcagcttcgcagccaacagccgccgggatcagcgatttcgttgatgtattctcaggctggcgtgcactacggccgagctttgctgtgcaacgccgggacgcgtaagtccgatttcacgctgctcggtgattgcattaacaccacatcccgcatcgccacgctggcgaagaagctgaagacaccactgctgttctcctttgaagtgcgctgtctgctgggggacgagatgcgcgaggagatcgagagcgctggcatgcaccaggtaaagggacgggacaaacccgtcgttgtgtaccaatttcccggagccacgctggatgtggaggttgtaaagcagaagatcgagcagttcaaccctggtcgattccggtgccagatgcccgtcgtggactacgacactttgcttccttctcagcgcccacccatttttgatgagacacagagggacgtcatcaagccgcgagtgcctgggatgcagaggcgggacagcctcgccgaccgtttggcgatgatcgccaaactggctggcacgactggtggagcagcgggggctggtgatagtactctggtcactctgacgtacatctcccagaccgtacgaccaatgtcacggttggacctcgcggccatcatgcgtgtggccacccgccgcaacgcccagcagggcatcacaggcactttgctgcacgtcagcggattgtttgtgcagacgctggagggcccgaggagtgctgttttgggcctctatatgcgcattcggcaagacccccgccacaaggatgtggtcactgtccacttggctccgatttcggaacgcgtgtacccgccagactggacactgaccactgccacggaacagatgctgtcgacctttccccctctccaagacgtgctgtcccaattggccaagtccttcacgtcgttagagacctacgtgccgtcgaccgtggtccgctacctgaccgccggcaacaacccaaggaatctagttcccgtgtcgtgcggcgtcgtgatgctggccacagatatctgctccttcaccagcctgaccgagaagtcttcgctgacggaggtttggctgatttgcaacacattcatcgacgcctgcaccaacgccatctgccaagccggtggagaggtcatcaagctgattggtgactgcgtgaccgcttattttcctgggactgccgctgacaatgccgtgtcggcggcgcaggagctgatcaccttctgtcgtgagctgcgagaggccttcgttgacgtgctggacgtccgcggctgcatctattgcggcgtcgggctggatttcggccaggtggtgatggcacagtgtggcacccttggtctcagcgagtacgccgtggcgggtgccgtcagcgcgcgtgtgatggaggtggaggccctgacgcgcgaggtcggtcgcgccattgttctaaccgagcccctggccgaccggctgtgccctcagatgcaggagatgggcattgttcccagtccacaagccatcgatgggttgccatgctatggagtggcgggcgacgagttcgtgctcgatgttgatactatcaagcgcagcattaagaacttccacgctgcacgctccggtgaaaagctcaatgtgtccgggcttgcgaatggcgatctggaggagagcatgacgggctcgcctcttcgagccacaatcaacgacttccgcgtgtccccgggccgtgtgcgccgcggcgactctgtgcgtcggtcaccgtccatcgcgggccggtatggcgtggcaacccgctgagaaatgggtctaggcttgggcaacctcgtggacagccacattttgccacctcgtttgggcagcctcgtggatgccgcagcagccctcacgcgggcgatgtcgtcatgtacgatggacg

SEQ ID NO: 59 - DNA, 1065 bp, unidentified
  misc_feature 1..1065: note = Pseudanabaena sp.
  misc_feature 1..1065: note = Beta subunit of photoactivated adenylyl cyclase ABRG53_0929, Pseudanabaena sp. ABRG5-3
  source 1..1065: mol_type = genomic DNA
atgaaaaggctaacttatattagtagcttctcgcgatcgcttagcaaaagagagattgaagccattggacatatttctcagactaataattctagggaaggaattacgggggttttattaagctgcaacggcattttctttcaaattttagaaggcgatgaagaccgcattgatcggctgtacgagaagattttacgagatgatcgtcatagcaatattttatgcttgaaatctgaattaaatgttttagcccgtaagtttcctgattggtcaatggaagtcatcgttcttgatgaaaataatgacattctcttgcaaccaattaaaattcttttgcaaactctaggcgaatctcaaaccattctcgctaactatacccagtcaggagttttgcaagcaattgagaatgggattaatcccctcaccctcaaaccactctatagcgataaaatcatcttatttgctgatattatctccttttctggcattactgaaaaattgcaagttgctgaagtagtagatttgctcaaccagttctttaccatttgcacgcgagcgattactgcaagaggtggtgaagttacgaaatttattggcgattgcgtaatggcatattttgatggcgatcgcgcagatgattcgctacaggcaagcctagatattttggcggaactagagcttttacgcaatcaagcccctgccaatagtccattacgagttctctacattggcattggtctatctaaaggaattgtgttagagggaaatatcggttcgcaaacaaagaaggaatatacagtgattggtgatgccgtcaatgttgccgccagaatcgaagctctcaccagacagttaaatcgatttctcgtattttctgcttctgtacgtgcaggattaagcgatcgctggaagcctgttaggcttggcgattttcataccaagggaagagaaagtatcgtcgaagtctattctgttgacgatgtattaaccttccaaaaatcagattttcataaattaaaacaagagatcaagcaacacctagagctaattagctgccaagcataa

SEQ ID NO: 60 - DNA, 1113 bp, unidentified
  misc_feature 1..1113: note = Pseudanabaena sp.
  misc_feature 1..1113: note = Beta subunit of photoactivated adenylyl cyclase ABRG53_0928, Pseudanabaena sp. ABRG5-3
  source 1..1113: mol_type = genomic DNA
atgaaaaggctaacctatattagtaacttctcgcgatcattgaccagaaaggaaatagaaaatattgagattatttcacaaaaaaataattttagggaagggattacaggaatattactgagttgcagtggtattttctttcaaattttggaaggggatgatcatcgagtagattcattgtacgaaaaaattttaaaagatgatcgtcaccatcaagttttatgtcttaagtcagaagcaaacattactgagagattctttcctgactggtcgatgaaagtaattacaatagatgaaaacagcgagattttattaaaaccaattaaagtattgctacaaagcttgggagagtcaaaaatggctttggcaaactatacgcattcaggggttttacaagccatcgaaaacggaatcaatcctcttaccctgaccccacgctataccgaaaaaattattttctttgcggatattatgggctttgccagtatgactgagaaactccaagttaccgaagttgcagagttactcaatcaattttttacaatttgcacttcagaaatcgcagctaaaggcggagaagtcacgaagtttatcggcgattgcgtgatggcatatttcgatggtgatcgtgcaaatgaagctttacaagcaagtctagacattctttcagaattagagattttacgagatcgatcgcctgttaatagcccgttgcgagttctttatgtggggattgggctatctaagggaacagttttagaaggaaatatcggttctcaaaccaaaaaagagtatgcaatcatcggcgatgcagttaatgttgccgcaagaattgaatcgctaacgcgaaagttaaatagatttttagtattttcggcagcagtacgagagggactcagtgatcgctggaaattgttaaaaatgggggatttctatgctaaaggaagagaagcgatcgtggaagtatattcaattgataatcctttggcatctcaaaaatcagatttccaccatctaaaacaagagattaacgagcagcttgaactcataggaaagcaaactctagtgtcttacaaacaacgcaaaacactatttaagctttacgatcgctaa

SEQ ID NO: 61 - DNA, 3547 bp, unidentified
  misc_feature 1..3547: note = Euglena gracilis
  misc_feature 1..3547: note = Putative membrane-bound adenylyl cyclase, 4.6.1.1
  source 1..3547: mol_type = genomic DNA
actgcaaaaaaatgcgaatttctgtatatgtgctgtacataggcgcttgccttctggcaattggagctgccattttgagtggggttatcctacttaacacgttcgcgggacagcagcaacagagcgaggatgcgcagtacaccaccggccggggccaggtgcaggccatccagggcgtcatcgccaaccaaatgtcccgcatggtggcctccgtggacgaccgtagtcggcagctcctcatgaccctgaaggagctgcccgacgacgcccttgagccccagaacgccgtggcctcgctgaaccacagcatcttcacgacctgggttccttctatcatggcaaacgatactgtgctcgggaacggcttgaccatcatgtacgtgaacgagagcaatgggcagctgtacgaccgcacgttttccgtgtaccgggagctgctgaagagcgaccagtggaattacttgtacggctacacctttcctgatgagttgtcccatgttttcttcatcaactggctggattattcaactccgcgacttgggaaggacgtgtaccaagtcaatttcacaacggtcaaggccccagtctatcagaaggacaacttctttaaccacgtgatgccatggggttcgactgacggcaacactttctggtatttcacgtacatgaggtcgttttacacccatggcgtgtggctcaatatgcagtgctgggatgtcacggtcaactggctgtcgctgatgcagagcggcttgacggccggggccagcgtggcctcctacgacagccaggggtatgtggtcaccgccaccaacaaggacgagctgcggcggctggcccagtgcattgagactggcgatgtgggctcgtcctgtatctctgaaggaggctgcaactgcctcaccactttcgcaaagtttcatccgattgcggagctccgagacttgtatctcgcattgcacacccccttgtgggacgacctggacgcaccaccaattccgctccagcagcaggacattcagctctctggtcagcggtacatggccatcgtggccacactgttcgctaaaagcggcttccgggtcaccgtcgtgtggtaccagccgtgggtcctcaccgacacctccgccgtgggcgtgaccgctctgatatgcgtcctgaccatgctgtcaacgttcctactcacgctgcttggcatcttcggcgtgctgcggccactgatggccctgggcagtgccatgcgagtggtcgcccacacgctgcggaagggcgatgccgagagggaggcggtgctgcagcctcggaggccgaacattttccgcgaggtggacaccatcgggcaggattttgagaccatcgtggtggacttcctcggcttctccagtacaaacgcgcgggacaacaagcacgcccccaaggatcccaacaagcctttcgtcgtcatcttcaccgacatccagtcgagtacggggctgtggggccgggaccccgcccagatgtcccgctgcgtgcaggtccaccacgagataatccggacgctcatcaaggatcaccacctctacgaagtgaaaactgcgggtgactcgttcatgatcaccaccgccagcccgcccgacgccctgcggttcgccctggacctgcagcaggcgctgtacgactacgactgggactgggacgagtgcgacgacttctaccgcgagaccaccctggcgctgtggccggcggctctgggcgccaagtacgccgacatgtggaacgggcttcgggtccgcgttgggatccattttgggttgggcgatgttgtgtacgacgaggtctcgaagggctacgactattacggcaacgtggtcaacaccgcggcccgcattgaggccctcgcccacggcgggcagatcgtggtgacggagtccctgctggccgccctgcccaccccgctggaccccagggttagccgcacggtgtcgctcggcactgtccccttgcgcggcgtgacggcgccgccggcattggtggaggtccagatggcctccctggcaacccgcacttttccgccgctgcgggtggagcacgctaatgaagcggcgaagcaggagtgcattaatgtgcccgacctcctcagtgacaaggctggttctgatggcctgctgaccggtcggctggggtttcagaccccaagtctgcaatccgattccctggtgcaggtggcggaggacttggccaagacccacgcgatggtgcggcggggctatttctcggcggaggcggtgacccaacacctgttggtcctctaccgcattgtggaggacttgttgacgcccctcgccccgtcacagttcgccacggtgacgaaggcgctggcccggggctggggcgtggcgccgcccaagtcgaaggcggagttcggcccggtcgggctgcggctgatgcagcgcatgtcggagacaaccaccgtcctgaagaacttagcggcgccgcaccacagagctcagtccgccgggccggacgcgcggctgctggacatggaagccacggcgtagctgccagggccagctttgcaaggttcctggtcgcccagtcgagtcgtgtctagtcgtatcgtctcctacccctacgcctgcctttcgggaaccgtccccagtcccccgccccccaactccccacacggggtttctgcacaaccactgtccagctgttctttccttgcgtgtaccgcgcaccccctgcaaccctagtgcttctgaacctgcccttttaaaattcggcccgagcccgagtcgtcgtcaggaaccgtcattgcatgtgtatcagtactgtgttctcttaaccctaagccgcggggcaagggagagagttctcctccctctcctcgcaacttgacacactccgtctcacccagtcacacaggtatacccacaacccccgaactgatagagcttaggcccactggcttagcatggtcactgccttctcatgccttcgggtcttttcatgtcatttcgcttccgtggccgcacccattgagcggccctgaaagtttgaggcacactgccgcatctccagcccgcaatcctcagcctgacctccctccggcccattgcaagcactgctgcctcctccttgccaggtgttgtgctccccatgcaacggactgcccatcagctttggtgtccatggccgcgctaccactggtgcaatgctgccagtgctcatcctgccagcatggcactctcccgtcgctggctgccactcctcggttgccccgacctgagaaacacctattggactcattgttcttcgctcagtctcgtgacaccggctattgtgtcccatattgattttcatgggccagccgcacgcctcatctcgcataacgcgcacgcatccactgacccagttgtcccttctccgcataagtcagcctcctcactccagttctgcctgcctttatatcgatgcgtggttgtgttccttttctttttctttgcaaaattttttccacacttccgatttacccgcccttgcaaccctctcaaaaaaaaaaaaaaaa

SEQ ID NO: 62 - DNA, 1053 bp, unidentified
  misc_feature 1..1053: note = Beggiatoa sp.
  misc_feature 1..1053: note = Beta subunit of photoactivated adenylyl cyclase BGP_1043
  source 1..1053: mol_type = genomic DNA
atgatgaaaagattagtttatattagcaaaatttcaggtcatttatcccttgaagaaatccaacgaattggtaaagtatccattaaaaataatcaacgggataacattaccggcgtattactgtatttacaaggactatttttccaaatattggagggagaaaatgagaaagtcgacaaattgtataaaaaaattttggttgatgatcgacataccaatattctatgtttaaaaacggaatatgatatcactgataggatgtttcctaattgggcgatgaaaacgattaatctgaatgaaaatagcgaattgatgattcaaccgattaaaagtttattacaaaccatcacacaatcgcatcgtgtgttagaaaaatatatgccggctcgtgttatttacctcattaatcaaggtattaatccactcaccgtcgaaccacaattggttgaaaaaatcatttttttcagtgatattctagctttttccaccttgacagaaaaactcccggtgaacgaagttgtcatcctcgttaatcgttattttagtatttgcacgcgaattatttcagcttatggtggcgaagtgaccaaatttattggcgattgtgtcatggcatcatttaccaaagaacagggagatgctgctatccgtaccagtttagacattatcagcgaattgaaacaattgcgtcatcatgttgaagcgactaatccattacatttattgtataccggcattgggctatcttacgggcatgtgattgaaggcaatatgggttcttcattaaaaatggatcataccttgttaggtgatgcggtgaatgtggctgcacgtttagaggctttaacgcgacaattgccgtatgctttggcattcacagccggtgttaaaaaatgttgtcaagcacaatggacttttatcaatctaggtgcccatcaagtcaaaggcaaacaagaagcgattgaagtgtataccgttaatgaagcacaaaaatattacgatactctgcaaatcacacaactgattaggcaaactcttgaaaatgataaatga

SEQ ID NO: 63 - DNA, 10735 bp, synthetic construct
  misc_feature 1..10735: note = Synthetic: Plasmid bPAC_S27A_T2A_mRFP_PGK_Hygro
  source 1..10735: mol_type = other DNA
agcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttggtctctgcaaaagtctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggatagctagagccagacatgataagatacattgatgagtttggacaaaccacaactagaatgcagtgaaaaaaatgctttatttgtgaaatttgtgatgctattgctttatttgtaaccattataagctgcaataaacaagttcctctcactctctgatattcatttctttgcaagttataaatactgaataataagatgacatgaactactactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttatcttcgttgggagtgaattagcccttccagtccccccttttcttttaaaaagtggctaagatctacagctgccttgtaagtcattggtcttaaaggtaccaggcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgtcgacttatccagcgcctgtgctatgtctgccctcagctctctcatattgttccacgatggtgtagtccccattatggctgataatgtcgagtttaatatcagtcatgtaggcgccaggcagctgcacttgtttcttggccttgtaggttgttttgacctcggcatcgtagtgtcctccgtctttgagtttcagtctcattttaatttcgcctttcagagcgccatcctcaggatacattctctcggtggaggcttcccatcccattgtttttttttgcatgacagggccatcggaggggaagttggttcctctgagtttcaccttataaataaactctccgtcctggagggtgctatcttgtgtgactgtcaccacgcctccgtcctcgaagttcatgaatctctcccacttgaagccttcagggaaggagagcttcagatagtcagggatgtcggcagggtgtttaacataggctttgcttccgtactggaactgagggctcagaatatcccaggagaaggggaggggtcctccttttgtgaccttgagcttagcggtttgtgtgccctcgtaaggccggccttctccttctccctcaatctcgaactcgtgtccgttgacgcttccctccatcttgaccttaaatctcataaactctttgatgacatcctcggagctggccattccggaagggccgggattctcctccacgtcaccgcatgttagaagacttcctctgccctcagcggccaacaggtcctcctccgagatcagcttttgctcgtacgtccgcggcttgtcgttttccagggtctgccggatcagctgggtgatctgcagggtgtcgtagtacttctgggcctcattcacggtgtagacctcgatggcttcctgcttgcccttgacctggtgggctcccaggttgatgaaggtccactgggcctggcagcacttcttcacgccggctgtaaaggccagggcgtagggcagctgtcttgtcagggcttccagcctggcggccacgttcacggcgtcccccagcagggtgtggtccatcttcaggctgctgcccatgttgccctcgatcacgtggccgtagctcaggccgatgccggtgtacagcaggtgcagggggttggtggcctccacgtggtgccgcagctgcttcagctcggagatgatgtccaggctggtccggatggcggcgtcgccctgctctttggtgaagctggccatcacgcagtcgccgatgaacttggtcacttcgccgccgtaggcgctgatgatccgggtgcagatgctgaagtaccggttgaccaggatgaccacctcgttcacgggcagcttctcggtcagggtgctgaaggccaggatgtcgctgaagaagatgatcttctcgaccagctggggctccacggtcagggggttgatgccctggttgatcaggtagatcactctggcgggcatgtacttttccagcacccggtggctctgggtgatggtctgcagcaggctcttgatgggctggatcatcagctcgctgttctcgttcaggttgatggttttcatggcccagttggggaacatccggtcggtgatgtcgtactcggttttcaggcacaggatgttggtgtgccggtcgtccaccaggattttcttgtacagcttgtccaccttctcgttctcgccttccaggatctggaagaacaggccctgcaggtacagcagcacgccggtgatgttgtcccgctggttgttcttgatggccaccttgccgatccgctggatctcttccaggctcaggtggccgctgatcttgctgatgtacaccagccgcttcatcatggccgcggatccgatttaaattcgaattcgctagctctagatcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaaggtcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccacaacttctcggggactgtgggcgatgtgcgctctgcccactgacgggcaccggagccgctagcttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctttaattaaatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaagtttaaactcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagataaaattttgaattttgtaatttgtttttgtaattctttagtttgtatgtctgttgctattatgtctactattctttcccctgcactgtaccccccaatccccccttttcttttaaaagttaaccgataccgtcgagatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggtttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagtggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccgtttatgtatcgagctaggcacttaaatacaatatctctgcaatgcggcaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacacgcgt

SEQ ID NO: 64 - protein, 350 aa, synthetic construct; identical to SEQ ID NO: 31
  REGION 1..350: note = Synthetic: Bluf Domain (aa)

SEQ ID NO: 65 - DNA, 1050 bp, synthetic construct
  misc_feature 1..1050: note = Synthetic: Bluf Domain (nt)
  source 1..1050: mol_type = other DNA
atgatgaagcggctggtgtacatcagcaagatcagcggccacctgagcctggaagagatccagcggatcggcaaggtgtccatcaagaacaaccagcgggacaacatcaccggcgtgctgctgtacctgcagggcctgttcttccagatcctggaaggcgagaacgagaaggtggacaagctgtacaagaaaatcctggtggacgaccggcacaccaacatcctgtgcctgaaaaccgagtacgacatcaccgaccggatgttccccaactgggccatgaaaaccatcaacctgaacgagaacagcgagctgatgatccagcccatcaagagcctgctgcagaccatcacccagagccaccgggtgctggaaaagtacatgcccgccagagtgatctacctgatcaaccagggcatcaaccccctgaccgtggagccccagctggtcgagaagatcatcttcttcagcgacatcctggccttcagcaccctgaccgagaagctgcccgtgaacgaggtggtcatcctggtcaaccggtacttcagcatctgcacccggatcatcagcgcctacggcggcgaagtgaccaagttcatcggcgactgcgtgatggccagcttcaccaaagagcagggcgacgccgccatccggaccagcctggacatcatctccgagctgaagcagctgcggcaccacgtggaggccaccaaccccctgcacctgctgtacaccggcatcggcctgagctacggccacgtgatcgagggcaacatgggcagcagcctgaagatggaccacaccctgctgggggacgccgtgaacgtggccgccaggctggaagccctgacaagacagctgccctacgccctggcctttacagccggcgtgaagaagtgctgccaggcccagtggaccttcatcaacctgggagcccaccaggtcaagggcaagcaggaagccatcgaggtctacaccgtgaatgaggcccagaagtactacgacaccctgcagatcacccagctgatccggcagaccctggaaaacgacaag

SEQ ID NO: 66 - DNA, 27 bp, synthetic construct
  misc_feature 1..27: note = Synthetic: Ucp1 primer
  source 1..27: mol_type = other DNA
cgactcagtccaagagtacttctcttc

SEQ ID NO: 67 - DNA, 20 bp, synthetic construct
  misc_feature 1..20: note = Synthetic: Ucp1 primer
  source 1..20: mol_type = other DNA
gccggctgagatcttgtttc

SEQ ID NO: 68 - DNA, 22 bp, synthetic construct
  misc_feature 1..22: note = Synthetic: Nr4a3 primer
  source 1..22: mol_type = other DNA
acgccgaaaccgatgtcagtac

SEQ ID NO: 69 - DNA, 23 bp, synthetic construct
  misc_feature 1..23: note = Synthetic: Nr4a3 primer
  source 1..23: mol_type = other DNA
ctcctgttgtagtgggctctttg

SEQ ID NO: 70 - DNA, 20 bp, synthetic construct
  misc_feature 1..20: note = Synthetic: bPAC primer
  source 1..20: mol_type = other DNA
ccagcggatcggcaaggtgg

SEQ ID NO: 71 - DNA, 20 bp, synthetic construct
  misc_feature 1..20: note = Synthetic: bPAC primer
  source 1..20: mol_type = other DNA
ggttttcaggcacaggatgt

SEQ ID NO: 72 - DNA, 23 bp, synthetic construct
  misc_feature 1..23: note = Synthetic: Pgc1a primer
  source 1..23: mol_type = other DNA
gaatcaagccactacagacaccg

SEQ ID NO: 73 - DNA, 22 bp, synthetic construct
  misc_feature 1..22: note = Synthetic: Pgc1a primer
  source 1..22: mol_type = other DNA
catccctcttgagcctttcgtg

SEQ ID NO: 74 - DNA, 22 bp, synthetic construct
  misc_feature 1..22: note = Synthetic: Tfam primer
  source 1..22: mol_type = other DNA
gaggcaaaggatgattcggctc

SEQ ID NO: 75 - DNA, 22 bp, synthetic construct
  misc_feature 1..22: note = Synthetic: Tfam primer
  source 1..22: mol_type = other DNA
gaggcaaaggatgattcggctc

SEQ ID NO: 76 - DNA, 22 bp, synthetic construct
  misc_feature 1..22: note = Synthetic: Atp5g1 primer
  source 1..22: mol_type = other DNA
catcgacacagcagccaagttc

SEQ ID NO: 77 - DNA, 23 bp, synthetic construct
  misc_feature 1..23: note = Synthetic: Atp5g1 primer
  source 1..23: mol_type = other DNA
ccagaatggcataggagaagagc

SEQ ID NO: 78 - DNA, 22 bp, synthetic construct
  misc_feature 1..22: note = Synthetic: Atp5g3 primer
  source 1..22: mol_type = other DNA
gtaggagttgctggttctggtg

SEQ ID NO: 79 - DNA, 23 bp, synthetic construct
  misc_feature 1..23: note = Synthetic: Atp5g3 primer
  source 1..23: mol_type = other DNA
gcttcagacaaggcaaatcccag

SEQ ID NO: 80 - DNA, 23 bp, synthetic construct
  misc_feature 1..23: note = Synthetic: Ndufs4 primer
  source 1..23: mol_type = other DNA
gctcgcaataacatgcagtctgg

SEQ ID NO: 81 - DNA, 22 bp, synthetic construct
  misc_feature 1..22: note = Synthetic: Ndufs4 primer
  source 1..22: mol_type = other DNA
ggtcagaaccatgttggagagg

SEQ ID NO: 82 - DNA, 23 bp, synthetic construct
  misc_feature 1..23: note = Synthetic: Ndufb8 primer
  source 1..23: mol_type = other DNA
cgccaagaagtataacatgcgag

SEQ ID NO: 83 - DNA, 22 bp, synthetic construct
  misc_feature 1..22: note = Synthetic: Ndufb8 primer
  source 1..22: mol_type = other DNA
cctctcatgctgtgatcggttg

SEQ ID NO: 84 - DNA, 22 bp, synthetic construct
  misc_feature 1..22: note = Synthetic: Sdhb primer
  source 1..22: mol_type = other DNA
tgcggacctatggtgttggatg

SEQ ID NO: 85 - DNA, 23 bp, synthetic construct
  misc_feature 1..23: note = Synthetic: Sdhb primer
  source 1..23: mol_type = other DNA
ccagagtattgcctccgttgatg

SEQ ID NO: 86 - DNA, 22 bp, synthetic construct
  misc_feature 1..22: note = Synthetic: Uqcr2 primer
  source 1..22: mol_type = other DNA
atgccaccttctaccgtccttc

SEQ ID NO: 87 - DNA, 22 bp, synthetic construct
  misc_feature 1..22: note = Synthetic: Uqcr2 primer
  source 1..22: mol_type = other DNA
gtttccactcgctgccattgac

SEQ ID NO: 88 - DNA, 22 bp, synthetic construct
  misc_feature 1..22: note = Synthetic: Cox4i2 primer
  source 1..22: mol_type = other DNA
acagtgatgggctgcgtcttct

SEQ ID NO: 89 - DNA, 22 bp, synthetic construct
  misc_feature 1..22: note = Synthetic: Cox4i2 primer
  source 1..22: mol_type = other DNA
ctgttgggctttccgttcttcc

SEQ ID NO: 90 - DNA, 23 bp, synthetic construct
  misc_feature 1..23: note = Synthetic: Bnip3 primer
  source 1..23: mol_type = other DNA
gctccaagagttctcactgtgac

SEQ ID NO: 91 - DNA, 23 bp, synthetic construct
  misc_feature 1..23: note = Synthetic: Bnip3 primer
  source 1..23: mol_type = other DNA
gtttttctcgccaaagctgtggc

SEQ ID NO: 92 - DNA, 22 bp, synthetic construct
  misc_feature 1..22: note = Synthetic: Bnip3l primer
  source 1..22: mol_type = other DNA
gcatgaggaagagtggagccat

SEQ ID NO: 93 - DNA, 23 bp, synthetic construct
  misc_feature 1..23: note = Synthetic: Bnip3l primer
  source 1..23: mol_type = other DNA
aaggtgtgctcagtcgttttcca

SEQ ID NO: 94 - DNA, 22 bp, synthetic construct
  misc_feature 1..22: note = Synthetic: Pink1 primer
  source 1..22: mol_type = other DNA
cgacaacatccttgtggagtgg

SEQ ID NO: 95 - DNA, 22 bp, synthetic construct
  misc_feature 1..22: note = Synthetic: Pink1 primer
  source 1..22: mol_type = other DNA
cattgccaccacgctctacact

SEQ ID NO: 96 - DNA, 22 bp, synthetic construct
  misc_feature 1..22: note = Synthetic: Mff primer
  source 1..22: mol_type = other DNA
ctactcgtagggcttaccagca

SEQ ID NO: 97 - DNA, 23 bp, synthetic construct
  misc_feature 1..23: note = Synthetic: Mff primer
  source 1..23: mol_type = other DNA
gctccttcaatggctgcatctag